SEQUENCE LISTING (993 sequences)

SEQ ID NO: 1 - protein, 11 aa, Artificial Sequence
  Description of Artificial Sequence Synthetic peptide
NSSCMGGMNLR

SEQ ID NO: 2 - protein, 11 aa, Artificial Sequence
  Description of Artificial Sequence Synthetic peptide
CNSSCMGGMNL

SEQ ID NO: 3 - protein, 12 aa, Artificial Sequence
  Description of Artificial Sequence Synthetic peptide
NSSCMGGMNLRP

SEQ ID NO: 4 - protein, 11 aa, Artificial Sequence
  Description of Artificial Sequence Synthetic peptide
SSCMGGMNLRP

SEQ ID NO: 5 - protein, 12 aa, Artificial Sequence
  Description of Artificial Sequence Synthetic peptide
SCMGGMNLRPIS

SEQ ID NO: 6 - protein, 11 aa, Artificial Sequence
  Description of Artificial Sequence Synthetic peptide
CMGGMNLRPIL

SEQ ID NO: 7 - protein, 11 aa, Artificial Sequence
  Description of Artificial Sequence Synthetic peptide
MGGMNLRPILT

SEQ ID NO: 8 - protein, 11 aa, Artificial Sequence
  Description of Artificial Sequence Synthetic peptide
GGMNLRPILTI

SEQ ID NO: 9 - protein, 11 aa, Artificial Sequence
  Description of Artificial Sequence Synthetic peptide
GMNLRPILTII

SEQ ID NO: 10 - protein, 11 aa, Artificial Sequence
  Description of Artificial Sequence Synthetic peptide
MNLRPILTIIT

SEQ ID NO: 11 - protein, 11 aa, Artificial Sequence
  Description of Artificial Sequence Synthetic peptide
NLRPILTIITL

SEQ ID NO: 12 - protein, 11 aa, Artificial Sequence
  Description of Artificial Sequence Synthetic peptide
LRPILTIITLE

SEQ ID NO: 13 - protein, 10 aa, Artificial Sequence
  Description of Artificial Sequence Synthetic peptide
CNSSCMGGMN

SEQ ID NO: 14 - protein, 10 aa, Artificial Sequence
  Description of Artificial Sequence Synthetic peptide
NSSCMGGMNL

SEQ ID NO: 15 - protein, 10 aa, Artificial Sequence
  Description of Artificial Sequence Synthetic peptide
SSCMGGMNLR

SEQ ID NO: 16 - protein, 10 aa, Artificial Sequence
  Description of Artificial Sequence Synthetic peptide
SCMGGMNLRP

SEQ ID NO: 17 - protein, 10 aa, Artificial Sequence
  Description of Artificial Sequence Synthetic peptide
CMGGMNLRPI

SEQ ID NO: 18 - protein, 10 aa, Artificial Sequence
  Description of Artificial Sequence Synthetic peptide
MGGMNLRPIL

SEQ ID NO: 19 - protein, 10 aa, Artificial Sequence
  Description of Artificial Sequence Synthetic peptide
GGMNLRPILT

SEQ ID NO: 20 - protein, 10 aa, Artificial Sequence
  Description of Artificial Sequence Synthetic peptide
GMNLRPILTI

SEQ ID NO: 21 - protein, 10 aa, Artificial Sequence
  Description of Artificial Sequence Synthetic peptide
MNLRPILTII

SEQ ID NO: 22 - protein, 10 aa, Artificial Sequence
  Description of Artificial Sequence Synthetic peptide
NLRPILTIIT

SEQ ID NO: 23 - protein, 10 aa, Artificial Sequence
  Description of Artificial Sequence Synthetic peptide
LRPILTIITL

SEQ ID NO: 24 - protein, 9 aa, Artificial Sequence
  Description of Artificial Sequence Synthetic peptide
SSCMGGMNL

SEQ ID NO: 25 - protein, 9 aa, Artificial Sequence
  Description of Artificial Sequence Synthetic peptide
SCMGGMNLR

SEQ ID NO: 26 - protein, 9 aa, Artificial Sequence
  Description of Artificial Sequence Synthetic peptide
CMGGMNLRP

SEQ ID NO: 27 - protein, 9 aa, Artificial Sequence
  Description of Artificial Sequence Synthetic peptide
MGGMNLRPI

SEQ ID NO: 28 - protein, 9 aa, Artificial Sequence
  Description of Artificial Sequence Synthetic peptide
GGMNLRPIL

SEQ ID NO: 29 - protein, 9 aa, Artificial Sequence
  Description of Artificial Sequence Synthetic peptide
GMNLRPILT

SEQ ID NO: 30 - protein, 9 aa, Artificial Sequence
  Description of Artificial Sequence Synthetic peptide
MNLRPILTI

SEQ ID NO: 31 - protein, 9 aa, Artificial Sequence
  Description of Artificial Sequence Synthetic peptide
NLRPILTII

SEQ ID NO: 32 - protein, 9 aa, Artificial Sequence
  Description of Artificial Sequence Synthetic peptide
LRPILTIIT

SEQ ID NO: 33 - protein, 8 aa, Artificial Sequence
  Description of Artificial Sequence Synthetic peptide
SCMGGMNL

SEQ ID NO: 34 - protein, 8 aa, Artificial Sequence
  Description of Artificial Sequence Synthetic peptide
CMGGMNLR

SEQ ID NO: 35 - protein, 8 aa, Artificial Sequence
  Description of Artificial Sequence Synthetic peptide
MGGMNLRP

SEQ ID NO: 36 - protein, 8 aa, Artificial Sequence
  Description of Artificial Sequence Synthetic peptide
GGMNLRPI

SEQ ID NO: 37 - protein, 8 aa, Artificial Sequence
  Description of Artificial Sequence Synthetic peptide
GMNLRPIL

SEQ ID NO: 38 - protein, 8 aa, Artificial Sequence
  Description of Artificial Sequence Synthetic peptide
MNLRPILT

SEQ ID NO: 39 - protein, 8 aa, Artificial Sequence
  Description of Artificial Sequence Synthetic peptide
NLRPILTI

SEQ ID NO: 40 - protein, 8 aa, Artificial Sequence
  Description of Artificial Sequence Synthetic peptide
LRPILTII

SEQ ID NO: 41 - protein, 11 aa, Artificial Sequence
  Description of Artificial Sequence Synthetic peptide
CILSGANNLFF

SEQ ID NO: 42 - protein, 13 aa, Artificial Sequence
  Description of Artificial Sequence Synthetic peptide
CILYGGATNKLIF

SEQ ID NO: 43 - protein, 10 aa, Artificial Sequence
  Description of Artificial Sequence Synthetic peptide
CILNNNDMRF

SEQ ID NO: 44 - protein, 14 aa, Artificial Sequence
  Description of Artificial Sequence Synthetic peptide
CILKTNSGNTPLVF

SEQ ID NO: 45 - protein, 13 aa, Artificial Sequence
  Description of Artificial Sequence Synthetic peptide
CATTGGQNTEAFF

SEQ ID NO: 46 - protein, 14 aa, Artificial Sequence
  Description of Artificial Sequence Synthetic peptide
CASQSGILPWEQFF

SEQ ID NO: 47 - protein, 16 aa, Artificial Sequence
  Description of Artificial Sequence Synthetic peptide
CAISEWRAGSTDTQYF

SEQ ID NO: 48 - protein, 16 aa, Artificial Sequence
  Description of Artificial Sequence Synthetic peptide
CASSEVQGASNEKLFF

SEQ ID NO: 49 - protein, 9 aa, Artificial Sequence
  Description of Artificial Sequence Synthetic peptide
EVIVPLSGW

SEQ ID NO: 50 - protein, 12 aa, Artificial Sequence
  Description of Artificial Sequence Synthetic peptide
CASSLDPYEQYF

SEQ ID NO: 51 - protein, 11 aa, Artificial Sequence
  Description of Artificial Sequence Synthetic peptide
AQAGAWKIYAV

SEQ ID NO: 52 - protein, 9 aa, Artificial Sequence
  Description of Artificial Sequence Synthetic peptide
AQAGAWKIY

SEQ ID NO: 53 - protein, 11 aa, Artificial Sequence
  Description of Artificial Sequence Synthetic peptide
FAAQAGAWKIY

SEQ ID NO: 54 - DNA, 20 bp, Artificial Sequence
  Description of Artificial Sequence Synthetic oligonucleotide
agagtctctcagctggtaca

SEQ ID NO: 55 - DNA, 20 bp, Artificial Sequence
  Description of Artificial Sequence Synthetic oligonucleotide
agaaggtggccgagaccctc

SEQ ID NO: 56 - protein, 8 aa, Artificial Sequence
  Description of Artificial Sequence Synthetic peptide
HVIENIYF

SEQ ID NO: 57 - protein, 9 aa, Artificial Sequence
  Description of Artificial Sequence Synthetic peptide
DVAAHLQPL

SEQ ID NO: 58 - protein, 8 aa, Artificial Sequence
  Description of Artificial Sequence Synthetic peptide
ETPNLDLM

SEQ ID NO: 59 - protein, 9 aa, Artificial Sequence
  Description of Artificial Sequence Synthetic peptide
SVFNTWNPM

SEQ ID NO: 60 - protein, 8 aa, Artificial Sequence
  Description of Artificial Sequence Synthetic peptide
EVQQFLRY

SEQ ID NO: 61 - protein, 9 aa, Artificial Sequence
  Description of Artificial Sequence Synthetic peptide
ETMQCSELY

SEQ ID NO: 62 - protein, 11 aa, Artificial Sequence
  Description of Artificial Sequence Synthetic peptide
ETMQCSELYHM

SEQ ID NO: 63 - protein, 8 aa, Artificial Sequence
  Description of Artificial Sequence Synthetic peptide
ETMQCSEL

SEQ ID NO: 64 - protein, 10 aa, Artificial Sequence
  Description of Artificial Sequence Synthetic peptide
ITRTVSANTV

SEQ ID NO: 65 - protein, 10 aa, Artificial Sequence
  Description of Artificial Sequence Synthetic peptide
ATKNNKVIMA

SEQ ID NO: 66 - protein, 10 aa, Artificial Sequence
  Description of Artificial Sequence Synthetic peptide
VAHFQLQMLK

SEQ ID NO: 67 - protein, 9 aa, Artificial Sequence
  Description of Artificial Sequence Synthetic peptide
EEDTFSYLI

SEQ ID NO: 68 - protein, 8 aa, Artificial Sequence
  Description of Artificial Sequence Synthetic peptide
AHFQLQML

SEQ ID NO: 69 - protein, 11 aa, Artificial Sequence
  Description of Artificial Sequence Synthetic peptide
LHAMIQAAGKL

SEQ ID NO: 70 - protein, 11 aa, Artificial Sequence
  Description of Artificial Sequence Synthetic peptide
LHEAQPWFEFL

SEQ ID NO: 71 - protein, 10 aa, Artificial Sequence
  Description of Artificial Sequence Synthetic peptide
LHEAQPWFEF

SEQ ID NO: 72 - protein, 10 aa, Artificial Sequence
  Description of Artificial Sequence Synthetic peptide
EHLSCPDNFL

SEQ ID NO: 73 - protein, 10 aa, Artificial Sequence
  Description of Artificial Sequence Synthetic peptide
NHARIDAAKV

SEQ ID NO: 74 - protein, 9 aa, Artificial Sequence
  Description of Artificial Sequence Synthetic peptide
QHQPNPFEV

SEQ ID NO: 75 - protein, 8 aa, Artificial Sequence
  Description of Artificial Sequence Synthetic peptide
TQLEKEAL

SEQ ID NO: 76 - protein, 11 aa, Artificial Sequence
  Description of Artificial Sequence Synthetic peptide
TRARNEYLLSL

SEQ ID NO: 77 - protein, 8 aa, Artificial Sequence
  Description of Artificial Sequence Synthetic peptide
NPMWVVLL

SEQ ID NO: 78 - protein, 8 aa, Artificial Sequence
  Description of Artificial Sequence Synthetic peptide
KHILVWAL

SEQ ID NO: 79 - protein, 8 aa, Artificial Sequence
  Description of Artificial Sequence Synthetic peptide
SQSDYIPM

SEQ ID NO: 80 - protein, 8 aa, Artificial Sequence
  Description of Artificial Sequence Synthetic peptide
VHDYFSVI

SEQ ID NO: 81 - protein, 9 aa, Artificial Sequence
  Description of Artificial Sequence Synthetic peptide
IYFPAAQTM

SEQ ID NO: 82 - protein, 11 aa, Artificial Sequence
  Description of Artificial Sequence Synthetic peptide
FSYLIWSNPRY

SEQ ID NO: 83 - protein, 10 aa, Artificial Sequence
  Description of Artificial Sequence Synthetic peptide
YSWSAQRQAL

SEQ ID NO: 84 - protein, 10 aa, Artificial Sequence
  Description of Artificial Sequence Synthetic peptide
FAVWTLAETI

SEQ ID NO: 85 - protein, 11 aa, Artificial Sequence
  Description of Artificial Sequence Synthetic peptide
FASLALARRYL

SEQ ID NO: 86 - protein, 11 aa, Artificial Sequence
  Description of Artificial Sequence Synthetic peptide
DVIQQDELDSY

SEQ ID NO: 87 - protein, 10 aa, Artificial Sequence
  Description of Artificial Sequence Synthetic peptide
KNRSSGTVSA

SEQ ID NO: 88 - protein, 9 aa, Artificial Sequence
  Description of Artificial Sequence Synthetic peptide
KLKRFNLSA

SEQ ID NO: 89 - protein, 9 aa, Artificial Sequence
  Description of Artificial Sequence Synthetic peptide
KSFAVWTLA

SEQ ID NO: 90 - protein, 9 aa, Artificial Sequence
  Description of Artificial Sequence Synthetic peptide
KWRLSLCTV

SEQ ID NO: 91 - protein, 10 aa, Artificial Sequence
  Description of Artificial Sequence Synthetic peptide
RSRPVAATAK

SEQ ID NO: 92 - protein, 9 aa, Artificial Sequence
  Description of Artificial Sequence Synthetic peptide
RSRPVAATA

SEQ ID NO: 93 - protein, 11 aa, Artificial Sequence
  Description of Artificial Sequence Synthetic peptide
TAKQAHLTTLK

SEQ ID NO: 94 - protein, 9 aa, Artificial Sequence
  Description of Artificial Sequence Synthetic peptide
SHCPSAMGI

SEQ ID NO: 95 - protein, 8 aa, Artificial Sequence
  Description of Artificial Sequence Synthetic peptide
FHASEGWL

SEQ ID NO: 96 - protein, 8 aa, Artificial Sequence
  Description of Artificial Sequence Synthetic peptide
THEVIVPL

SEQ ID NO: 97 - protein, 8 aa, Artificial Sequence
  Description of Artificial Sequence Synthetic peptide
SRHCLQPL

SEQ ID NO: 98 - protein, 10 aa, Artificial Sequence
  Description of Artificial Sequence Synthetic peptide
FASLALARRY

SEQ ID NO: 99 - protein, 11 aa, Artificial Sequence
  Description of Artificial Sequence Synthetic peptide
SVFNTWNPMWV

SEQ ID NO: 100 - protein, 9 aa, Artificial Sequence
  Description of Artificial Sequence Synthetic peptide
LTHEVIVPL

SEQ ID NO: 101 - protein, 10 aa, Artificial Sequence
  Description of Artificial Sequence Synthetic peptide
YTVMARKSPV

SEQ ID NO: 102 - protein, 9 aa, Artificial Sequence
  Description of Artificial Sequence Synthetic peptide
LSEKGIEDY

SEQ ID NO: 103 - protein, 8 aa, Artificial Sequence
  Description of Artificial Sequence Synthetic peptide
MSDVRTVF

SEQ ID NO: 104 - protein, 10 aa, Artificial Sequence
  Description of Artificial Sequence Synthetic peptide
NSDEPVNLTF

SEQ ID NO: 105 - protein, 9 aa, Artificial Sequence
  Description of Artificial Sequence Synthetic peptide
MSDVRTVFL

SEQ ID NO: 106 - protein, 10 aa, Artificial Sequence
  Description of Artificial Sequence Synthetic peptide
ANDVNDALGY

SEQ ID NO: 107 - protein, 9 aa, Artificial Sequence
  Description of Artificial Sequence Synthetic peptide
LLASVAPRY

SEQ ID NO: 108 - protein, 9 aa, Artificial Sequence
  Description of Artificial Sequence Synthetic peptide
ALMAVIVLV

SEQ ID NO: 109 - protein, 11 aa, Artificial Sequence
  Description of Artificial Sequence Synthetic peptide
ALMAVIVLVAL

SEQ ID NO: 110 - protein, 11 aa, Artificial Sequence
  Description of Artificial Sequence Synthetic peptide
FLNGLEETAGV

SEQ ID NO: 111 - protein, 8 aa, Artificial Sequence
  Description of Artificial Sequence Synthetic peptide
ALMAVIVL

SEQ ID NO: 112 - protein, 8 aa, Artificial Sequence
  Description of Artificial Sequence Synthetic peptide
LMAVIVLV

SEQ ID NO: 113 - protein, 8 aa, Artificial Sequence
  Description of Artificial Sequence Synthetic peptide
VLVALMAV

SEQ ID NO: 114 - protein, 9 aa, Artificial Sequence
  Description of Artificial Sequence Synthetic peptide
MLAACAGEV

SEQ ID NO: 115 - protein, 10 aa, Artificial Sequence
  Description of Artificial Sequence Synthetic peptide
YPMCSGEKAY

SEQ ID NO: 116 - protein, 8 aa, Artificial Sequence
  Description of Artificial Sequence Synthetic peptide
MPSNIQNF

SEQ ID NO: 117 - protein, 10 aa, Artificial Sequence
  Description of Artificial Sequence Synthetic peptide
LPVAVLVALM

SEQ ID NO: 118 - protein, 8 aa, Artificial Sequence
  Description of Artificial Sequence Synthetic peptide
MTSGVYAF

SEQ ID NO: 119 - protein, 8 aa, Artificial Sequence
  Description of Artificial Sequence Synthetic peptide
LPTPTYPL

SEQ ID NO: 120 - protein, 10 aa, Artificial Sequence
  Description of Artificial Sequence Synthetic peptide
YSMSDVRTVF

SEQ ID NO: 121 - protein, 11 aa, Artificial Sequence
  Description of Artificial Sequence Synthetic peptide
DANDVNDALGY

SEQ ID NO: 122 - protein, 10 aa, Artificial Sequence
  Description of Artificial Sequence Synthetic peptide
AEAGAEAASL

SEQ ID NO: 123 - protein, 10 aa, Artificial Sequence
  Description of Artificial Sequence Synthetic peptide
AEAASLNASL

SEQ ID NO: 124 - protein, 11 aa, Artificial Sequence
  Description of Artificial Sequence Synthetic peptide
LENCAEVMRLL

SEQ ID NO: 125 - protein, 10 aa, Artificial Sequence
  Description of Artificial Sequence Synthetic peptide
AETQSRFQLL

SEQ ID NO: 126 - protein, 8 aa, Artificial Sequence
  Description of Artificial Sequence Synthetic peptide
LENCAEVM

SEQ ID NO: 127 - protein, 10 aa, Artificial Sequence
  Description of Artificial Sequence Synthetic peptide
LENCAEVMRL

SEQ ID NO: 128 - protein, 10 aa, Artificial Sequence
  Description of Artificial Sequence Synthetic peptide
SENSDEPVNL

SEQ ID NO: 129 - protein, 11 aa, Artificial Sequence
  Description of Artificial Sequence Synthetic peptide
TEDEIYSRICL

SEQ ID NO: 130 - protein, 10 aa, Artificial Sequence
  Description of Artificial Sequence Synthetic peptide
AESEAHRDSM

SEQ ID NO: 131 - protein, 11 aa, Artificial Sequence
  Description of Artificial Sequence Synthetic peptide
YSMSDVRTVFL

SEQ ID NO: 132 - protein, 9 aa, Artificial Sequence
  Description of Artificial Sequence Synthetic peptide
YSMSDVRTV

SEQ ID NO: 133 - protein, 9 aa, Artificial Sequence
  Description of Artificial Sequence Synthetic peptide
FASGADVQV

SEQ ID NO: 134 - protein, 9 aa, Artificial Sequence
  Description of Artificial Sequence Synthetic peptide
MPRQPSCPL

SEQ ID NO: 135 - protein, 10 aa, Artificial Sequence
  Description of Artificial Sequence Synthetic peptide
TPLCQHLAAL

SEQ ID NO: 136 - protein, 8 aa, Artificial Sequence
  Description of Artificial Sequence Synthetic peptide
SLHEFHLV

SEQ ID NO: 137 - protein, 11 aa, Artificial Sequence
  Description of Artificial Sequence Synthetic peptide
HLVDLSRRFLV

SEQ ID NO: 138 - protein, 11 aa, Artificial Sequence
  Description of Artificial Sequence Synthetic peptide
RLSDETLIDIV

SEQ ID NO: 139 - protein, 9 aa, Artificial Sequence
  Description of Artificial Sequence Synthetic peptide
VLFDTQDPL

SEQ ID NO: 140 - protein, 11 aa, Artificial Sequence
  Description of Artificial Sequence Synthetic peptide
VLFDTQDPLNA

SEQ ID NO: 141 - protein, 10 aa, Artificial Sequence
  Description of Artificial Sequence Synthetic peptide
LMSAAAIYTV

SEQ ID NO: 142 - protein, 11 aa, Artificial Sequence
  Description of Artificial Sequence Synthetic peptide
CLMSAAAIYTV

SEQ ID NO: 143 - protein, 10 aa, Artificial Sequence
  Description of Artificial Sequence Synthetic peptide
ILAGLCLMSA

SEQ ID NO: 144 - protein, 9 aa, Artificial Sequence
  Description of Artificial Sequence Synthetic peptide
LLLPELCSA

SEQ ID NO: 145 - protein, 8 aa, Artificial Sequence
  Description of Artificial Sequence Synthetic peptide
KMALLQYL

SEQ ID NO: 146 - protein, 8 aa, Artificial Sequence
  Description of Artificial Sequence Synthetic peptide
LMSAAAIY

SEQ ID NO: 147 - protein, 9 aa, Artificial Sequence
  Description of Artificial Sequence Synthetic peptide
CLMSAAAIY

SEQ ID NO: 148 - protein, 11 aa, Artificial Sequence
  Description of Artificial Sequence Synthetic peptide
LLLPELCSAFY

SEQ ID NO: 149 - protein, 10 aa, Artificial Sequence
  Description of Artificial Sequence Synthetic peptide
HLGKPGHLSY

SEQ ID NO: 150 - protein, 8 aa, Artificial Sequence
  Description of Artificial Sequence Synthetic peptide
MSLCVLLY

SEQ ID NO: 151 - protein, 9 aa, Artificial Sequence
  Description of Artificial Sequence Synthetic peptide
DMSLCVLLY

SEQ ID NO: 152 - protein, 10 aa, Artificial Sequence
  Description of Artificial Sequence Synthetic peptide
LDMSLCVLLY

SEQ ID NO: 153 - protein, 11 aa, Artificial Sequence
  Description of Artificial Sequence Synthetic peptide
SLDMSLCVLLY

SEQ ID NO: 154 - protein, 11 aa, Artificial Sequence
  Description of Artificial Sequence Synthetic peptide
TTYRDWLGLDY

SEQ ID NO: 155 - protein, 10 aa, Artificial Sequence
  Description of Artificial Sequence Synthetic peptide
LMCQKFLARY

SEQ ID NO: 156 - protein, 11 aa, Artificial Sequence
  Description of Artificial Sequence Synthetic peptide
APTGNFCPQPL

SEQ ID NO: 157 - protein, 10 aa, Artificial Sequence
  Description of Artificial Sequence Synthetic peptide
CPQPLLNSSM

SEQ ID NO: 158 - protein, 9 aa, Artificial Sequence
  Description of Artificial Sequence Synthetic peptide
TPNYSVSML

SEQ ID NO: 159 - protein, 8 aa, Artificial Sequence
  Description of Artificial Sequence Synthetic peptide
TPNYSVSM

SEQ ID NO: 160 - protein, 8 aa, Artificial Sequence
  Description of Artificial Sequence Synthetic peptide
YPNGTSSL

SEQ ID NO: 161 - protein, 9 aa, Artificial Sequence
  Description of Artificial Sequence Synthetic peptide
KPGHLSYAL

SEQ ID NO: 162 - protein, 10 aa, Artificial Sequence
  Description of Artificial Sequence Synthetic peptide
FTRKLLGSAL

SEQ ID NO: 163 - protein, 9 aa, Artificial Sequence
  Description of Artificial Sequence Synthetic peptide
GPASYPIPV

SEQ ID NO: 164 - protein, 10 aa, Artificial Sequence
  Description of Artificial Sequence Synthetic peptide
LPTESPHSSL

SEQ ID NO: 165 - protein, 11 aa, Artificial Sequence
  Description of Artificial Sequence Synthetic peptide
IPHFTATSDAF

SEQ ID NO: 166 - protein, 10 aa, Artificial Sequence
  Description of Artificial Sequence Synthetic peptide
VEIKAVPEGF

SEQ ID NO: 167 - protein, 10 aa, Artificial Sequence
  Description of Artificial Sequence Synthetic peptide
DEVSATETCY

SEQ ID NO: 168 - protein, 10 aa, Artificial Sequence
  Description of Artificial Sequence Synthetic peptide
GELDNQLTTY

SEQ ID NO: 169 - protein, 9 aa, Artificial Sequence
  Description of Artificial Sequence Synthetic peptide
AELGQVLIY

SEQ ID NO: 170 - protein, 8 aa, Artificial Sequence
  Description of Artificial Sequence Synthetic peptide
AELGQVLI

SEQ ID NO: 171 - protein, 10 aa, Artificial Sequence
  Description of Artificial Sequence Synthetic peptide
AELGQVLIYL

SEQ ID NO: 172 - protein, 8 aa, Artificial Sequence
  Description of Artificial Sequence Synthetic peptide
PELCSAFY

SEQ ID NO: 173 - protein, 10 aa, Artificial Sequence
  Description of Artificial Sequence Synthetic peptide
EEFLNHSKAW

SEQ ID NO: 174 - protein, 11 aa, Artificial Sequence
  Description of Artificial Sequence Synthetic peptide
TEEFLNHSKAW

SEQ ID NO: 175 - protein, 10 aa, Artificial Sequence
  Description of Artificial Sequence Synthetic peptide
IENLHDDSCY

SEQ ID NO: 176 - protein, 10 aa, Artificial Sequence
  Description of Artificial Sequence Synthetic peptide
SRMHRGGLRL

SEQ ID NO: 177 - protein, 11 aa, Artificial Sequence
  Description of Artificial Sequence Synthetic peptide
MYHSRMHRGGL

SEQ ID NO: 178 - protein, 8 aa, Artificial Sequence
  Description of Artificial Sequence Synthetic peptide
FFFTRKLL

SEQ ID NO: 179 - protein, 11 aa, Artificial Sequence
  Description of Artificial Sequence Synthetic peptide
IRLQILRQVSL

SEQ ID NO: 180 - protein, 8 aa, Artificial Sequence
  Description of Artificial Sequence Synthetic peptide
FYYTGVGM

SEQ ID NO: 181 - protein, 9 aa, Artificial Sequence
  Description of Artificial Sequence Synthetic peptide
FYYTGVGML

SEQ ID NO: 182 - protein, 10 aa, Artificial Sequence
  Description of Artificial Sequence Synthetic peptide
FYYTGVGMLI

SEQ ID NO: 183 - protein, 8 aa, Artificial Sequence
  Description of Artificial Sequence Synthetic peptide
YYTGVGML

SEQ ID NO: 184 - protein, 9 aa, Artificial Sequence
  Description of Artificial Sequence Synthetic peptide
YYTGVGMLI

SEQ ID NO: 185 - protein, 9 aa, Artificial Sequence
  Description of Artificial Sequence Synthetic peptide
FYPATFGIL

SEQ ID NO: 186 - protein, 11 aa, Artificial Sequence
  Description of Artificial Sequence Synthetic peptide
YAPPQDGPASY

SEQ ID NO: 187 - protein, 8 aa, Artificial Sequence
  Description of Artificial Sequence Synthetic peptide
FTATSDAF

SEQ ID NO: 188 - protein, 11 aa, Artificial Sequence
  Description of Artificial Sequence Synthetic peptide
FQMDDYSLCVL

SEQ ID NO: 189 - protein, 10 aa, Artificial Sequence
  Description of Artificial Sequence Synthetic peptide
MTFSNPPDWL

SEQ ID NO: 190 - protein, 10 aa, Artificial Sequence
  Description of Artificial Sequence Synthetic peptide
YAAHLLDIAM

SEQ ID NO: 191 - protein, 8 aa, Artificial Sequence
  Description of Artificial Sequence Synthetic peptide
FFYAAHLL

SEQ ID NO: 192 - protein, 11 aa, Artificial Sequence
  Description of Artificial Sequence Synthetic peptide
FYAAHLLDIAM

SEQ ID NO: 193 - protein, 9 aa, Artificial Sequence
  Description of Artificial Sequence Synthetic peptide
FMDSCTMRF

SEQ ID NO: 194 - protein, 10 aa, Artificial Sequence
  Description of Artificial Sequence Synthetic peptide
KSMERDCATF

SEQ ID NO: 195 - protein, 8 aa, Artificial Sequence
  Description of Artificial Sequence Synthetic peptide
RTWRRTRR

SEQ ID NO: 196 - protein, 10 aa, Artificial Sequence
  Description of Artificial Sequence Synthetic peptide
RTWRRTRRGR

SEQ ID NO: 197 - protein, 11 aa, Artificial Sequence
  Description of Artificial Sequence Synthetic peptide
RTWRRTRRGRR

SEQ ID NO: 198 - protein, 10 aa, Artificial Sequence
  Description of Artificial Sequence Synthetic peptide
RTRRGRRSSR

SEQ ID NO: 199 - protein, 8 aa, Artificial Sequence
  Description of Artificial Sequence Synthetic peptide
RSSRTLSR

SEQ ID NO: 200 - protein, 10 aa, Artificial Sequence
  Description of Artificial Sequence Synthetic peptide
VMYDGFSVQR

SEQ ID NO: 201 - protein, 9 aa, Artificial Sequence
  Description of Artificial Sequence Synthetic peptide
CVKVCAYIR

SEQ ID NO: 202 - protein, 10 aa, Artificial Sequence
  Description of Artificial Sequence Synthetic peptide
KSTSISTAMR

SEQ ID NO: 203 - protein, 8 aa, Artificial Sequence
  Description of Artificial Sequence Synthetic peptide
GASSIWYR

SEQ ID NO: 204 - protein, 9 aa, Artificial Sequence
  Description of Artificial Sequence Synthetic peptide
AGASSIWYR

SEQ ID NO: 205 - protein, 11 aa, Artificial Sequence
  Description of Artificial Sequence Synthetic peptide
YVMYDGFSVQR

SEQ ID NO: 206 - protein, 11 aa, Artificial Sequence
  Description of Artificial Sequence Synthetic peptide
ELFLVKAKIHK

SEQ ID NO: 207 - protein, 9 aa, Artificial Sequence
  Description of Artificial Sequence Synthetic peptide
STSISTAMR

SEQ ID NO: 208 - protein, 8 aa, Artificial Sequence
  Description of Artificial Sequence Synthetic peptide
TSISTAMR

SEQ ID NO: 209 - protein, 10 aa, Artificial Sequence
  Description of Artificial Sequence Synthetic peptide
FIFTSIAGIR

SEQ ID NO: 210 - protein, 8 aa, Artificial Sequence
  Description of Artificial Sequence Synthetic peptide
FTSIAGIR

SEQ ID NO: 211 - protein, 11 aa, Artificial Sequence
  Description of Artificial Sequence Synthetic peptide
FTNRKVPYCFK

SEQ ID NO: 212 - protein, 9 aa, Artificial Sequence
  Description of Artificial Sequence Synthetic peptide
EAFHQSCFR

SEQ ID NO: 213 - protein, 10 aa, Artificial Sequence
  Description of Artificial Sequence Synthetic peptide
HPNVILNSLY

SEQ ID NO: 214 - protein, 8 aa, Artificial Sequence
  Description of Artificial Sequence Synthetic peptide
FPNVVSGL

SEQ ID NO: 215 - protein, 11 aa, Artificial Sequence
  Description of Artificial Sequence Synthetic peptide
MAENTEGDLNF

SEQ ID NO: 216 - protein, 9 aa, Artificial Sequence
  Description of Artificial Sequence Synthetic peptide
MLVELTPPY

SEQ ID NO: 217 - protein, 10 aa, Artificial Sequence
  Description of Artificial Sequence Synthetic peptide
EPSDVTETLM

SEQ ID NO: 218 - protein, 9 aa, Artificial Sequence
  Description of Artificial Sequence Synthetic peptide
EPSDVTETL

SEQ ID NO: 219 - protein, 11 aa, Artificial Sequence
  Description of Artificial Sequence Synthetic peptide
SPAMTSTSFFF

SEQ ID NO: 220 - protein, 8 aa, Artificial Sequence
  Description of Artificial Sequence Synthetic peptide
MTSTSFFF

SEQ ID NO: 221 - protein, 8 aa, Artificial Sequence
  Description of Artificial Sequence Synthetic peptide
MAIEDILF

SEQ ID NO: 222 - protein, 8 aa, Artificial Sequence
  Description of Artificial Sequence Synthetic peptide
IPEELEYF

SEQ ID NO: 223 - protein, 10 aa, Artificial Sequence
  Description of Artificial Sequence Synthetic peptide
MPICPTYNEV

SEQ ID NO: 224 - protein, 9 aa, Artificial Sequence
  Description of Artificial Sequence Synthetic peptide
LAQEGTTVI

SEQ ID NO: 225 - protein, 11 aa, Artificial Sequence
  Description of Artificial Sequence Synthetic peptide
HPNVILNSLYV

SEQ ID NO: 226 - protein, 10 aa, Artificial Sequence
  Description of Artificial Sequence Synthetic peptide
LPDHFGLGPV

SEQ ID NO: 227 - protein, 9 aa, Artificial Sequence
  Description of Artificial Sequence Synthetic peptide
MAIEDILFV

SEQ ID NO: 228 - protein, 9 aa, Artificial Sequence
  Description of Artificial Sequence Synthetic peptide
IPEELEYFI

SEQ ID NO: 229 - protein, 10 aa, Artificial Sequence
  Description of Artificial Sequence Synthetic peptide
EPQNFIDSLI

SEQ ID NO: 230 - protein, 9 aa, Artificial Sequence
  Description of Artificial Sequence Synthetic peptide
CPTYNEVHL

SEQ ID NO: 231 - protein, 10 aa, Artificial Sequence
  Description of Artificial Sequence Synthetic peptide
MYDGFSVQRL

SEQ ID NO: 232 - protein, 11 aa, Artificial Sequence
  Description of Artificial Sequence Synthetic peptide
MYDGFSVQRLV

SEQ ID NO: 233 - protein, 10 aa, Artificial Sequence
  Description of Artificial Sequence Synthetic peptide
AYDASTFRGL

SEQ ID NO: 234 - protein, 9 aa, Artificial Sequence
  Description of Artificial Sequence Synthetic peptide
FTDCGRPPL

SEQ ID NO: 235 - protein, 8 aa, Artificial Sequence
  Description of Artificial Sequence Synthetic peptide
KFDLFARL

SEQ ID NO: 236 - protein, 9 aa, Artificial Sequence
  Description of Artificial Sequence Synthetic peptide
STYLIAQSI

SEQ ID NO: 237 - protein, 11 aa, Artificial Sequence
  Description of Artificial Sequence Synthetic peptide
KSTSISTAMRL

SEQ ID NO: 238 - protein, 9 aa, Artificial Sequence
  Description of Artificial Sequence Synthetic peptide
QTFGKMFFV

SEQ ID NO: 239 - protein, 11 aa, Artificial Sequence
  Description of Artificial Sequence Synthetic peptide
WAYDASTFRGL

SEQ ID NO: 240 - protein, 9 aa, Artificial Sequence
  Description of Artificial Sequence Synthetic peptide
STHPPGASL

SEQ ID NO: 241 - protein, 10 aa, Artificial Sequence
  Description of Artificial Sequence Synthetic peptide
RADPRAGPSV

SEQ ID NO: 242 - protein, 10 aa, Artificial Sequence
  Description of Artificial Sequence Synthetic peptide
MTSTSFFFTL

SEQ ID NO: 243 - protein, 9 aa, Artificial Sequence
  Description of Artificial Sequence Synthetic peptide
RSAEPQNFI

SEQ ID NO: 244 - protein, 9 aa, Artificial Sequence
  Description of Artificial Sequence Synthetic peptide
LTSSDDLLI

SEQ ID NO: 245 - protein, 11 aa, Artificial Sequence
  Description of Artificial Sequence Synthetic peptide
KYMLNSVLENF

SEQ ID NO: 246 - protein, 10 aa, Artificial Sequence
  Description of Artificial Sequence Synthetic peptide
YMLNSVLENF

SEQ ID NO: 247 - protein, 9 aa, Artificial Sequence
  Description of Artificial Sequence Synthetic peptide
GYACAEPSF

SEQ ID NO: 248 - protein, 11 aa, Artificial Sequence
  Description of Artificial Sequence Synthetic peptide
FFAAQAGAWKI

SEQ ID NO: 249 - protein, 10 aa, Artificial Sequence
  Description of Artificial Sequence Synthetic peptide
SFFAAQAGAW

SEQ ID NO: 250 - protein, 9 aa, Artificial Sequence
  Description of Artificial Sequence Synthetic peptide
YMLKAKSQF

SEQ ID NO: 251 - protein, 8 aa, Artificial Sequence
  Description of Artificial Sequence Synthetic peptide
RYFVPKML

SEQ ID NO: 252 - protein, 9 aa, Artificial Sequence
  Description of Artificial Sequence Synthetic peptide
ATLNGRMYF

SEQ ID NO: 253 - protein, 8 aa, Artificial Sequence
  Description of Artificial Sequence Synthetic peptide
YTISFLFW

SEQ ID NO: 254 - protein, 9 aa, Artificial Sequence
  Description of Artificial Sequence Synthetic peptide
DYTISFLFW

SEQ ID NO: 255 - protein, 8 aa, Artificial Sequence
  Description of Artificial Sequence Synthetic peptide
KYMLNSVL

SEQ ID NO: 256 - protein, 8 aa, Artificial Sequence
  Description of Artificial Sequence Synthetic peptide
RYPAKVTL

SEQ ID NO: 257 - protein, 9 aa, Artificial Sequence
  Description of Artificial Sequence Synthetic peptide
FFAAQAGAW

SEQ ID NO: 258 - protein, 10 aa, Artificial Sequence
  Description of Artificial Sequence Synthetic peptide
EYMLKAKSQF

SEQ ID NO: 259 - protein, 9 aa, Artificial Sequence
  Description of Artificial Sequence Synthetic peptide
KESFGPQAL

SEQ ID NO: 260 - protein, 9 aa, Artificial Sequence
  Description of Artificial Sequence Synthetic peptide
CEVAPNNVV

SEQ ID NO: 261 - protein, 11 aa, Artificial Sequence
  Description of Artificial Sequence Synthetic peptide
KEMHPNKLNAV

SEQ ID NO: 262 - protein, 8 aa, Artificial Sequence
  Description of Artificial Sequence Synthetic peptide
CEVAPNNV

SEQ ID NO: 263 - protein, 8 aa, Artificial Sequence
  Description of Artificial Sequence Synthetic peptide
KQFFYNII

SEQ ID NO: 264 - protein, 8 aa, Artificial Sequence
  Description of Artificial Sequence Synthetic peptide
SQLQGLQL

SEQ ID NO: 265 - protein, 11 aa, Artificial Sequence
  Description of Artificial Sequence Synthetic peptide
TEYKLVVVGAC

SEQ ID NO: 266 - protein, 8 aa, Artificial Sequence
  Description of Artificial Sequence Synthetic peptide
FEDGPYAV

SEQ ID NO: 267 - protein, 8 aa, Artificial Sequence
  Description of Artificial Sequence Synthetic peptide
AQAGAWKI

SEQ ID NO: 268 - protein, 9 aa, Artificial Sequence
  Description of Artificial Sequence Synthetic peptide
AERLVGPGY

SEQ ID NO: 269 - protein, 11 aa, Artificial Sequence
  Description of Artificial Sequence Synthetic peptide
IEYMLKAKSQF

SEQ ID NO: 270 - protein, 10 aa, Artificial Sequence
  Description of Artificial Sequence Synthetic peptide
SDYTISFLFW

SEQ ID NO: 271 - protein, 11 aa, Artificial Sequence
  Description of Artificial Sequence Synthetic peptide
GELGWENPNQW

SEQ ID NO: 272 - protein, 10 aa, Artificial Sequence
  Description of Artificial Sequence Synthetic peptide
SEMTAVTQKI

SEQ ID NO: 273 - protein, 11 aa, Artificial Sequence
  Description of Artificial Sequence Synthetic peptide
SEMTAVTQKIV

SEQ ID NO: 274 - protein, 8 aa, Artificial Sequence
  Description of Artificial Sequence Synthetic peptide
NKMDMNQW

SEQ ID NO: 275 - protein, 9 aa, Artificial Sequence
  Description of Artificial Sequence Synthetic peptide
YTSSEVSTV

SEQ ID NO: 276 - protein, 11 aa, Artificial Sequence
  Description of Artificial Sequence Synthetic peptide
YTSSEVSTVEL

SEQ ID NO: 277 - protein, 9 aa, Artificial Sequence
  Description of Artificial Sequence Synthetic peptide
YSPDILPTV

SEQ ID NO: 278 - protein, 10 aa, Artificial Sequence
  Description of Artificial Sequence Synthetic peptide
FAAQAGAWKI

SEQ ID NO: 279 - protein, 8 aa, Artificial Sequence
  Description of Artificial Sequence Synthetic peptide
FAAQAGAW

SEQ ID NO: 280 - protein, 11 aa, Artificial Sequence
  Description of Artificial Sequence Synthetic peptide
KTATLNGRMYF

SEQ ID NO: 281 - protein, 9 aa, Artificial Sequence
  Description of Artificial Sequence Synthetic peptide
KTATLNGRM

SEQ ID NO: 282 - protein, 10 aa, Artificial Sequence
  Description of Artificial Sequence Synthetic peptide
YTISFLFWIL

SEQ ID NO: 283 - protein, 9 aa, Artificial Sequence
  Description of Artificial Sequence Synthetic peptide
YTISFLFWI

SEQ ID NO: 284 - protein, 9 aa, Artificial Sequence
  Description of Artificial Sequence Synthetic peptide
IALRPSGTM

SEQ ID NO: 285 - protein, 10 aa, Artificial Sequence
  Description of Artificial Sequence Synthetic peptide
IALRPSGTML

SEQ ID NO: 286 - protein, 9 aa, Artificial Sequence
  Description of Artificial Sequence Synthetic peptide
FAVEAHQCI

SEQ ID NO: 287 - protein, 8 aa, Artificial Sequence
  Description of Artificial Sequence Synthetic peptide
MSSLPCPL

SEQ ID NO: 288 - protein, 8 aa, Artificial Sequence
  Description of Artificial Sequence Synthetic peptide
YACAEPSF

SEQ ID NO: 289 - protein, 8 aa, Artificial Sequence
  Description of Artificial Sequence Synthetic peptide
IVDPDPVL

SEQ ID NO: 290 - protein, 10 aa, Artificial Sequence
  Description of Artificial Sequence Synthetic peptide
RALKEKAQPL

SEQ ID NO: 291 - protein, 9 aa, Artificial Sequence
  Description of Artificial Sequence Synthetic peptide
YLNSRQFPM

SEQ ID NO: 292 - protein, 9 aa, Artificial Sequence
  Description of Artificial Sequence Synthetic peptide
SIMALSTSI

SEQ ID NO: 293 - protein, 8 aa, Artificial Sequence
  Description of Artificial Sequence Synthetic peptide
SLTDISTL

SEQ ID NO: 294 - protein, 10 aa, Artificial Sequence
  Description of Artificial Sequence Synthetic peptide
FLISYWSEQI

SEQ ID NO: 295 - protein, 8 aa, Artificial Sequence
  Description of Artificial Sequence Synthetic peptide
SMLSLPRV

SEQ ID NO: 296 - protein, 11 aa, Artificial Sequence
  Description of Artificial Sequence Synthetic peptide
AVASVLPLWPA

SEQ ID NO: 297 - protein, 11 aa, Artificial Sequence
  Description of Artificial Sequence Synthetic peptide
MLLVIIVSVGI

SEQ ID NO: 298 - protein, 9 aa, Artificial Sequence
  Description of Artificial Sequence Synthetic peptide
SQHQVLFFL

SEQ ID NO: 299 - protein, 9 aa, Artificial Sequence
  Description of Artificial Sequence Synthetic peptide
RLRTDLFSK

SEQ ID NO: 300 - protein, 8 aa, Artificial Sequence
  Description of Artificial Sequence Synthetic peptide
KQRTSSEK

SEQ ID NO: 301 - protein, 10 aa, Artificial Sequence
  Description of Artificial Sequence Synthetic peptide
RLKYNLQGYK

SEQ ID NO: 302 - protein, 8 aa, Artificial Sequence
  Description of Artificial Sequence Synthetic peptide
RSRRSTTA

SEQ ID NO: 303 - protein, 8 aa, Artificial Sequence
  Description of Artificial Sequence Synthetic peptide
RMRAMATA

SEQ ID NO: 304 - protein, 8 aa, Artificial Sequence
  Description of Artificial Sequence Synthetic peptide
LTSPIVCF

SEQ ID NO: 305 - protein, 11 aa, Artificial Sequence
  Description of Artificial Sequence Synthetic peptide
LARASPALASL

SEQ ID NO: 306 - protein, 10 aa, Artificial Sequence
  Description of Artificial Sequence Synthetic peptide
LRNGALTSPI

SEQ ID NO: 307 - protein, 11 aa, Artificial Sequence
  Description of Artificial Sequence Synthetic peptide
VLRNGALTSPI

SEQ ID NO: 308 - protein, 11 aa, Artificial Sequence
  Description of Artificial Sequence Synthetic peptide
LRNGALTSPIV

SEQ ID NO: 309 - protein, 8 aa, Artificial Sequence
  Description of Artificial Sequence Synthetic peptide
LARASPAL

SEQ ID NO: 310 - protein, 9 aa, Artificial Sequence
  Description of Artificial Sequence Synthetic peptide
SLARASPAL

SEQ ID NO: 311 - protein, 9 aa, Artificial Sequence
  Description of Artificial Sequence Synthetic peptide
LRSLTFSLV

SEQ ID NO: 312 - protein, 10 aa, Artificial Sequence
  Description of Artificial Sequence Synthetic peptide
SAITSKVSTV

SEQ ID NO: 313 - protein, 9 aa, Artificial Sequence
  Description of Artificial Sequence Synthetic peptide
AITSKVSTV

SEQ ID NO: 314 - protein, 10 aa, Artificial Sequence
  Description of Artificial Sequence Synthetic peptide
SAITSKVSTV

SEQ ID NO: 315 - protein, 9 aa, Artificial Sequence
  Description of Artificial Sequence Synthetic peptide
ASLARASPA

SEQ ID NO: 316 - protein, 8 aa, Artificial Sequence
  Description of Artificial Sequence Synthetic peptide
SLARASPA

SEQ ID NO: 317 - protein, 10 aa, Artificial Sequence
  Description of Artificial Sequence Synthetic peptide
ASLARASPAL

SEQ ID NO: 318 - protein, 10 aa, Artificial Sequence
  Description of Artificial Sequence Synthetic peptide
QASLARASPA

SEQ ID NO: 319 - protein, 9 aa, Artificial Sequence
  Description of Artificial Sequence Synthetic peptide
LARASPALA

SEQ ID NO: 320 - protein, 10 aa, Artificial Sequence
  Description of Artificial Sequence Synthetic peptide
KLRSLTFSLV

SEQ ID NO: 321 - protein, 9 aa, Artificial Sequence
  Description of Artificial Sequence Synthetic peptide
LLADATVEL

SEQ ID NO: 322 - protein, 11 aa, Artificial Sequence
  Description of Artificial Sequence Synthetic peptide
LLADATVELSL

SEQ ID NO: 323 - protein, 8 aa, Artificial Sequence
  Description of Artificial Sequence Synthetic peptide
HMAFSPAV

SEQ ID NO: 324 - protein, 8 aa, Artificial Sequence
  Description of Artificial Sequence Synthetic peptide
YLDSIVFL

SEQ ID NO: 325 - protein, 8 aa, Artificial Sequence
  Description of Artificial Sequence Synthetic peptide
FLEDLSPL

SEQ ID NO: 326 - protein, 11 aa, Artificial Sequence
  Description of Artificial Sequence Synthetic peptide
YLDSIVFLEDL

SEQ ID NO: 327 - protein, 10 aa, Artificial Sequence
  Description of Artificial Sequence Synthetic peptide
FLEDLSPLEA

SEQ ID NO: 328 - protein, 10 aa, Artificial Sequence
  Description of Artificial Sequence Synthetic peptide
LLLHGAEPKL

SEQ ID NO: 329 - protein, 8 aa, Artificial Sequence
  Description of Artificial Sequence Synthetic peptide
TLIDVPKV

SEQ ID NO: 330 - protein, 8 aa, Artificial Sequence
  Description of Artificial Sequence Synthetic peptide
TMACINLA

SEQ ID NO: 331 - protein, 10 aa, Artificial Sequence
  Description of Artificial Sequence Synthetic peptide
LSKDIMFHFK

SEQ ID NO: 332 - protein, 11 aa, Artificial Sequence
  Description of Artificial Sequence Synthetic peptide
VTMACINLASK

SEQ ID NO: 333 - protein, 10 aa, Artificial Sequence
  Description of Artificial Sequence Synthetic peptide
TMACINLASK

SEQ ID NO: 334 - protein, 10 aa, Artificial Sequence
  Description of Artificial Sequence Synthetic peptide
MSYDNNLFIK

SEQ ID NO: 335 - protein, 9 aa, Artificial Sequence
  Description of Artificial Sequence Synthetic peptide
KTWKEKTLK

SEQ ID NO: 336 - protein, 8 aa, Artificial Sequence
  Description of Artificial Sequence Synthetic peptide
VTLIDVPK

SEQ ID NO: 337 - protein, 8 aa, Artificial Sequence
  Description of Artificial Sequence Synthetic peptide
MPLVHMAF

SEQ ID NO: 338 - protein, 11 aa, Artificial Sequence
  Description of Artificial Sequence Synthetic peptide
MPLVHMAFSPA

SEQ ID NO: 339 - protein, 10 aa, Artificial Sequence
  Description of Artificial Sequence Synthetic peptide
YPDYLDSIVF

SEQ ID NO: 340 - protein, 11 aa, Artificial Sequence
  Description of Artificial Sequence Synthetic peptide
YPDYLDSIVFL

SEQ ID NO: 341 - protein, 8 aa, Artificial Sequence
  Description of Artificial Sequence Synthetic peptide
MSYDNNLF

SEQ ID NO: 342 - protein, 8 aa, Artificial Sequence
  Description of Artificial Sequence Synthetic peptide
MPLVHMAF

SEQ ID NO: 343 - protein, 9 aa, Artificial Sequence
  Description of Artificial Sequence Synthetic peptide
MAFSPAVDV

SEQ ID NO: 344 - protein, 8 aa, Artificial Sequence
  Description of Artificial Sequence Synthetic peptide
AVQWLRPK

SEQ ID NO: 345 - protein, 11 aa, Artificial Sequence
  Description of Artificial Sequence Synthetic peptide
HVMPDTPDILK

SEQ ID NO: 346 - protein, 8 aa, Artificial Sequence
  Description of Artificial Sequence Synthetic peptide
KVMYILFY

SEQ ID NO: 347 - protein, 11 aa, Artificial Sequence
  Description of Artificial Sequence Synthetic peptide
VQNAVQWLRPK

SEQ ID NO: 348 - protein, 9 aa, Artificial Sequence
  Description of Artificial Sequence Synthetic peptide
VQNAVQWLR

SEQ ID NO: 349 - protein, 11 aa, Artificial Sequence
  Description of Artificial Sequence Synthetic peptide
TLFQIIYDNLR

SEQ ID NO: 350 - protein, 8 aa, Artificial Sequence
  Description of Artificial Sequence Synthetic peptide
CLASLHPR

SEQ ID NO: 351 - protein, 10 aa, Artificial Sequence
  Description of Artificial Sequence Synthetic peptide
RSLGCLASLH

SEQ ID NO: 352 - protein, 11 aa, Artificial Sequence
  Description of Artificial Sequence Synthetic peptide
KLLHEYWMSLR

SEQ ID NO: 353 - protein, 10 aa, Artificial Sequence
  Description of Artificial Sequence Synthetic peptide
LLHEYWMSLR

SEQ ID NO: 354 - protein, 9 aa, Artificial Sequence
  Description of Artificial Sequence Synthetic peptide
EVKEEDEPF

SEQ ID NO: 355 - protein, 11 aa, Artificial Sequence
  Description of Artificial Sequence Synthetic peptide
QVNKVMYILFY

SEQ ID NO: 356 - protein, 9 aa, Artificial Sequence
  Description of Artificial Sequence Synthetic peptide
EVQNAVQWL

SEQ ID NO: 357 - protein, 8 aa, Artificial Sequence
  Description of Artificial Sequence Synthetic peptide
LHEYWMSL

SEQ ID NO: 358 - protein, 11 aa, Artificial Sequence
  Description of Artificial Sequence Synthetic peptide
YKLLHEYWMSL

SEQ ID NO: 359 - protein, 9 aa, Artificial Sequence
  Description of Artificial Sequence Synthetic peptide
MEESNNSTL

SEQ ID NO: 360 - protein, 11 aa, Artificial Sequence
  Description of Artificial Sequence Synthetic peptide
MEESNNSTLFI

SEQ ID NO: 361 - protein, 11 aa, Artificial Sequence
  Description of Artificial Sequence Synthetic peptide
CAYIRKQVEKI

SEQ ID NO: 362 - protein, 15 aa, Artificial Sequence
  Description of Artificial Sequence Synthetic peptide
CAWESSRDIDDPEAF

SEQ ID NO: 363 - protein, 11 aa, Artificial Sequence
  Description of Artificial Sequence Synthetic peptide
CASGGTDTQYF

SEQ ID NO: 364 - protein, 15 aa, Artificial Sequence
  Description of Artificial Sequence Synthetic peptide
CASRPGQRYNSPLHF

SEQ ID NO: 365 - protein, 14 aa, Artificial Sequence
  Description of Artificial Sequence Synthetic peptide
CASSEQGFWNGYTF

SEQ ID NO: 366 - protein, 15 aa, Artificial Sequence
  Description of Artificial Sequence Synthetic peptide
CASTPSAGANQPQHF

SEQ ID NO: 367 - protein, 16 aa, Artificial Sequence
  Description of Artificial Sequence Synthetic peptide
CASSLGDDSMNTEAFF

SEQ ID NO: 368 - protein, 14 aa, Artificial Sequence
  Description of Artificial Sequence Synthetic peptide
CASSIGTGSKPQHF

SEQ ID NO: 369 - protein, 14 aa, Artificial Sequence
  Description of Artificial Sequence Synthetic peptide
CASIPTGQNYGYTF

SEQ ID NO: 370 - protein, 13 aa, Artificial Sequence
  Description of Artificial Sequence Synthetic peptide
CASSSGLRNIQYF

SEQ ID NO: 371 - protein, 15 aa, Artificial Sequence
  Description of Artificial Sequence Synthetic peptide
CASSLSGSSYNEQFF

SEQ ID NO: 372 - protein, 15 aa, Artificial Sequence
  Description of Artificial Sequence Synthetic peptide
CASSLETTGANVLTF

SEQ ID NO: 373 - protein, 16 aa, Artificial Sequence
  Description of Artificial Sequence Synthetic peptide
CASSYPSAAAYNEQFF

SEQ ID NO: 374 - protein, 13 aa, Artificial Sequence
  Description of Artificial Sequence Synthetic peptide
CASSWTGNQPQHF

SEQ ID NO: 375 - protein, 14 aa, Artificial Sequence
  Description of Artificial Sequence Synthetic peptide
CASSLGANTGELFF

SEQ ID NO: 376 - protein, 15 aa, Artificial Sequence
  Description of Artificial Sequence Synthetic peptide
CASSEALGPGNTIYF

SEQ ID NO: 377 - protein, 13 aa, Artificial Sequence
  Description of Artificial Sequence Synthetic peptide
CASRPYGTYGYTF

SEQ ID NO: 378 - protein, 13 aa, Artificial Sequence
  Description of Artificial Sequence Synthetic peptide
CASSVDWGAEAFF

SEQ ID NO: 379 - protein, 16 aa, Artificial Sequence
  Description of Artificial Sequence Synthetic peptide
CATSDLRDRGNNEQFF

SEQ ID NO: 380 - protein, 15 aa, Artificial Sequence
  Description of Artificial Sequence Synthetic peptide
CASSRSGSSYNEQFF

SEQ ID NO: 381 - protein, 12 aa, Artificial Sequence
  Description of Artificial Sequence Synthetic peptide
CASSLYTGELFF

SEQ ID NO: 382 - protein, 15 aa, Artificial Sequence
  Description of Artificial Sequence Synthetic peptide
CAISGGTGANYGYTF

SEQ ID NO: 383 - protein, 13 aa, Artificial Sequence
  Description of Artificial Sequence Synthetic peptide
CASSFQQNTEAFF

SEQ ID NO: 384 - protein, 13 aa, Artificial Sequence
  Description of Artificial Sequence Synthetic peptide
CASSLTGGSEAFF

SEQ ID NO: 385 - protein, 13 aa, Artificial Sequence
  Description of Artificial Sequence Synthetic peptide
CASSLAPGNEQFF

SEQ ID NO: 386 - protein, 15 aa, Artificial Sequence
  Description of Artificial Sequence Synthetic peptide
CASSFAGGGNQPQHF

SEQ ID NO: 387 - protein, 17 aa, Artificial Sequence
  Description of Artificial Sequence Synthetic peptide
CASSYLVGAGSNYGYTF

SEQ ID NO: 388 - protein, 13 aa, Artificial Sequence
  Description of Artificial Sequence Synthetic peptide
CASSFWGNTEAFF

SEQ ID NO: 389 - protein, 11 aa, Artificial Sequence
  Description of Artificial Sequence Synthetic peptide
CAWKEVRGQFF

SEQ ID NO: 390 - protein, 16 aa, Artificial Sequence
  Description of Artificial Sequence Synthetic peptide
CASSPLPSGGTYEQYF

SEQ ID NO: 391 - protein, 13 aa, Artificial Sequence
  Description of Artificial Sequence Synthetic peptide
CASSSGPSYEQYF

SEQ ID NO: 392 - protein, 16 aa, Artificial Sequence
  Description of Artificial Sequence Synthetic peptide
CASSRGEGGSNQPQHF

SEQ ID NO: 393 - protein, 15 aa, Artificial Sequence
  Description of Artificial Sequence Synthetic peptide
CASSVDGGQSDEQYF

SEQ ID NO: 394 - protein, 14 aa, Artificial Sequence
  Description of Artificial Sequence Synthetic peptide
CASSLPGDPYEQYF

SEQ ID NO: 395 - protein, 11 aa, Artificial Sequence
  Description of Artificial Sequence Synthetic peptide
CASSYNNEQFF

SEQ ID NO: 396 - protein, 14 aa, Artificial Sequence
  Description of Artificial Sequence Synthetic peptide
CAWSEGRGNEKLFF

SEQ ID NO: 397 - protein, 15 aa, Artificial Sequence
  Description of Artificial Sequence Synthetic peptide
CASSLSGPVEDEQYF

SEQ ID NO: 398 - protein, 13 aa, Artificial Sequence
  Description of Artificial Sequence Synthetic peptide
CASSSISTGELFF

SEQ ID NO: 399 - protein, 19 aa, Artificial Sequence
  Description of Artificial Sequence Synthetic peptide
CASSLAGLAGPLAKNIQYF

SEQ ID NO: 400 - protein, 15 aa, Artificial Sequence
  Description of Artificial Sequence Synthetic peptide
CASSDLAGLNYGYTF

SEQ ID NO: 401 - protein, 14 aa, Artificial Sequence
  Description of Artificial Sequence Synthetic peptide
CASSLTSATGELFF

SEQ ID NO: 402 - protein, 14 aa, Artificial Sequence
  Description of Artificial Sequence Synthetic peptide
CAISEVVGLQPQHF

SEQ ID NO: 403 - protein, 15 aa, Artificial Sequence
  Description of Artificial Sequence Synthetic peptide
CASSAGRYSTYEQYF

SEQ ID NO: 404 - protein, 13 aa, Artificial Sequence
  Description of Artificial Sequence Synthetic peptide
CASSWGGNTEAFF

SEQ ID NO: 405 - protein, 14 aa, Artificial Sequence
  Description of Artificial Sequence Synthetic peptide
CASSPTQGAVEQYF

SEQ ID NO: 406 - protein, 12 aa, Artificial Sequence
  Description of Artificial Sequence Synthetic peptide
CASSIGSHGYTF

SEQ ID NO: 407 - protein, 16 aa, Artificial Sequence
  Description of Artificial Sequence Synthetic peptide
CASSLAVVKSGNTIYF

SEQ ID NO: 408 - protein, 14 aa, Artificial Sequence
  Description of Artificial Sequence Synthetic peptide
CSARDREYGNTIYF

SEQ ID NO: 409 - protein, 16 aa, Artificial Sequence
  Description of Artificial Sequence Synthetic peptide
CSARGGQTEVGNTIYF

SEQ ID NO: 410 - protein, 15 aa, Artificial Sequence
  Description of Artificial Sequence Synthetic peptide
CASSEGAKAPGEQYF

SEQ ID NO: 411 - protein, 13 aa, Artificial Sequence
  Description of Artificial Sequence Synthetic peptide
CSARDETGNGYTF

SEQ ID NO: 412 - protein, 13 aa, Artificial Sequence
  Description of Artificial Sequence Synthetic peptide
CASSTRSAYEQYF

SEQ ID NO: 413 - protein, 16 aa, Artificial Sequence
  Description of Artificial Sequence Synthetic peptide
CSARDIQGAQVYEQYF

SEQ ID NO: 414 - protein, 13 aa, Artificial Sequence
  Description of Artificial Sequence Synthetic peptide
CSARDGTGNGYTF

SEQ ID NO: 415 - protein, 13 aa, Artificial Sequence
  Description of Artificial Sequence Synthetic peptide
CSARDTVGNTIYF

SEQ ID NO: 416 - protein, 13 aa, Artificial Sequence
  Description of Artificial Sequence Synthetic peptide
CASSVRSSYEQYF

SEQ ID NO: 417 - protein, 15 aa, Artificial Sequence
  Description of Artificial Sequence Synthetic peptide
CASSFSSGTTDTQYF

SEQ ID NO: 418 - protein, 14 aa, Artificial Sequence
  Description of Artificial Sequence Synthetic peptide
CASSVGGEAYEQYF

SEQ ID NO: 419 - protein, 13 aa, Artificial Sequence
  Description of Artificial Sequence Synthetic peptide
CASSVGSVGEAFF

SEQ ID NO: 420 - protein, 14 aa, Artificial Sequence
  Description of Artificial Sequence Synthetic peptide
CSVGTGGTNEKLFF

SEQ ID NO: 421 - protein, 12 aa, Artificial Sequence
  Description of Artificial Sequence Synthetic peptide
CSSQEGGYGYTF

SEQ ID NO: 422 - protein, 14 aa, Artificial Sequence
  Description of Artificial Sequence Synthetic peptide
CSVGQGGTNEKLFF

SEQ ID NO: 423 - protein, 15 aa, Artificial Sequence
  Description of Artificial Sequence Synthetic peptide
CASSDGRVAPGELFF

SEQ ID NO: 424 - protein, 13 aa, Artificial Sequence
  Description of Artificial Sequence Synthetic peptide
CSARDSTGNGYTF

SEQ ID NO: 425 - protein, 13 aa, Artificial Sequence
  Description of Artificial Sequence Synthetic peptide
CASSLSSYTEAFF

SEQ ID NO: 426 - protein, 13 aa, Artificial Sequence
  Description of Artificial Sequence Synthetic peptide
CASSVVGGDEQYF

SEQ ID NO: 427 - protein, 15 aa, Artificial Sequence
  Description of Artificial Sequence Synthetic peptide
CSVGGRGDRDQPQHF

SEQ ID NO: 428 - protein, 13 aa, Artificial Sequence
  Description of Artificial Sequence Synthetic peptide
CASSIRSSYEQYF

SEQ ID NO: 429 - protein, 14 aa, Artificial Sequence
  Description of Artificial Sequence Synthetic peptide
CASSSPGINYGYTF

SEQ ID NO: 430 - protein, 16 aa, Artificial Sequence
  Description of Artificial Sequence Synthetic peptide
CASSQPDKAAYNEQFF

SEQ ID NO: 431 - protein, 15 aa, Artificial Sequence
  Description of Artificial Sequence Synthetic peptide
CASSGGQVAPSEQYF

SEQ ID NO: 432 - protein, 14 aa, Artificial Sequence
  Description of Artificial Sequence Synthetic peptide
CASSSPGIDYGYTF

SEQ ID NO: 433 - protein, 17 aa, Artificial Sequence
  Description of Artificial Sequence Synthetic peptide
CASSLGVEDHRVYGYTF

SEQ ID NO: 434 - protein, 15 aa, Artificial Sequence
  Description of Artificial Sequence Synthetic peptide
CASSPIGMWGNEQFF

SEQ ID NO: 435 - protein, 17 aa, Artificial Sequence
  Description of Artificial Sequence Synthetic peptide
CASSLAGLAGPLAKNIQ

SEQ ID NO: 436 - protein, 17 aa, Artificial Sequence
  Description of Artificial Sequence Synthetic peptide
CATSRDGTSGLTGELFF

SEQ ID NO: 437 - protein, 15 aa, Artificial Sequence
  Description of Artificial Sequence Synthetic peptide
CASSLRIGGRETQYF

SEQ ID NO: 438 - protein, 8 aa, Artificial Sequence
  Description of Artificial Sequence Synthetic peptide
CASGADTF

SEQ ID NO: 439 - protein, 15 aa, Artificial Sequence
  Description of Artificial Sequence Synthetic peptide
CASSPGMGGADGYTF

SEQ ID NO: 440 - protein, 15 aa, Artificial Sequence
  Description of Artificial Sequence Synthetic peptide
CASSIDRGINYGYTF

SEQ ID NO: 441 - protein, 15 aa, Artificial Sequence
  Description of Artificial Sequence Synthetic peptide
CASSEMTGGQDTQYF

SEQ ID NO: 442 - protein, 15 aa, Artificial Sequence
  Description of Artificial Sequence Synthetic peptide
CASSLEPRPHTEAFF

SEQ ID NO: 443 - protein, 13 aa, Artificial Sequence
  Description of Artificial Sequence Synthetic peptide
CASATLQGDGYTF

SEQ ID NO: 444 - protein, 15 aa, Artificial Sequence
  Description of Artificial Sequence Synthetic peptide
CASSQDSGYNSPLHF

SEQ ID NO: 445 - protein, 7 aa, Artificial Sequence
  Description of Artificial Sequence Synthetic peptide
CSVEAFF

SEQ ID NO: 446 - protein, 11 aa, Artificial Sequence
  Description of Artificial Sequence Synthetic peptide
CSADLNTEAFF

SEQ ID NO: 447 - protein, 14 aa, Artificial Sequence
  Description of Artificial Sequence Synthetic peptide
CSVAEGQGAYEQFF

SEQ ID NO: 448 - protein, 12 aa, Artificial Sequence
  Description of Artificial Sequence Synthetic peptide
CASSSARRGYTF

SEQ ID NO: 449 - protein, 13 aa, Artificial Sequence
  Description of Artificial Sequence Synthetic peptide
CSASPSGAVEQFF

SEQ ID NO: 450 - protein, 12 aa, Artificial Sequence
  Description of Artificial Sequence Synthetic peptide
CASRRSNQPQHF

SEQ ID NO: 451 - protein, 15 aa, Artificial Sequence
  Description of Artificial Sequence Synthetic peptide
CASSPLEDSSYGYTF

SEQ ID NO: 452 - protein, 15 aa, Artificial Sequence
  Description of Artificial Sequence Synthetic peptide
CASSLTGGYTGELFF

SEQ ID NO: 453 - protein, 13 aa, Artificial Sequence
  Description of Artificial Sequence Synthetic peptide
CASNKLGYQPQHF

SEQ ID NO: 454 - protein, 9 aa, Artificial Sequence
  Description of Artificial Sequence Synthetic peptide
EVIVPHSGW

SEQ ID NO: 455 - protein, 8 aa, Unknown
  Description of Unknown MANA restriction peptide
ETPNLDRM

SEQ ID NO: 456 - protein, 9 aa, Unknown
  Description of Unknown MANA restriction peptide
SVFNTWKPM

SEQ ID NO: 457 - protein, 11 aa, Artificial Sequence
  Description of Artificial Sequence Synthetic peptide
NSSCMGGMNWR

SEQ ID NO: 458 - protein, 11 aa, Unknown
  Description of Unknown MANA restriction peptide
NSSCMGGMNRR

SEQ ID NO: 459 - protein, 16 aa, Artificial Sequence
  Description of Artificial Sequence Synthetic peptide
CASSHTFDRGGETQYF

SEQ ID NO: 460 - protein, 14 aa, Artificial Sequence
  Description of Artificial Sequence Synthetic peptide
CASSASLAGVEQYF

SEQ ID NO: 461 - protein, 13 aa, Artificial Sequence
  Description of Artificial Sequence Synthetic peptide
CASSPRSTQTQYF

SEQ ID NO: 462 - protein, 13 aa, Artificial Sequence
  Description of Artificial Sequence Synthetic peptide
CASSIFSRDTQYF

SEQ ID NO: 463 - protein, 13 aa, Artificial Sequence
  Description of Artificial Sequence Synthetic peptide
CATGKSPEETQYF

SEQ ID NO: 464 - protein, 13 aa, Artificial Sequence
  Description of Artificial Sequence Synthetic peptide
CASSPDGADEQFF

SEQ ID NO: 465 - protein, 13 aa, Artificial Sequence
  Description of Artificial Sequence Synthetic peptide
CTSSRKVQETQYF

SEQ ID NO: 466 - protein, 16 aa, Artificial Sequence
  Description of Artificial Sequence Synthetic peptide
CASRLTGGGLAYEQYF

SEQ ID NO: 467 - protein, 12 aa, Artificial Sequence
  Description of Artificial Sequence Synthetic peptide
TFLAPNAAEAFF

SEQ ID NO: 468 - protein, 12 aa, Artificial Sequence
  Description of Artificial Sequence Synthetic peptide
CASSFGGNEQFF

SEQ ID NO: 469 - protein, 15 aa, Artificial Sequence
  Description of Artificial Sequence Synthetic peptide
CASSPNRDTYRQYTF

SEQ ID NO: 470 - protein, 13 aa, Artificial Sequence
  Description of Artificial Sequence Synthetic peptide
CASSVGLGGELFF

SEQ ID NO: 471 - protein, 14 aa, Artificial Sequence
  Description of Artificial Sequence Synthetic peptide
CASSLTGGGTEAFF

SEQ ID NO: 472 - protein, 14 aa, Artificial Sequence
  Description of Artificial Sequence Synthetic peptide
CASSVAGVTDTQYF

SEQ ID NO: 473 - protein, 14 aa, Artificial Sequence
  Description of Artificial Sequence Synthetic peptide
CASRSTGGGTEAFF

SEQ ID NO: 474 - protein, 15 aa, Artificial Sequence
  Description of Artificial Sequence Synthetic peptide
CSAQKNTGGGEKLFF

SEQ ID NO: 475 - protein, 15 aa, Artificial Sequence
  Description of Artificial Sequence Synthetic peptide
CASSWEDRDADTQYF

SEQ ID NO: 476 - protein, 12 aa, Artificial Sequence
  Description of Artificial Sequence Synthetic peptide
CASSINSFEAFF

SEQ ID NO: 477 - protein, 19 aa, Artificial Sequence
  Description of Artificial Sequence Synthetic peptide
CSARYRDRGQGGSTDYQYF

SEQ ID NO: 478 - protein, 15 aa, Artificial Sequence
  Description of Artificial Sequence Synthetic peptide
CATSDLNQPNQPQHF

SEQ ID NO: 479 - protein, 13 aa, Artificial Sequence
  Description of Artificial Sequence Synthetic peptide
CAAGKDWSGELFF

SEQ ID NO: 480 - protein, 16 aa, Artificial Sequence
  Description of Artificial Sequence Synthetic peptide
CAWESSRDIDDPEAFF

SEQ ID NO: 481 - protein, 13 aa, Artificial Sequence
  Description of Artificial Sequence Synthetic peptide
CSARDRSDEKLFF

SEQ ID NO: 482 - protein, 18 aa, Artificial Sequence
  Description of Artificial Sequence Synthetic peptide
CASSLEGGLGSLNTEAFF

SEQ ID NO: 483 - protein, 13 aa, Artificial Sequence
  Description of Artificial Sequence Synthetic peptide
CSALDRGNEKLFF

SEQ ID NO: 484 - protein, 11 aa, Artificial Sequence
  Description of Artificial Sequence Synthetic peptide
CASSPGLAEYF

SEQ ID NO: 485 - protein, 17 aa, Artificial Sequence
  Description of Artificial Sequence Synthetic peptide
CASSLPGQGTHLETQYF

SEQ ID NO: 486 - protein, 16 aa, Artificial Sequence
  Description of Artificial Sequence Synthetic peptide
CASSFVLAISGNTIYF

SEQ ID NO: 487 - protein, 15 aa, Artificial Sequence
  Description of Artificial Sequence Synthetic peptide
CASSMDGTLLYEQYF

SEQ ID NO: 488 - protein, 15 aa, Artificial Sequence
  Description of Artificial Sequence Synthetic peptide
CASSLSSTGLQPQHF

SEQ ID NO: 489 - protein, 17 aa, Artificial Sequence
  Description of Artificial Sequence Synthetic peptide
CASSELAGQTYSYEQYF

SEQ ID NO: 490 - protein, 15 aa, Artificial Sequence
  Description of Artificial Sequence Synthetic peptide
CASSKGNRRSYGYTF

SEQ ID NO: 491 - protein, 12 aa, Artificial Sequence
  Description of Artificial Sequence Synthetic peptide
CATSAGSETQYF

SEQ ID NO: 492 - protein, 16 aa, Artificial Sequence
  Description of Artificial Sequence Synthetic peptide
CASSSDRAVGDTEAFF

SEQ ID NO: 493 - protein, 15 aa, Artificial Sequence
  Description of Artificial Sequence Synthetic peptide
CASSDNVQGIGGYTF

SEQ ID NO: 494 - protein, 14 aa, Artificial Sequence
  Description of Artificial Sequence Synthetic peptide
CASGGQGAGNTIYF

SEQ ID NO: 495 - protein, 14 aa, Artificial Sequence
  Description of Artificial Sequence Synthetic peptide
CASKTGGANTEAFF

SEQ ID NO: 496 - protein, 15 aa, Artificial Sequence
  Description of Artificial Sequence Synthetic peptide
CASSLLGQGSYGYTF

SEQ ID NO: 497 - protein, 16 aa, Artificial Sequence
  Description of Artificial Sequence Synthetic peptide
CASSQTGGGQGNRAFF

SEQ ID NO: 498 - protein, 12 aa, Artificial Sequence
  Description of Artificial Sequence Synthetic peptide
CASSPDVGRVYF

SEQ ID NO: 499 - protein, 13 aa, Artificial Sequence
  Description of Artificial Sequence Synthetic peptide
CASRTQGNTEAFF

SEQ ID NO: 500 - protein, 14 aa, Artificial Sequence
  Description of Artificial Sequence Synthetic peptide
CASSSPQGDYEQYF

SEQ ID NO: 501 - protein, 14 aa, Artificial Sequence
  Description of Artificial Sequence Synthetic peptide
CASRLGQTSYEQYF

SEQ ID NO: 502 - protein, 15 aa, Artificial Sequence
  Description of Artificial Sequence Synthetic peptide
CASSPSGGDTGELFF

SEQ ID NO: 503 - protein, 13 aa, Artificial Sequence
  Description of Artificial Sequence Synthetic peptide
CASSLDVAVEQYF

SEQ ID NO: 504 - protein, 13 aa, Artificial Sequence
  Description of Artificial Sequence Synthetic peptide
CASSPDDLYGYTF

SEQ ID NO: 505 - protein, 15 aa, Artificial Sequence
  Description of Artificial Sequence Synthetic peptide
CASSLVGDTYNEQFF

SEQ ID NO: 506 - protein, 14 aa, Artificial Sequence
  Description of Artificial Sequence Synthetic peptide
CASNAWGGNQPQHF

SEQ ID NO: 507 - protein, 16 aa, Artificial Sequence
  Description of Artificial Sequence Synthetic peptide
CASSDPDGGGTGELFF

SEQ ID NO: 508 - protein, 15 aa, Artificial Sequence
  Description of Artificial Sequence Synthetic peptide
CASSWSTGGDSPLHF

SEQ ID NO: 509 - protein, 13 aa, Artificial Sequence
  Description of Artificial Sequence Synthetic peptide
CASSLDLNTEAFF

SEQ ID NO: 510 - protein, 13 aa, Artificial Sequence
  Description of Artificial Sequence Synthetic peptide
CASSLDVNTEAFF

SEQ ID NO: 511 - protein, 13 aa, Artificial Sequence
  Description of Artificial Sequence Synthetic peptide
CASSFDLNTEAFF

SEQ ID NO: 512 - protein, 16 aa, Artificial Sequence
  Description of Artificial Sequence Synthetic peptide
CASSPRFRSPDSPLHF

SEQ ID NO: 513 - protein, 16 aa, Artificial Sequence
  Description of Artificial Sequence Synthetic peptide
CASSDPDGGDTGELFF

SEQ ID NO: 514 - protein, 13 aa, Artificial Sequence
  Description of Artificial Sequence Synthetic peptide
CSARDRSDEKLFF

SEQ ID NO: 515 - protein, 17 aa, Artificial Sequence
  Description of Artificial Sequence Synthetic peptide
CASSELAGDTYSYEQYF

SEQ ID NO: 516 - protein, 17 aa, Artificial Sequence
  Description of Artificial Sequence Synthetic peptide
CASSLPGQDTHLETQYF

SEQ ID NO: 517 - protein, 15 aa, Artificial Sequence
  Description of Artificial Sequence Synthetic peptide
CASSPNRDTYRGYTF

SEQ ID NO: 518 - protein, 12 aa, Artificial Sequence
  Description of Artificial Sequence Synthetic peptide
CASGEGNQPQHF

SEQ ID NO: 519 - protein, 10 aa, Artificial Sequence
  Description of Artificial Sequence Synthetic peptide
CSARDSGYTF

SEQ ID NO: 520 - protein, 19 aa, Artificial Sequence
  Description of Artificial Sequence Synthetic peptide
CSARYRDRGQGGSTDTQYF

SEQ ID NO: 521 - protein, 13 aa, Artificial Sequence
  Description of Artificial Sequence Synthetic peptide
CASSPRSTDTQYF

SEQ ID NO: 522 - protein, 15 aa, Artificial Sequence
  Description of Artificial Sequence Synthetic peptide
CASTFLAPNAAEAFF

SEQ ID NO: 523 - protein, 10 aa, Artificial Sequence
  Description of Artificial Sequence Synthetic peptide
CATTGGQNTE

SEQ ID NO: 524 - protein, 10 aa, Artificial Sequence
  Description of Artificial Sequence Synthetic peptide
CASSEVQGAS

SEQ ID NO: 525 - protein, 10 aa, Artificial Sequence
  Description of Artificial Sequence Synthetic peptide
CASQSGILPW

SEQ ID NO: 526 - protein, 10 aa, Artificial Sequence
  Description of Artificial Sequence Synthetic peptide
CAISEWRAGS

SEQ ID NO: 527 - protein, 10 aa, Artificial Sequence
  Description of Artificial Sequence Synthetic peptide
CASSLTSATG

SEQ ID NO: 528 - protein, 8 aa, Unknown
  Description of Unknown MANA restriction peptide
HVIENIYL

SEQ ID NO: 529 - protein, 9 aa, Unknown
  Description of Unknown MANA restriction peptide
DVAAHLQLL

SEQ ID NO: 530 - protein, 8 aa, Unknown
  Description of Unknown MANA restriction peptide
EAQQFLRY

SEQ ID NO: 531 - protein, 9 aa, Unknown
  Description of Unknown MANA restriction peptide
EAMQCSELY

SEQ ID NO: 532 - protein, 11 aa, Unknown
  Description of Unknown MANA restriction peptide
EAMQCSELYHM

SEQ ID NO: 533 - protein, 8 aa, Unknown
  Description of Unknown MANA restriction peptide
EAMQCSEL

SEQ ID NO: 534 - protein, 10 aa, Unknown
  Description of Unknown MANA restriction peptide
ITRTVSGNTV

SEQ ID NO: 535 - protein, 10 aa, Unknown
  Description of Unknown MANA restriction peptide
ATKNIKVIMA

SEQ ID NO: 536 - protein, 10 aa, Unknown
  Description of Unknown MANA restriction peptide
VAHFQRQMLK

SEQ ID NO: 537 - protein, 9 aa, Unknown
  Description of Unknown MANA restriction peptide
EEDTFSDLI

SEQ ID NO: 538 - protein, 8 aa, Unknown
  Description of Unknown MANA restriction peptide
AHFQRQML

SEQ ID NO: 539 - protein, 11 aa, Unknown
  Description of Unknown MANA restriction peptide
LQAMIQAAGKL

SEQ ID NO: 540 - protein, 11 aa, Unknown
  Description of Unknown MANA restriction peptide
LHEAHPWFEFL

SEQ ID NO: 541 - protein, 10 aa, Unknown
  Description of Unknown MANA restriction peptide
LHEAHPWFEF

SEQ ID NO: 542 - protein, 10 aa, Unknown
  Description of Unknown MANA restriction peptide
EHLSYPDNFL

SEQ ID NO: 543 - protein, 10 aa, Unknown
  Description of Unknown MANA restriction peptide
NHARIDAAEG

SEQ ID NO: 544 - protein, 9 aa, Unknown
  Description of Unknown MANA restriction peptide
LHQPNPFEV

SEQ ID NO: 545 - protein, 8 aa, Unknown
  Description of Unknown MANA restriction peptide
TQQEKEAL

SEQ ID NO: 546 - protein, 11 aa, Unknown
  Description of Unknown MANA restriction peptide
TKARNEYLLSL

SEQ ID NO: 547 - protein, 8 aa, Unknown
  Description of Unknown MANA restriction peptide
KPMWVVLL

SEQ ID NO: 548 - protein, 8 aa, Unknown
  Description of Unknown MANA restriction peptide
KHILVWAP

SEQ ID NO: 549 - protein, 8 aa, Unknown
  Description of Unknown MANA restriction peptide
SQSVYIPM

SEQ ID NO: 550 - protein, 8 aa, Unknown
  Description of Unknown MANA restriction peptide
VHDDFSVI

SEQ ID NO: 551 - protein, 9 aa, Unknown
  Description of Unknown MANA restriction peptide
IYLPAAQTM

SEQ ID NO: 552 - protein, 11 aa, Unknown
  Description of Unknown MANA restriction peptide
FSDLIWSNPRY

SEQ ID NO: 553 - protein, 10 aa, Unknown
  Description of Unknown MANA restriction peptide
YSWSAQRRAL

SEQ ID NO: 554 - protein, 10 aa, Unknown
  Description of Unknown MANA restriction peptide
FAVGTLAETI

SEQ ID NO: 555 - protein, 11 aa, Unknown
  Description of Unknown MANA restriction peptide
FASLALVRRYL

SEQ ID NO: 556 - protein, 11 aa, Unknown
  Description of Unknown MANA restriction peptide
DVLQQDELDSY

SEQ ID NO: 557 - protein, 10 aa, Unknown
  Description of Unknown MANA restriction peptide
KNHSSGTVSA

SEQ ID NO: 558 - protein, 9 aa, Unknown
  Description of Unknown MANA restriction peptide
KLKSFNLSA

SEQ ID NO: 559 - protein, 9 aa, Unknown
  Description of Unknown MANA restriction peptide
KSFAVGTLA

SEQ ID NO: 560 - protein, 9 aa, Unknown
  Description of Unknown MANA restriction peptide
KWRLSHCTV

SEQ ID NO: 561 - protein, 10 aa, Unknown
  Description of Unknown MANA restriction peptide
RSRPVAAKAK

SEQ ID NO: 562 - protein, 9 aa, Unknown
  Description of Unknown MANA restriction peptide
RSRPVAAKA

SEQ ID NO: 563 - protein, 11 aa, Unknown
  Description of Unknown MANA restriction peptide
KAKQAHLTTLK

SEQ ID NO: 564 - protein, 9 aa, Unknown
  Description of Unknown MANA restriction peptide
SRCPSAMGI

SEQ ID NO: 565 - protein, 8 aa, Unknown
  Description of Unknown MANA restriction peptide
FHASQGWL

SEQ ID NO: 566 - protein, 8 aa, Unknown
  Description of Unknown MANA restriction peptide
THEVIVPH

SEQ ID NO: 567 - protein, 8 aa, Unknown
  Description of Unknown MANA restriction peptide
GRHCLQPL

SEQ ID NO: 568 - protein, 10 aa, Unknown
  Description of Unknown MANA restriction peptide
FASLALVRRY

SEQ ID NO: 569 - protein, 11 aa, Unknown
  Description of Unknown MANA restriction peptide
SVFNTWKPMWV

SEQ ID NO: 570 - protein, 9 aa, Unknown
  Description of Unknown MANA restriction peptide
LTHEVIVPH

SEQ ID NO: 571 - protein, 10 aa, Unknown
  Description of Unknown MANA restriction peptide
VYRYGQKKPC

SEQ ID NO: 572 - protein, 9 aa, Unknown
  Description of Unknown MANA restriction peptide
LSEKGIEDD

SEQ ID NO: 573 - protein, 8 aa, Unknown
  Description of Unknown MANA restriction peptide
MSDVRRVF

SEQ ID NO: 574 - protein, 10 aa, Unknown
  Description of Unknown MANA restriction peptide
NSDEPLNLTF

SEQ ID NO: 575 - protein, 9 aa, Unknown
  Description of Unknown MANA restriction peptide
MSDVRRVFL

SEQ ID NO: 576 - protein, 10 aa, Unknown
  Description of Unknown MANA restriction peptide
ATDVNDALGY

SEQ ID NO: 577 - protein, 9 aa, Unknown
  Description of Unknown MANA restriction peptide
LLASGAPRY

SEQ ID NO: 578 - protein, 9 aa, Unknown
  Description of Unknown MANA restriction peptide
ALLAVIVLV

SEQ ID NO: 579 - protein, 11 aa, Unknown
  Description of Unknown MANA restriction peptide
ALLAVIVLVAL

SEQ ID NO: 580 - protein, 11 aa, Unknown
  Description of Unknown MANA restriction peptide
FLNGQEETAGV

SEQ ID NO: 581 - protein, 8 aa, Unknown
  Description of Unknown MANA restriction peptide
ALLAVIVL

SEQ ID NO: 582 - protein, 8 aa, Unknown
  Description of Unknown MANA restriction peptide
LLAVIVLV

SEQ ID NO: 583 - protein, 8 aa, Unknown
  Description of Unknown MANA restriction peptide
VLVALLAV

SEQ ID NO: 584 - protein, 10 aa, Unknown
  Description of Unknown MANA restriction peptide
MLAASAGEVV

SEQ ID NO: 585 - protein, 10 aa, Unknown
  Description of Unknown MANA restriction peptide
YPMYSGEKAY

SEQ ID NO: 586 - protein, 8 aa, Unknown
  Description of Unknown MANA restriction peptide
CPQTYRTS

SEQ ID NO: 587 - protein, 10 aa, Unknown
  Description of Unknown MANA restriction peptide
LPVAVLVALL

SEQ ID NO: 588 - protein, 8 aa, Unknown
  Description of Unknown MANA restriction peptide
MTSGDYAF

SEQ ID NO: 589 - protein, 8 aa, Unknown
  Description of Unknown MANA restriction peptide
LPTPTSPL

SEQ ID NO: 590 - protein, 10 aa, Unknown
  Description of Unknown MANA restriction peptide
YSMSDVRRVF

SEQ ID NO: 591 - protein, 11 aa, Unknown
  Description of Unknown MANA restriction peptide
DATDVNDALGY

SEQ ID NO: 592 - protein, 10 aa, Unknown
  Description of Unknown MANA restriction peptide
AEAGAGAASL

SEQ ID NO: 593 - protein, 10 aa, Unknown
  Description of Unknown MANA restriction peptide
AGAASLNASL

SEQ ID NO: 594 - protein, 11 aa, Unknown
  Description of Unknown MANA restriction peptide
LENCAEIMRLL

SEQ ID NO: 595 - protein, 10 aa, Unknown
  Description of Unknown MANA restriction peptide
AETQGRFQLL

SEQ ID NO: 596 - protein, 8 aa, Unknown
  Description of Unknown MANA restriction peptide
LENCAEIM

SEQ ID NO: 597 - protein, 10 aa, Unknown
  Description of Unknown MANA restriction peptide
LENCAEIMRL

SEQ ID NO: 598 - protein, 10 aa, Unknown
  Description of Unknown MANA restriction peptide
SENSDEPLNL

SEQ ID NO: 599 - protein, 10 aa, Unknown
  Description of Unknown MANA restriction peptide
AESEADRDSM

SEQ ID NO: 600 - protein, 11 aa, Unknown
  Description of Unknown MANA restriction peptide
YSMSDVRRVFL

SEQ ID NO: 601 - protein, 9 aa, Unknown
  Description of Unknown MANA restriction peptide
YSMSDVRRV

SEQ ID NO: 602 - protein, 9 aa, Unknown
  Description of Unknown MANA restriction peptide
FANGADVQV

SEQ ID NO: 603 - protein, 9 aa, Unknown
  Description of Unknown MANA restriction peptide
MPRRPSCPL

SEQ ID NO: 604 - protein, 10 aa, Unknown
  Description of Unknown MANA restriction peptide
TPLGQHLAAL

SEQ ID NO: 605 - protein, 8 aa, Unknown
  Description of Unknown MANA restriction peptide
SLHEFQLV

SEQ ID NO: 606 - protein, 11 aa, Unknown
  Description of Unknown MANA restriction peptide
QLVDLSRRFLV

SEQ ID NO: 607 - protein, 11 aa, Unknown
  Description of Unknown MANA restriction peptide
RLSDETLIDIM

SEQ ID NO: 608 - protein, 9 aa, Unknown
  Description of Unknown MANA restriction peptide
VLFDTQAPL

SEQ ID NO: 609 - protein, 11 aa, Unknown
  Description of Unknown MANA restriction peptide
VLFDTQAPLNA

SEQ ID NO: 610 - protein, 10 aa, Unknown
  Description of Unknown MANA restriction peptide
VMSAAAIYTV

SEQ ID NO: 611 - protein, 11 aa, Unknown
  Description of Unknown MANA restriction peptide
CVMSAAAIYTV

SEQ ID NO: 612 - protein, 10 aa, Unknown
  Description of Unknown MANA restriction peptide
ILAGLCVMSA

SEQ ID NO: 613 - protein, 9 aa, Unknown
  Description of Unknown MANA restriction peptide
LLLPELGSA

SEQ ID NO: 614 - protein, 8 aa, Unknown
  Description of Unknown MANA restriction peptide
KMALQQYL

SEQ ID NO: 615 - protein, 8 aa, Unknown
  Description of Unknown MANA restriction peptide
VMSAAAIY

SEQ ID NO: 616 - protein, 9 aa, Unknown
  Description of Unknown MANA restriction peptide
CVMSAAAIY

SEQ ID NO: 617 - protein, 11 aa, Unknown
  Description of Unknown MANA restriction peptide
LLLPELGSAFY

SEQ ID NO: 618 - protein, 10 aa, Unknown
  Description of Unknown MANA restriction peptide
HLGKPGHRSY

SEQ ID NO: 619 - protein, 8 aa, Unknown
  Description of Unknown MANA restriction peptide
MSLCVVLY

SEQ ID NO: 620 - protein, 9 aa, Unknown
  Description of Unknown MANA restriction peptide
DMSLCVVLY

SEQ ID NO: 621 - protein, 10 aa, Unknown
  Description of Unknown MANA restriction peptide
LDMSLCVVLY

SEQ ID NO: 622 - protein, 11 aa, Unknown
  Description of Unknown MANA restriction peptide
SLDMSLCVVLY

SEQ ID NO: 623 - protein, 11 aa, Unknown
  Description of Unknown MANA restriction peptide
TTYRGWLGLDY

SEQ ID NO: 624 - protein, 10 aa, Unknown
  Description of Unknown MANA restriction peptide
LLCQKFLARY

SEQ ID NO: 625 - protein, 11 aa, Unknown
  Description of Unknown MANA restriction peptide
APTGNFYPQPL

SEQ ID NO: 626 - protein, 10 aa, Unknown
  Description of Unknown MANA restriction peptide
YPQPLLNSSM

SEQ ID NO: 627 - protein, 9 aa, Unknown
  Description of Unknown MANA restriction peptide
TPNYSVSRL

SEQ ID NO: 628 - protein, 8 aa, Unknown
  Description of Unknown MANA restriction peptide
TPNYSVSR

SEQ ID NO: 629 - protein, 8 aa, Unknown
  Description of Unknown MANA restriction peptide
DPNGTSSL

SEQ ID NO: 630 - protein, 9 aa, Unknown
  Description of Unknown MANA restriction peptide
KPGHRSYAL

SEQ ID NO: 631 - protein, 10 aa, Unknown
  Description of Unknown MANA restriction peptide
YTRKLLGSAL

SEQ ID NO: 632 - protein, 9 aa, Unknown
  Description of Unknown MANA restriction peptide
GPAAYPIPV

SEQ ID NO: 633 - protein, 10 aa, Unknown
  Description of Unknown MANA restriction peptide
LPTESPHSSV

SEQ ID NO: 634 - protein, 11 aa, Unknown
  Description of Unknown MANA restriction peptide
IPHFTATADAF

SEQ ID NO: 635 - protein, 10 aa, Unknown
  Description of Unknown MANA restriction peptide
IEIKAVPEGF

SEQ ID NO: 636 - protein, 10 aa, Unknown
  Description of Unknown MANA restriction peptide
DEDSATETCY

SEQ ID NO: 637 - protein, 10 aa, Unknown
  Description of Unknown MANA restriction peptide
GELDKQLTTY

SEQ ID NO: 638 - protein, 9 aa, Unknown
  Description of Unknown MANA restriction peptide
AKLGQVLIY

SEQ ID NO: 639 - protein, 8 aa, Unknown
  Description of Unknown MANA restriction peptide
AKLGQVLI

SEQ ID NO: 640 - protein, 10 aa, Unknown
  Description of Unknown MANA restriction peptide
AKLGQVLIYL

SEQ ID NO: 641 - protein, 8 aa, Unknown
  Description of Unknown MANA restriction peptide
PELGSAFY

SEQ ID NO: 642 - protein, 10 aa, Unknown
  Description of Unknown MANA restriction peptide
EEFLNHSKAR

SEQ ID NO: 643 - protein, 11 aa, Unknown
  Description of Unknown MANA restriction peptide
TEEFLNHSKAR

SEQ ID NO: 644 - protein, 10 aa, Unknown
  Description of Unknown MANA restriction peptide
IKNLHDDSCY

SEQ ID NO: 645 - protein, 10 aa, Unknown
  Description of Unknown MANA restriction peptide
SRMQRGGLRL

SEQ ID NO: 646 - protein, 11 aa, Unknown
  Description of Unknown MANA restriction peptide
MYHSRMQRGGL

SEQ ID NO: 647 - protein, 8 aa, Unknown
  Description of Unknown MANA restriction peptide
FFYTRKLL

SEQ ID NO: 648 - protein, 11 aa, Unknown
  Description of Unknown MANA restriction peptide
SRLQILRQVSL

SEQ ID NO: 649 - protein, 8 aa, Unknown
  Description of Unknown MANA restriction peptide
FYYTGVGV

SEQ ID NO: 650 - protein, 9 aa, Unknown
  Description of Unknown MANA restriction peptide
FYYTGVGVL

SEQ ID NO: 651 - protein, 10 aa, Unknown
  Description of Unknown MANA restriction peptide
FYYTGVGVLI

SEQ ID NO: 652 - protein, 8 aa, Unknown
  Description of Unknown MANA restriction peptide
YYTGVGVL

SEQ ID NO: 653 - protein, 9 aa, Unknown
  Description of Unknown MANA restriction peptide
YYTGVGVLI

SEQ ID NO: 654 - protein, 9 aa, Unknown
  Description of Unknown MANA restriction peptide
FYPATFGIV

SEQ ID NO: 655 - protein, 11 aa, Unknown
  Description of Unknown MANA restriction peptide
YAPPQDGPAAY

SEQ ID NO: 656 - protein, 8 aa, Unknown
  Description of Unknown MANA restriction peptide
FTATADAF

SEQ ID NO: 657 - protein, 11 aa, Unknown
  Description of Unknown MANA restriction peptide
FQMDDYSLCGL

SEQ ID NO: 658 - protein, 10 aa, Unknown
  Description of Unknown MANA restriction peptide
MTFSNPPDWS

SEQ ID NO: 659 - protein, 10 aa, Unknown
  Description of Unknown MANA restriction peptide
FAAHLLDIAM

SEQ ID NO: 660 - protein, 8 aa, Unknown
  Description of Unknown MANA restriction peptide
FFFAAHLL

SEQ ID NO: 661 - protein, 11 aa, Unknown
  Description of Unknown MANA restriction peptide
FFAAHLLDIAM

SEQ ID NO: 662 - protein, 9 aa, Unknown
  Description of Unknown MANA restriction peptide
FMDSYTMRF

SEQ ID NO: 663 - protein, 10 aa, Unknown
  Description of Unknown MANA restriction peptide
ESMERDCATF

SEQ ID NO: 664 - protein, 8 aa, Unknown
  Description of Unknown MANA restriction peptide
EKDLAEDA

SEQ ID NO: 665 - protein, 10 aa, Unknown
  Description of Unknown MANA restriction peptide
EKDLAEDAPW

SEQ ID NO: 666 - protein, 11 aa, Unknown
  Description of Unknown MANA restriction peptide
EKDLAEDAPWK

SEQ ID NO: 667 - protein, 10 aa, Unknown
  Description of Unknown MANA restriction peptide
AEDAPWKKIQ

SEQ ID NO: 668 - protein, 8 aa, Unknown
  Description of Unknown MANA restriction peptide
KKIQQNTF

SEQ ID NO: 669 - protein, 10 aa, Unknown
  Description of Unknown MANA restriction peptide
VMYDGFSMQR

SEQ ID NO: 670 - protein, 9 aa, Unknown
  Description of Unknown MANA restriction peptide
CVKVCAYVR

SEQ ID NO: 671 - protein, 10 aa, Unknown
  Description of Unknown MANA restriction peptide
KSTSISTAKR

SEQ ID NO: 672 - protein, 8 aa, Unknown
  Description of Unknown MANA restriction peptide
GASSIWYT

SEQ ID NO: 673 - protein, 9 aa, Unknown
  Description of Unknown MANA restriction peptide
AGASSIWYT

SEQ ID NO: 674 - protein, 11 aa, Unknown
  Description of Unknown MANA restriction peptide
YVMYDGFSMQR

SEQ ID NO: 675 - protein, 11 aa, Unknown
  Description of Unknown MANA restriction peptide
ELFLVKAKIYK

SEQ ID NO: 676 - protein, 9 aa, Unknown
  Description of Unknown MANA restriction peptide
STSISTAKR

SEQ ID NO: 677 - protein, 8 aa, Unknown
  Description of Unknown MANA restriction peptide
TSISTAKR

SEQ ID NO: 678 - protein, 10 aa, Unknown
  Description of Unknown MANA restriction peptide
FIFTSMAGIR

SEQ ID NO: 679 - protein, 8 aa, Unknown
  Description of Unknown MANA restriction peptide
FTSMAGIR

SEQ ID NO: 680 - protein, 11 aa, Unknown
  Description of Unknown MANA restriction peptide
FTNRKVPYCVK

SEQ ID NO: 681 - protein, 9 aa, Unknown
  Description of Unknown MANA restriction peptide
EACHQSCFR

SEQ ID NO: 682 - protein, 10 aa, Unknown
  Description of Unknown MANA restriction peptide
HPNVILNPLY

SEQ ID NO: 683 - protein, 8 aa, Unknown
  Description of Unknown MANA restriction peptide
FPNVVSGS

SEQ ID NO: 684 - protein, 11 aa, Unknown
  Description of Unknown MANA restriction peptide
MAENTEGDLNS

SEQ ID NO: 685 - protein, 9 aa, Unknown
  Description of Unknown MANA restriction peptide
MLVELAPPY

SEQ ID NO: 686 - protein, 10 aa, Unknown
  Description of Unknown MANA restriction peptide
EPSNVTETLM

SEQ ID NO: 687 - protein, 9 aa, Unknown
  Description of Unknown MANA restriction peptide
EPSNVTETL

SEQ ID NO: 688 - protein, 11 aa, Unknown
  Description of Unknown MANA restriction peptide
SPAMTSTSFFS

SEQ ID NO: 689 - protein, 8 aa, Unknown
  Description of Unknown MANA restriction peptide
MTSTSFFS

SEQ ID NO: 690 - protein, 8 aa, Unknown
  Description of Unknown MANA restriction peptide
MAIADILF

SEQ ID NO: 691 - protein, 8 aa, Unknown
  Description of Unknown MANA restriction peptide
IPEKLEYF

SEQ ID NO: 692 - protein, 10 aa, Unknown
  Description of Unknown MANA restriction peptide
MPIYPTYNEV

SEQ ID NO: 693 - protein, 11 aa, Unknown
  Description of Unknown MANA restriction peptide
CAYVRKQVEKI

SEQ ID NO: 694 - protein, 9 aa, Unknown
  Description of Unknown MANA restriction peptide
LAQEETTVI

SEQ ID NO: 695 - protein, 11 aa, Unknown
  Description of Unknown MANA restriction peptide
HPNVILNPLYV

SEQ ID NO: 696 - protein, 10 aa, Unknown
  Description of Unknown MANA restriction peptide
LPDHFGPGPV

SEQ ID NO: 697 - protein, 9 aa, Unknown
  Description of Unknown MANA restriction peptide
MAIADILFV

SEQ ID NO: 698 - protein, 9 aa, Unknown
  Description of Unknown MANA restriction peptide
IPEKLEYFI

SEQ ID NO: 699 - protein, 10 aa, Unknown
  Description of Unknown MANA restriction peptide
EAQNFIDSLI

SEQ ID NO: 700 - protein, 9 aa, Unknown
  Description of Unknown MANA restriction peptide
YPTYNEVHL

SEQ ID NO: 701 - protein, 10 aa, Unknown
  Description of Unknown MANA restriction peptide
MYDGFSMQRL

SEQ ID NO: 702 - protein, 11 aa, Unknown
  Description of Unknown MANA restriction peptide
MYDGFSMQRLV

SEQ ID NO: 703 - protein, 10 aa, Unknown
  Description of Unknown MANA restriction peptide
AYDASTFRGR

SEQ ID NO: 704 - protein, 9 aa, Unknown
  Description of Unknown MANA restriction peptide
VTDCGRPPL

SEQ ID NO: 705 - protein, 8 aa, Unknown
  Description of Unknown MANA restriction peptide
EVRLVCKV

SEQ ID NO: 706 - protein, 9 aa, Unknown
  Description of Unknown MANA restriction peptide
STDLIAQSI

SEQ ID NO: 707 - protein, 11 aa, Unknown
  Description of Unknown MANA restriction peptide
KSTSISTAKRL

SEQ ID NO: 708 - protein, 9 aa, Unknown
  Description of Unknown MANA restriction peptide
QMFGKMFFV

SEQ ID NO: 709 - protein, 11 aa, Unknown
  Description of Unknown MANA restriction peptide
WAYDASTFRGR

SEQ ID NO: 710 - protein, 9 aa, Unknown
  Description of Unknown MANA restriction peptide
STPSTGGFS

SEQ ID NO: 711 - protein, 10 aa, Unknown
  Description of Unknown MANA restriction peptide
RAAPRAGPSV

SEQ ID NO: 712 - protein, 10 aa, Unknown
  Description of Unknown MANA restriction peptide
MTSTSFFSTL

SEQ ID NO: 713 - protein, 9 aa, Unknown
  Description of Unknown MANA restriction peptide
RSAEAQNFI

SEQ ID NO: 714 - protein, 9 aa, Unknown
  Description of Unknown MANA restriction peptide
LTSSDDMLI

SEQ ID NO: 715 - protein, 11 aa, Unknown
  Description of Unknown MANA restriction peptide
KYMMNSVLENF

SEQ ID NO: 716 - protein, 10 aa, Unknown
  Description of Unknown MANA restriction peptide
YMMNSVLENF

SEQ ID NO: 717 - protein, 9 aa, Unknown
  Description of Unknown MANA restriction peptide
GHACAEPSF

SEQ ID NO: 718 - protein, 11 aa, Unknown
  Description of Unknown MANA restriction peptide
FFAAQAGARKI

SEQ ID NO: 719 - protein, 10 aa, Unknown
  Description of Unknown MANA restriction peptide
SFFAAQAGAR

SEQ ID NO: 720 - protein, 9 aa, Unknown
  Description of Unknown MANA restriction peptide
YMIKAKSQF

SEQ ID NO: 721 - protein, 8 aa, Unknown
  Description of Unknown MANA restriction peptide
RYFDPKML

SEQ ID NO: 722 - protein, 9 aa, Unknown
  Description of Unknown MANA restriction peptide
ATLNGLMYF

SEQ ID NO: 723 - protein, 8 aa, Unknown
  Description of Unknown MANA restriction peptide
YTISFLFR

SEQ ID NO: 724 - protein, 9 aa, Unknown
  Description of Unknown MANA restriction peptide
DYTISFLFR

SEQ ID NO: 725 - protein, 8 aa, Unknown
  Description of Unknown MANA restriction peptide
KYMMNSVL

SEQ ID NO: 726 - protein, 8 aa, Unknown
  Description of Unknown MANA restriction peptide
RYPAKLTL

SEQ ID NO: 727 - protein, 9 aa, Unknown
  Description of Unknown MANA restriction peptide
FFAAQAGAR

SEQ ID NO: 728 - protein, 10 aa, Unknown
  Description of Unknown MANA restriction peptide
EYMIKAKSQF

SEQ ID NO: 729 - protein, 9 aa, Unknown
  Description of Unknown MANA restriction peptide
KEAFGPQAL

SEQ ID NO: 730 - protein, 9 aa, Unknown
  Description of Unknown MANA restriction peptide
CEVAPNDVV

SEQ ID NO: 731 - protein, 11 aa, Unknown
  Description of Unknown MANA restriction peptide
KEMHPNKLKAV

SEQ ID NO: 732 - protein, 8 aa, Unknown
  Description of Unknown MANA restriction peptide
CEVAPNDV

SEQ ID NO: 733 - protein, 8 aa, Unknown
  Description of Unknown MANA restriction peptide
KQFFYNLI

SEQ ID NO: 734 - protein, 8 aa, Unknown
  Description of Unknown MANA restriction peptide
TQLQGLQL

SEQ ID NO: 735 - protein, 11 aa, Unknown
  Description of Unknown MANA restriction peptide
TEYKLVVVGAG

SEQ ID NO: 736 - protein, 8 aa, Unknown
  Description of Unknown MANA restriction peptide
FEDGPYEV

SEQ ID NO: 737 - protein, 8 aa, Unknown
  Description of Unknown MANA restriction peptide
AQAGARKI

SEQ ID NO: 738 - protein, 11 aa, Unknown
  Description of Unknown MANA restriction peptide
AQAGARKIYAV

SEQ ID NO: 739 - protein, 9 aa, Unknown
  Description of Unknown MANA restriction peptide
AERLVGPGH

SEQ ID NO: 740 - protein, 11 aa, Unknown
  Description of Unknown MANA restriction peptide
IEYMIKAKSQF

SEQ ID NO: 741 - protein, 10 aa, Unknown
  Description of Unknown MANA restriction peptide
SDYTISFLFR

SEQ ID NO: 742 - protein, 11 aa, Unknown
  Description of Unknown MANA restriction peptide
GELGWENPNEW

SEQ ID NO: 743 - protein, 10 aa, Unknown
  Description of Unknown MANA restriction peptide
SEMMAVTQKI

SEQ ID NO: 744 - protein, 11 aa, Unknown
  Description of Unknown MANA restriction peptide
SEMMAVTQKIV

SEQ ID NO: 745 - protein, 9 aa, Unknown
  Description of Unknown MANA restriction peptide
AQAGARKIY

SEQ ID NO: 746 - protein, 8 aa, Unknown
  Description of Unknown MANA restriction peptide
NQMDMNQW

SEQ ID NO: 747 - protein, 9 aa, Unknown
  Description of Unknown MANA restriction peptide
DTSSEVSTV

SEQ ID NO: 748 - protein, 11 aa, Unknown
  Description of Unknown MANA restriction peptide
DTSSEVSTVEL

SEQ ID NO: 749 - protein, 9 aa, Unknown
  Description of Unknown MANA restriction peptide
YSPDILPMV

SEQ ID NO: 750 - protein, 11 aa, Unknown
  Description of Unknown MANA restriction peptide
FAAQAGARKIY

SEQ ID NO: 751 - protein, 10 aa, Unknown
  Description of Unknown MANA restriction peptide
FAAQAGARKI

SEQ ID NO: 752 - protein, 8 aa, Unknown
  Description of Unknown MANA restriction peptide
FAAQAGAR

SEQ ID NO: 753 - protein, 11 aa, Unknown
  Description of Unknown MANA restriction peptide
KTATLNGLMYF

SEQ ID NO: 754 - protein, 9 aa, Unknown
  Description of Unknown MANA restriction peptide
KTATLNGLM

SEQ ID NO: 755 - protein, 10 aa, Unknown
  Description of Unknown MANA restriction peptide
YTISFLFRIL

SEQ ID NO: 756 - protein, 9 aa, Unknown
  Description of Unknown MANA restriction peptide
YTISFLFRI

SEQ ID NO: 757 - protein, 9 aa, Unknown
  Description of Unknown MANA restriction peptide
IALSPSGTM

SEQ ID NO: 758 - protein, 10 aa, Unknown
  Description of Unknown MANA restriction peptide
IALSPSGTML

SEQ ID NO: 759 - protein, 9 aa, Unknown
  Description of Unknown MANA restriction peptide
FAVEAHQWI

SEQ ID NO: 760 - protein, 8 aa, Unknown
  Description of Unknown MANA restriction peptide
MTSLPCPL

SEQ ID NO: 761 - protein, 8 aa, Unknown
  Description of Unknown MANA restriction peptide
HACAEPSF

SEQ ID NO: 762 - protein, 8 aa, Unknown
  Description of Unknown MANA restriction peptide
IVDPDPLL

SEQ ID NO: 763 - protein, 10 aa, Unknown
  Description of Unknown MANA restriction peptide
RAPKEKAQPL

SEQ ID NO: 764 - protein, 9 aa, Unknown
  Description of Unknown MANA restriction peptide
YLNSRQFPT

SEQ ID NO: 765 - protein, 9 aa, Unknown
  Description of Unknown MANA restriction peptide
SIMAQSTSI

SEQ ID NO: 766 - protein, 8 aa, Unknown
  Description of Unknown MANA restriction peptide
SLDRHLYT

SEQ ID NO: 767 - protein, 10 aa, Unknown
  Description of Unknown MANA restriction peptide
FLISYWGEQI

SEQ ID NO: 768 - protein, 8 aa, Unknown
  Description of Unknown MANA restriction peptide
TINVEFAK

SEQ ID NO: 769 - protein, 11 aa, Unknown
  Description of Unknown MANA restriction peptide
EGSRISAASVA

SEQ ID NO: 770 - protein, 11 aa, Unknown
  Description of Unknown MANA restriction peptide
MLLVIIVSVGV

SEQ ID NO: 771 - protein, 9 aa, Unknown
  Description of Unknown MANA restriction peptide
SQHQVLFLL

SEQ ID NO: 772 - protein, 9 aa, Unknown
  Description of Unknown MANA restriction peptide
RLRTDLFPK

SEQ ID NO: 773 - protein, 8 aa, Unknown
  Description of Unknown MANA restriction peptide
KQRTSSKK

SEQ ID NO: 774 - protein, 10 aa, Unknown
  Description of Unknown MANA restriction peptide
RLKDNLQGYK

SEQ ID NO: 775 - protein, 8 aa, Unknown
  Description of Unknown MANA restriction peptide
SEEPPVDY

SEQ ID NO: 776 - protein, 8 aa, Unknown
  Description of Unknown MANA restriction peptide
QQDEGYGN

SEQ ID NO: 777 - protein, 8 aa, Unknown
  Description of Unknown MANA restriction peptide
LTRPNCMF

SEQ ID NO: 778 - protein, 11 aa, Unknown
  Description of Unknown MANA restriction peptide
PARASPALASL

SEQ ID NO: 779 - protein, 10 aa, Unknown
  Description of Unknown MANA restriction peptide
LRNGALTRPN

SEQ ID NO: 780 - protein, 11 aa, Unknown
  Description of Unknown MANA restriction peptide
VLRNGALTRPN

SEQ ID NO: 781 - protein, 11 aa, Unknown
  Description of Unknown MANA restriction peptide
LRNGALTRPNC

SEQ ID NO: 782 - protein, 8 aa, Unknown
  Description of Unknown MANA restriction peptide
PARASPAL

SEQ ID NO: 783 - protein, 9 aa, Unknown
  Description of Unknown MANA restriction peptide
SPARASPAL

SEQ ID NO: 784 - protein, 9 aa, Unknown
  Description of Unknown MANA restriction peptide
LRSLTFSLA

SEQ ID NO: 785 - protein, 10 aa, Unknown
  Description of Unknown MANA restriction peptide
SAITSKVSTI

SEQ ID NO: 786 - protein, 9 aa, Unknown
  Description of Unknown MANA restriction peptide
AITSKVSTI

SEQ ID NO: 787 - protein, 9 aa, Unknown
  Description of Unknown MANA restriction peptide
ASPARASPA

SEQ ID NO: 788 - protein, 8 aa, Unknown
  Description of Unknown MANA restriction peptide
SPARASPA

SEQ ID NO: 789 - protein, 10 aa, Unknown
  Description of Unknown MANA restriction peptide
ASPARASPAL

SEQ ID NO: 790 - protein, 10 aa, Unknown
  Description of Unknown MANA restriction peptide
QASPARASPA

SEQ ID NO: 791 - protein, 9 aa, Unknown
  Description of Unknown MANA restriction peptide
PARASPALA

SEQ ID NO: 792 - protein, 10 aa, Unknown
  Description of Unknown MANA restriction peptide
KLRSLTFSLA

SEQ ID NO: 793 - protein, 9 aa, Unknown
  Description of Unknown MANA restriction peptide
LFADATVEL

SEQ ID NO: 794 - protein, 11 aa, Unknown
  Description of Unknown MANA restriction peptide
LFADATVELSL

SEQ ID NO: 795 - protein, 8 aa, Unknown
  Description of Unknown MANA restriction peptide
HMASSPAV

SEQ ID NO: 796 - protein, 8 aa, Unknown
  Description of Unknown MANA restriction peptide
YLDSIVVL

SEQ ID NO: 797 - protein, 8 aa, Unknown
  Description of Unknown MANA restriction peptide
VLEDLSPL

SEQ ID NO: 798 - protein, 11 aa, Unknown
  Description of Unknown MANA restriction peptide
YLDSIVVLEDL

SEQ ID NO: 799 - protein, 10 aa, Unknown
  Description of Unknown MANA restriction peptide
VLEDLSPLEA

SEQ ID NO: 800 - protein, 10 aa, Unknown
  Description of Unknown MANA restriction peptide
LLLHGAEPNC

SEQ ID NO: 801 - protein, 8 aa, Unknown
  Description of Unknown MANA restriction peptide
ILIDVPKV

SEQ ID NO: 802 - protein, 8 aa, Unknown
  Description of Unknown MANA restriction peptide
AMACINLA

SEQ ID NO: 803 - protein, 10 aa, Unknown
  Description of Unknown MANA restriction peptide
LSKDIMVHFK

SEQ ID NO: 804 - protein, 11 aa, Unknown
  Description of Unknown MANA restriction peptide
VAMACINLASK

SEQ ID NO: 805 - protein, 10 aa, Unknown
  Description of Unknown MANA restriction peptide
AMACINLASK

SEQ ID NO: 806 - protein, 10 aa, Unknown
  Description of Unknown MANA restriction peptide
MSYDNNPFIK

SEQ ID NO: 807 - protein, 9 aa, Unknown
  Description of Unknown MANA restriction peptide
KTWKERTLK

SEQ ID NO: 808 - protein, 8 aa, Unknown
  Description of Unknown MANA restriction peptide
VILIDVPK

SEQ ID NO: 809 - protein, 8 aa, Unknown
  Description of Unknown MANA restriction peptide
MPLVHMAS

SEQ ID NO: 810 - protein, 11 aa, Unknown
  Description of Unknown MANA restriction peptide
MPLVHMASSPA

SEQ ID NO: 811 - protein, 10 aa, Unknown
  Description of Unknown MANA restriction peptide
YPDYLDSIVV

SEQ ID NO: 812 - protein, 11 aa, Unknown
  Description of Unknown MANA restriction peptide
YPDYLDSIVVL

SEQ ID NO: 813 - protein, 8 aa, Unknown
  Description of Unknown MANA restriction peptide
MSYDNNPF

SEQ ID NO: 814 - protein, 9 aa, Unknown
  Description of Unknown MANA restriction peptide
MASSPAVDV

SEQ ID NO: 815 - protein, 8 aa, Unknown
  Description of Unknown MANA restriction peptide
AVQRLRPK

SEQ ID NO: 816 - protein, 11 aa, Unknown
  Description of Unknown MANA restriction peptide
HVMPDTPDILQ

SEQ ID NO: 817 - protein, 8 aa, Unknown
  Description of Unknown MANA restriction peptide
KVMSILFY

SEQ ID NO: 818 - protein, 11 aa, Unknown
  Description of Unknown MANA restriction peptide
VQNAVQRLRPK

SEQ ID NO: 819 - protein, 9 aa, Unknown
  Description of Unknown MANA restriction peptide
VQNAVQRLR

SEQ ID NO: 820 - protein, 11 aa, Unknown
  Description of Unknown MANA restriction peptide
TLFEIIYDNLR

SEQ ID NO: 821 - protein, 8 aa, Unknown
  Description of Unknown MANA restriction peptide
SLASLHPR

SEQ ID NO: 822 - protein, 10 aa, Unknown
  Description of Unknown MANA restriction peptide
RSLGSLASLH

SEQ ID NO: 823 - protein, 11 aa, Unknown
  Description of Unknown MANA restriction peptide
KLLHEYWMALR

SEQ ID NO: 824 - protein, 10 aa, Unknown
  Description of Unknown MANA restriction peptide
LLHEYWMALR

SEQ ID NO: 825 - protein, 9 aa, Unknown
  Description of Unknown MANA restriction peptide
EVKEEDETF

SEQ ID NO: 826 - protein, 11 aa, Unknown
  Description of Unknown MANA restriction peptide
QVNKVMSILFY

SEQ ID NO: 827 - protein, 9 aa, Unknown
  Description of Unknown MANA restriction peptide
EVQNAVQRL

SEQ ID NO: 828 - protein, 8 aa, Unknown
  Description of Unknown MANA restriction peptide
LHEYWMAL

SEQ ID NO: 829 - protein, 11 aa, Unknown
  Description of Unknown MANA restriction peptide
YKLLHEYWMAL

SEQ ID NO: 830 - protein, 9 aa, Unknown
  Description of Unknown MANA restriction peptide
MEKSNNSTL

SEQ ID NO: 831 - protein, 11 aa, Unknown
  Description of Unknown MANA restriction peptide
MEKSNNSTLFI

SEQ ID NO: 832 - protein, 18 aa, Artificial Sequence
  Description of Artificial Sequence Synthetic peptide
CASSLSTGGPRVNSPLHF

SEQ ID NO: 833 - protein, 12 aa, Artificial Sequence
  Description of Artificial Sequence Synthetic peptide
CASNGEAETQYF

SEQ ID NO: 834 - protein, 13 aa, Artificial Sequence
  Description of Artificial Sequence Synthetic peptide
CASSLLENQPQHF

SEQ ID NO: 835 - protein, 15 aa, Artificial Sequence
  Description of Artificial Sequence Synthetic peptide
CASSPFRSPDSPLHF

SEQ ID NO: 836 - protein, 14 aa, Artificial Sequence
  Description of Artificial Sequence Synthetic peptide
CASSLGGSFYGYTF

SEQ ID NO: 837 - protein, 14 aa, Artificial Sequence
  Description of Artificial Sequence Synthetic peptide
CAWEGQGPYYGYTF

SEQ ID NO: 838 - DNA, 54 bp, Artificial Sequence
  Description of Artificial Sequence Synthetic oligonucleotide
tgtgccatcagtgagtggcgagcgggcagcacagatacgcagtattttggccca

SEQ ID NO: 839 - DNA, 51 bp, Artificial Sequence
  Description of Artificial Sequence Synthetic oligonucleotide
tgtgccatcagtggggggacaggggctaactatggctacaccttcggttcg

SEQ ID NO: 840 - DNA, 48 bp, Artificial Sequence
  Description of Artificial Sequence Synthetic oligonucleotide
tgtgccagcatcccgacagggcagaactatggctacaccttcggttcg

SEQ ID NO: 841 - DNA, 58 bp, Artificial Sequence
  Description of Artificial Sequence Synthetic oligonucleotide
tgtgtatttctgcgccagccaaagcggaatactcccatgggagcagttcttcgggcca

SEQ ID NO: 842 - DNA, 51 bp, Artificial Sequence
  Description of Artificial Sequence Synthetic oligonucleotide
tgtgccagcagacccggacagcgctataattcacccctccactttgggaac

SEQ ID NO: 843 - DNA, 45 bp, Artificial Sequence
  Description of Artificial Sequence Synthetic oligonucleotide
tgtgccagcaggccttatgggacctatggctacaccttcggttcg

SEQ ID NO: 844 - DNA, 51 bp, Artificial Sequence
  Description of Artificial Sequence Synthetic oligonucleotide
tgtgccagcagtgatttagcagggctcaactatggctacaccttcggttcg

SEQ ID NO: 845 - DNA, 51 bp, Artificial Sequence
  Description of Artificial Sequence Synthetic oligonucleotide
tgtgccagcagtgaagcgctcggtccaggaaacaccatatattttggagag

SEQ ID NO: 846 - DNA, 48 bp, Artificial Sequence
  Description of Artificial Sequence Synthetic oligonucleotide
tgtgccagcagcgaacaggggttttggaatggctacaccttcggttcg

SEQ ID NO: 847 - DNA, 54 bp, Artificial Sequence
  Description of Artificial Sequence Synthetic oligonucleotide
tgtgccagcagtgaggtacagggggcgtccaatgaaaaactgttttttggcagt

SEQ ID NO: 848 - DNA, 51 bp, Artificial Sequence
  Description of Artificial Sequence Synthetic oligonucleotide
tgtgccagcagtttcgcagggggaggcaatcagccccagcattttggtgat

SEQ ID NO: 849 - DNA, 45 bp, Artificial Sequence
  Description of Artificial Sequence Synthetic oligonucleotide
tgtgccagcagttttcagcagaacactgaagctttctttggacaa

SEQ ID NO: 850 - DNA, 45 bp, Artificial Sequence
  Description of Artificial Sequence Synthetic oligonucleotide
tgtgccagcagcttttggggcaacactgaagctttctttggacaa

SEQ ID NO: 851 - DNA, 48 bp, Artificial Sequence
  Description of Artificial Sequence Synthetic oligonucleotide
tgtgccagtagtatagggacagggagtaagccccagcattttggtgat

SEQ ID NO: 852 - DNA, 63 bp, Artificial Sequence
  Description of Artificial Sequence Synthetic oligonucleotide
tgtgccagcagcttagccggactagcgggacccctagccaaaaacattcagtacttcggcgcc

SEQ ID NO: 853 - DNA, 45 bp, Artificial Sequence
  Description of Artificial Sequence Synthetic oligonucleotide
tgtgccagcagcttagcgccaggcaatgagcagttcttcgggcca

SEQ ID NO: 854 - DNA, 51 bp, Artificial Sequence
  Description of Artificial Sequence Synthetic oligonucleotide
tgtgccagcagcttagagacaactggggccaacgtcctgactttcggggcc

SEQ ID NO: 855 - DNA, 48 bp, Artificial Sequence
  Description of Artificial Sequence Synthetic oligonucleotide
tgtgccagcagcttgggtgcgaacaccggggagctgttttttggagaa

SEQ ID NO: 856 - DNA, 54 bp, Artificial Sequence
  Description of Artificial Sequence Synthetic oligonucleotide
tgtgccagcagcctcggggacgattctatgaacactgaagctttctttggacaa

SEQ ID NO: 857 - DNA, 48 bp, Artificial Sequence
  Description of Artificial Sequence Synthetic oligonucleotide
tgtgccagcagtttaccaggggacccctacgagcagtacttcgggccg

SEQ ID NO: 858 - DNA, 51 bp, Artificial Sequence
  Description of Artificial Sequence Synthetic oligonucleotide
tgtgccagcagcttatcaggtccggtcgaggacgagcagtacttcgggccg

SEQ ID NO: 859 - DNA, 51 bp, Artificial Sequence
  Description of Artificial Sequence Synthetic oligonucleotide
tgtgccagcagcctaagcgggagctcctacaatgagcagttcttcgggcca

SEQ ID NO: 860 - DNA, 45 bp, Artificial Sequence
  Description of Artificial Sequence Synthetic oligonucleotide
tgtgccagcagtttgacagggggctctgaagctttctttggacaa

SEQ ID NO: 861 - DNA, 42 bp, Artificial Sequence
  Description of Artificial Sequence Synthetic oligonucleotide
tgcgccagcagcttgtacaccggggagctgttttttggagaa

SEQ ID NO: 862 - DNA, 54 bp, Artificial Sequence
  Description of Artificial Sequence Synthetic oligonucleotide
tgtgccagcagtccacttcctagcggggggacctacgagcagtacttcgggccg

SEQ ID NO: 863 - DNA, 54 bp, Artificial Sequence
  Description of Artificial Sequence Synthetic oligonucleotide
tgcgccagcagccgcggggagggggggagcaatcagccccagcattttggtgat

SEQ ID NO: 864 - DNA, 51 bp, Artificial Sequence
  Description of Artificial Sequence Synthetic oligonucleotide
tgtgccagtagtcgatcggggagctcctacaatgagcagttcttcgggcca

SEQ ID NO: 865 - DNA, 45 bp, Artificial Sequence
  Description of Artificial Sequence Synthetic oligonucleotide
tgtgccagcagttcgggacttcgaaacattcagtacttcggcgcc

SEQ ID NO: 866 - DNA, 45 bp, Artificial Sequence
  Description of Artificial Sequence Synthetic oligonucleotide
tgtgccagcagctcgggaccaagctacgagcagtacttcgggccg

SEQ ID NO: 867 - DNA, 45 bp, Artificial Sequence
  Description of Artificial Sequence Synthetic oligonucleotide
tgtgccagcagttccatatctaccggggagctgttttttggagaa

SEQ ID NO: 868 - DNA, 51 bp, Artificial Sequence
  Description of Artificial Sequence Synthetic oligonucleotide
tgtgccagcagcgtagatggggggcagagcgacgagcagtacttcgggccg

SEQ ID NO: 869 - DNA, 45 bp, Artificial Sequence
  Description of Artificial Sequence Synthetic oligonucleotide
tgtgccagcagcgtagattggggtgctgaagctttctttggacaa

SEQ ID NO: 870 - DNA, 45 bp, Artificial Sequence
  Description of Artificial Sequence Synthetic oligonucleotide
tgcgccagcagctggacaggcaatcagccccagcattttggtgat

SEQ ID NO: 871 - DNA, 57 bp, Artificial Sequence
  Description of Artificial Sequence Synthetic oligonucleotide
tgtgccagcagttacctcgttggggcagggtctaactatggctacaccttcggttcg

SEQ ID NO: 872 - DNA, 39 bp, Artificial Sequence
  Description of Artificial Sequence Synthetic oligonucleotide
tgtgccagcagttataacaatgagcagttcttcgggcca

SEQ ID NO: 873 - DNA, 54 bp, Artificial Sequence
  Description of Artificial Sequence Synthetic oligonucleotide
tgtgccagcagttacccgtcagcggccgcttacaatgagcagttcttcgggcca

SEQ ID NO: 874 - DNA, 51 bp, Artificial Sequence
  Description of Artificial Sequence Synthetic oligonucleotide
tgtgccagcaccccctcggcaggggctaatcagccccagcattttggtgat

SEQ ID NO: 875 - DNA, 54 bp, Artificial Sequence
  Description of Artificial Sequence Synthetic oligonucleotide
tgtgccaccagtgatctccgggacagggggaacaatgagcagttcttcgggcca

SEQ ID NO: 876 - DNA, 55 bp, Artificial Sequence
  Description of Artificial Sequence Synthetic oligonucleotide
tgtgtatttctgcgccaccacagggggccaaaacactgaagctttctttggacaa

SEQ ID NO: 877 - DNA, 39 bp, Artificial Sequence
  Description of Artificial Sequence Synthetic oligonucleotide
tgtgcctggaaagaggtacgggggcagttcttcgggcca

SEQ ID NO: 878 - DNA, 48 bp, Artificial Sequence
  Description of Artificial Sequence Synthetic oligonucleotide
tgtgcctggagtgaggggcgggggaatgaaaaactgttttttggcagt

SEQ ID NO: 879 - DNA, 39 bp, Artificial Sequence
  Description of Artificial Sequence Synthetic oligonucleotide
tgtgccagcgggggaacagatacgcagtattttggccca

SEQ ID NO: 880 - DNA, 54 bp, Artificial Sequence
  Description of Artificial Sequence Synthetic oligonucleotide
tgtgcctgggaatcctcccgggacattgatgatcctgaagctttctttggacaa

SEQ ID NO: 881 - DNA, 36 bp, Artificial Sequence
  Description of Artificial Sequence Synthetic oligonucleotide
tgtgccagcagtttcggcggaaatgagcagttcttc

SEQ ID NO: 882 - DNA, 36 bp, Artificial Sequence
  Description of Artificial Sequence Synthetic oligonucleotide
tgcgccagcggcgagggcaatcagccccagcatttt

SEQ ID NO: 883 - DNA, 45 bp, Artificial Sequence
  Description of Artificial Sequence Synthetic oligonucleotide
tgtgccagcagtttatcgtcgacaggtttgcagccccagcatttt

SEQ ID NO: 884 - DNA, 45 bp, Artificial Sequence
  Description of Artificial Sequence Synthetic oligonucleotide
tgtgccagcagtccaaaccgggacacctatcggggctacaccttc

SEQ ID NO: 885 - DNA, 45 bp, Artificial Sequence
  Description of Artificial Sequence Synthetic oligonucleotide
tgtgccaccagtgatttgaaccagccgaatcagccccagcatttt

SEQ ID NO: 886 - DNA, 39 bp, Artificial Sequence
  Description of Artificial Sequence Synthetic oligonucleotide
tgtgccagcagcgtaggtctaggcggggagctgtttttt

SEQ ID NO: 887 - DNA, 45 bp, Artificial Sequence
  Description of Artificial Sequence Synthetic oligonucleotide
tgcgccagcagctgggaagacagggacgcagatacgcagtatttt

SEQ ID NO: 888 - DNA, 48 bp, Artificial Sequence
  Description of Artificial Sequence Synthetic oligonucleotide
tgtgcctgggaatcctcccgggacattgatgatcctgaagctttcttt

SEQ ID NO: 889 - DNA, 45 bp, Artificial Sequence
  Description of Artificial Sequence Synthetic oligonucleotide
tgtgccagtagtatggatgggactcttctctacgagcagtacttc

SEQ ID NO: 890 - DNA, 54 bp, Artificial Sequence
  Description of Artificial Sequence Synthetic oligonucleotide
tgtgccagcagtttatctacgggagggcctagggttaattcacccctccacttt

SEQ ID NO: 891 - DNA, 42 bp, Artificial Sequence
  Description of Artificial Sequence Synthetic oligonucleotide
tgcgccagcagcttaacaggggggggaactgaagctttcttt

SEQ ID NO: 892 - DNA, 51 bp, Artificial Sequence
  Description of Artificial Sequence Synthetic oligonucleotide
tgtgccagcagcttaccgggacaggatacgcatctggagacccagtacttc

SEQ ID NO: 893 - DNA, 42 bp, Artificial Sequence
  Description of Artificial Sequence Synthetic oligonucleotide
tgtgccagcagcgtagcgggagtcacagatacgcagtatttt

SEQ ID NO: 894 - DNA, 36 bp, Artificial Sequence
  Description of Artificial Sequence Synthetic oligonucleotide
tgtgccagcagtattaacagctttgaagctttcttt

SEQ ID NO: 895 - DNA, 48 bp, Artificial Sequence
  Description of Artificial Sequence Synthetic oligonucleotide
tgtgccagcagtttcgtgctcgccatctctggaaacaccatatatttt

SEQ ID NO: 896 - DNA, 54 bp, Artificial Sequence
  Description of Artificial Sequence Synthetic oligonucleotide
tgtgccagcagcttagaaggcgggttgggctccctgaacactgaagctttcttt

SEQ ID NO: 897 - DNA, 51 bp, Artificial Sequence
  Description of Artificial Sequence Synthetic oligonucleotide
tgtgccagcagtgaactggcgggggacacatactcctacgagcagtacttc

SEQ ID NO: 898 - DNA, 39 bp, Artificial Sequence
  Description of Artificial Sequence Synthetic oligonucleotide
tgtgccgcggggaaggattggtccggggagctgtttttt

SEQ ID NO: 899 - DNA, 39 bp, Artificial Sequence
  Description of Artificial Sequence Synthetic oligonucleotide
tgcagtgccctggacaggggtaatgaaaaactgtttttt

SEQ ID NO: 900 - DNA, 45 bp, Artificial Sequence
  Description of Artificial Sequence Synthetic oligonucleotide
tgcagtgcccagaaaaatacagggggaggcgaaaaactgtttttt

SEQ ID NO: 901 - DNA, 39 bp, Artificial Sequence
  Description of Artificial Sequence Synthetic oligonucleotide
tgcagtgctagagatcgaagcgatgaaaaactgtttttt

SEQ ID NO: 902 - DNA, 30 bp, Artificial Sequence
  Description of Artificial Sequence Synthetic oligonucleotide
tgcagtgctagagattcgggctacaccttc

SEQ ID NO: 903 - DNA, 57 bp, Artificial Sequence
  Description of Artificial Sequence Synthetic oligonucleotide
tgcagtgcccgttaccgggacagggggcagggcgggagcaccgatacgcagtatttt

SEQ ID NO: 904 - DNA, 42 bp, Artificial Sequence
  Description of Artificial Sequence Synthetic oligonucleotide
tgtgccagcgggggacagggggctggaaacaccatatatttt

SEQ ID NO: 905 - DNA, 45 bp, Artificial Sequence
  Description of Artificial Sequence Synthetic oligonucleotide
tgtgccagcagcccaagtgggggggacaccggggagctgtttttt

SEQ ID NO: 906 - DNA, 42 bp, Artificial Sequence
  Description of Artificial Sequence Synthetic oligonucleotide
tgtgccagcaatgcatgggggggcaatcagccccagcatttt

SEQ ID NO: 907 - DNA, 52 bp, Artificial Sequence
  Description of Artificial Sequence Synthetic oligonucleotide
tgtgtatttctgtgccagcaacggggaggcggagacccagtacttcgggcca

SEQ ID NO: 908 - DNA, 45 bp, Artificial Sequence
  Description of Artificial Sequence Synthetic oligonucleotide
tgtgccagcagcttgctggagaatcagccccagcattttggtgat

SEQ ID NO: 909 - DNA, 45 bp, Artificial Sequence
  Description of Artificial Sequence Synthetic oligonucleotide
tgtgccagcaacaaactggggtatcagccccagcattttggtgat

SEQ ID NO: 910 - DNA, 51 bp, Artificial Sequence
  Description of Artificial Sequence Synthetic oligonucleotide
tgtgccagcagcttaacgggaggctacaccggggagctgttttttggagaa

SEQ ID NO: 911 - DNA, 42 bp, Artificial Sequence
  Description of Artificial Sequence Synthetic oligonucleotide
tgtgccagcaggcttggacagacctcctacgagcagtacttc

SEQ ID NO: 912 - DNA, 39 bp, Artificial Sequence
  Description of Artificial Sequence Synthetic oligonucleotide
tgtgccagcagctttgatctgaacactgaagctttcttt

SEQ ID NO: 913 - DNA, 39 bp, Artificial Sequence
  Description of Artificial Sequence Synthetic oligonucleotide
tgtgccagcagcttggaccttaacactgaagctttcttt

SEQ ID NO: 914 - DNA, 39 bp, Artificial Sequence
  Description of Artificial Sequence Synthetic oligonucleotide
tgtgccagcagcttggacgtggctgtggagcagtacttc

SEQ ID NO: 915 - DNA, 39 bp, Artificial Sequence
  Description of Artificial Sequence Synthetic oligonucleotide
tgtgccagcagcttggacgtgaacactgaagctttcttt

SEQ ID NO: 916 - DNA, 39 bp, Artificial Sequence
  Description of Artificial Sequence Synthetic oligonucleotide
tgcgccagcagcccggacgatctatatggctacaccttc

SEQ ID NO: 917 - DNA, 45 bp, Artificial Sequence
  Description of Artificial Sequence Synthetic oligonucleotide
tgtgccagtagccccttcaggagccctgattcacccctccacttt

SEQ ID NO: 918 - DNA, 39 bp, Artificial Sequence
  Description of Artificial Sequence Synthetic oligonucleotide
tgtgccagcaggacacaggggaacactgaagctttcttt

SEQ ID NO: 919 - DNA, 45 bp, Artificial Sequence
  Description of Artificial Sequence Synthetic oligonucleotide
tgtgccagcagcgataatgtccaggggattggcggctacaccttc

SEQ ID NO: 920 - DNA, 48 bp, Artificial Sequence
  Description of Artificial Sequence Synthetic oligonucleotide
tgtgccagcagcgacccggacgggggcgacaccggggagctgtttttt

SEQ ID NO: 921 - DNA, 45 bp, Artificial Sequence
  Description of Artificial Sequence Synthetic oligonucleotide
tgtgccagcagcaaagggaacaggcgatcctatggctacaccttc

SEQ ID NO: 922 - DNA, 42 bp, Artificial Sequence
  Description of Artificial Sequence Synthetic oligonucleotide
tgtgccagcagcttgggggggagtttttatggctacaccttc

SEQ ID NO: 923 - DNA, 45 bp, Artificial Sequence
  Description of Artificial Sequence Synthetic oligonucleotide
tgtgccagcagtctattgggacagggctcctatggctacaccttc

SEQ ID NO: 924 - DNA, 45 bp, Artificial Sequence
  Description of Artificial Sequence Synthetic oligonucleotide
tgtgccagcagcttagtgggggacacctacaatgagcagttcttc

SEQ ID NO: 925 - DNA, 36 bp, Artificial Sequence
  Description of Artificial Sequence Synthetic oligonucleotide
tgtgccagcagccctgacgttggtcgggtatatttt

SEQ ID NO: 926 - DNA, 48 bp, Artificial Sequence
  Description of Artificial Sequence Synthetic oligonucleotide
tgtgccagcagccaaaccggggggggacagggaaatcgagctttcttt

SEQ ID NO: 927 - DNA, 48 bp, Artificial Sequence
  Description of Artificial Sequence Synthetic oligonucleotide
tgtgccagtagttccgacagagcagtgggggacactgaagctttcttt

SEQ ID NO: 928 - DNA, 42 bp, Artificial Sequence
  Description of Artificial Sequence Synthetic oligonucleotide
tgtgccagcagctcgccacagggtgattacgagcagtacttc

SEQ ID NO: 929 - DNA, 45 bp, Artificial Sequence
  Description of Artificial Sequence Synthetic oligonucleotide
tgtgccagcagttggagtacaggcggggattcacccctccacttt

SEQ ID NO: 930 - DNA, 36 bp, Artificial Sequence
  Description of Artificial Sequence Synthetic oligonucleotide
tgtgccacatcggcggggtcggagacccagtacttc

SEQ ID NO: 931 - DNA, 42 bp, Artificial Sequence
  Description of Artificial Sequence Synthetic oligonucleotide
tgtgcctgggaaggacagggcccttactatggctacaccttc

SEQ ID NO: 932 - DNA, 48 bp, Artificial Sequence
  Description of Artificial Sequence Synthetic oligonucleotide
tgtgccagcagcgttgggggggaggcctacgagcagtacttcgggccg

SEQ ID NO: 933 - DNA, 45 bp, Artificial Sequence
  Description of Artificial Sequence Synthetic oligonucleotide
tgtgccagcagcgtaggaagcgtgggtgaagctttctttggacaa

SEQ ID NO: 934 - DNA, 51 bp, Artificial Sequence
  Description of Artificial Sequence Synthetic oligonucleotide
tgtgccagcagtgaaggggcgaaggctcctggggagcagtacttcgggccg

SEQ ID NO: 935 - DNA, 51 bp, Artificial Sequence
  Description of Artificial Sequence Synthetic oligonucleotide
tgtgccagcagcttctctagcggtaccacagatacgcagtattttggccca

SEQ ID NO: 936 - DNA, 42 bp, Artificial Sequence
  Description of Artificial Sequence Synthetic oligonucleotide
tgtgccagtagtatagggagccatggctacaccttcggttcg

SEQ ID NO: 937 - DNA, 54 bp, Artificial Sequence
  Description of Artificial Sequence Synthetic oligonucleotide
tgtgccagcagcttggccgtggtcaaatctggaaacaccatatattttggagag

SEQ ID NO: 938 - DNA, 45 bp, Artificial Sequence
  Description of Artificial Sequence Synthetic oligonucleotide
tgtgccagtagtactcgttccgcctacgagcagtacttcgggccg

SEQ ID NO: 939 - DNA, 45 bp, Artificial Sequence
  Description of Artificial Sequence Synthetic oligonucleotide
tgtgccagtagtgttaggagctcctacgagcagtacttcgggccg

SEQ ID NO: 940 - DNA, 45 bp, Artificial Sequence
  Description of Artificial Sequence Synthetic oligonucleotide
tgcagtgctagagatgagacagggaatggctacaccttcggttcg

SEQ ID NO: 941 - DNA, 40 bp, Artificial Sequence
  Description of Artificial Sequence Synthetic oligonucleotide
tgctagggacgggacaggcaatggctacaccttcggttcg

SEQ ID NO: 942 - DNA, 49 bp, Artificial Sequence
  Description of Artificial Sequence Synthetic oligonucleotide
tgctagagatatacagggggcgcaggtctacgagcagtacttcgggccg

SEQ ID NO: 943 - DNA, 48 bp, Artificial Sequence
  Description of Artificial Sequence Synthetic oligonucleotide
tgcagtgcccgggacagggagtacggaaacaccatatattttggagag

SEQ ID NO: 944 - DNA, 45 bp, Artificial Sequence
  Description of Artificial Sequence Synthetic oligonucleotide
tgcagtgctagagataccgttggaaacaccatatattttggagag

SEQ ID NO: 945 - DNA, 54 bp, Artificial Sequence
  Description of Artificial Sequence Synthetic oligonucleotide
tgcagtgctaggggtggacagacggaagttggaaacaccatatattttggagag

SEQ ID NO: 946 - DNA, 51 bp, Artificial Sequence
  Description of Artificial Sequence Synthetic oligonucleotide
tgtgccagcagtgacggcagggtggcccccggggagctgttttttggagaa

SEQ ID NO: 947 - DNA, 51 bp, Artificial Sequence
  Description of Artificial Sequence Synthetic oligonucleotide
tgtgccagcagtggtggtcaggtcgccccctccgagcagtacttcgggccg

SEQ ID NO: 948 - DNA, 45 bp, Artificial Sequence
  Description of Artificial Sequence Synthetic oligonucleotide
tgtgccagtagtattaggagctcctacgagcagtacttcgggccg

SEQ ID NO: 949 - DNA, 45 bp, Artificial Sequence
  Description of Artificial Sequence Synthetic oligonucleotide
tgtgccagcagtttatcgtcctacactgaagctttctttggacaa

SEQ ID NO: 950 - DNA, 54 bp, Artificial Sequence
  Description of Artificial Sequence Synthetic oligonucleotide
tgtgccagcagccaacccgacaaggcagcctacaatgagcagttcttcgggcca

SEQ ID NO: 951 - DNA, 48 bp, Artificial Sequence
  Description of Artificial Sequence Synthetic oligonucleotide
tgtgccagcagctcccctggtatcgactatggctacaccttcggttcg

SEQ ID NO: 952 - DNA, 48 bp, Artificial Sequence
  Description of Artificial Sequence Synthetic oligonucleotide
tgtgccagcagctctccggggataaactatggctacaccttcggttcg

SEQ ID NO: 953 - DNA, 45 bp, Artificial Sequence
  Description of Artificial Sequence Synthetic oligonucleotide
tgtgccagcagcgtagtggggggcgacgagcagtacttcgggccg

SEQ ID NO: 954 - DNA, 45 bp, Artificial Sequence
  Description of Artificial Sequence Synthetic oligonucleotide
tgcagtgctagagactctacagggaatggctacaccttcggttcg

SEQ ID NO: 955 - DNA, 42 bp, Artificial Sequence
  Description of Artificial Sequence Synthetic oligonucleotide
tgcagctcacaggaggggggatatggctacaccttcggttcg

SEQ ID NO: 956 - DNA, 51 bp, Artificial Sequence
  Description of Artificial Sequence Synthetic oligonucleotide
tgcagcgtggggggcaggggagatcgagaccagccccagcattttggtgat

SEQ ID NO: 957 - DNA, 48 bp, Artificial Sequence
  Description of Artificial Sequence Synthetic oligonucleotide
tgcagcgttggccaggggggaactaatgaaaaactgttttttggcagt

SEQ ID NO: 958 - DNA, 48 bp, Artificial Sequence
  Description of Artificial Sequence Synthetic oligonucleotide
tgcagcgttgggacagggggaactaatgaaaaactgttttttggcagt

SEQ ID NO: 959 - DNA, 48 bp, Artificial Sequence
  Description of Artificial Sequence Synthetic oligonucleotide
tgtgccagcagcgcgagcttagcgggagtcgagcagtacttcgggccg

SEQ ID NO: 960 - DNA, 54 bp, Artificial Sequence
  Description of Artificial Sequence Synthetic oligonucleotide
tgcgccagcagccatacgttcgacaggggaggggagacccagtacttcgggcca

SEQ ID NO: 961 - DNA, 42 bp, Artificial Sequence
  Description of Artificial Sequence Synthetic oligonucleotide
tgtgccagcagcttggacccctacgagcagtacttcgggccg

SEQ ID NO: 962 - DNA, 39 bp, Artificial Sequence
  Description of Artificial Sequence Synthetic oligonucleotide
tgtgccagtagtatcttttctcgcgatacgcagtatttt

SEQ ID NO: 963 - DNA, 39 bp, Artificial Sequence
  Description of Artificial Sequence Synthetic oligonucleotide
tgtgccagtagtattcggagctcctacgagcagtacttc

SEQ ID NO: 964 - DNA, 48 bp, Artificial Sequence
  Description of Artificial Sequence Synthetic oligonucleotide
tgtgccagcagattaacagggggagggctggcctacgagcagtacttc

SEQ ID NO: 965 - DNA, 39 bp, Artificial Sequence
  Description of Artificial Sequence Synthetic oligonucleotide
tgcgccagcagccccgacggggcggatgagcagttcttc

SEQ ID NO: 966 - DNA, 39 bp, Artificial Sequence
  Description of Artificial Sequence Synthetic oligonucleotide
tgtgccagtagccccaggtccacagatacgcagtatttt

SEQ ID NO: 967 - DNA, 39 bp, Artificial Sequence
  Description of Artificial Sequence Synthetic oligonucleotide
tgtgccagtagtactcgttccgcctacgagcagtacttc

SEQ ID NO: 968 - DNA, 39 bp, Artificial Sequence
  Description of Artificial Sequence Synthetic oligonucleotide
tgtgccagtagtgtcagaagctcctacgagcagtacttc

SEQ ID NO: 969 - DNA, 45 bp, Artificial Sequence
  Description of Artificial Sequence Synthetic oligonucleotide
tgtgccagcaccttccttgccccaaatgccgctgaagctttcttt

SEQ ID NO: 970 - DNA, 39 bp, Artificial Sequence
  Description of Artificial Sequence Synthetic oligonucleotide
tgtgccaccgggaaaagccctgaagagacccagtacttc

SEQ ID NO: 971 - DNA, 39 bp, Artificial Sequence
  Description of Artificial Sequence Synthetic oligonucleotide
tgcaccagcagccgcaaagtccaagagacccagtacttc

SEQ ID NO: 972 - DNA, 51 bp, Artificial Sequence
  Description of Artificial Sequence Synthetic oligonucleotide
tgtgccagcagcttaggtgtagaggatcacagggtgtatggctacaccttc

SEQ ID NO: 973 - DNA, 45 bp, Artificial Sequence
  Description of Artificial Sequence Synthetic oligonucleotide
tgtgccagcagtcccattggaatgtggggaaatgagcagttcttc

SEQ ID NO: 974 - DNA, 39 bp, Artificial Sequence
  Description of Artificial Sequence Synthetic oligonucleotide
tgtgccagcgccacactccagggggatggctacaccttc

SEQ ID NO: 975 - DNA, 24 bp, Artificial Sequence
  Description of Artificial Sequence Synthetic oligonucleotide
tgtgcttcgggggcagacaccttc

SEQ ID NO: 976 - DNA, 36 bp, Artificial Sequence
  Description of Artificial Sequence Synthetic oligonucleotide
tgtgccagcagacgtagcaatcagccccagcatttt

SEQ ID NO: 977 - DNA, 45 bp, Artificial Sequence
  Description of Artificial Sequence Synthetic oligonucleotide
tgcgccagcagtgagatgacgggggggcaagatacgcagtatttt

SEQ ID NO: 978 - DNA, 45 bp, Artificial Sequence
  Description of Artificial Sequence Synthetic oligonucleotide
tgtgccagtagtatcgacagggggattaactatggctacaccttc

SEQ ID NO: 979 - DNA, 45 bp, Artificial Sequence
  Description of Artificial Sequence Synthetic oligonucleotide
tgtgccagcagcttagaaccccgccctcatactgaagctttcttt

SEQ ID NO: 980 - DNA, 45 bp, Artificial Sequence
  Description of Artificial Sequence Synthetic oligonucleotide
tgtgccagcagcttaaggataggggggcgagagacccagtacttc

SEQ ID NO: 981 - DNA, 45 bp, Artificial Sequence
  Description of Artificial Sequence Synthetic oligonucleotide
tgtgccagcagccccgggatggggggcgcggatggctacaccttc

SEQ ID NO: 982 - DNA, 45 bp, Artificial Sequence
  Description of Artificial Sequence Synthetic oligonucleotide
tgtgccagcagccctttagaggacagttcctatggctacaccttc

SEQ ID NO: 983 - DNA, 45 bp, Artificial Sequence
  Description of Artificial Sequence Synthetic oligonucleotide
tgcgccagcagccaagatagtggttataattcacccctccacttt

SEQ ID NO: 984 - DNA, 36 bp, Artificial Sequence
  Description of Artificial Sequence Synthetic oligonucleotide
tgtgccagcagttcagccaggcgtggctacaccttc

SEQ ID NO: 985 - DNA, 51 bp, Artificial Sequence
  Description of Artificial Sequence Synthetic oligonucleotide
tgtgccaccagcagagatgggactagcgggctaaccggggagctgtttttt

SEQ ID NO: 986 - DNA, 33 bp, Artificial Sequence
  Description of Artificial Sequence Synthetic oligonucleotide
tgcagcgcagatctgaacactgaagctttcttt

SEQ ID NO: 987 - DNA, 39 bp, Artificial Sequence
  Description of Artificial Sequence Synthetic oligonucleotide
tgcagtgctagcccctcgggagcagttgagcagttcttc

SEQ ID NO: 988 - DNA, 42 bp, Artificial Sequence
  Description of Artificial Sequence Synthetic oligonucleotide
tgcagtgtcgcagaaggacagggtgcttatgagcagttcttc

SEQ ID NO: 989 - DNA, 21 bp, Artificial Sequence
  Description of Artificial Sequence Synthetic oligonucleotide
tgcagcgttgaagctttcttt

SEQ ID NO: 990 - DNA, 42 bp, Artificial Sequence
  Description of Artificial Sequence Synthetic oligonucleotide
tgtgccatcagtgaagtcgtcggactgcagccccagcatttt

SEQ ID NO: 991 - DNA, 45 bp, Artificial Sequence
  Description of Artificial Sequence Synthetic oligonucleotide
tgtgccagcagtgccggcaggtattcgacctacgagcagtacttc

SEQ ID NO: 992 - DNA, 42 bp, Artificial Sequence
  Description of Artificial Sequence Synthetic oligonucleotide
tgtgccagcagcccgacccagggggcagtagagcagtacttc

SEQ ID NO: 993 - DNA, 39 bp, Artificial Sequence
  Description of Artificial Sequence Synthetic oligonucleotide
tgtgccagcagttggggcggcaacactgaagctttcttt

(19) United States
(12) Patent Application Publication
(10) Pub. No.: US 2024/0165155 A1
(43) Pub. Date: May 23, 2024

(54) METHODS AND MATERIALS FOR TARGETING TUMOR ANTIGENS

(71) Applicant: The Johns Hopkins University, Baltimore, MD (US)

(72) Inventors: Kellie N. Smith, Baltimore, MD (US); Justina Caushi, Baltimore, MD (US); Emily Han-Chung Hsiue, Baltimore, MD (US); Andrew M. Pardoll, Baltimore, MD (US); Shibin Zhou, Owings Mills, MD (US); Bert Vogelstein, Baltimore, MD (US); Kenneth W. Kinzler, Frankford, DE (US); Nickolas Papadopoulos, Towson, MD (US)

(21) Appl. No.: 18/283,887

(22) PCT Filed: Mar. 31, 2022
(86) PCT No.: PCT/US2022/022791

§ 371 Date: Sep. 25, 2023

Related U.S. Application Data
(60) Provisional application No. 63/168,878, filed on Mar. 31, 2021.

(51) Int. Cl. A61K 35/17; A61K 39/00; A61K 45/06; C07K 14/47; C07K 14/725; C12N 15/86

(52) U.S. Cl. CPC A61K 35/17; A61K 39/4611; A61K 39/4632; A61K 39/464411; A61K 39/464451; A61K 45/06; C07K 14/4746; C07K 14/7051; C12N 15/86

(57) ABSTRACT

This document relates to methods and materials for treating a mammal having cancer. For example, this document provides T cell receptors (TCRs) that can bind to a modified peptide (e.g., a tumor antigen). In some cases, methods of using T cells expressing one or more TCRs that can bind to a modified peptide (e.g., a tumor antigen) to treat a mammal having cancer are provided.

### CROSS-REFERENCE To RELATED APPLICATIONS

This application claims the benefit of U.S. Patent Application Ser. No. 63/168,878, filed on Mar. 31, 2021. The disclosure of the prior application is considered part of (and is incorporated by reference in) the disclosure of this application.

### BACKGROUND

### 1. Technical Field

This document relates to methods and materials for treating a mammal having cancer. For example, this document provides T cell receptors (TCRs) that can bind to a modified peptide (e.g., a tumor antigen). In some cases, T cells expressing one or more TCRs provided herein can be administered to a mammal having cancer to treat the mammal.

### 2. Background Information

Immune checkpoint blockade (ICB) has revolutionized cancer treatment; however, the efficacy of ICB agents, such as programmed cell death protein 1 (PD-1) signaling inhibitors (e.g., anti-PD-1 antibodies and anti-PD-L1 antibodies), is predicated upon CD8 T cell-mediated anti-tumor immunity (Tumeh et al., Nature 515:568-571 (2014)), and most patients do not respond ICB agents. PD-1 blockade “unleashes” CD8 T cells, including those specific for mutation-associated neoantigens (MANAs), but factors in the tumor microenvironment can inhibit responses by dampening MANA-specific T cell function. Recent advances in single cell transcriptomics are revealing global T cell dysfunction programs in tumor-infiltrating lymphocytes (TIL). However, the vast majority of TIL do not recognize tumor antigens.

### SUMMARY

There is a continuing need in the art to develop new methods to diagnose, monitor, and effectively treat cancers. For example, the identification of therapeutic targets highly specific to cancer cells is one of the greatest challenges for developing an effective cancer therapy.

This document provides methods and materials for treating a mammal having cancer. For example, this document provides TCRs that can bind to a modified p53 peptide (e.g., a modified p53 peptide present in a peptide-human leukocyte antigen (HLA) complex) such as a p53 polypeptide having a R to L substitution at amino acid residue 248 (e.g., p53 R248L peptide). In some cases, T cells expressing one or more TCRs that can bind to a modified p53 peptide (e.g., a modified p53 peptide present in a peptide-HLA complex) can be administered to a mammal having a cancer (e.g., a cancer containing one or more cancer cells expressing the modified p53 peptide) to treat the mammal.

As demonstrated herein, T cell receptors (TCRs) were identified that target (e.g., target and bind to) the p53 R248L MANA. MANAs can be used as highly specific cancer targets because they are not present in normal tissue(s). The ability to specifically target MANAs provides a tumor-specific method to diagnose and/or treat cancer. For example, TCRs specifically targeting MANAs can be used in T cells (e.g., T cells expressing a chimeric antigen receptor (CARTs)) to treat a mammal having cancer. Further, TCRs that can bind to a MANA can be used to provide a widely applicable and genetically predictable off-the-shelf targeted cancer immunotherapy.

In general, one aspect of this document features TCRs that can bind to a modified p53 polypeptide comprising a R to L substitution at amino acid residue 248 (R248L). The modified p53 polypeptide can include a p53 R248L peptide comprising, consisting essentially of, or consisting of the amino acid sequence set forth in any one of SEQ ID NOs:1-40. The TCR can include an alpha (α) chain including a TCRα-CDR3 set forth in any one of SEQ ID NOs:41-44. The TCR can include a beta ((β) chain including a TCRβ-CDR3 set forth in any one of SEQ ID NOs:45-48. The TCR can include an a chain that includes a TCRα-CDR3 set forth in any one of SEQ ID NOs:41-44, and can include a β chain that includes a TCRβ-CDR3 set forth in any one of SEQ ID NOs:45-48.

In another aspect, this document features T cells comprising a TRC that can bind to a modified p53 polypeptide comprising a R248L substitution. The modified p53 polypeptide can include a p53 R248L peptide comprising, consisting essentially of, or consisting of the amino acid sequence set forth in any one of SEQ ID NOs:1-40. The TCR can include an alpha (α) chain including a TCRα-CDR3 set forth in any one of SEQ ID NOs:41-44. The TCR can include a beta (β) chain including a TCRβ-CDR3 set forth in any one of SEQ ID NOs:45-48. The TCR can include an α chain that includes a TCRα-CDR3 set forth in any one of SEQ ID NOs:41-44, and can include a β chain that includes a TCRβ-CDR3 set forth in any one of SEQ ID NOs:45-48. The T cell can be a human T cell. The T cell can be a non-human T cell.

In another aspect, this document features nucleic acids encoding a TRC that can bind to a modified p53 polypeptide comprising a R248L substitution. The modified p53 polypeptide can include a p53 R248L peptide comprising, consisting essentially of, or consisting of the amino acid sequence set forth in any one of SEQ ID NOs:1-40. The TCR can include an alpha (α) chain including a TCRα-CDR3 set forth in any one of SEQ ID NOs:41-44. The TCR can include a beta (β) chain including a TCRβ-CDR3 set forth in any one of SEQ ID NOs:45-48. The TCR can include an α chain that includes a TCRα-CDR3 set forth in any one of SEQ ID NOs:41-44, and can include a β chain that includes a TCRβ-CDR3 set forth in any one of SEQ ID NOs:45-48. The nucleic acid can be in the form of a vector. The vector can be an expression vector. The vector can be a viral vector.

In another aspect, this document features T cells including a nucleic acid encoding a TRC that can bind to a modified p53 polypeptide comprising a R248L substitution, where the nucleic acid encodes the TCR. The T cell can be a human T cell. The T cell can be a non-human T cell.

In another aspect, this document features methods for treating a mammal having cancer. The methods can include, or consist essentially of, administering to a mammal having cancer a T cell including a TRC that can bind to a modified p53 polypeptide comprising a R248L substitution or a T cell including nucleic acid encoding a TRC that can bind to a modified p53 polypeptide comprising a R248L substitution, where the cancer includes a cancer cell expressing the modified p53 polypeptide. The cancer cell expressing the modified p53 polypeptide can presents a p53 R248L peptide in a peptide-HLA complex. The p53 R248L peptide comprising, consisting essentially of, or consisting of the amino acid sequence set forth in any one of SEQ ID NOs:1-40. The mammal can be a human. The cancer can be a non-small cell lung cancer (NSCLC), a colon adenocarcinoma, a rectal adenocarcinoma, a head and neck squamous cell carcinoma, a pancreatic adenocarcinoma, melanomas, a urothelial carcinoma, a uterine corpus endometrial carcinoma, or a uterine carcinoma. The method also can include administering a checkpoint inhibitor to the mammal. The checkpoint inhibitor can be an anti-CTLA-4 (cytotoxic T-lymphocyte-associated protein 4) antibody, an anti-PD-1 (programmed death 1) antibody, an anti-PD-L1 (programmed death 1 ligand) antibody, an anti-LAG3 (lymphocyte activation gene 3) antibody, an anti-Tim3 (T cell immunoglobulin and mucin domain-containing protein 3) antibody, an anti-TIGIT (T cell immunoreceptor with Ig and ITIM domains) antibody, an anti-VISTA (V-domain Ig suppressor of T cell activation) antibody, an anti-CD47 (cluster of differentiation 47) antibody, an anti-SIRPalpha (signal regulatory protein alpha) antibody, an anti-B7-H3 (B7 homolog 3) antibody, an anti-B7-H4 (B7 homolog 4) antibody, an anti-neuritin antibody, an anti-neuropilin antibody, an anti-IL-35 (interleukin 35), an IDO (indoleamine-pyrrole 2,3-dioxygenase) inhibitor, an A2AR (adenosine A2A receptor) inhibitor, an arginase inhibitor, or a glutaminase inhibitor. The method also can include administering a co-stimulatory molecule to the mammal. The co-stimulator molecule can be an agonist of a co-stimulatory receptor. The agonist of a co-stimulatory receptor can be an anti-GITR (glucocorticoid-induced TNFR-related) antibody, an anti-CD27 (cluster of differentiation 27) antibodies antibody, an anti-4-1BB (CD137; cluster of differentiation 137) antibody, an anti-OX40 (CD134; cluster of differentiation 134) antibody, an anti-ICOS (inducible T-cell costimulator) antibody, or an anti-CD40 (cluster of differentiation 40) antibody.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1E. Profiling single immune cells in anti-PD-1-treated lung cancer with combined scRNA-Seq/TCRSeq. FIG. 1A: Graphical overview of the experimental design. Single cell RNAseq/TCRseq was performed on T cells derived from resected tumor, adjacent NL, and tumor-draining lymph nodes (TDLN) of lung cancer patients treated with neoadjuvant PD-1 blockade. The MANAFEST and ViraFEST assays were performed to identify mutation associated neoantigen (MANA)/EBV/Influenza A-specific T cells. Antigen specific T cell clones were linked with transcriptomic profiles by using the TCRb chain as a biologic barcode. FIG. 1B: 2D projection of expression profiles of 560,916 T cells from post-treatment tumor, adjacent normal lung, and tumor-draining lymph node using UMAP. Broad immune cell subsets were annotated and marked by color code. FIG. 1C: Heatmap of the top 5 differential genes for respective immune cell subsets. FIG. 1D: 2D UMAP red-scale projection of canonical T cell subset marker genes (CD8A, CD4, and FOXP3), cell subset selective genes (GZMK, TCF7, ZNF683, CXCL13, SLC4A10, and MKI67), and well defined immune checkpoints (PDCD1, CTLA4, HAVCR2, TIGIT, ENTPD1, LAG3). FIG. 1E: Principal component analysis (PCA) of pseudobulk gene expression for post treatment tumor (yellow) vs adjacent NL (dark blue) and MPR (light blue) vs non-MPR (red).

FIGS. 2A-2I. Transcriptional characterization of antigen specific T cells in NSCLC treated with neoadjuvant PD-1 blockade. FIG. 2A: MANAFEST assays were performed on the peripheral blood of 4 MPR and 5 non-MPR. An example MANAFEST assay is shown for patient MD01-004. Data are shown as the frequency of MANAFEST+clonotypes after in vitro culture for clonotypes found only in the MANAFEST assay (blue) and clonotypes found in the MANAFEST assay and also detected in single cell analysis of TIL (green). Red bars represent 4 individual clonotypes tested in the Jurkat reporter system shown in FIG. 2B. FIG. 2B: Specificity of four clones positive by MANAFEST for the p53 R248L-derived NSSCMGGMNLR (SEQ ID NO:1) neoantigen (MANA 12, red box and red bars) were confirmed in a dose-dependent manner by cloning and transfection into a Jurkat NFAT luciferase reporter system (FIG. 2B, top). A known HLA A*11-restricted EBV-specific TCR was also cloned as a control (green). To enable ligand-dependent TCR signaling capacity comparisons between TCRs with variable transfection efficiencies, data are shown as the log fold change in luminescence relative to TCR-transfected Jurkats cultured without peptide. Pre- and post-treatment tissue (FIG. 2B, middle) and peripheral blood (FIG. 2B, bottom) representation were also visualized for these four clonotypes and these data are shown as the frequency among all TCRs detected by bulk TCRseq. Figure C: 2D projection of expression profiles of 235,851 CD8 T cells from tumor, adjacent normal lung, and draining lymph node using UMAP. CD8 T cell subsets are annotated and marked by color code. FIG. 2D: 2D projection of MANA/EBV/Influenza A specific T cells on total merged CD8 UMAP. TRB aa sequence was used as a biological barcode to match MANA/EBV/Influenza A specific T cell clonotypes identified from the FEST assay with single cell VDJ profile. FIG. 2E: 2D projection of MANA/EBV/Influenza A specific T cells on CD8 UMAP of post treatment tumor and adjacent NL. TIL and NL T cells were down-sampled to equal number of cells before visualization. Bar plot shows proportion of antigen specific T cells among total CD8 T cells by tissue compartment (blue bar, adjacent NL; yellow bar, post treatment tumor). Dot plot shows proportion of antigen specific T cells, stratified by CD8 T cell subsets, with size of the dot representing proportion among total CD8 T cells (blue dot, adjacent NL; yellow dot, post treatment tumor). FIG. 2F: MANA/EBV/Influenza A specific gene programs in the TIL expressed as a heatmap. FIG. 2G: Expression levels of key transcriptional regulators, memory makers, tissue resident markers, T cell immune checkpoints and CD8 effector/activation genes among MANA-specific T cells (red), Influenza A-specific T cells (blue) and EBV-specific T cells (purple). FIG. 2H: Waterfall plot showing the top 30 differential genes comparing flu-specific T cells and MANA-specific T cells. FIG. 2I: IL7 functional experiment for MANA- and influenza A-specific T cells. Ridge plot shows the composite IL7-upregulated gene set score for MANA-specific T cells vs Influenza A specific T cells within TIL cultured with MANA/Influenza A peptide at titrating concentrations of IL7 (0 μg, 0.1 μg, 1 μg, and 10 μg, left panel). A dose response curve of the mean (with standard error) IL7-upregulated gene set score against different titrations of IL7 is shown (right panel).

FIGS. 3A-3F. Differential MANA-specific gene programs in MPR vs. non-MPR tumors. FIG. 3A: Heatmap of differential genes of tumor infiltrating MANA-specific T from MPR and non-MPR. FIG. 3B: IL7R expression of MANA specific CD8 clones in MRPs and non-MPR at clonal level (each dot representing a unique clone). Wilcoxon rank sum test, **: 0.001<P<0.01. FIG. 3C: T cell immune checkpoint score (derived from expression of CTLA4, PDCD1, LAG3, HAVCR2, TIGIT, ENTPD1) of single cell RNA-Seq/TCR-Seq profiled MANA specific CD8 cells and influenza A specific CD8 cells in MRPs and non-MPR. Each dot represents a single cell. Wilcoxon rank sum test, ****: P<0.0001, ns: P>0.05. FIG. 3D: Top 30 ranked genes that are expression correlated with T cell immune checkpoints comprising the checkpoint score in MPR and non-MPR. FIG. 3E: Gene program differences between MPR and non-MPR in top genes that are expression correlated with T cell immune checkpoints (derived from FIG. 3D). FIG. 3F: MANA specific T cell tracking across tumor, adjacent NL, tumor draining LN, and longitudinal blood from a patient achieving a pathologic complete response after 4 weeks of neoadjuvant nivolumab. MANA-specific T cells were labeled as red triangle and corresponding cell types were annotated with dashed lines.

FIG. 4. Neoantigen-specific TCRs identified by the MANAFEST assay. MANAFEST assay for 3 MPR and 3 non-MPR patients. MANAFEST+expansions observed in each patient are shown for clonotypes only found in the MANAFEST assays (blue), clonotypes found in the MANAFEST assay and detected in single cell TIL (green) and clonotypes detected in single cell TIL and validated by cloning and transfection into a Jurkat NFAT luciferase reporter system (red). MANAFEST data are shown as the frequency of MANAFEST+clonotypes among CD8+ T cells after 10 day culture. Significant MANAFEST+expansions were not observed in nonMPR patient, MD01-019 (data not shown). MANAFEST+expansions for MPR patient, MD01-005, have been previously shown.

FIGS. 5A-5D. Validation of MANA-specific TCRs identified by the MANAFEST assay. Seven MANA-specific clonotypes identified by MANAFEST in three patients were confirmed by cloning and transfection into a Jurkat NFAT luciferase reporter system in a dose-dependent manner. FIG. 5A: In MD01-005, two clonotypes recognize ARVCF-derived EVIVPLSGW (SEQ ID NO:49) MANA. In vitro binding and stability assays demonstrate the affinity kinetics of each relevant MANA, the corresponding wild-type peptide for their restricting HLA class I allele. Blank, or no peptide was used as a negative control for each assay; known HLA-matched epitopes were used as positive controls. Data are shown as counts per second with increasing peptide concentration for binding assays (top) or absorbance in presence of urea for stability assays (bottom). Data points indicate the mean of two independent experiments±SD. FIG. 5B: In MD01-004, four clonotypes recognize p53 R248L-derived NSSCMGGMNLR (SEQ ID NO:1) MANA. In vitro binding and stability assays demonstrate the affinity kinetics of each relevant MANA, the corresponding wild-type peptide for their restricting HLA class I allele. Blank, or no peptide was used as a negative control for each assay; known HLA-matched epitopes were used as positive controls. Data are shown as counts per second with increasing peptide concentration for binding assays (top) or absorbance in presence of urea for stability (bottom). Data points indicate the mean of two independent experiments±SD. FIG. 5C: In MD043-011, one clonotype recognizes CARM1-derived neoantigens. Low level expansion of the CASSLDPYEQYF (SEQ ID NO:50) clone, which did not meet our standard cutoff for antigen specificity, was observed in response to three neoantigens, MD043-011-MANA 24 -MANA31 and -MANA 36, which all encompassed the same core epitope resulting from a CARM1 R208W mutation, AQAGAWKIYAV (SEQ ID NO:51), AQAGAWKIY (SEQ ID NO:52), FAAQAGAWKIY (SEQ ID NO:53), respectively. FIG. 5D: The COS-7 cell line was transfected with HLA-A*68:01 plasmid and p53 R248L mutant plasmid or p53 wild type plasmid. HLA- and p53-transfected COS-7 cells, autologous APC loaded with MD01-004-MANA12, and HLA-A*68:01-transfected COS-7 were co-cultured with CD8+ Jurkat reporter cells expression the MD01-004-MANA12-reactive TCR, CATTGGQNTEAFF (SEQ ID NO:45).

FIG. 6. Peripheral dynamics and cross-compartment representation of MANA-specific T cells. Bulk TCRseq was performed on pre- and post-treatment tissue and peripheral blood. MANA-specific TCRβ clone representation is shown as the frequency among all TCRs detected by TCRseq. TDLN, tumor draining lymph node; DLN, draining lymph node.

FIG. 7. Virus-specific TCR identified by the ViraFEST assay. The ViraFEST assay was performed on 1MPR and 2 non-MPR to identify influenza-specific TCRβ clones. Influenza A pools consisting of overlapping peptides from the matrix protein of H1N1 and H3N2 and the nucleocapsid protein of H1N1 and H3N2 were used to stimulate peripheral blood T cells in vitro for 10 days. viraFEST+expansions are shown for each patient. Clonotypes only found in the MANAFEST assays (blue) and clonotypes found in the MANAFEST assay and detected in single cell TIL (green) are shown as the frequency among all CD8+ T cells detected by TCRseq.

FIG. 8. Peripheral dynamics and cross-compartment representation of CEF-specific T cells. CEF+TCRβ clonotypes were identified in three MPR and one non-MPR (data previously shown as positive controls in MANAFEST assays in FIG. 2 and FIG. 5). Pre- and post-treatment tissue and peripheral blood representation were visualized for each clonotype and these data are shown as the frequency among all TCRs detected by TCRseq. TDLN, tumor draining lymph node; DLN, draining lymph node.

FIG. 9. Peripheral dynamics and cross-compartment representation of flu-specific T cells. Peripheral blood T cells were tested for reactivity against peptide pools representing the matric protein and nucleoprotein of H1N1 and H3N2 using the ViraFEST assay. Flu-specific cells were identified in one MPR and two no-MPR. Pre- and post-treatment tissue and peripheral blood representation were visualized for flu-specific clonotypes. Data are shown as the frequency among all TCRs detected by TCRseq. TDLN, tumor draining lymph node; DLN, draining lymph node.

FIG. 10. 2D UMAP red-scale projection of canonical T cell subset marker genes, cell subset selective genes and immune checkpoints on CD8 T cell subsets.

FIGS. 11A-11B. Clonal tracking of MANA-specific T cells across tissue compartments. FIG. 11A: MANA specific T cells were found in tumor, adjacent NL, and tumor draining LN at tumor resection and in a distant brain metastasis from a patient with 75% residual tumor at resection and early relapse. The scatterplot shows the average expression of genes comparing the post-treatment tumor at resection vs. the distant brain metastasis. The top differential genes are labeled in red. FIG. 11B: MANA specific T cells were detected in the post-treatment tumor and tumor draining LN from a patient with partial response (40% percent residual tumor).

FIGS. 12A-12D. Identification and characterization of T cell receptors specific for a p53 R248L-derived neoantgien in NSCLC treated with neoadjuvant PD-1 blockade. FIG. 12A: MANAFEST assay was performed in non-MPR patient, MD01-004, in which 41 neoantigen-specific and 2 CMV/EBV/flu (CEF)-specific TCRb CDR3 clonotypes were identified. FIG. 12B: Four of these clones were specific for the hotspot p53 R248L-derived MANA (MD01-004-MANA12), whose specificities were validated by TCR cloning into the Jurkat/NFAT-luciferase system. Additionally, clones specific for p53 R248L-derived MANA were found at appreciable frequency in the pre- and post-treatment tumor, despite the tumor not attaining MPR. Notably, these MANA-specific clones were detected at very low frequency (median: 0.001%, range: 0-0.038%) in the peripheral blood across all available timepoints, thereby highlighting the sensitivity of the MANAFEST assay. Peptide dose-response curves were comparable to the positive control EBV-specific TCR, suggesting these TCRs were capable of strong ligand-dependent signaling (sometimes referred to as functional avidity). FIG. 12C: Endogenous processing and HLA A*68:01-restricted presentation of MD01-004-MANA12 was confirmed by transfection of HLA*A6801 and R248L-mutated p53 into a COS-7 cell line and co-culture with a MD01-004-MANA12-reactive TCR. FIG. 12D: in vitro binding and stability assays demonstrate the affinity kinetics of each relevant MANA, the corresponding wild-type peptide for their restricting HLA class I allele.

FIG. 13. Table 6. MANAs tested by MANAFEST.

FIG. 14. Table 8. Antigen-specific TCR clonotypes identified by the MANAFEST and viraFEST assays.

### DETAILED DESCRIPTION

This document provides methods and materials for treating a mammal having cancer. For example, this document provides TCRs that can bind to a modified p53 peptide (e.g., a modified p53 peptide present in a peptide-HLA complex) such as a p53 R248L peptide. In some cases, T cells expressing TCRs that can bind to a modified p53 peptide (e.g., a modified p53 peptide present in a peptide-HLA complex) can be administered to a mammal having a cancer (e.g., a cancer containing one or more cancer cells expressing the modified p53 peptide) to treat the mammal.

As used herein, a modified peptide is a peptide derived from a modified polypeptide. A modified polypeptide can be any appropriate modified polypeptide (e.g., a polypeptide having a disease-causing mutation such as a mutation in an oncogenic or a mutation in a tumor suppressor gene). A modified peptide can have one or more amino acid modifications (e.g., substitutions) relative to a WT peptide (e.g., a peptide derived from a WT polypeptide from which the modified polypeptide is derived). A modified peptide also can be referred to as a mutant peptide. In some cases, a modified peptide can be a tumor antigen. Examples of tumor antigens include, without limitation, MANAs, tumor-associated antigens, and tumor-specific antigens. A modified peptide can be any appropriate length. In some cases, a modified peptide can be from about 8 amino acids to about 11 amino acids in length. For example, a modified peptide can be about 11 amino acids in length. A modified peptide can be derived from any modified polypeptide. In some cases, a modified peptide described herein can be derive R248L d from a p53 polypeptide. A modified peptide can include any appropriate modification. In some cases, modified peptides described herein can include one or more modifications (e.g., mutations) shown in Table 1.

TABLE 1
Modified peptides.

| Protein of | | Mutant | SEQ ID |
|---|---|---|---|
| origin | Mutation | Peptide | NO: |
| p53 | R248L | NSSCMGGMNLR | 1 |
| p53 | R248L | CNSSCMGGMNL | 2 |
| p53 | R248L | NSSCMGGMNLRP | 3 |
| p53 | R248L | SSCMGGMNLRP | 4 |
| p53 | R248L | SCMGGMNLRPIS | 5 |
| p53 | R248L | CMGGMNLRPIL | 6 |
| p53 | R248L | MGGMNLRPILT | 7 |
| p53 | R248L | GGMNLRPILTI | 8 |
| p53 | R248L | GMNLRPILTII | 9 |
| p53 | R248L | MNLRPILTIIT | 10 |
| p53 | R248L | NLRPILTIITL | 11 |
| p53 | R248L | LRPILTIITLE | 12 |
| p53 | R248L | CNSSCMGGMN | 13 |
| p53 | R248L | NSSCMGGMNL | 14 |
| p53 | R248L | SSCMGGMNLR | 15 |
| p53 | R248L | SCMGGMNLRP | 16 |
| p53 | R248L | CMGGMNLRPI | 17 |
| p53 | R248L | MGGMNLRPIL | 18 |
| p53 | R248L | GGMNLRPILT | 19 |
| p53 | R248L | GMNLRPILTI | 20 |
| p53 | R248L | MNLRPILTII | 21 |
| p53 | R248L | NLRPILTIIT | 22 |
| p53 | R248L | LRPILTIITL | 23 |
| p53 | R248L | SSCMGGMNL | 24 |
| p53 | R248L | SCMGGMNLR | 25 |
| p53 | R248L | CMGGMNLRP | 26 |
| p53 | R248L | MGGMNLRPI | 27 |
| p53 | R248L | GGMNLRPIL | 28 |
| p53 | R248L | GMNLRPILT | 29 |
| p53 | R248L | MNLRPILTI | 30 |
| p53 | R248L | NLRPILTII | 31 |
| p53 | R248L | LRPILTIIT | 32 |
| p53 | R248L | SCMGGMNL | 33 |
| p53 | R248L | CMGGMNLR | 34 |
| p53 | R248L | MGGMNLRP | 35 |
| p53 | R248L | GGMNLRPI | 36 |
| p53 | R248L | GMNLRPIL | 37 |
| p53 | R248L | MNLRPILT | 38 |
| p53 | R248L | NLRPILTI | 39 |
| p53 | R248L | LRPILTII | 40 |

In some cases, a modified p53 peptide described herein (e.g., a p53 R248L peptide) can be a peptide that is not 100% identical to the mutant peptides set forth in Table 1, but retains the R to L substitution at amino acid residue 248. For example, a modified p53 peptide can include one or more (e.g., one, two, three, four, five, or more) amino acid substitutions relative to a peptide set forth in Table 1.

A modified peptide described herein (e.g., a p53 R248L peptide) can be in a complex with an HLA. An HLA can be any appropriate HLA allele. In some cases, an HLA can be a class I HLA (e.g., HLA-A, HLA-B, and HLA-C) allele. In some cases, an HLA can be a class II HLA (e.g., HLA-DP, HLA-DM, HLA-DOA, HLA-DOB, HLA-DQ, and HLA-DR) allele. An example of a HLA allele that a modified peptide described herein can complex with includes, without limitation, A*68 (e.g., A*68:01).

This document provides TCRs that can bind to a modified peptide described herein (e.g., a p53 R248L peptide). In some cases, a TCR that can bind to a modified peptide described herein does not target (e.g., does not bind to) an uncomplexed modified peptide described herein (e.g., a modified peptide described herein that is not present in a complex (e.g., a peptide-HLA complex)). In some cases, a TCR that can bind to a modified peptide described herein does not target (e.g., does not bind to) a WT peptide (e.g., a peptide derived from a WT polypeptide from which the modified polypeptide is derived).

A TCR that can bind to a modified p53 peptide described herein (e.g., a p53 R248L peptide) can be any appropriate type of TCR. Examples of TCRs that can bind to a modified peptide described herein (e.g., can be designed to bind to a modified peptide described herein) such as a p53 R248L peptide include, without limitation, chimeric antigen receptors (CARs), TCRs, and TCR mimics.

A TCR that can bind to a modified p53 peptide described herein (e.g., a p53 R248L peptide) can include any appropriate alpha (α) chain and any appropriate beta (β) chain. For example, a TCR that can bind to a modified p53 peptide described herein can include an α chain having three complementarity determining regions (TCRα CDRs) and a β chain having three CDRs (TCRβ CDRs).

An α chain of a TCR that can bind to a modified p53 peptide described herein (e.g., a p53 R248L peptide) can include any appropriate CDRs. For example, an a chain of a TCR that can bind to a modified p53 peptide described herein can include can include one of the CDR3s set forth below:

TABLE 2
TCRα-CDR sequences

| | Sequence | SEQ ID NO |
|---|---|---|
| TCRα CDR3 | CILSGANNLFF | 41 |
| TCRα CDR3 | CILYGGATNKLIF | 42 |
| TCRα CDR3 | CILNNNDMRF | 43 |
| TCRα CDR3 | CILKTNSGNTPLVF | 44 |

A β chain of a TCR that can bind to a modified p53 peptide described herein (e.g., a p53 R248L peptide) can include any appropriate CDRs. For example, a β chain of a TCR that can bind to a modified p53 peptide described herein can include can include one of the CDR3s set forth below:

TABLE 3
TCRβ-CDR sequences

| | Sequence | SEQ ID NO |
|---|---|---|
| TCRβ CDR3 | CATTGGQNTEAFF | 45 |
| TCRβ CDR3 | CASQSGILPWEQFF | 46 |
| TCRβ CDR3 | CAISEWRAGSTDTQYF | 47 |
| TCRβ CDR3 | CASSEVQGASNEKLFF | 48 |

In some cases, a TCR that can bind to a modified p53 peptide described herein (e.g., a p53 R248L peptide) can have one or more CDRs that are not 100% identical to the CDRs set forth in Table 2 and Table 3, but retain the ability to bind to the modified p53 peptide. For example, a CDR that includes one or more (e.g., one, two, three, four, five, or more) amino acid substitutions relative to a CDR set forth in Table 2 or Table 3 can be used in TCR provided herein (e.g., a TCR that can bind to a modified p53 peptide such as a p53 R248L peptide). An amino acid substitution can be made, in some cases, by selecting a substitution that does not differ significantly in its effect on maintaining (a) the structure of the peptide backbone in the area of the substitution, (b) the charge or hydrophobicity of the molecule at particular sites, or (c) the bulk of the side chain. For example, naturally occurring residues can be divided into groups based on side-chain properties: (1) hydrophobic amino acids (methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of conservative substitutions that can be made within a CDR of a TCR provided herein include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine.

In some cases, a TCR provided herein (e.g., a TCR that can bind to a modified p53 peptide such as a p53 R248L peptide) can include an α chain that includes a TCRα-CDR3 set forth in any one of SEQ ID NOs:41-44. For example, an α chain that can be included in a TCR that can bind to a modified p53 peptide described herein (e.g., a p53 R248L peptide) can include the amino acid sequence set forth in SEQ ID NO:41-44.

In some cases, a TCR provided herein (e.g., a TCR that can bind to a modified p53 peptide such as a p53 R248L peptide) can include a β chain that includes a TCRβ-CDR3 set forth in any one of SEQ ID NOs:45-48. For example, a β chain that can be included in a TCR that can bind to a modified p53 peptide described herein (e.g., a p53 R248L peptide) can include the amino acid sequence set forth in SEQ ID NO:45-48.

In some cases, a TCR provided herein (e.g., a TCR that can bind to a modified p53 peptide such as a p53 R248L peptide) can include an a chain that includes a TCRα-CDR3 set forth in any one of SEQ ID NOs:41-44, and a β chain that includes a TCRβ-CDR3 set forth in any one of SEQ ID NOs:45-48. For example, a TCR that can bind to a modified p53 peptide described herein (e.g., a p53 R248L peptide) can include an α chain including the amino acid sequence set forth in SEQ ID NO:41-44 and a β chain including the amino acid sequence set forth in SEQ ID NO:45-48.

This document also provides nucleic acid (e.g., nucleic acid vectors) that can encode a TCR provided herein (e.g., a TCR that can bind to a modified p53 peptide such as a p53 R248L peptide). Nucleic acid (e.g., nucleic acid vectors) that can encode a TCR provided herein can be any type of nucleic acid. Nucleic acid can be DNA (e.g., a DNA construct), RNA (e.g., mRNA), or a combination thereof. In some cases, nucleic acid that can encode a TCR provided herein can be a vector (e.g., an expression vector or a viral vector).

In some cases, nucleic acid that can encode a TCR provided herein (e.g., a TCR that can bind to a modified p53 peptide such as a p53 R248L peptide) can also include one or more regulatory elements (e.g., to regulate expression of the amino acid chain). Examples of regulatory elements that can be included in nucleic acid that can encode a TCR provided herein include, without limitation, promoters (e.g., constitutive promoters, tissue/cell-specific promoters, and inducible promoters such as chemically-activated promoters and light-activated promoters), and enhancers.

This document also provides cells (e.g., host cells) expressing one or more TCRs provided herein (e.g., one or more TCRs that can bind to a modified p53 peptide such as a p53 R248L peptide). A cell expressing one or more TCRs provided herein can be any appropriate type of cell. In some cases, a cell expressing one or more TCRs provided herein can be a T cell (e.g., a CD4⁺ T cell or a CD8⁺ T cell). A cell expressing one or more TCRs provided herein can obtained from any type of animal. In some cases, a cell expressing one or more TCRs provided herein can be obtained from a human or a non-human mammal such as a mouse. When using a cell expressing one or more TCRs provided herein to treat a mammal having a cancer (e.g., a cancer containing one or more cancer cells expressing a modified p53 peptide such as a p53 R248L peptide), the cell can be obtained from the mammal to be treated or from another source.

This document also provides methods for using TCRs (e.g., T cells expressing one or more TCRs that can bind to a modified p53 peptide such as a p53 R248L peptide). For example, T cells expressing one or more TCRs provided herein (e.g., one or more TCRs that can bind to a modified p53 peptide such as a p53 R248L peptide) that can target (e.g., bind to) cancer cells expressing the modified p53 peptide. In some cases, T cells expressing one or more TCRs provided herein (e.g., one or more TCRs that can bind to a modified p53 peptide such as a p53 R248L peptide) can be administered to a mammal having a cancer (e.g., a cancer containing cancer cells expressing a modified p53 peptide such as a p53 R248L peptide) to treat the mammal. Administration of T cells expressing one or more TCRs provided herein (e.g., one or more TCRs that can bind to a modified p53 peptide such as a p53 R248L peptide) to a mammal (e.g., human) having a cancer can be effective to treat the mammal.

In some cases, T cells expressing one or more TCRs provided herein (e.g., one or more TCRs that can bind to a modified p53 peptide such as a p53 R248L peptide) can be administered to a mammal (e.g., a human) in need thereof (e.g., a human having cancer such as a cancer containing cancer cells that express a p53 R248L peptide) to reduce or eliminate the number of cancer cells present within a mammal. For example, the materials and methods described herein can be used to reduce the number of cancer cells present within a mammal having cancer by, for example, 10, 20, 30, 40, 50, 60, 70, 80, 90, 95, or more percent. For example, the materials and methods described herein can be used to reduce the size (e.g., volume) of one or more tumors present within a mammal having cancer by, for example, 10, 20, 30, 40, 50, 60, 70, 80, 90, 95, or more percent.

In some cases, T cells expressing one or more TCRs provided herein (e.g., one or more TCRs that can bind to a modified p53 peptide such as a p53 R248L peptide) can be administered to a mammal (e.g., a human) in need thereof (e.g., a human having cancer such as a cancer containing cancer cells that express a p53 R248L peptide) to improve survival of the mammal. For example, disease-free survival (e.g., relapse-free survival) can be improved using the materials and methods described herein. For example, progression-free survival can be improved using the materials and methods described herein. In some cases, the materials and methods described herein can be used to improve the survival of a mammal having cancer by, for example, 10, 20, 30, 40, 50, 60, 70, 80, 90, 95, or more percent.

In some cases, T cells expressing one or more TCRs provided herein (e.g., one or more TCRs that can bind to a modified p53 peptide such as a p53 R248L peptide) can be administered to a mammal (e.g., a human) in need thereof (e.g., a human having cancer such as a cancer containing cancer cells that express a p53 R248L peptide) to increase the number of tumor-infiltrating lymphocytes (e.g., T cells present in within the tumor microenvironment of a cancer) within the mammal. For example, the materials and methods described herein can be used to increase the number of tumor-infiltrating lymphocytes within a mammal having cancer by, for example, 10, 20, 30, 40, 50, 60, 70, 80, 90, 95, or more percent.

Any type of mammal can be treated as described herein. Examples of mammals that can be treated as described herein include, without limitation, primates (e.g., humans and non-human primates such as chimpanzees, baboons, or monkeys), dogs, cats, pigs, sheep, rabbits, mice, and rats. In some cases, a mammal can be a human.

A mammal can be treated for any appropriate cancer. In some cases, a cancer can include one or more cancers cells expressing one or more modified peptides (e.g., one or more MANAs) described herein (e.g., a modified p53 peptide such as a p53 R248L peptide). A cancer can be a primary cancer. A cancer can be a metastatic cancer. A cancer can include one or more solid tumors. A cancer can include one or more non-solid tumors. Examples of cancers that can be treated as described herein (e.g., by administering T cells expressing one or more TCRs that can bind to a modified p53 peptide such as a p53 R248L peptide) include, without limitation, lung cancers (e.g., non-small cell lung cancers (NSCLCs)), colon adenocarcinomas, rectal adenocarcinomas, head and neck squamous cell carcinomas, pancreatic adenocarcinomas, melanomas, urothelial carcinomas, uterine corpus endometrial carcinomas, and uterine carcinomas.

In some cases, the methods described herein also can include identifying a mammal as having cancer. Examples of methods for identifying a mammal as having cancer include, without limitation, physical examination, laboratory tests (e.g., blood and/or urine), biopsy, imaging tests (e.g., X-ray, PET/CT, Mill, and/or ultrasound), nuclear medicine scans (e.g., bone scans), endoscopy, and/or genetic tests. Once identified as having cancer, a mammal can be administered or instructed to self-administer T cells expressing one or more TCRs provided herein (e.g., one or more TCRs that can bind to a modified p53 peptide such as a p53 R248L peptide).

When treating a mammal having cancer, T cells expressing one or more TCRs provided herein (e.g., one or more TCRs that can bind to a modified p53 peptide such as a p53 R248L peptide) can be administered to a mammal having cancer to treat the mammal. In some cases, a mammal can have a cancer that includes one or more cancer cells expressing one or more modified peptides described herein. For example, T cells expressing one or more TCRs provided herein can be administered to a mammal having a cancer that includes one or more cancer cells expressing that modified peptide to treat the mammal. For example.

In some cases, T cells expressing one or more TCRs provided herein (e.g., one or more TCRs that can bind to a modified p53 peptide such as a p53 R248L peptide) can be administered to a mammal having cancer (e.g., a cancer containing one or more cancer cells expressing a modified p53 peptide such as a p53 R248L peptide) once.

In some cases, T cells expressing one or more TCRs provided herein (e.g., one or more TCRs that can bind to a modified p53 peptide such as a p53 R248L peptide) can be administered to a mammal having cancer (e.g., a cancer containing one or more cancer cells expressing a modified p53 peptide such as a p53 R248L peptide) multiple times (e.g., over a period of time ranging from days to weeks to months).

In some cases, T cells expressing one or more TCRs provided herein (e.g., one or more TCRs that can bind to a modified p53 peptide such as a p53 R248L peptide) can be formulated into a composition (e.g., a pharmaceutically acceptable composition) for administration to a mammal having a cancer (e.g., a cancer containing one or more cancer cells expressing a p53 R248L peptide). For example, T cells expressing one or more TCRs provided herein (e.g., one or more TCRs that can bind to a modified p53 peptide such as a p53 R248L peptide) can be formulated together with one or more pharmaceutically acceptable carriers (additives), excipients, and/or diluents. In some cases, a pharmaceutically acceptable carrier, excipient, or diluent can be a naturally occurring pharmaceutically acceptable carrier, excipient, or diluent. In some cases, a pharmaceutically acceptable carrier, excipient, or diluent can be a non-naturally occurring (e.g., an artificial or synthetic) pharmaceutically acceptable carrier, excipient, or diluent. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, benzyl alcohol, lysine hydrochloride, trehalose dihydrate, sodium hydroxide, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil. In some cases, a pharmaceutically acceptable carrier, excipient, or diluent can be an antiadherent, a binder, a colorant, a disintegrant, a flavor (e.g., a natural flavor such as a fruit extract or an artificial flavor), a glidant, a lubricant, a preservative, a sorbent, and/or a sweetener.

A composition (e.g., a pharmaceutical composition) containing T cells expressing one or more TCRs provided herein (e.g., one or more TCRs that can bind to a modified p53 peptide such as a p53 R248L peptide) can be formulated into any appropriate dosage form. Examples of dosage forms include liquid forms including, without limitation, suspensions, solutions (e.g., sterile solutions), sustained-release formulations, and delayed-release formulations.

A composition containing T cells expressing one or more TCRs provided herein (e.g., one or more TCRs that can bind to a modified p53 peptide such as a p53 R248L peptide) can be designed for oral, parenteral (including subcutaneous, intramuscular, intravenous, and intradermal), or intratumoral administration. Compositions suitable for parenteral administration include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules, and tablets.

A composition containing T cells expressing one or more TCRs provided herein (e.g., one or more TCRs that can bind to a modified p53 peptide such as a p53 R248L peptide) can be administered using any appropriate technique and to any appropriate location. A composition including T cells expressing one or more TCRs provided herein (e.g., one or more TCRs that can bind to a modified p53 peptide such as a p53 R248L peptide) can be administered locally or systemically. For example, a composition provided herein can be administered locally by intratumoral administration (e.g., injection into tumors) or by administration into biological spaces infiltrated by tumors (e.g. intraspinal administration, intracerebellar administration, intraperitoneal administration and/or pleural administration). For example, a composition provided herein can be administered systemically by oral administration or by intravenous administration (e.g., injection or infusion) to a mammal (e.g., a human).

Effective doses can vary depending on the risk and/or the severity of the cancer, the route of administration, the age and general health condition of the subject, excipient usage, the possibility of co-usage with other therapeutic treatments such as use of other agents, and the judgment of the treating physician. An effective amount of T cells expressing one or more TCRs provided herein (e.g., one or more TCRs that can bind to a modified p53 peptide such as a p53 R248L peptide) can be any amount that treats a cancer present within the subject without producing significant toxicity to the subject. If a particular subject fails to respond to a particular amount, then the amount of one or more molecules including one or more antigen-binding domains (e.g., scFvs) that can bind to a modified peptide described herein can be increased (e.g., by two-fold, three-fold, four-fold, or more). After receiving this higher amount, the mammal can be monitored for both responsiveness to the treatment and toxicity symptoms, and adjustments made accordingly. The effective amount can remain constant or can be adjusted as a sliding scale or variable dose depending on the subject's response to treatment. Various factors can influence the actual effective amount used for a particular application. For example, the frequency of administration, duration of treatment, use of multiple treatment agents, route of administration, and severity of the condition (e.g., cancer) may require an increase or decrease in the actual effective amount administered.

The frequency of administration of T cells expressing one or more TCRs provided herein (e.g., one or more TCRs that can bind to a modified p53 peptide such as a p53 R248L peptide) can be any frequency that effectively treats a mammal having a cancer without producing significant toxicity to the mammal. For example, the frequency of administration of T cells expressing one or more TCRs provided herein (e.g., one or more TCRs that can bind to a modified p53 peptide such as a p53 R248L peptide) can be from about two to about three times a week to about two to about three times a year. In some cases, a mammal having cancer can receive a single administration of T cells expressing one or more TCRs provided herein (e.g., one or more TCRs that can bind to a modified p53 peptide such as a p53 R248L peptide). The frequency of administration of T cells expressing one or more TCRs provided herein (e.g., one or more TCRs that can bind to a modified p53 peptide such as a p53 R248L peptide) can remain constant or can be variable during the duration of treatment. A course of treatment with T cells expressing one or more TCRs provided herein (e.g., one or more TCRs that can bind to a modified p53 peptide such as a p53 R248L peptide) can include rest periods. For example, T cells expressing one or more TCRs provided herein (e.g., one or more TCRs that can bind to a modified p53 peptide such as a p53 R248L peptide) can be administered every other month over a two-year period followed by a six-month rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., cancer) may require an increase or decrease in administration frequency.

An effective duration for administering T cells expressing one or more TCRs provided herein (e.g., one or more TCRs that can bind to a modified p53 peptide such as a p53 R248L peptide) can be any duration that effectively treats a cancer present within the mammal without producing significant toxicity to the mammal. In some cases, the effective duration can vary from several months to several years. In general, the effective duration for treating a mammal having a cancer can range in duration from about one or two months to five or more years. Multiple factors can influence the actual effective duration used for a particular treatment. For example, an effective duration can vary with the frequency of administration, effective amount, use of multiple treatment agents, route of administration, and severity of the condition being treated.

In certain instances, a cancer within a mammal can be monitored to evaluate the effectiveness of the cancer treatment. Any appropriate method can be used to determine whether or not a mammal having cancer is treated. For example, imaging techniques or laboratory assays can be used to assess the number of cancer cells and/or the size of a tumor present within a mammal. For example, imaging techniques or laboratory assays can be used to assess the location of cancer cells and/or a tumor present within a mammal.

In some cases, T cells expressing one or more TCRs provided herein (e.g., one or more TCRs that can bind to a modified p53 peptide such as a p53 R248L peptide) can be administered to a mammal having a cancer as a combination therapy with one or more co-stimulatory molecules. In some cases, a co-stimulatory molecule can be an agonist of one or more co-stimulatory receptors. Examples of co-stimulatory molecules that can be administered to mammal having cancer together with T cells expressing one or more TCRs provided herein include, without limitation, anti-GITR antibodies, anti-CD27 antibodies, anti-4-1BB antibodies, anti-OX40 antibodies, anti-ICOS antibodies, and anti-CD40 antibodies.

In some cases, T cells expressing one or more TCRs provided herein (e.g., one or more TCRs that can bind to a modified p53 peptide such as a p53 R248L peptide) can be administered to a mammal having a cancer as a combination therapy with one or more additional cancer treatments. A cancer treatment can include any appropriate cancer treatments. For example, a cancer treatment can include surgery. For example, a cancer treatment can include radiation therapy. For example, a cancer treatment can include administration of one or more therapeutic agents (e.g., one or more anti-cancer agents). In some cases, an anti-cancer agent can be an immunotherapy (e.g., a checkpoint inhibitor). Examples of anti-cancer agents that can be administered together with T cells expressing one or more TCRs provided herein include, without limitation, anti-CTLA-4 antibodies, anti-PD-1 antibodies, anti-PD-L1 antibodies, anti-LAG3 antibodies, anit-Tim3 antibodies, anti-TIGIT antibodies, anti-CD39 antibodies, anti-VISTA antibodies, anti-CD47 antibodies, anti-SIRPalpha antibodies, anti-B7-H3 antibodies, anti-B7-H4 antibodies, anti-neuritin antibodies, anti-neuropilin antibodies, anti-IL-35 antibodies, inhibitors of IDO, inhibitors of A2AR, inhibitors of arginase, and inhibitors of glutaminase. In cases where an immunotherapy is administered to mammal having cancer together with T cells expressing one or more TCRs provided herein, the mammal also can be administered one or more co-stimulatory molecules (e.g., one or more agonists of one or more co-stimulatory receptors such as anti-GITR antibodies, anti-CD27 antibodies, anti-4-1BB antibodies, anti-OX40 antibodies, anti-ICOS antibodies, and anti-CD40 antibodies).

In cases where T cells expressing one or more TCRs provided herein (e.g., one or more TCRs that can bind to a modified p53 peptide such as a p53 R248L peptide) are used in combination with one or more additional cancer treatments, the one or more additional cancer treatments can be administered at the same time or independently. For example, T cells expressing one or more TCRs provided herein (e.g., one or more TCRs that can bind to a modified p53 peptide such as a p53 R248L peptide) can be administered first, and the one or more additional cancer treatments administered second, or vice versa. In cases, where T cells expressing one or more TCRs provided herein (e.g., one or more TCRs that can bind to a modified p53 peptide such as a p53 R248L peptide) and one or more anti-cancer agents are administered at the same time, the T cells expressing one or more TCRs provided herein and the one or more anti-cancer agents can be formulated into a single composition.

Also provided herein are kits that include one or more TCRs provided herein (e.g., one or more TCRs that can bind to a modified p53 peptide such as a p53 R248L peptide) and/or nucleic acid that can encode a TCR provided herein (e.g., a TCR that can bind to a modified p53 peptide such as a p53 R248L peptide). For example, a kit can include one or more vectors that can encode a TCR provided herein (e.g., a TCR that can bind to a modified p53 peptide such as a p53 R248L peptide) and can be used to generate T cells expressing one or more TCRs provided herein (e.g., one or more TCRs that can bind to a modified p53 peptide such as a p53 R248L peptide). In some cases, a kit can include instructions for generating T cells expressing one or more TCRs provided herein (e.g., one or more TCRs that can bind to a modified p53 peptide such as a p53 R248L peptide). For example a kit can include one or more vectors that can encode a TCR provided herein (e.g., a TCR that can bind to a modified p53 peptide such as a p53 R248L peptide) and can be used to generate T cells expressing one or more TCRs provided herein (e.g., one or more TCRs that can bind to a modified p53 peptide such as a p53 R248L peptide) and can include T cells. In some cases, a kit also can include instructions for generating T cells expressing one or more TCRs provided herein (e.g., one or more TCRs that can bind to a modified p53 peptide such as a p53 R248L peptide) and for using the generated T cells (e.g., for performing any of the methods described herein). In some cases, a kit can provide a means (e.g., a syringe) for administering T cells expressing one or more TCRs provided herein (e.g., one or more TCRs that can bind to a modified p53 peptide such as a p53 R248L peptide) to a mammal.

The invention will be further described in the following examples, which do not limit the scope of the invention described in the claims.

### EXAMPLES

### Example 1: Distinct Transcriptional Programs Characterize Neoantigen-Specific T Cells in Lung Cancers Treated with Neoadjuvant PD-1 Blockade

TP53 is the most commonly mutated cancer driver gene, but despite extensive efforts, no drug targeting mutant TP53 has been approved for treatment of the large number of patients whose tumor contain p53 mutations.

This Example describes the identification of MANA specific T cell clones and their function in the tumor microenvironment.

### Methods

### Patients and Biospecimens

All biospecimens were obtained from patients with stage I-IIIA NSCLC who were enrolled to a phase II clinical trial evaluating the safety and feasibility of administering two doses of anti-PD-1 (nivolumab) prior to surgical resection. Pathological response assessments of primary tumors were as reported elsewhere (see, e.g., Forde et al., N. Engl. J. Med., 378:1976-1986 (2018); and Cottrell et al., Ann. Oncol., 29:1853-1860 (2018)). Tumors with no more than 10% residual viable tumor cells were considered to have a major pathologic response.

### Single Cell TCRseq/RNAseq

Cryobanked T cells were thawed and washed twice with pre-warmed RPMI with 20% FBS and gentamicin. Cells were resuspended in PBS and stained with a viability marker (LIVE/DEAD™ Fixable Near-IR; ThermoFisher) for 15 minutes at room temperature (RT) in the dark. Cells were the incubated with FC block for 15 minutes on ice and stained with antibody against CD3 (BV605, clone SK7) for 30 minutes on ice. After staining, highly viable CD3⁺T cells were sorted into 0.04% BSA in PBS using a BD FACSAria II Cell Sorter. Sorted cells were manually counted using a hemocytometer and prepared at the desired cell concentration (1000 cells/μL), when possible. The Single Cell 5′ V(D)J and 5′ DGE kits (10X Genomics) were used to capture immune repertoire information and gene expression from the same cell in an emulsion-based protocol at the single cell level. Cells and barcoded gel beads were partitioned into nanoliter scale droplets using the 10X Genomics Chromium platform to partition up to 10,000 cells per sample followed by RNA capture and cell-barcoded cDNA synthesis using the manufacturer's standard protocols. Libraries were generated and sequenced on an Illumina HiSeq or NovaSeq instrument using 2×150 bp paired end sequencing. 5′ VDJ libraries were sequenced to a depth of ˜5,000 reads per cell, for a total of 5 million to 25 million reads. The 5′ DGE libraries were sequenced to a target depth of ˜5,000 reads per cell. The 5′ DGE libraries were sequenced to a target depth of ˜50,000 reads per cell.

### Single Cell VDJ and DGE Data Processing

Cell Ranger v3.1.0 was used to demultiplex the FASTQ reads, align them to the GRCh38 human transcriptome, and extract their “cell” and “UMI” barcodes. The output of this pipeline is a digital gene expression (DGE) matrix for each sample, which records the number of UMIs for each gene that are associated with each cell barcode. Quality of cells were then assessed based on (1) the number of detected genes per cell and (2) the proportion of mitochondrial gene/ribosomal gene counts. Low-quality cells were filtered if the number of detected genes was below 250 or above the medians of all cells plus 3×the median absolute deviation. Cells were filtered out if the proportion of mitochondrial gene counts was higher than 10% or the percent of ribosomal genes was less than 10%. For single-cell VDJ sequencing, only cells with full-length sequences were retained. The SAVER algorithm was used to impute dropouts and adjust unreliable gene expression quantification caused by sparse data by borrowing information across similar genes and cells. After appropriate transformation (e.g., log2), gene expression values were quantile normalized across samples. Using the normalized single-cell data, cells were projected to a common low-dimensional space (e.g., by UMAP49). The Mutual Nearest Neighbors (MNN) approach was used to align cells so that cells of the same cell type from different samples are matched in an unsupervised fashion. Unsupervised clustering of cells was then performed to systematically identify cell subpopulations, including potential new cell subtypes. The TCR beta chain (at the nucleotide level) was used to match MANAFEST positive T cell clones on the UMAP. A “clonotype” was defined by a unique combination of a TCR alpha and beta chain. Single cell data were pre-processed and normalized separately and UMAPs were generated for each patient.

### Whole Exome Sequencing (WES), Mutation Calling, and Neoantigen Prediction

Genomic data for most patients in the study was as reported elsewhere (see, e.g., Forde et al., N. Engl. J Med., 378:1976-1986 (2018)). Tumor mutational burden and neoantigen predictions for patients MD043-003 and NY016-025 were performed. Whole exome sequencing was performed on pre-treatment tumor for NY016-025 and resected tumor for MD043-003 and matched normal samples. DNA was extracted from patients' tumors and matched peripheral blood using the Qiagen DNA kit (Qiagen, CA). Fragmented genomic DNA from tumor and normal samples was used for Illumina TruSeq library construction (Illumina, San Diego, CA) and exonic regions were captured in solution using the Agilent SureSelect v.4 kit (Agilent, Santa Clara, CA) according to the manufacturers' instructions. Paired-end sequencing, resulting in 100 bases from each end of the fragments for the exome libraries was performed using Illumina HiSeq 2000/2500 instrumentation (Illumina, San Diego, CA). Somatic mutations, consisting of point mutations, insertions, and deletions across the whole exome were identified using the VariantDx custom software for identifying mutations in matched tumor and normal samples. Somatic mutations, consisting of nonsynonymous single base substitutions, insertions and deletions, were evaluated for putative MHC class I neoantigens using the ImmunoSelect-R pipeline (Personal Genome Diagnostics, Baltimore, MD).

### Identification of Neoantigen-Specific TCR V/3 CDR3 Clonotypes

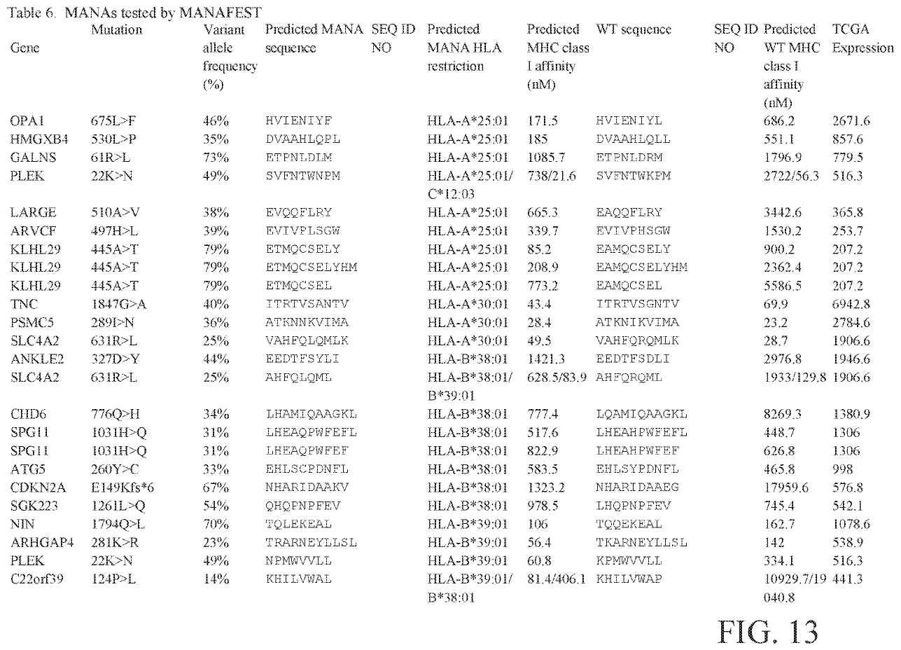

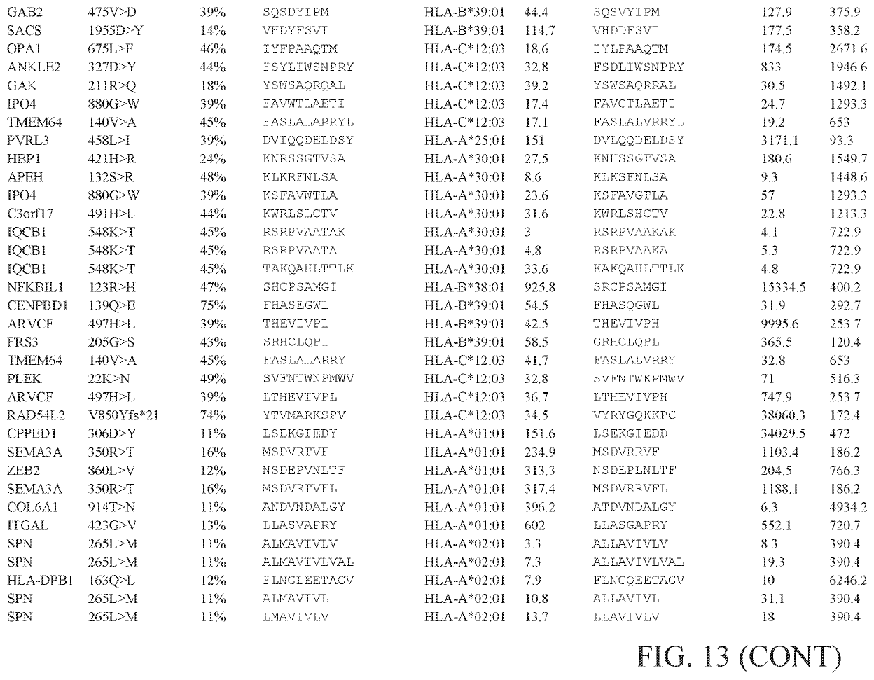

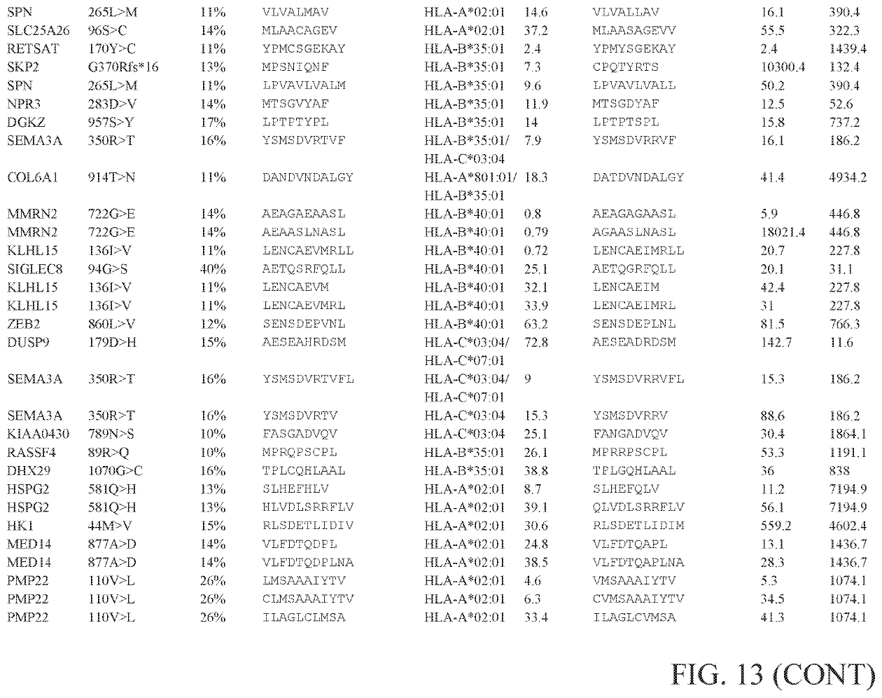

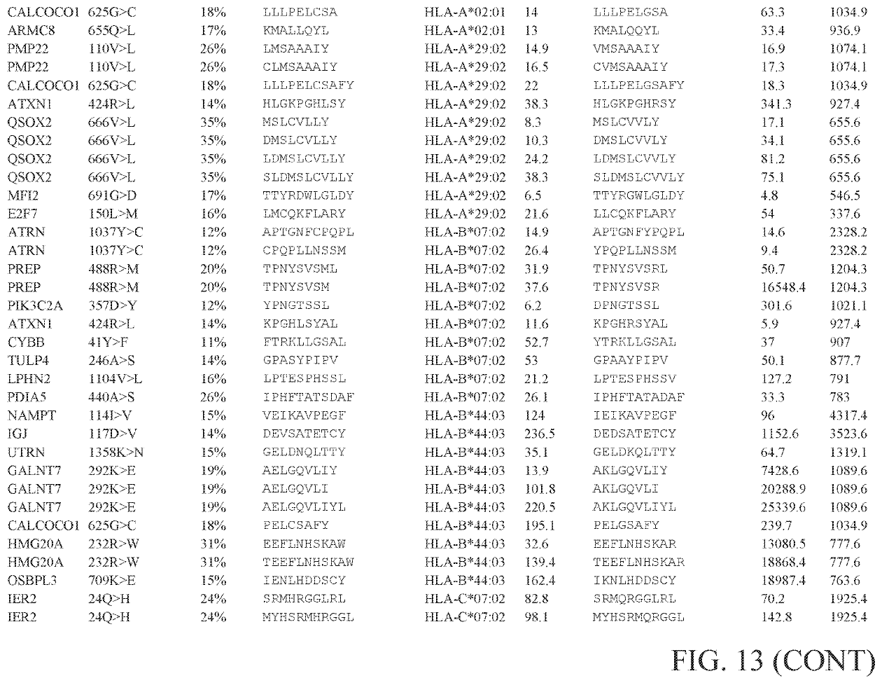

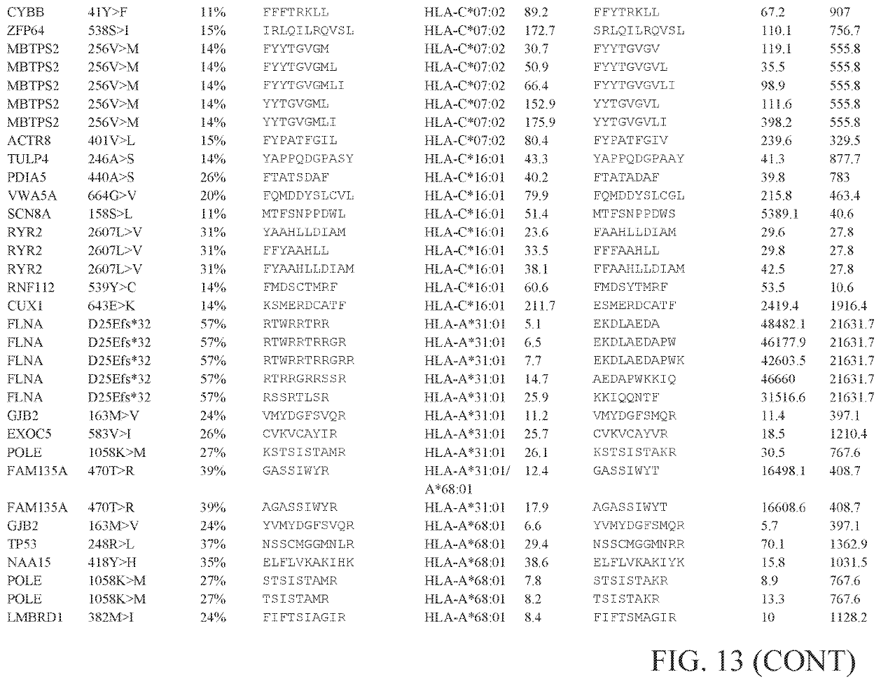

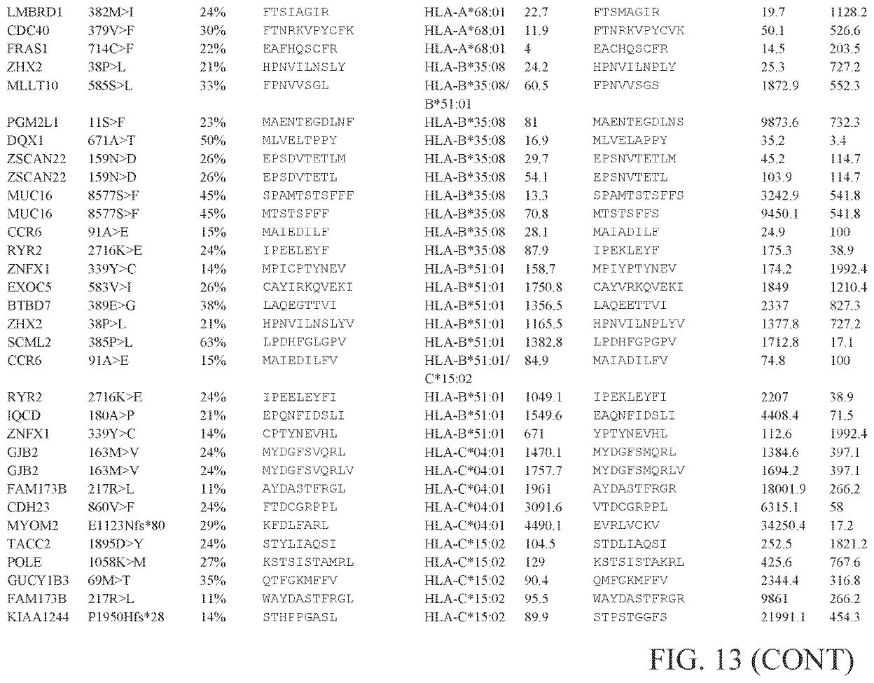

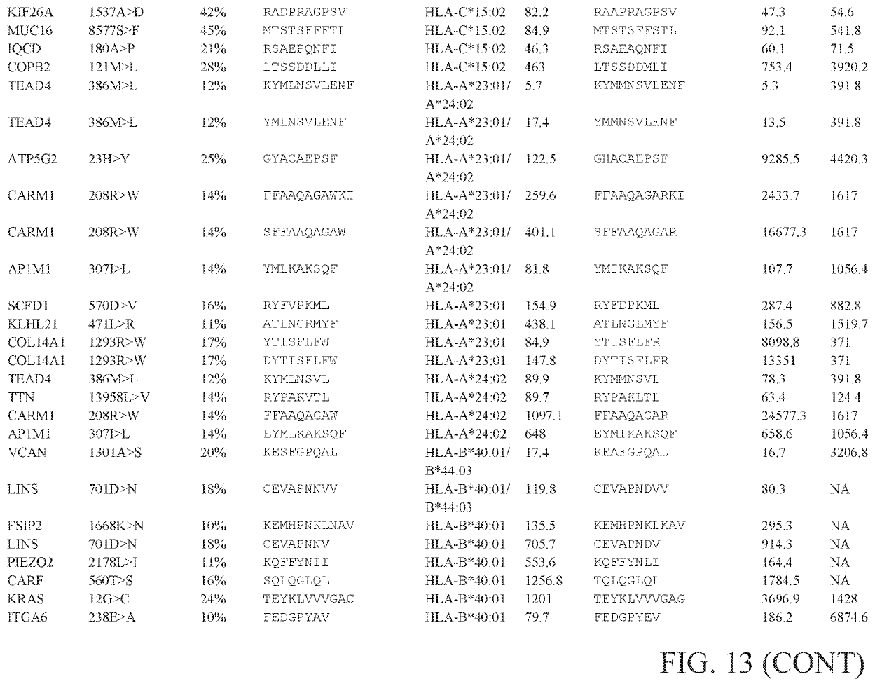

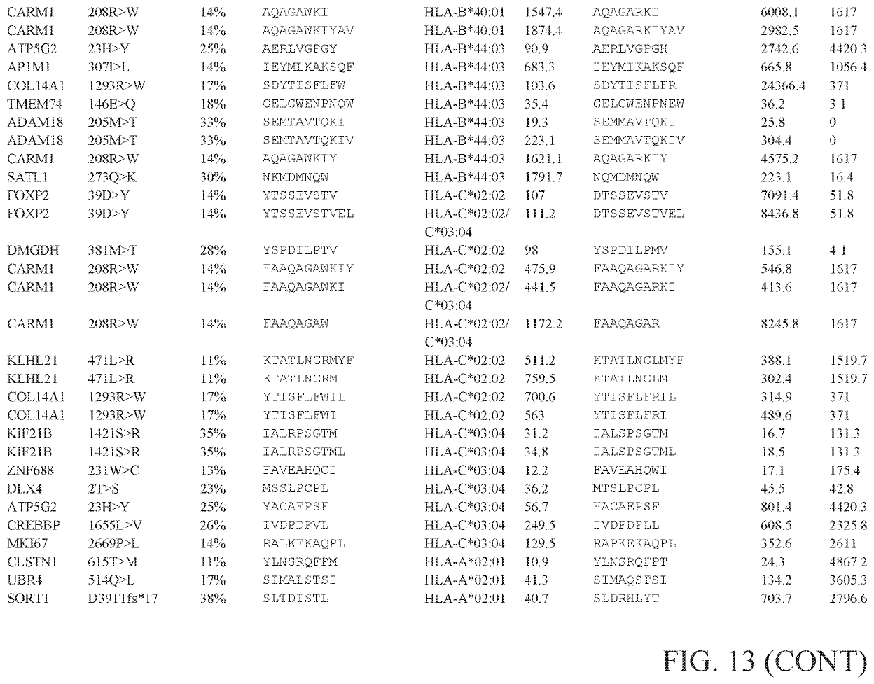

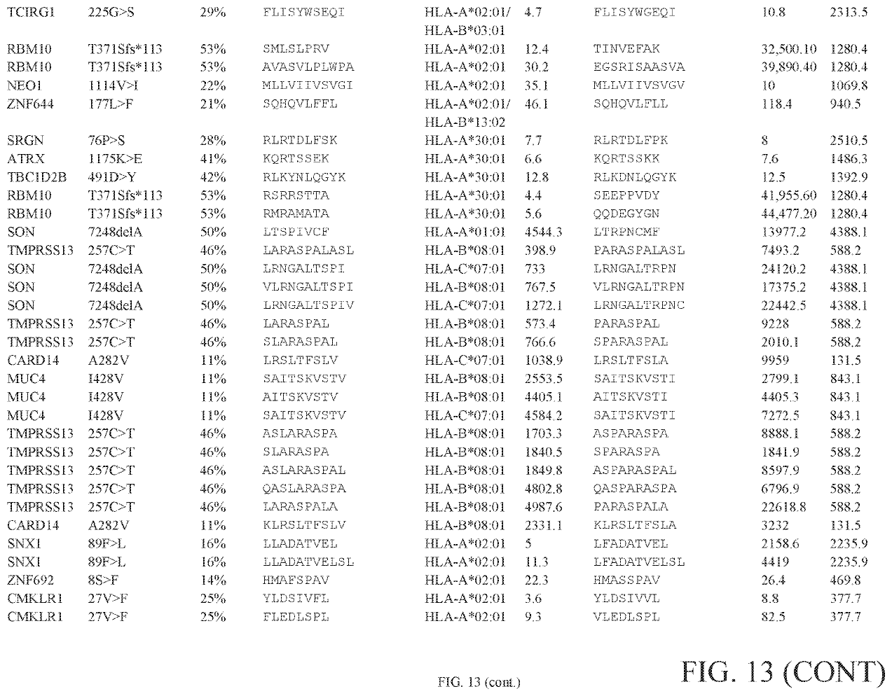

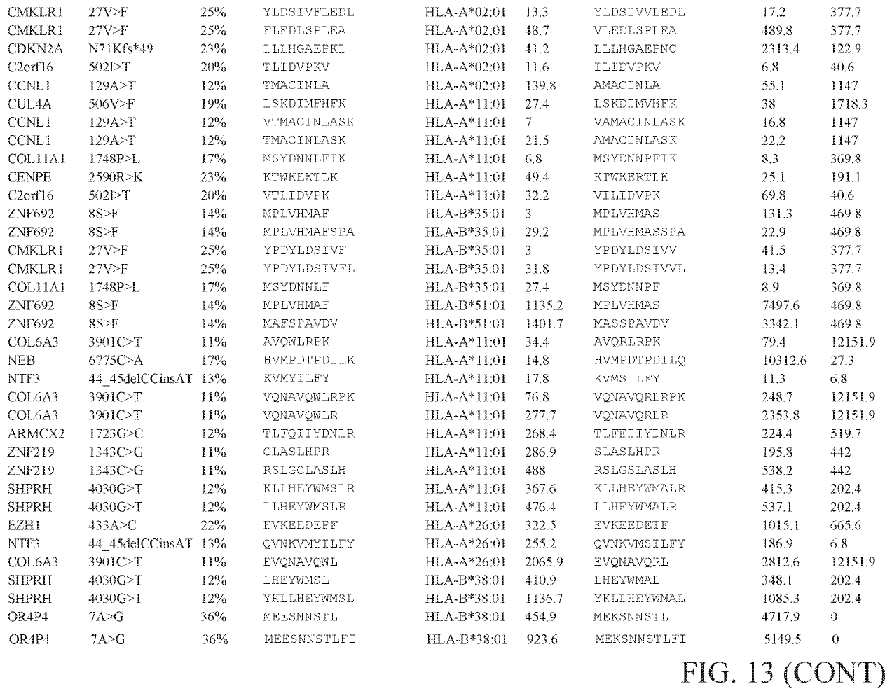

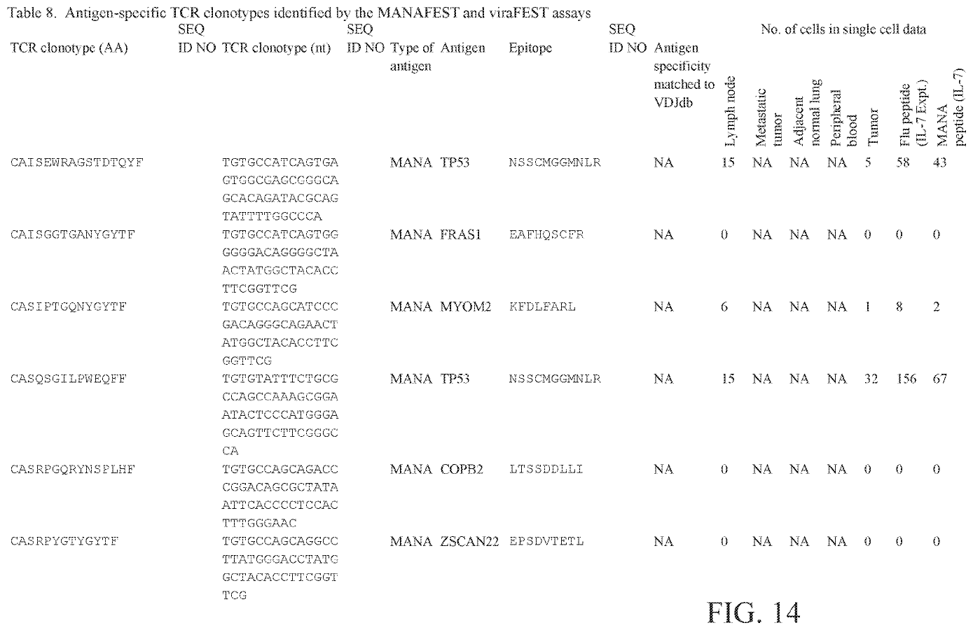

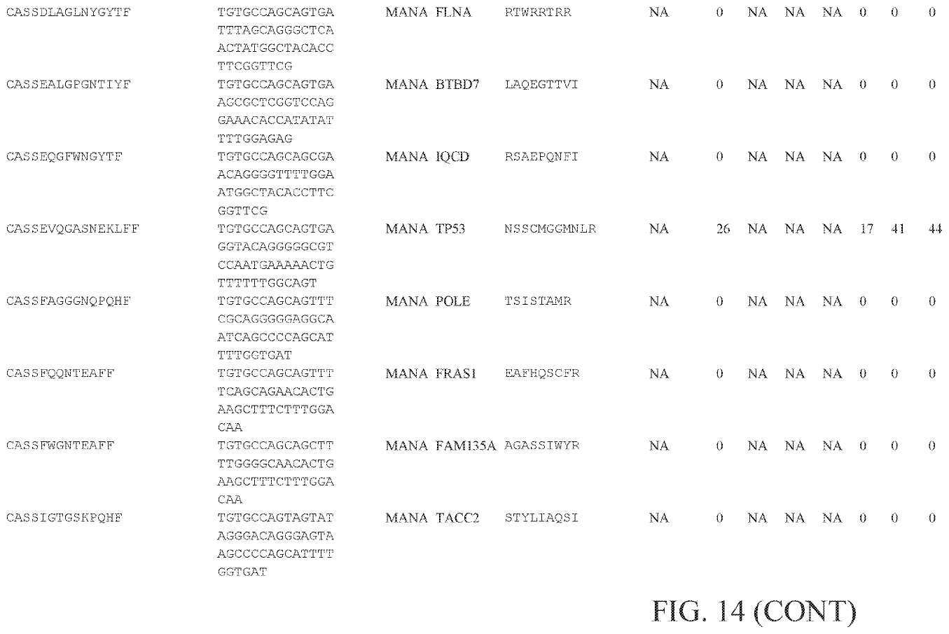

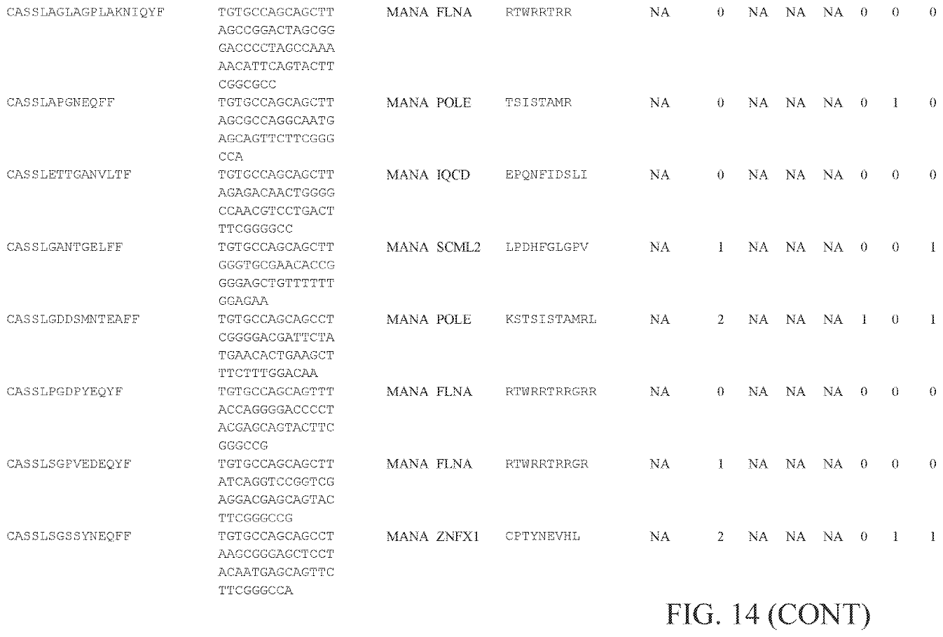

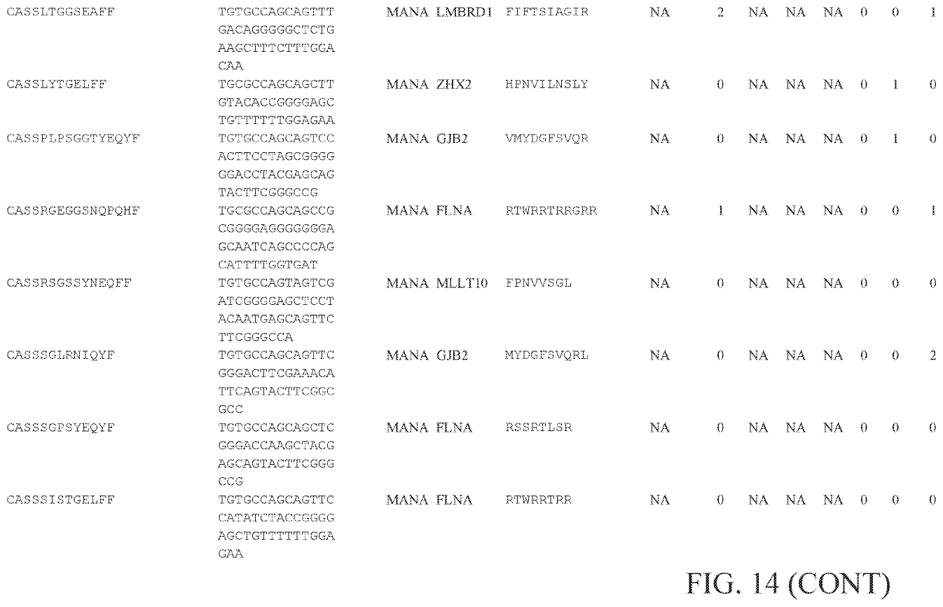

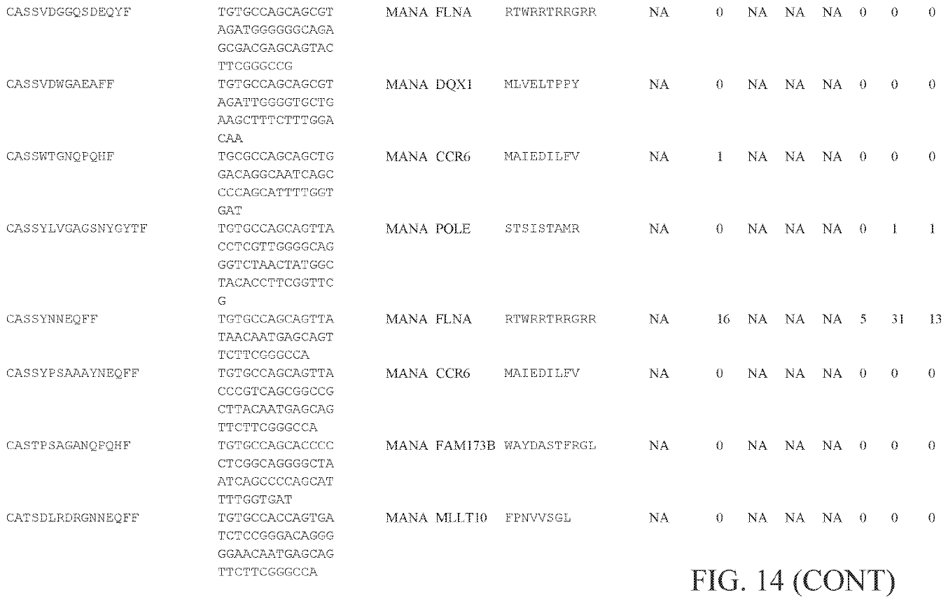

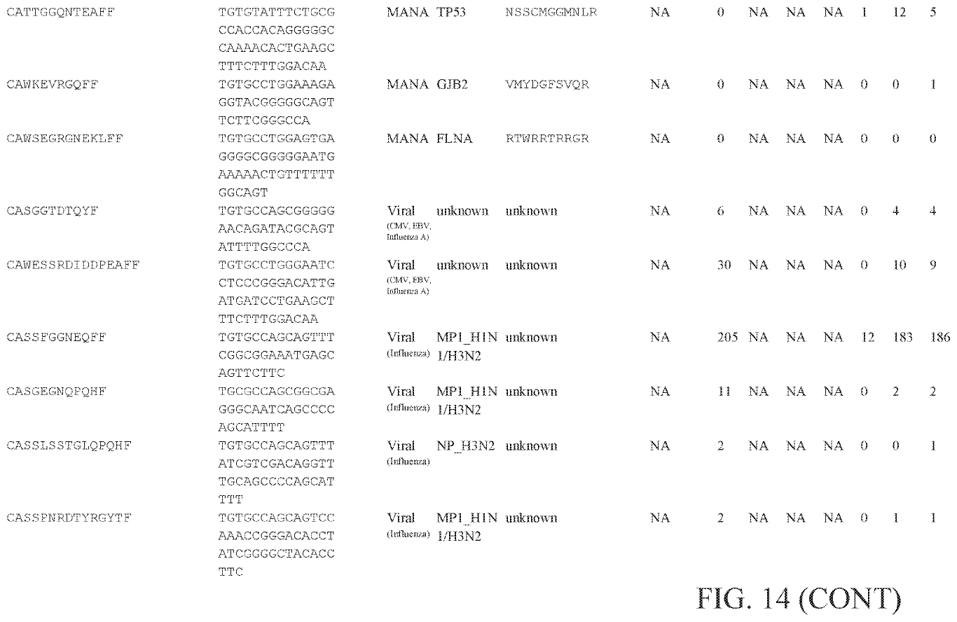

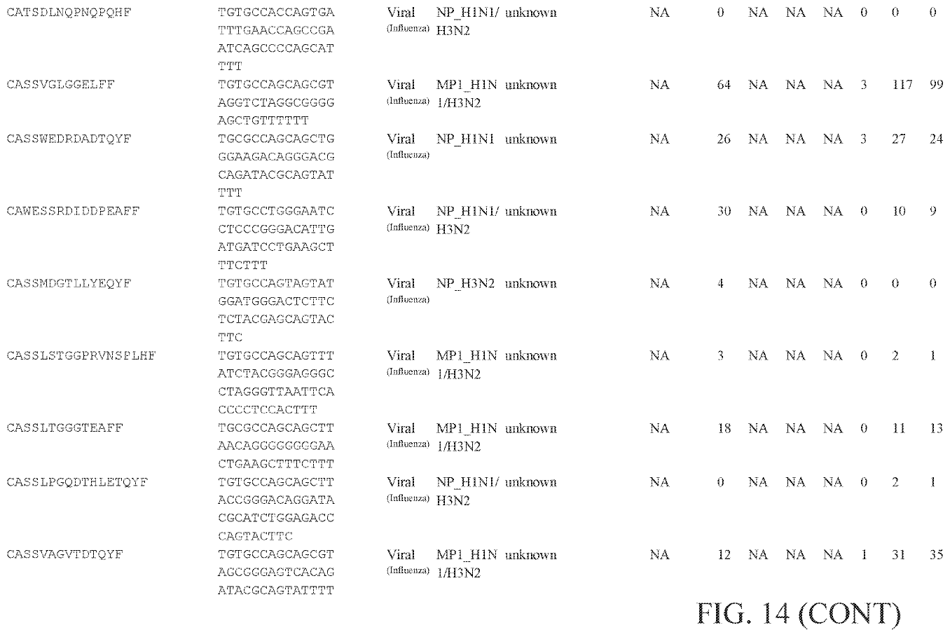

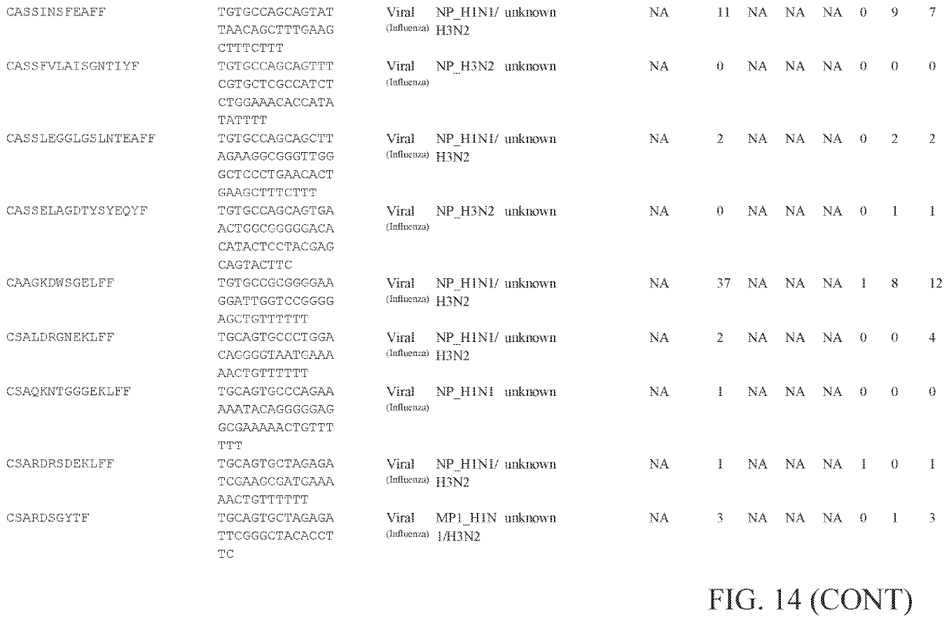

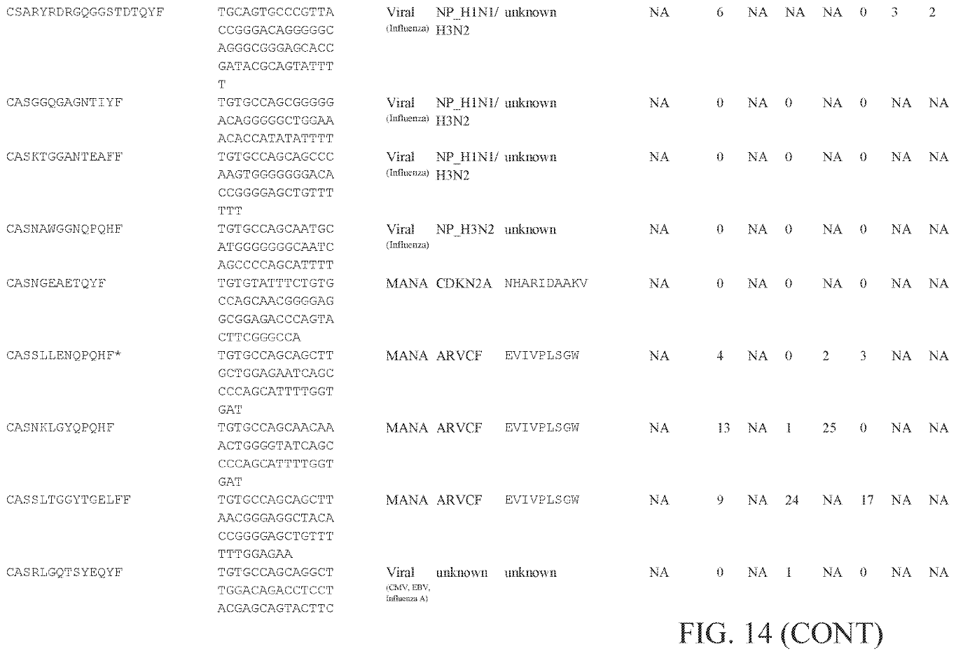

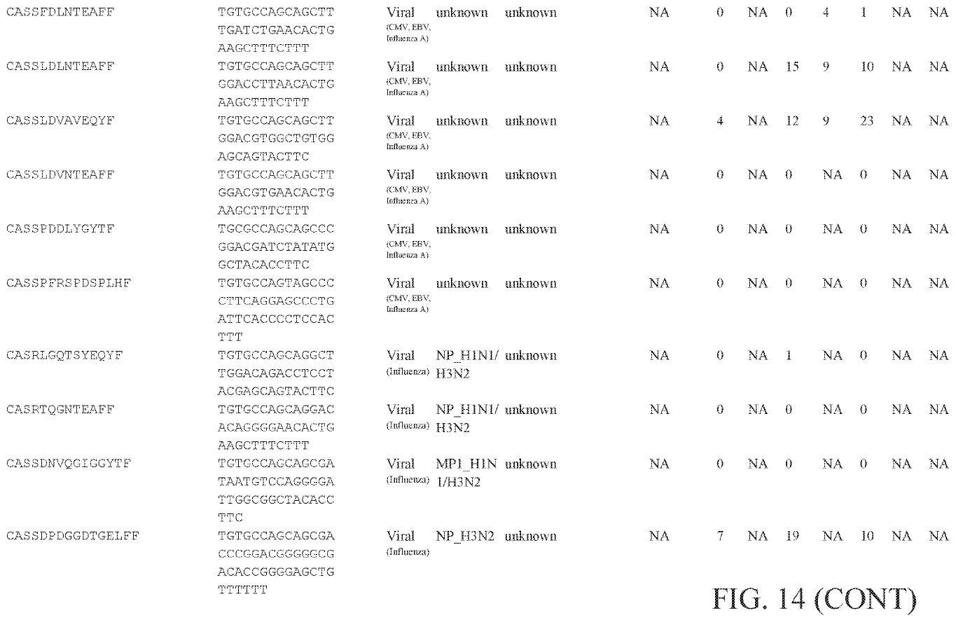

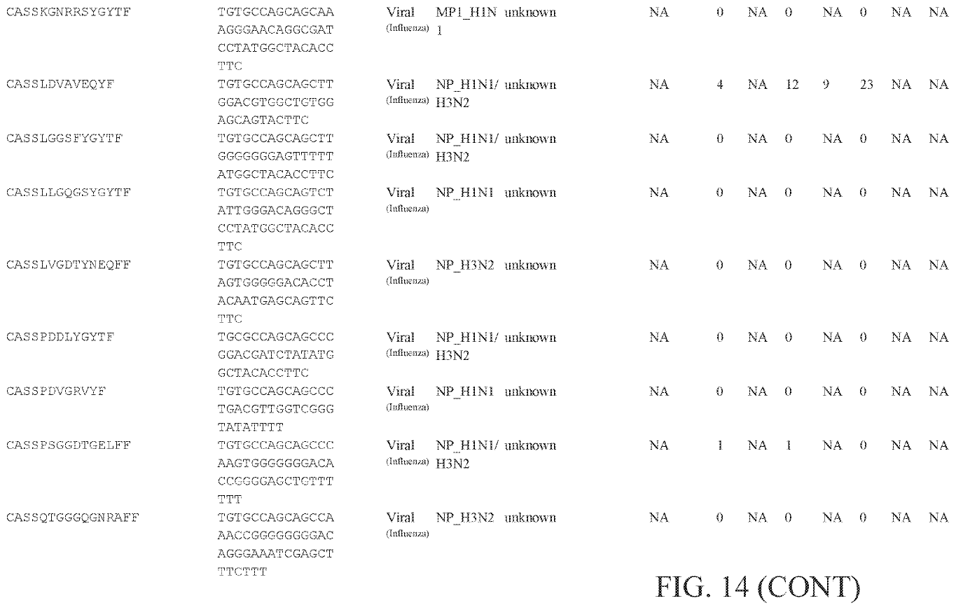

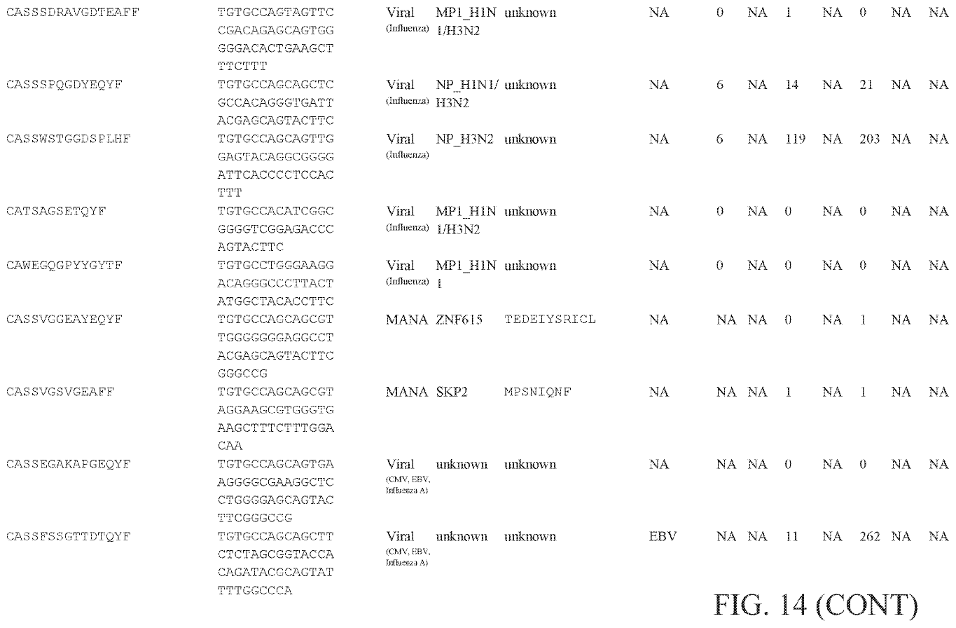

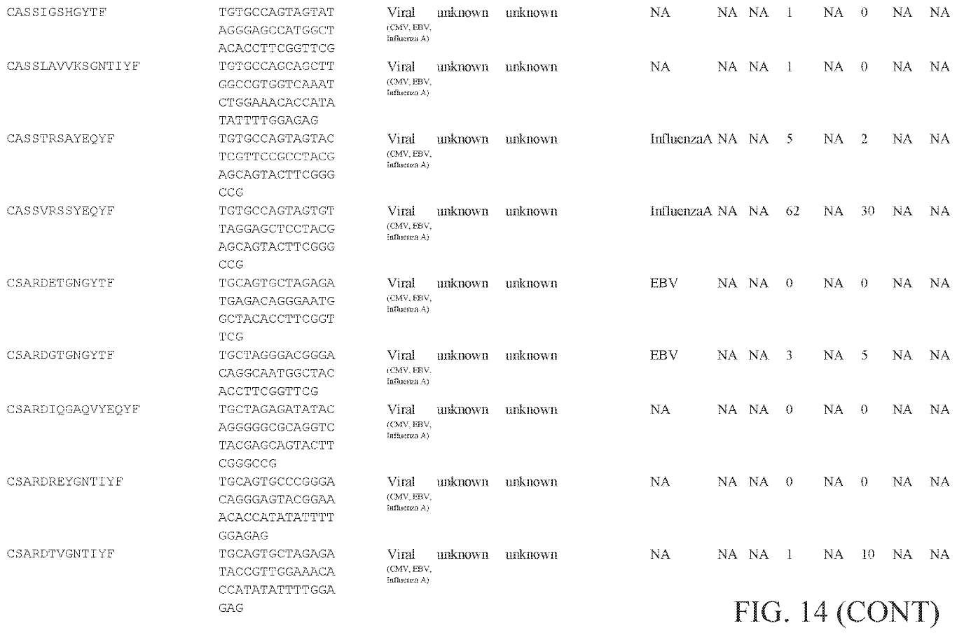

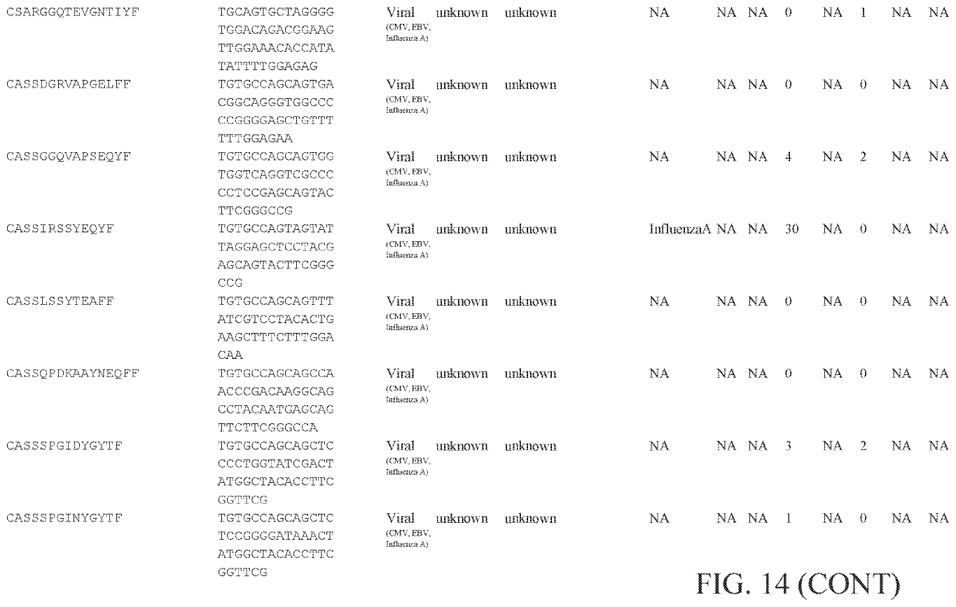

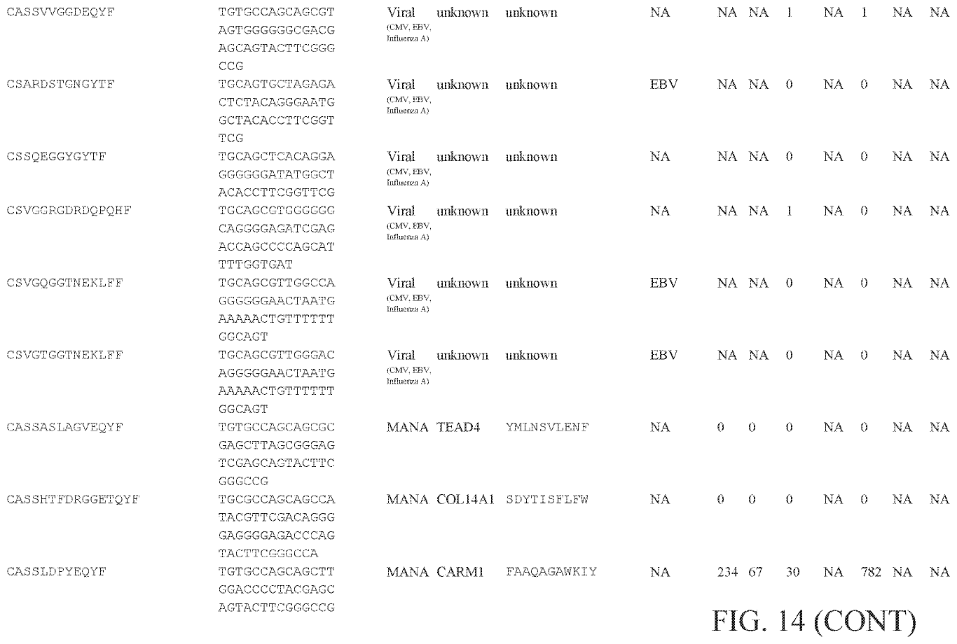

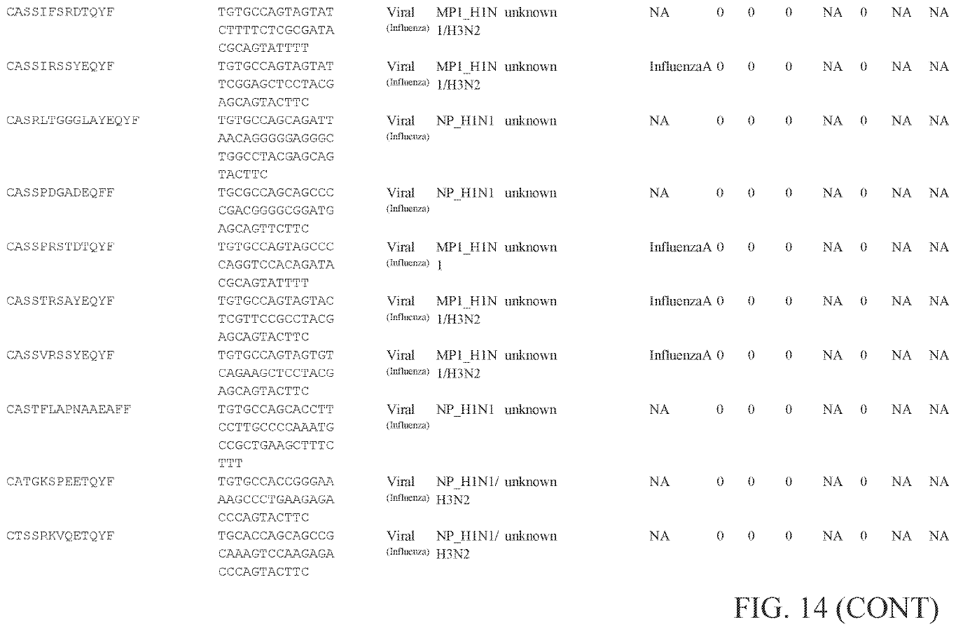

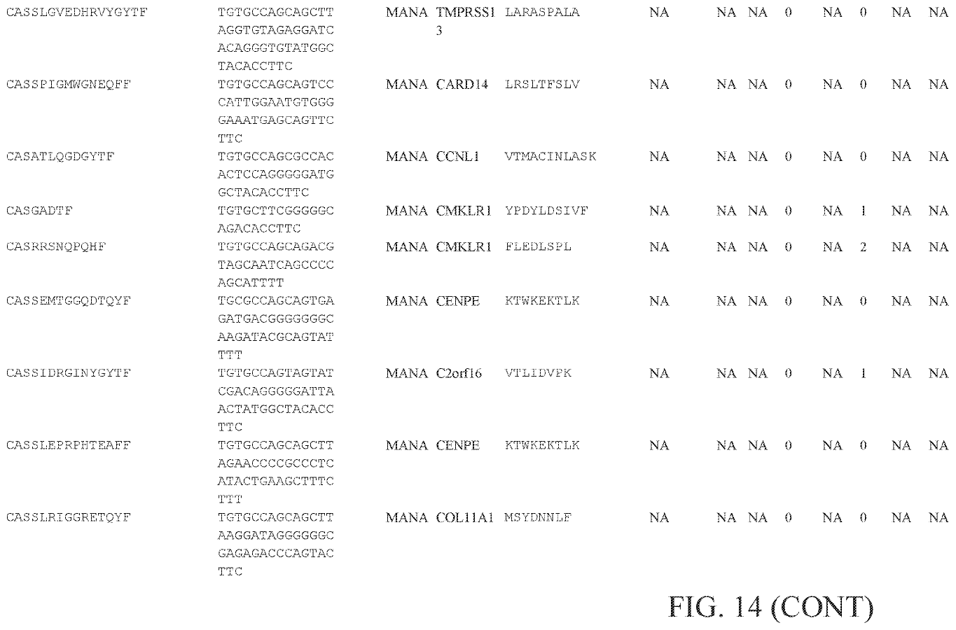

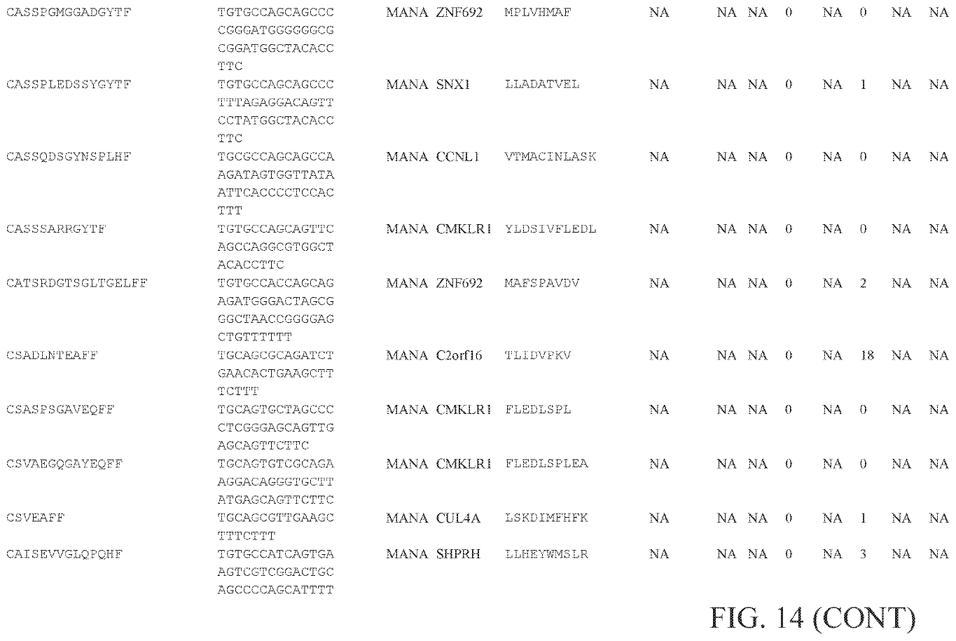

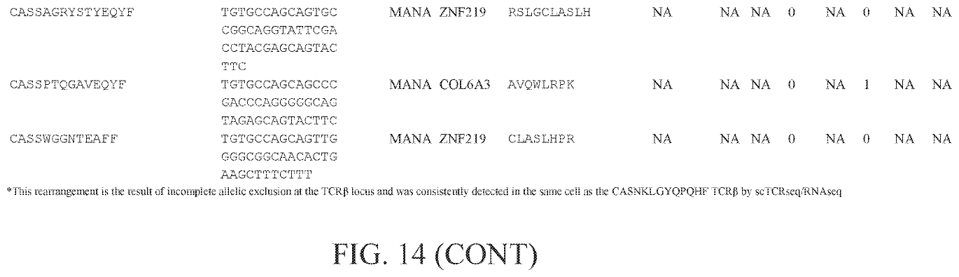

The MANAFEST (Mutation Associated NeoAntigen Functional Expansion of Specific T-cells) assay was used to evaluate T cell responsiveness to MANA and viral antigens. Briefly, pools of MHC class I-restricted CMV, EBV, and flu peptide epitopes (CEFX, jpt Peptide Technologies), pools representing the matrix protein and nucleoprotein from H1N1 and H3N2 (jpt Peptide Technologies), and putative neoantigenic peptides defined by the ImmunoSelect-R pipeline (jpt Peptide Technologies; Table 6 (FIG. 13) and Table 8 (FIG. 14)) were used to stimulate T cells in vitro for 10 days. T cells were also cultured without peptide to use as a reference for non-specific clonotypic expansion. On day 10, T cell receptor sequencing was performed on each individual peptide-stimulated T cell culture by the Sidney Kimmel Comprehensive Cancer Center FEST and TCR Immunogenomics Core (FTIC) facility or Adaptive Biotechnologies. Bioinformatic analysis of productive clones was performed to identify antigen-specific T-cell clonotypes meeting the following criteria: 1) significant expansion (Fisher's exact test with Benjamini-Hochberg correction for FDR, p<0.05) compared to T cells cultured without peptide, 2) significant expansion compared to every other peptide-stimulated culture (FDR<0.0001) except for conditions stimulated with similar neoantigens derived from the same mutation, 3) an odds ratio >5 compared to the “no peptide” control, and 4) present in at least 10% of the cultured wells to ensure adequate distribution among culture wells. A lower read threshold of 300 was used for assays sequenced by the FTIC and a lower threshold of 30 was used for samples sequenced by Adaptive Biotechnologies. In MANAFEST assays testing less than 10 peptides or peptide pools, cultures were performed in triplicate and reactive clonotypes were defined as being significantly expanded relative to T cells cultured without peptide (FDR<0.05) in two out of three triplicates, and not significantly expanded in any other well tested. When available, TCRseq was also performed on DNA extracted from tumor, normal lung, and lymph node tissue obtained before treatment and at the time of surgical resection, as well as serial peripheral blood samples.

### Peptide Affinity and Stability Measurements

Peptide affinity was measured as described elsewhere (see, e.g., Harndahl et al., J. Biomol. Screen, 14:173-180 (2009)). The stability of peptide loaded complexes was measured by refolding MHC with peptide and subsequently challenging complexes with a titration of urea. The denaturation of MHC was monitored by ELISA.

### TCR Reconstruction and Cloning

Ten MANAFEST+TCR sequences for which the TCRα chain could be enumerated (>3 cells in single cell data with the same α/β pair) and MANA score^{hi} TCRs were selected for cloning. Relevant TCRs were analyzed with the IMGT/V-Quest database (imgt.org/IMGT). The database allows us to identify the TRAV and TRBV families with the highest likelihood to contain the identified segments which match the sequencing data. To generate the TCRs, the identified TCRA V-J region sequences were fused to the human TRA constant chain, and the TCRB V-D-J regions to the human TRB constant chain. The full-length TCRA and TCRB chains were then synthesized as individual gene blocks (IDT) and cloned into the pCI mammalian expression vector, containing a CMV promoter, and transformed into competent E. coli cells according to manufacturer's instructions (NEBuilder HiFi DNA Assembly, NEB). Post transformation and plasmid miniprep, the plasmids were sent for Sanger sequencing to ensure no mutations were introduced (Genewiz).

### T Cell Transfection, Transient TCR Expression, and MANA Recognition Assays

To generate a Jurkat reporter cell which could transfer the TCRs of interest, the endogenous T cell receptor (TCR) α and β chains were knocked out of a specific Jurkat line that contains a luciferase reporter driven by an NFAT-response element (Promega) using the Alt-R CRISPR system (Integrated DNA Technologies, IDT). Two sequential rounds of CRISPR knockout were performed using crDNA targeting the TCRα constant region (AGAGTCTCTCAGCTGGTACA; SEQ ID NO:54) and the TCRβ constant region (AGAAGGTGGCCGAGACCCTC; SEQ ID NO:55). Limiting dilution was then used to acquire single cell clones and clones with both TCRα and TCRβ knocked out, as confirmed by Sanger sequencing and restoration of CD3 expression only by the co-transfection of TCRα or TCRβ chains, were chosen. CD8α and CD8β chains were then transduced into the TCRα⁻/β⁻ Jurkat reporter cells using the MSCV retroviral expression system (Clontech). Jurkat reporter cells were then co-electroporated with the pCI vector encoding the TCRB and TCRA gene blocks, respectively, using ECM830 Square wave electroporation system (BTX) at 275 volts for 10 ms in OptiMem media in a 4 mm cuvette. Post electroporation, cells were rested overnight by incubating in in RPMI 10% FBS at 37° C., 5% CO₂. TCR expression was confirmed by flow cytometric staining for CD3 on a BD FACSCelesta. Reactivity of the TCR transduced Jurkat T cells was assessed by co-culturing the cells with autologous EBV-transformed B cells or autologous PBMC, loaded with titrating concentrations of MANA peptides, viral peptide pools, or negative controls. After overnight incubation, activation of the NFAT reporter gene was measured by the Bio-Glo Luciferase Assay per manufacturer's instructions (Promega).

In Vitro Short-Term TIL Stimulation with IL-7

Cryopreserved patient TIL were thawed, counted and stained with viability marker, LIVE/DEAD™ Fixable Aqua (ThermoFisher), and surface markers, CD3 (PE, clone SK1) and CD8 (BV786, clone RPA-T8). 30 thousand CD8+ T cells per each TIL population were sorted on a BD FACSAria II Cell Sorter into a 96-well plate. Autologous peripheral blood mononuclear cells (PBMC) were added as antigen presenting cells (APC) at 1:1 ratio. The cells were stimulated with respective antigen and recombinant human IL-7 (Miltenyi) for 12 hours in a round-bottomed 96-well plate.

### Gene Expression Analysis of IL-7 Stimulated TIL

Following 12 hours of antigen and IL-7 stimulation, cells were spun down, counted and re-suspended in 1% BSA at desired concentration. Single-cell RNA seq and VDJ libraries were prepared using 10× Chromium single cell platform using 5′ DGE library preparation reagents and kits according to manufacturer's protocols (10× Genomics, Pleasonton, CA) and as described above.

COS-7 Transfection with HLA Allele and P53 Plasmids

gBlocks (IDT) encoding HLA A*6801, p53 R248L and p53 WT were cloned into pcDNA3.4 vector (Thermo Fisher Scientific, A14697). COS-7 cells were transfected with plasmids at 70-80% confluency using Lipofectamine 3000 (Thermo Fisher Scientific, L3000015) and incubated at 37° C. overnight in T75 flasks. A total of 30 μg plasmid (1:1 ratio of HLA plasmid/target protein plasmid in co-transfections) was used. Post transfection, COS-7 cells were plated with TCRαβ transfected Jurkat cells containing NFAT reporter gene at a 1:1 ratio. After overnight incubation, activation of the NFAT reporter gene was measured by the Bio-Glo Luciferase Assay per manufacturer's instructions (Promega).

### Single Cell Data Preprocessing and Quality Control

Cell Ranger v3.1.0 was used to demultiplex the FASTQ reads, align them to the GRCh38 human transcriptome, and extract their “cell” and “UMI” barcodes. The output of this pipeline is a digital gene expression (DGE) matrix for each sample, which records the number of UMIs for each gene that are associated with each cell barcode. The quality of cells was then assessed based on (1) the number of genes detected per cell and (2) the proportion of mitochondrial gene/ribosomal gene counts. Low-quality cells were filtered if the number of detected genes was below 250 or above 3×the median absolute deviation away from the median gene number of all cells. Cells were filtered out if the proportion of mitochondrial gene counts was higher than 10% or the proportion of ribosomal genes was less than 10%. For single-cell VDJ sequencing, only cells with full-length sequences were retained. Dissociation/stress associated genes, mitochondrial genes (annotated with the prefix “MT-”), high abundance lincRNA genes, genes linked with poorly supported transcriptional models (annotated with the prefix “RP-”), and TCR (TR) genes (TRA/TRB/TRD/TRG) were removed from further analysis. In addition, genes that were expressed in less than five cells were excluded.

### Single Cell Data Integration and Clustering

Seurat (3.1.5) was used to normalize the raw count data, identify highly variable features, scale features, and integrate samples. Principal component analysis (PCA) was performed based on the 3,000 most variable features identified using the vst method implemented in Seurat. Gene features associated with type I Interferon (IFN) response, immunoglobulin genes and specific mitochondrial related genes were excluded from clustering to avoid cell subsets driven by the above genes. Dimension reduction was done using the RunUMAP function. Cell markers were identified by using a Wilcoxon test. Genes with adjusted p.value<0.05 were retained. Clusters were labeled based on the expression of the top differential gene in each cluster as well as canonical immune cell markers. Global clustering on all CD3 T cells and refined clustering on CD8 T cells were performed using same procedure. To select for CD8+ T cells, SAVER was used to impute dropouts by borrowing information across similar genes and cells. A density curve was fitted to the log2-transformed SAVER imputed CD8A expression values (using ‘density’ function in R) of all cells from all samples. A cutoff is determined as the trough of the bimodal density curve (i.e., the first location where the first derivative is zero and the second derivative is positive). All cells with log2-transformed SAVER imputed CD8A expression larger than the cutoff are defined as CD8⁺ T cells. TRB amino acid (aa) sequences were used as a biological barcode to match MANA/EBV/Influenza A specific T cell clonotypes identified from the FEST assay with single-cell VDJ profile and were projected onto CD8⁺ T cell refined UMAP.

### Single Cell Subset Pseudobulk Gene Expression Analysis

PCA was performed on a standardized pseudobulk gene expression profile, where each feature was standardized to have a mean of zero and unit variance. In the global clustering analysis, counts were aggregated at the sample level for each cell cluster and normalized by library size. Combat function in the “sva” R package was applied to address potential batch effects on the normalized pseudobulk profile. Highly variable genes (HVGs) were selected for each cell cluster by fitting a locally weighted scatterplot smoothing (LOESS) regression of standard deviation against the mean for each gene and identifying genes with positive residuals. All cell clusters were then concatenated by retaining cluster-specific HVGs to construct a pseudobulk gene expression matrix. Canonical correlation between the first two PCs (i.e., PC1 and PC2) and a covariate of interest (i.e., tissue type or response status) was calculated. Permutation test was used to assess the significance by randomly permuting the sample labels 10,000 times.

### Differential Expression Tests and Antigen-Specific T Cell Marker Genes

Differential expression (DE) tests were performed using FindAllMarkers functions in Seurat with Wilcoxon Rank Sum test on SAVER imputed expression values. Genes with >0.25 log2-fold changes, at least 25% expressed in tested groups, and Bonferroni-corrected p values<0.05 were regarded as significantly differentially expressed genes (DEGs). Antigen-specific (MANA vs flu vs EBV) T cell marker genes were identified by applying the DE tests for upregulated genes between cells of one antigen specificity to all other antigen specific-T cells in the dataset. Top ranked genes (by log-fold changes) with a log2-fold changes >0.6 from each antigen-specificity type of interest were extracted for further visualization in heatmap using pheatmap package. Saver imputed expression values of selective marker genes (transcriptional regulators/memory markers/tissue resident markers/T cell checkpoints/effector/activation markers) were plotted using the RidgePlot function in Seurat.

### Gene Expression Analysis of IL-7 Stimulated MANA/Flu-Specific TIL

MANA/flu-specific T cell clonotypes from single-cell dataset were identified by using TRB aa sequences as a biological barcode. SAVER imputed gene expression was scaled and centered using “ScaleData” function in Seurat. A composite score for IL7 upregulated gene set expression was computed using the AddModuleScore function and subsequently visualized using ridgeplot. Mean±standard error was used to show dose response curve of IL7 upregulated gene set score by antigen-specific T cells+peptide stimulation groups.

### Immune Checkpoint Score Generation and Highly Correlated Genes

To characterize dysfunctional CD8 MANA TIL, 6 best characterized (and clinically targeted) checkpoints: CTLA4, PDCD1, LAG3, HAVCR2, TIGIT and ENTPD1, were used to compute the T cell checkpoint score using AddModuleScore function in Seurat. Applying T cell checkpoint score as an anchor, genes that were maximally correlated to the score were identified using linear correlation in MANA-specific TIL from MPR and non-MPR, respectively. Top 30 genes with the highest correlation coefficients were plotted using barplot. The difference of the above genes was additionally computed between MPR and non-MPR and visualized using waterfall plot.

### Results

The efficacy of immune checkpoint blockade (ICB) agents, such as anti-PD(L)-1, is predicated upon CD8 T cell-mediated anti-tumor immunity (see, e.g., Tumeh et al., Nature, 515:568-571 (2014)). The association of improved anti-PD(L)-1 clinical responses with high mutational burden tumors (see, e.g., Le et al., Science, 357:409-413 (2017); Snyder et al., N. Engl. J. Med., 371:2189-2199 (2014); Van Allen et al., Science, 350:207-211 (2015); Rizvi et al., Science, 348:124-128 (2015)) strongly suggests that MANA are important targets of anti-tumor immunity induced by PD-1 blockade (see, e.g., Rizvi et al., Science, 348:124-128 (2015); Schumacher et al., Science 348:69-74 (2015); and Ward et al., Adv Immunol 130:25-74 (2016)).

Improving ICB response rate will require an understanding of the functional state of tumor-specific T cells, particularly in the tumor microenvironment. A fundamental limitation of the understanding of the T cell functional programs underpinning response to ICB has been the absence of transcriptional profiling of true MANA-specific TIL. A related problem is the paucity of information regarding the differences between MANA-specific TIL in ICB responsive vs resistant tumors. Indeed, MANA-specific T cells represent a small fraction of total TIL, highlighting the challenges confronting characterization of the cells responsible for the activity of T cell-targeting immunotherapies.

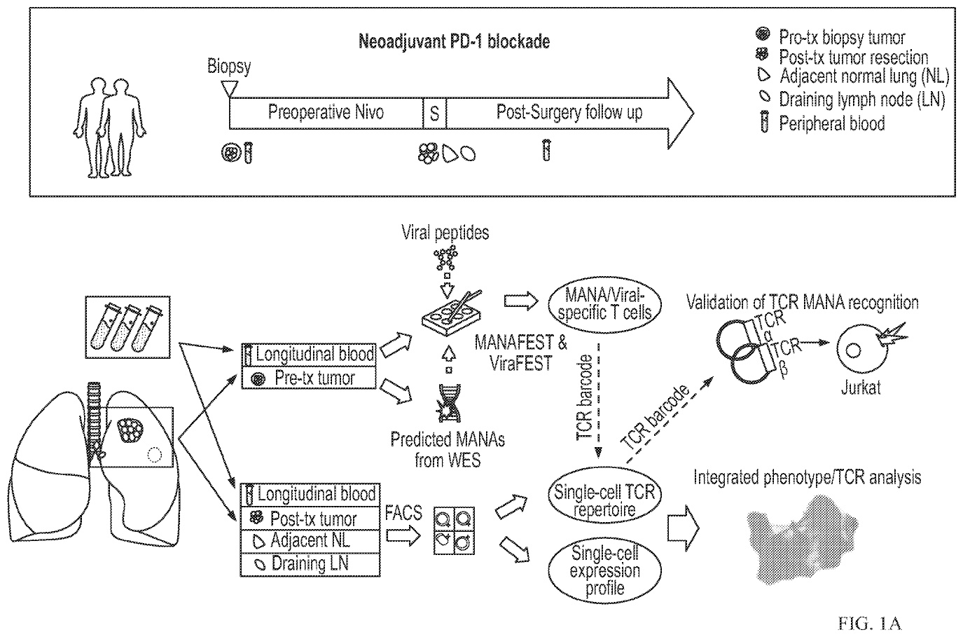

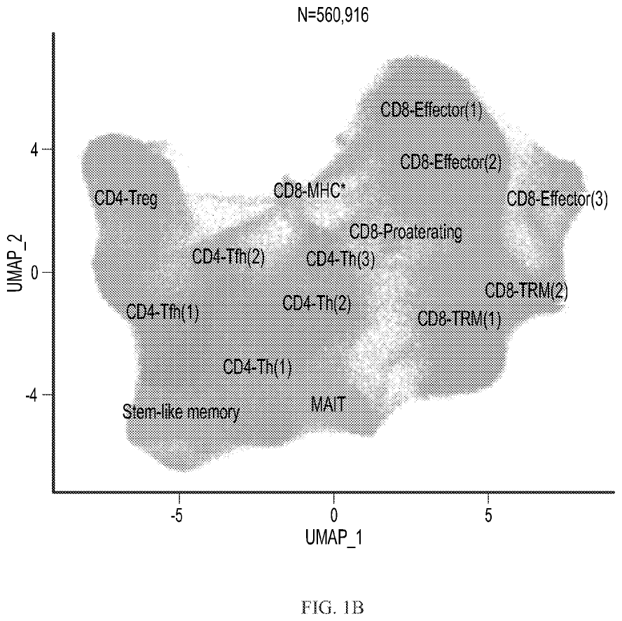

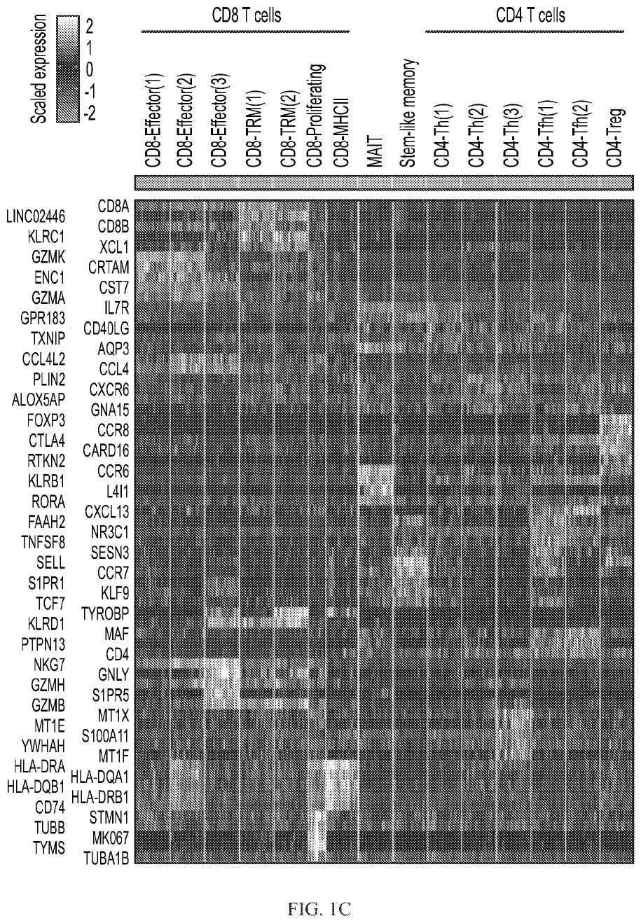

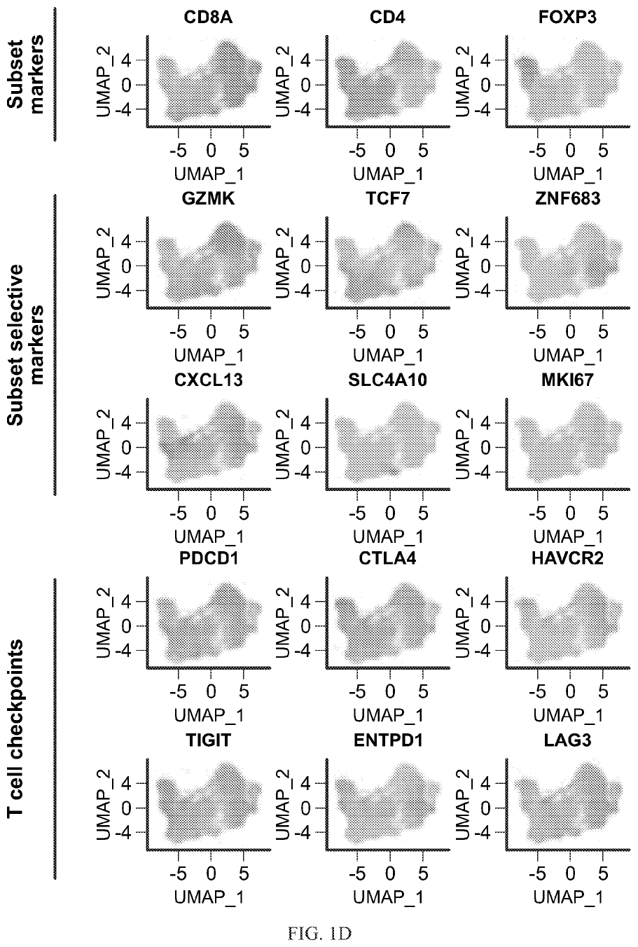

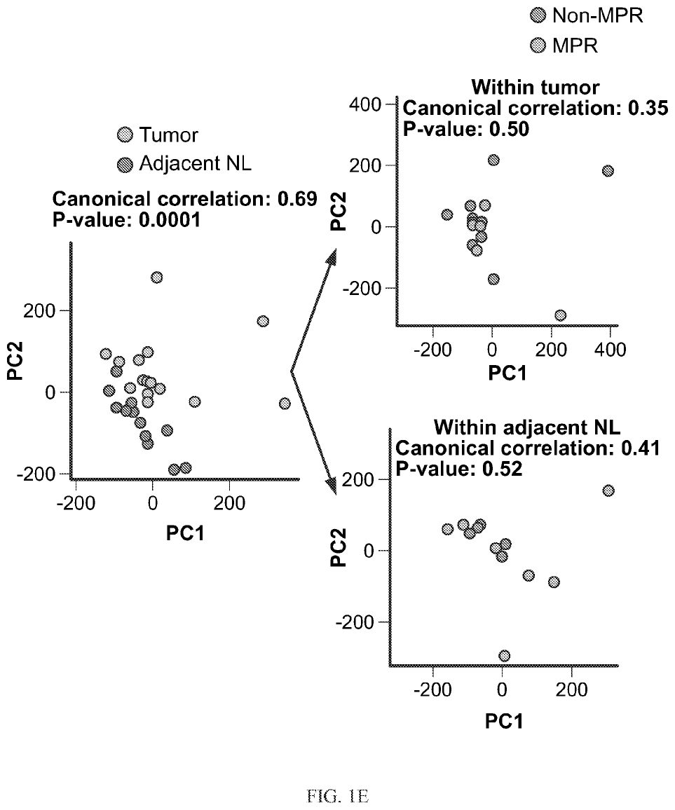

For the present study, peripheral blood and surgical resection specimens obtained from the first-in-human clinical trial of neoadjuvant anti-PD-1 (nivolumab) in resectable non-small cell lung cancer NSCLC (NCT02259621) were utilized. After 4 weeks of nivolumab (FIG. 1A, top), 45% of the patients had a major pathologic response (MPR) at time of resection, defined as ≤10% viable tumor at the time of surgery. To identify MANA-specific CD8 T cells after PD-1 blockade, candidate MANA peptides, derived from application of an MHC I binding prediction algorithm to whole exome tumor sequencing, were tested for peripheral blood CD8 T cell recognition using a recently developed high throughput TCRseq-based platform, MANAFEST (Mutation Associated NeoAntigen Functional Expansion of Specific T cells, FIG. 1A, bottom). In parallel, simultaneous coupled single cell (sc) RNAseq/scTCRseq analysis of purified T cells from tumor, adjacent NL, and tumor-draining lymph nodes (TDLN), when available, were performed. The TCRβ CDR3 was then used as a barcode to identify MANA-specific CD8 T cells among TIL, adjacent NL, and TDLN. With these single cell data, the paired TCRα for MANA-specific TCRβ clonotypes were identified which in turn enabled validation of MANA recognition by individual clones via transfer of both TCR genes into an engineered Jurkat reporter cell line. Influenza A (flu)- and EBV-specific clones were also identified among TIL that were expanded in the assay in response to pools of viral peptides (ViraFEST) and further validated by matching with a public database. In all, MANAFEST was performed on 9 patients and scTCRseq/scRNAseq was performed on 16 patients in the trial, including TIL (n=15) together with T cells from paired adjacent normal lung (NL, n=12), and T cells from tumor-draining lymph nodes (TDLN, n=3) which we could sort MANA-specific T cells (Table 4 and Table 5). A total of 560,916 T cells passed quality control (FIG. 1B and Table 5) and were carried forward in the analyses.

TABLE 4
Clinical and histopathological features of patients included in this study*

| | | | | | | Pre- | | | | | |
|---|---|---|---|---|---|---|---|---|---|---|---|
| | Age | | | | | Treat- | | | | | |
| | at | | | | | ment | % | % | | | |
| | diag- | | | | | Clinical | PD- | PD-L1 | % | | |
| | nosis | | Smoking | Pack | | Tumor | L1 | (re- | residual | MPR | |
| Study ID | (years) | Gender | history | Years | Histology | Stage | (pre) | section) | tumor | status | HLA haplotype |
| MD01-005 | 61 | M | Current | 45 | Squamous Cell | T3N0 | NA | 17 | 0 | MPR | HLA-A*25:01; HLA-A*30:01; |
| | | | Smoker | | Carcinoma | | | | | | HLA-B*39:01; HLA-B*38:01; |
| | | | | | | | | | | | HLA-C*12:03 |
| MD01-004 | 67 | M | Former | 20 | Adenosquamous | T4N1 | 1 | 65 | 40 | non- | HLA-A*31:01; HLA-A*68:01; |
| | | | Smoker | | | | | | | MPR | HLA-B*35:08; HLA-B*51:01; |
| | | | | | | | | | | | HLA-C*15:02; HLA-C*04:01 |
| MD043-008 | 72 | F | Former | 50 | Squamous Cell | T1bN0 | 0 | 0 | 10 | MPR | HLA-A*02:01; HLA-A*29:02; |
| | | | Smoker | | Carcinoma | | | | | | HLA-B*07:02; HLA-B*44:03; |
| | | | | | | | | | | | HLA-C*07:02; HLA-C*16:01 |
| MD043-011 | 55 | M | Former | 40 | Adenocarcinoma | T2aN1 | NA | 0 | 75 | non- | HLA-A*24:02; HLA-A*23:01; |
| | | | Smoker | | | | | | | MPR | HLA-B*40:01; HLA-B*44:03; |
| | | | | | | | | | | | HLA-C*03:04; HLA-C*02:02 |
| MD01-019 | 70 | M | Former | 50 | Adenocarcinoma | T2aN0 | 0 | NA | 95 | non- | HLA-A*02:01; HLA-A*30:01; |
| | | | Smoker | | | | | | | MPR | HLA-B*13:02; HLA-C*06:02 |
| MD043-003 | 62 | M | Former | 40 | Adenocarcinoma | T2aN0 | NA | 2 | 5 | MPR | HLA-A*02:01; HLA-A*01:01; |
| | | | Smoker | | | | | | | | HLA-B*40:01; HLA-B*35:01; |
| | | | | | | | | | | | HLA-C*03:04; HLA-C*07:01 |
| MD043-006 | 69 | M | Former | 90 | Squamous Cell | T2AN1 | 20 | 70 | 50 | non- | HLA-A*24:03; HLA-B*35:08; |
| | | | Smoker | | Carcinoma | | | | | MPR | HLA-B*18:01; HLA-C*12:03; |
| | | | | | | | | | | | HLA-C*04:01 |
| MD01-024 | 70 | F | Never | 0 | Adenocarcinoma | T1AN0 | NA | NA | 100 | non- | HLA-A*68:02; HLA-A*68:01; |
| | | | Smoker | | | | | | | MPR | HLA-B*40:01; HLA-B*53:01; |
| | | | | | | | | | | | HLA-C*03:19; HLA-C*04:01 |
| MD01-010 | 78 | F | Former | 20 | Adenocarcinoma | T3N0 | NA | NA | 5 | MPR | HLA-A*02:01; HLA-A*11:01; |
| | | | Smoker | | | | | | | | HLA-B*07:02; HLA-B*55:01; |
| | | | | | | | | | | | HLA-C*07:02; HLA-C*03:03 |
| NY016-007 | 68 | F | Former | 10 | Squamous Cell | T2aN1 | 0 | 2 | 60 | non- | HLA-A*01:01; HLA-B*08:01; |
| | | | Smoker | | Carcinoma | | | | | MPR | HLA-C*07:01 |
| NY016-014 | 58 | F | Never | 0 | Adenocarcinoma | T2N2 | 60 | 25 | 95 | non- | HLA-A*02:01; HLA-A*11:01; |
| | | | Smoker | | | | | | | MPR | HLA-B*51:01; HLA-B*35:01; |
| | | | | | | | | | | | HLA-C*05:01; HLA-C*04:01 |
| NY016-016 | 79 | F | Current | 20 | Adenocarcinoma | TlbN1 | NA | NA | 0 | MPR | HLA-A*29:02; HLA-A*01:01; |
| | | | Smoker | | | | | | | | HLA-B*07:02; HLA-B*08:01; |
| | | | | | | | | | | | HLA-C*07:02; HLA-C*07:01 |
| NY016-015 | 58 | F | Former | 10 | Adenocarcinoma | T2bN1 | NA | NA | 80 | non- | HLA-A*02:01; HLA-A*32:01; |
| | | | Smoker | | | | | | | MPR | HLA-B*27:07; HLA-C*15:02 |
| NY016-021 | 74 | M | Former | 50 | Adenocarcinoma | T3N0 | NA | NA | 100 | non- | HLA-A*24:02; HLA-A*31:01; |
| | | | Smoker | | | | | | | MPR | HLA-B*18:01; HLA-B*35:08; |
| | | | | | | | | | | | HLA-C*04:01; HLA-C*07:01 |
| NY016-022 | 66 | F | Former | 20 | Adenocarcinoma | T2bN0 | AN | NA | 5 | MPR | HLA-A*02:01; HLA-A*68:02; |
| | | | Smoker | | | | | | | | HLA-B*37:01; HLA-B*44:02; |
| | | | | | | | | | | | HLA-C*06:02 |
| NY016-025 | 74 | F | Never | 0 | Adenosquamous | T3N1 | NA | NA | 0 | MPR | HLA-A*11:01; HLA-A*26:01; |
| | | | Smoker | | | | | | | | HLA-B*38:01; HLA-B*40:02; |
| | | | | | | | | | | | HLA-C*03:04; HLA-C*12:03 |

*Treated as part of a clinical trial described in Forde et al., N. Engl. J. Med., 378: 1976-1986 (2018)

TABLE 5
Single cell TCRseq/RNAseq sequencing information and metrics

| | | No. cells | | No. cells |
|---|---|---|---|---|
| Patient | Sample | sequenced | No. cells | with matching |
| Study ID | Source | by DGE | after QC | VDJ |
| MD01-024 | Tumor | 3611 | 3197 | 2153 |
| MD01-010 | Normal | 3502 | 3271 | 1707 |
| MD01-010 | Tumor | 6327 | 4627 | 3537 |
| MD01-004 | Lymph Node | 24659 | 21965 | 15151 |
| MD01-004 | Tumor | 3210 | 3082 | 1995 |
| MD043-011 | Lymph Node | 17515 | 10963 | 9107 |
| MD043-011 | Normal | 39648 | 33824 | 25370 |
| MD043-011 | Tumor | 29338 | 22196 | 18940 |
| MD043-011 | Metastatic Tumor | 37106 | 25696 | 21329 |
| MD01-019 | Normal | 12855 | 11631 | 8366 |
| MD01-019 | Tumor | 52021 | 41684 | 31314 |
| NY016-007 | Normal | 5416 | 3645 | 2435 |
| NY016-007 | Tumor | 20652 | 16136 | 13146 |
| NY016-014 | Tumor | 35655 | 29482 | 23947 |
| NY016-015 | Normal | 6042 | 4467 | 3580 |
| NY016-015 | Tumor | 35807 | 24126 | 19971 |
| NY016-016 | Normal | 10646 | 9093 | 7423 |
| NY016-021 | Tumor | 3675 | 1407 | 785 |
| NY016-022 | Normal | 3395 | 2974 | 1585 |
| NY016-022 | Tumor | 46348 | 40089 | 31402 |
| NY016-025 | Normal | 11609 | 9376 | 4786 |
| NY016-025 | Tumor | 55786 | 48541 | 27268 |
| MD043-008 | Normal | 2854 | 2321 | 1653 |
| MD043-008 | Tumor | 2115 | 1897 | 1347 |
| MD043-003 | Normal | 10014 | 8541 | 6749 |
| MD043-003 | Tumor | 34007 | 29051 | 23712 |
| MD01-005 | Lymph Node | 42570 | 32874 | 23404 |
| MD01-005 | Normal | 34248 | 28379 | 21804 |
| MD01-005 | Tumor | 75705 | 68181 | 53719 |
| MD043-006 | Normal | 10316 | 7787 | 6354 |
| MD043-006 | Tumor | 11958 | 10413 | 8686 |

A uniform manifold approximation and projection (UMAP) of filtered and normalized transcript counts for the aggregated T cells from tumor and adjacent NL from all 16 patients defined 15 unique T cell clusters (FIG. 1B). At this resolution, multiple CD8 effector T cell (Teff) subsets, CD4 T helper cell (Th) subsets and tissue resident memory (TRM) subsets were evident. Top expressed genes for each cluster were visualized in FIG. 1C. Interestingly, a previously undescribed lincRNA, LINCO2246, was selectively expressed in all TRM clusters, though at differing levels for different TRM subsets. Expression of T cell subset defining markers (CD8A, CD4, and FOXP3), T cell subset selective genes (GZMK—Teff cells; TCF7—stem-like/memory cells, which could be resolved into CD4 and CD8 subsets; ZNF683 (HOBIT)—TRM cells; CXCL13—Tfh cells; SLC4A10—MAIT cells; and MKI67—proliferating cells) and major T cell checkpoints being targeted clinically (PDCD1, HAVCR2, TIGIT, ENTPD1, LAG3, and CTLA4) were visualized in red-scale on the UMAP (FIG. 1D). Principal component analysis (PCA) of pseudo-bulk gene expression (obtained by first computing the average gene expression vector of each T cell subset and then concatenating these vectors from all cell subsets into one long vector) distinguished adjacent NL T cells from TIL (FIG. 1E), but did not separate MPR from non-MPR, indicating that gene expression profiling of total TIL has limited sensitivity in distinguishing pathologic response to PD-1 blockade.

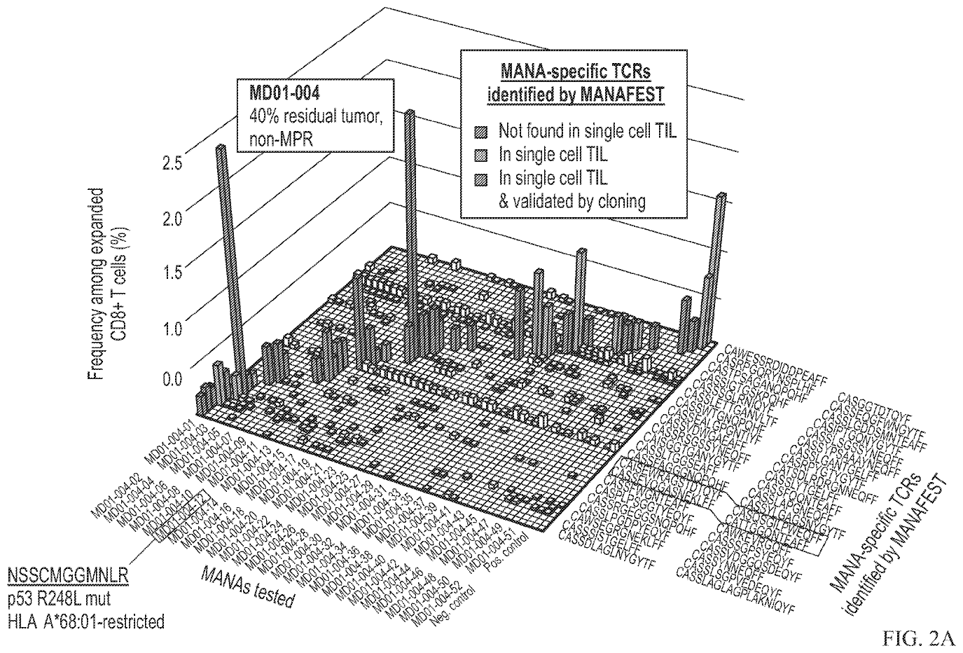

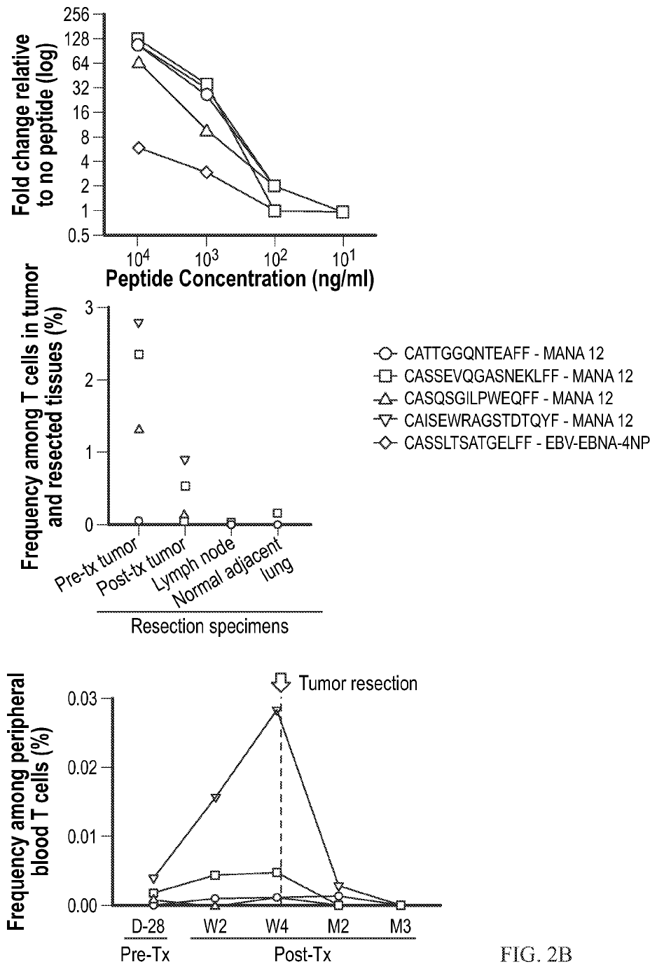

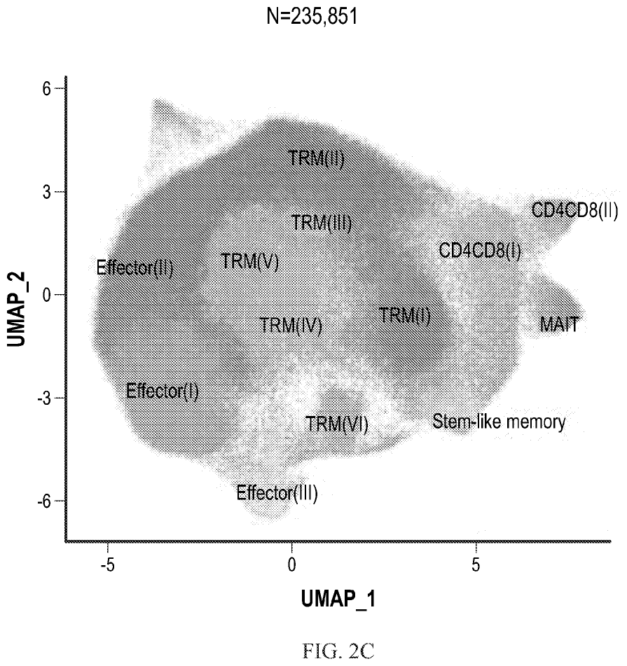

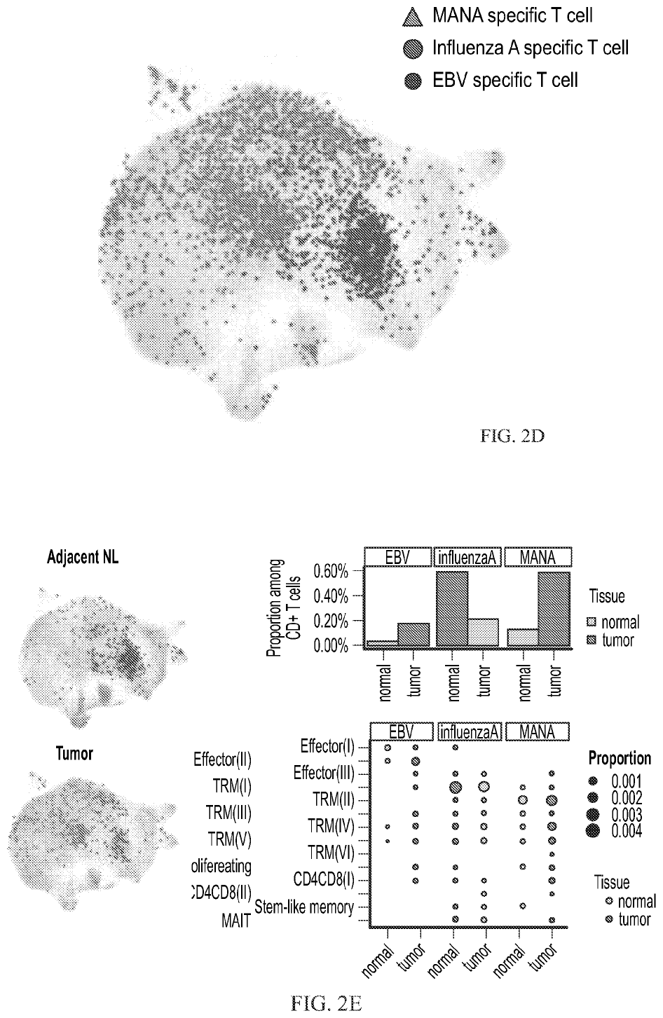

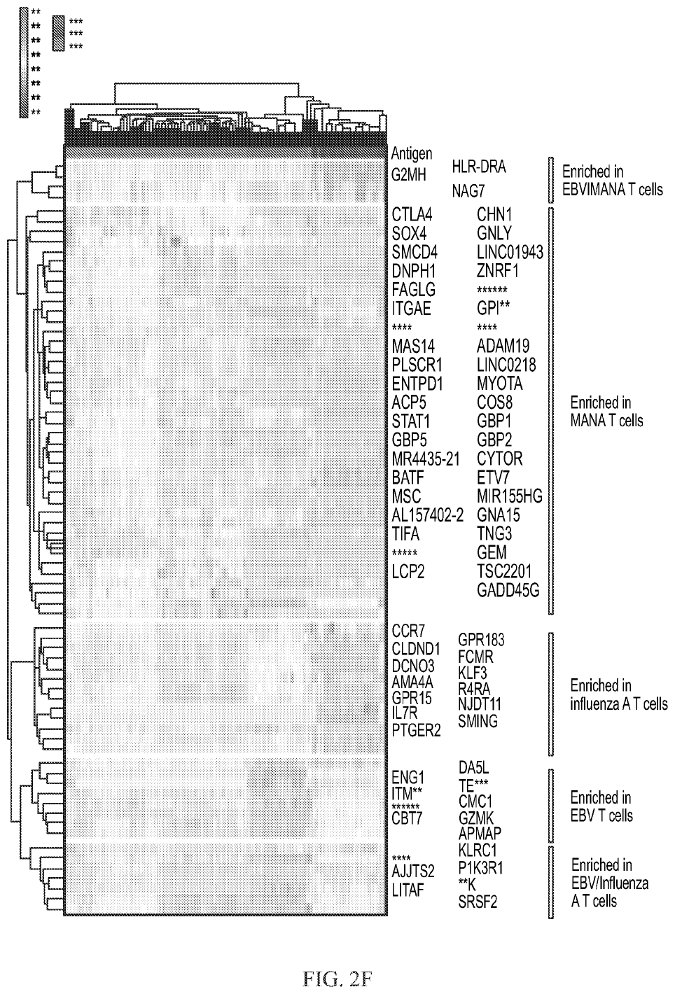

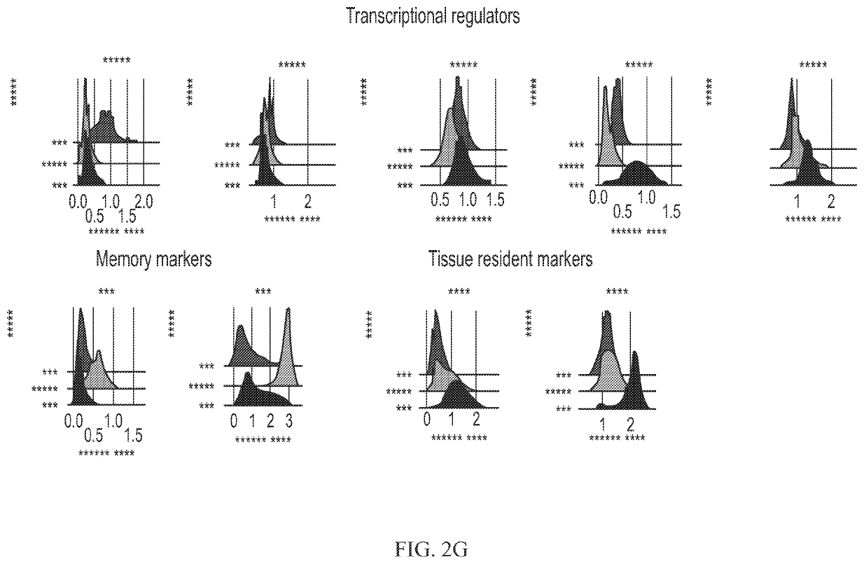

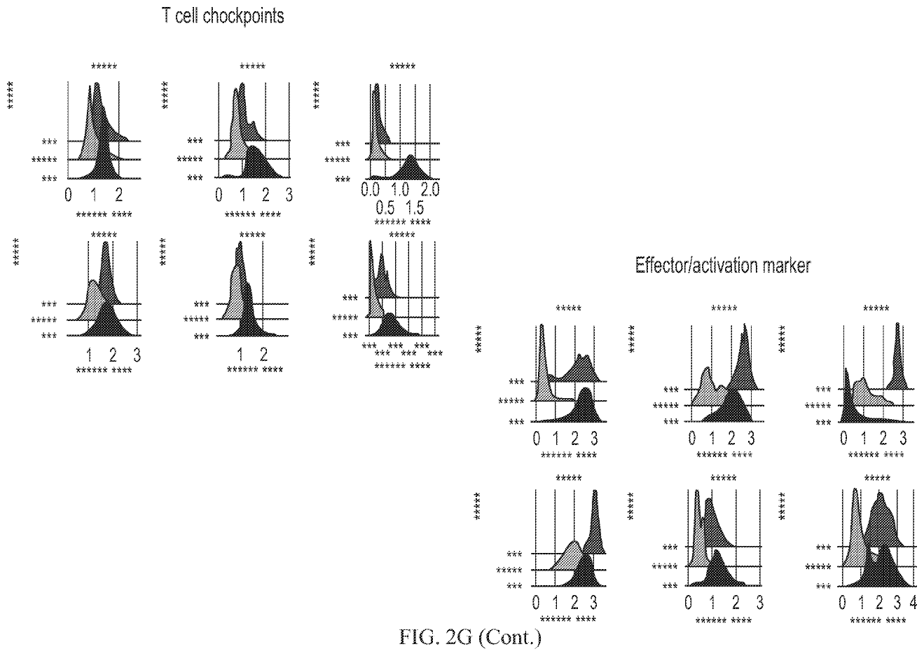

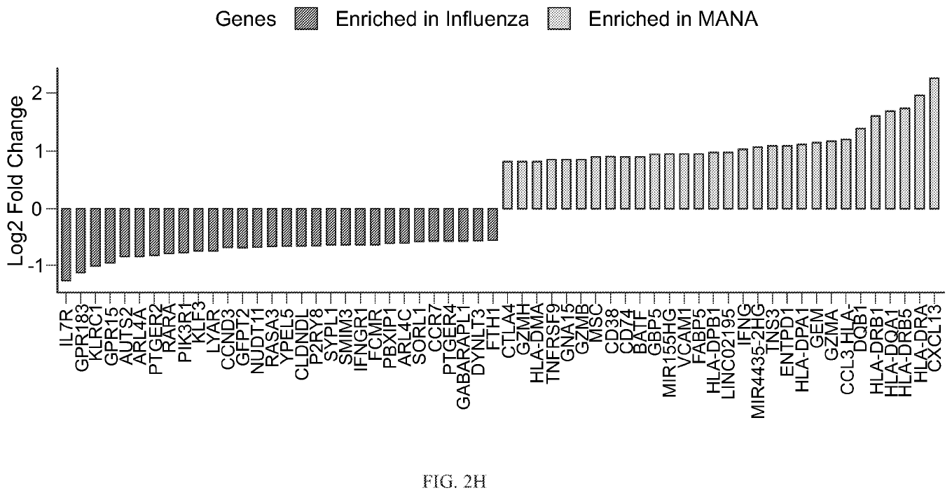

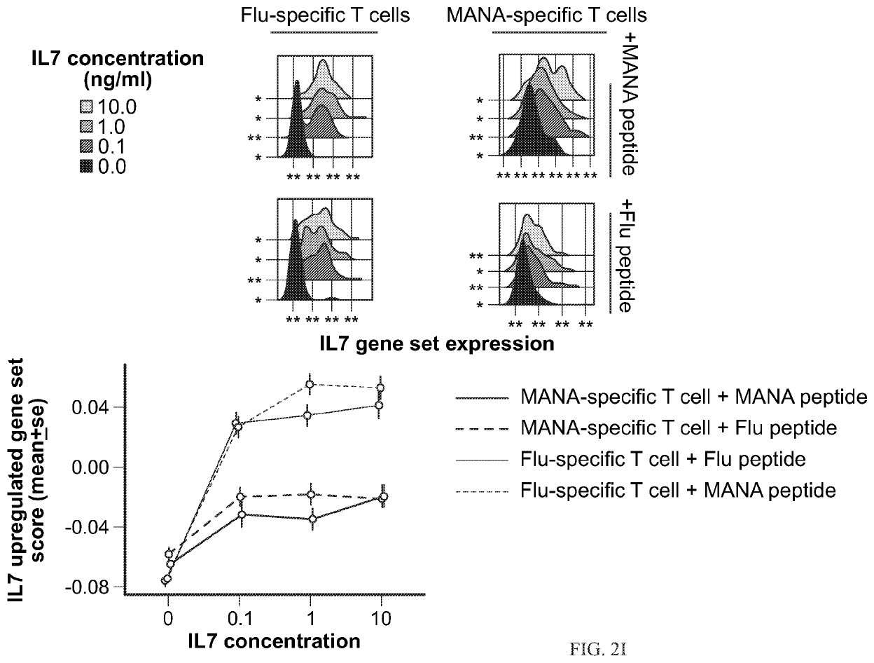

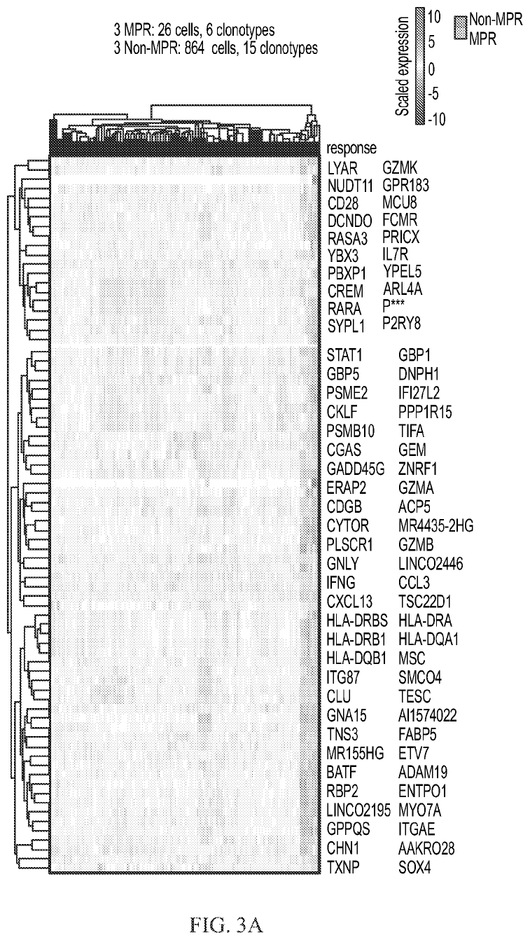

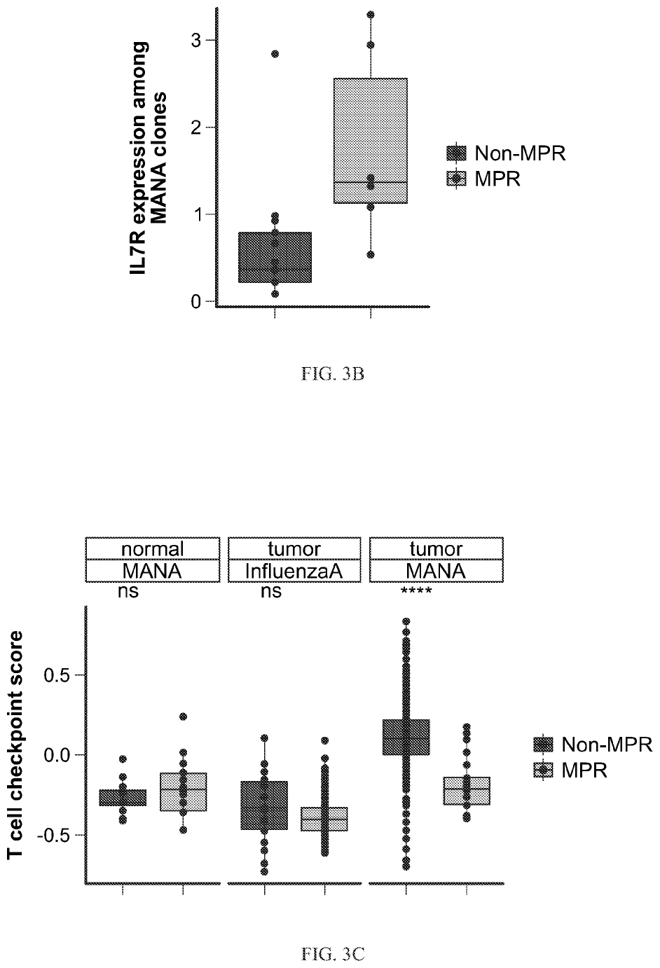

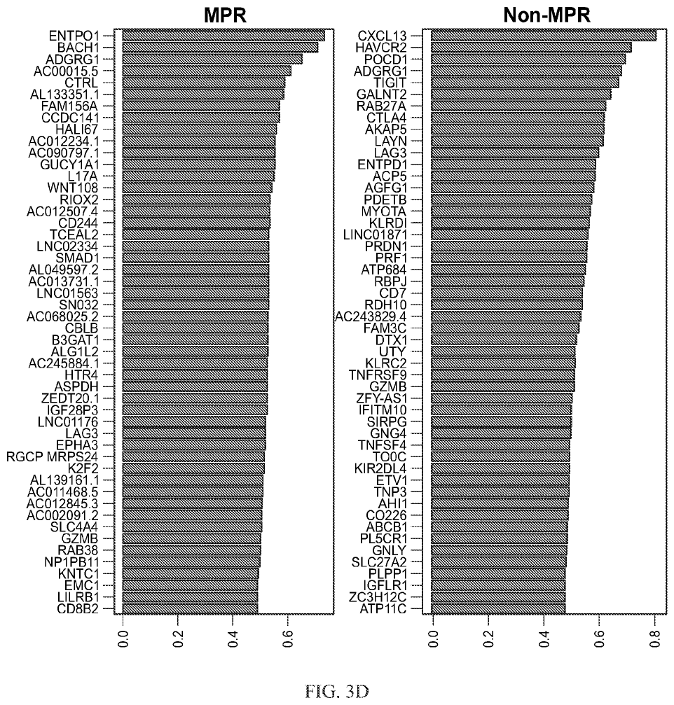

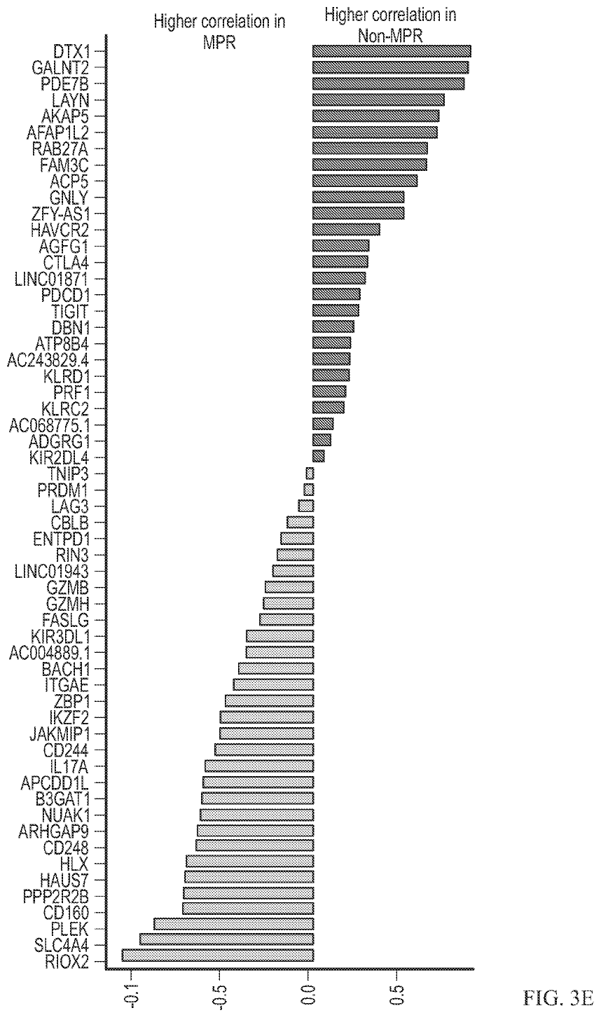

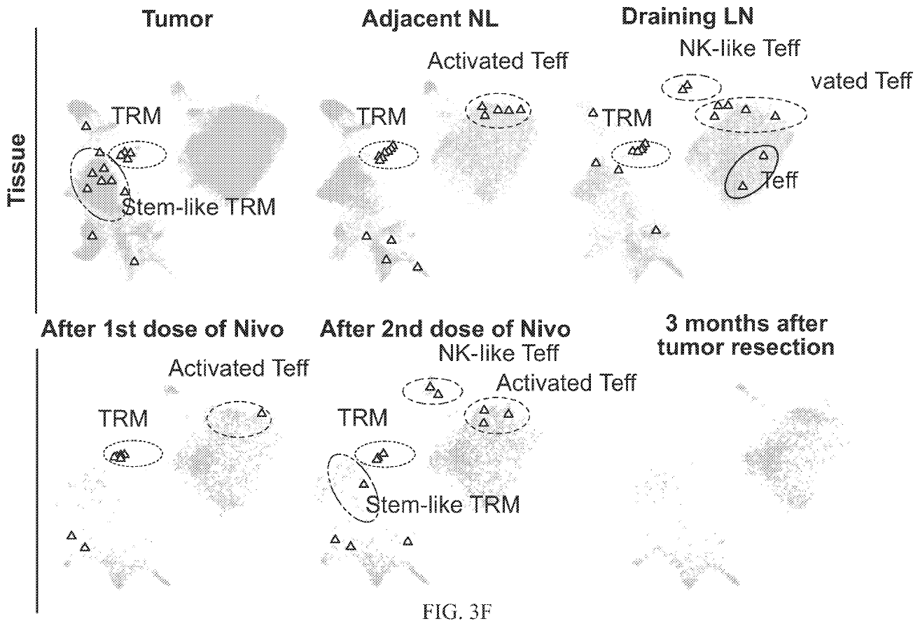

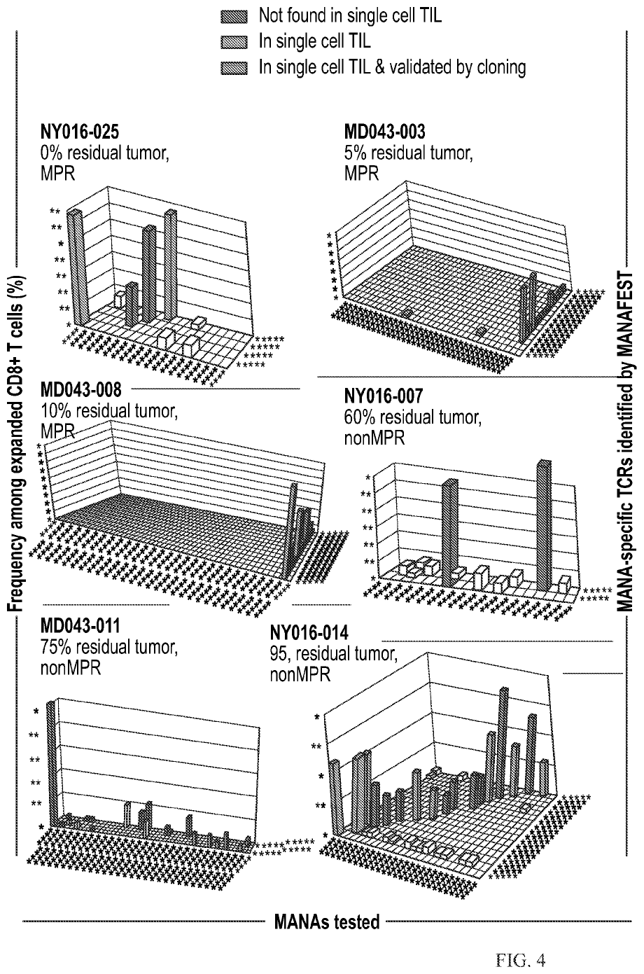

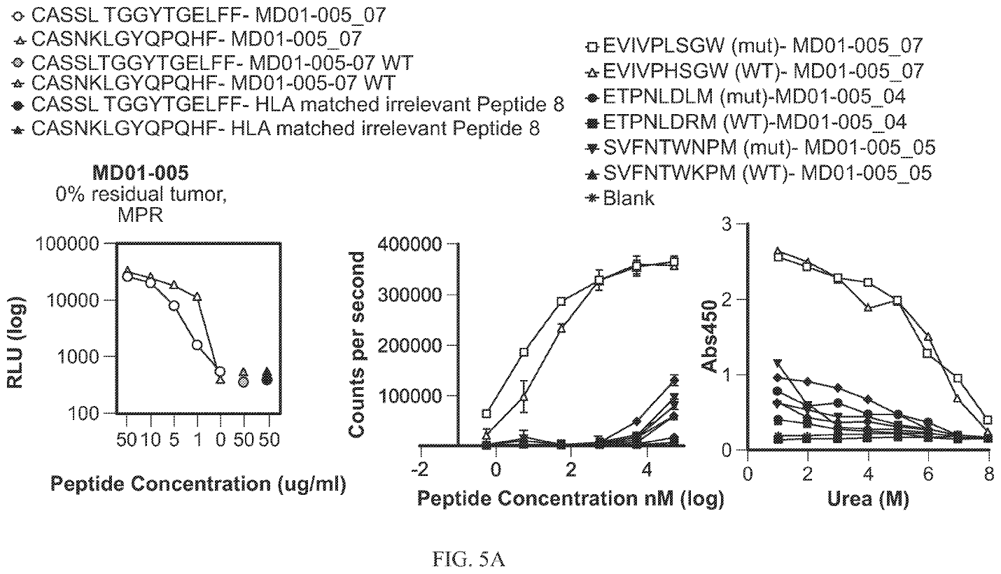

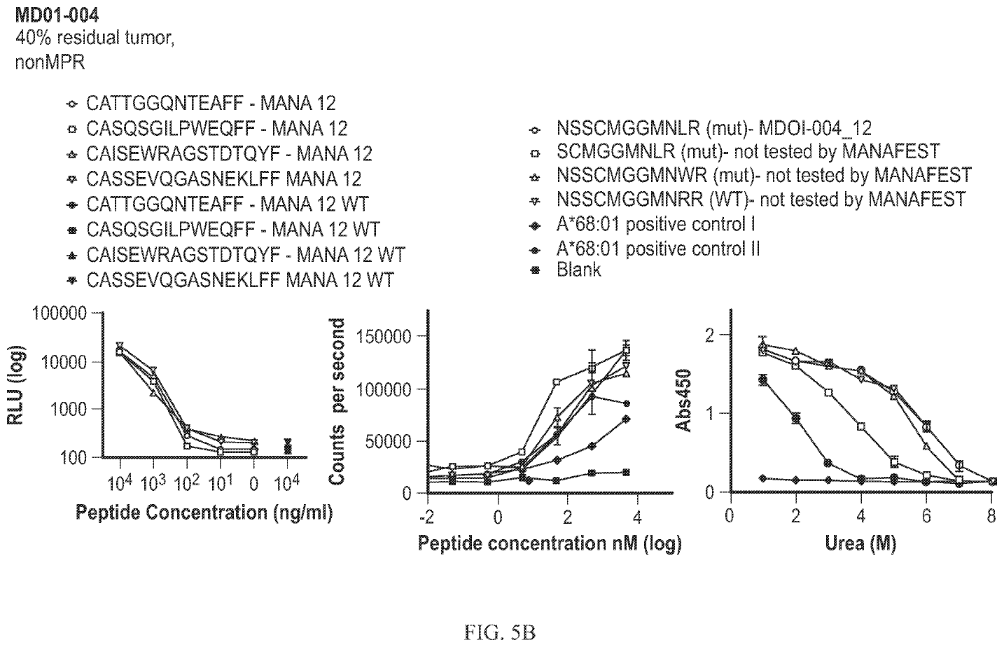

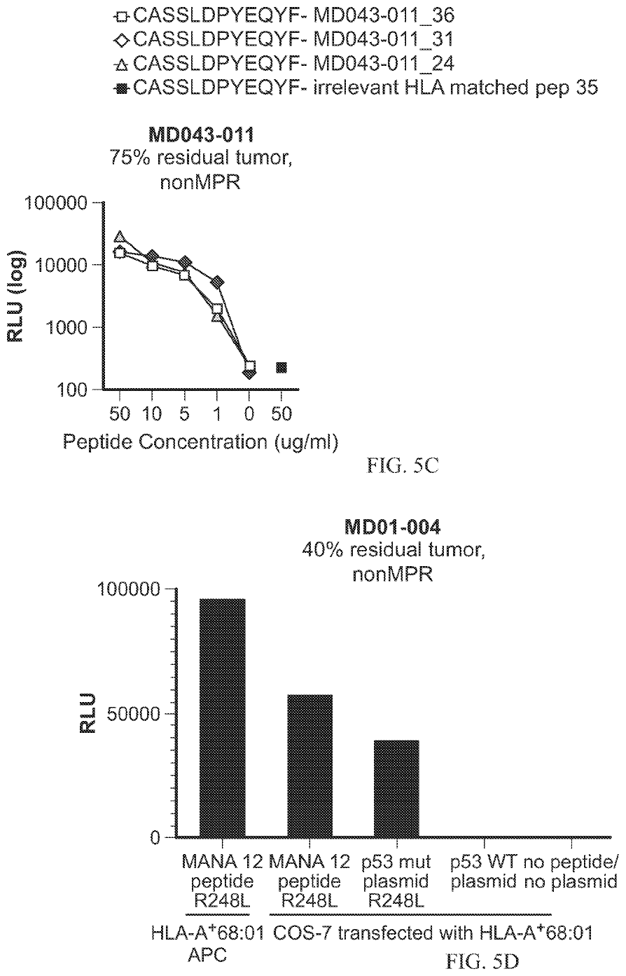

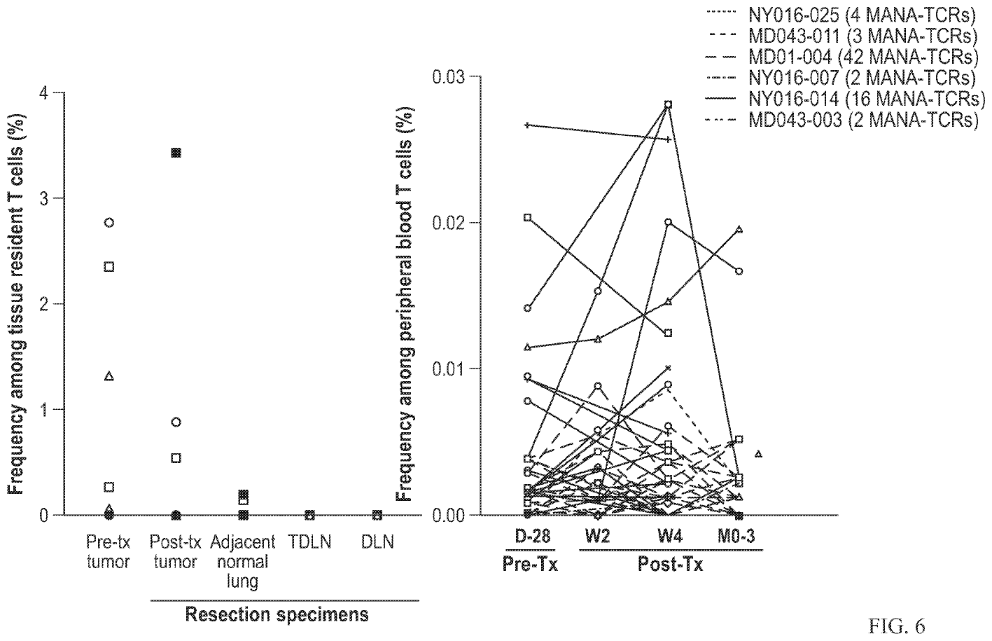

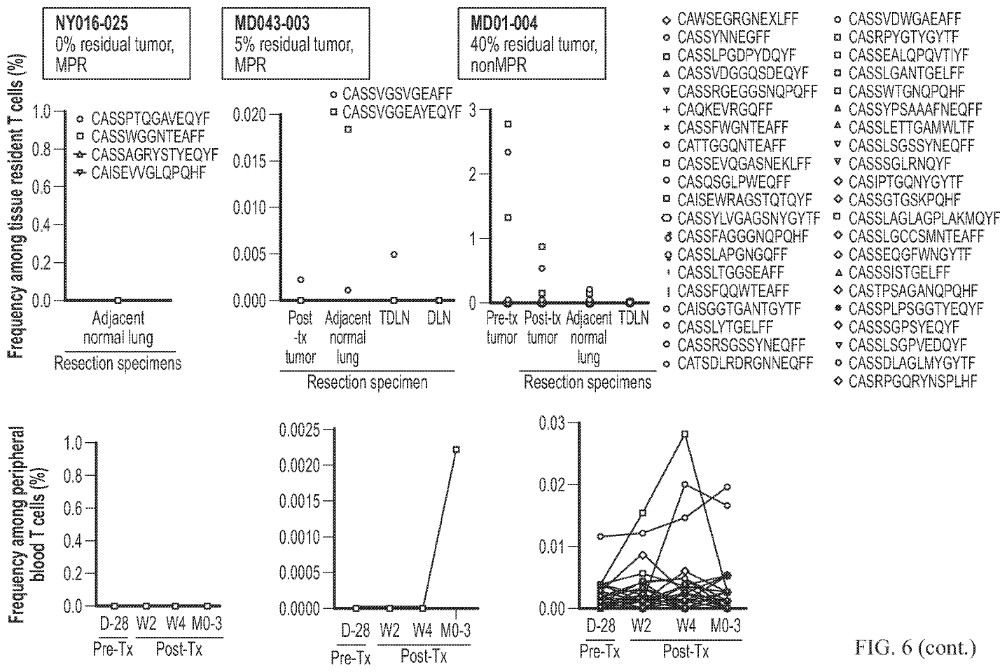

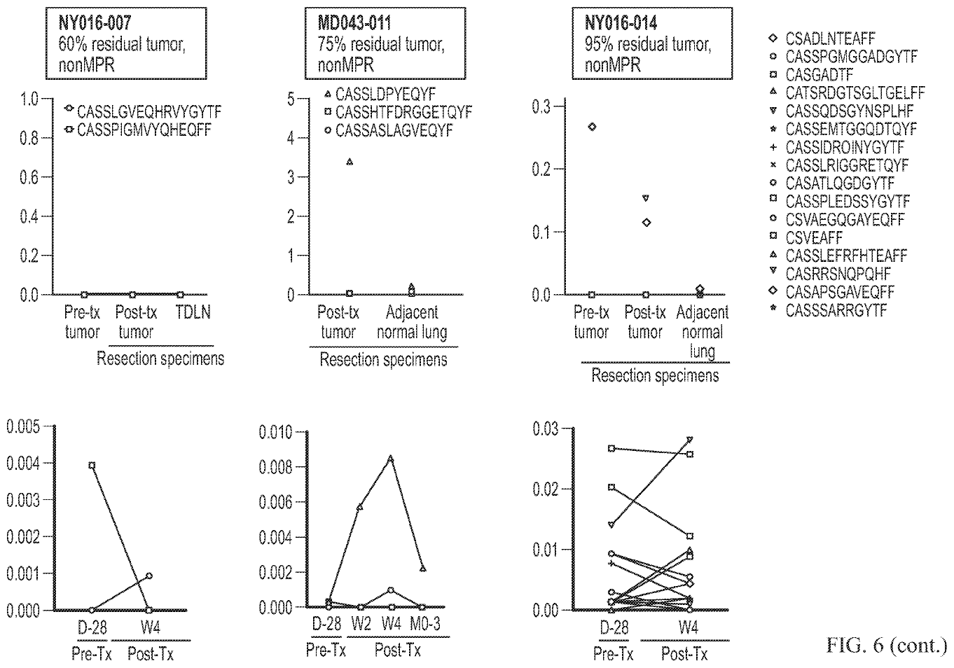

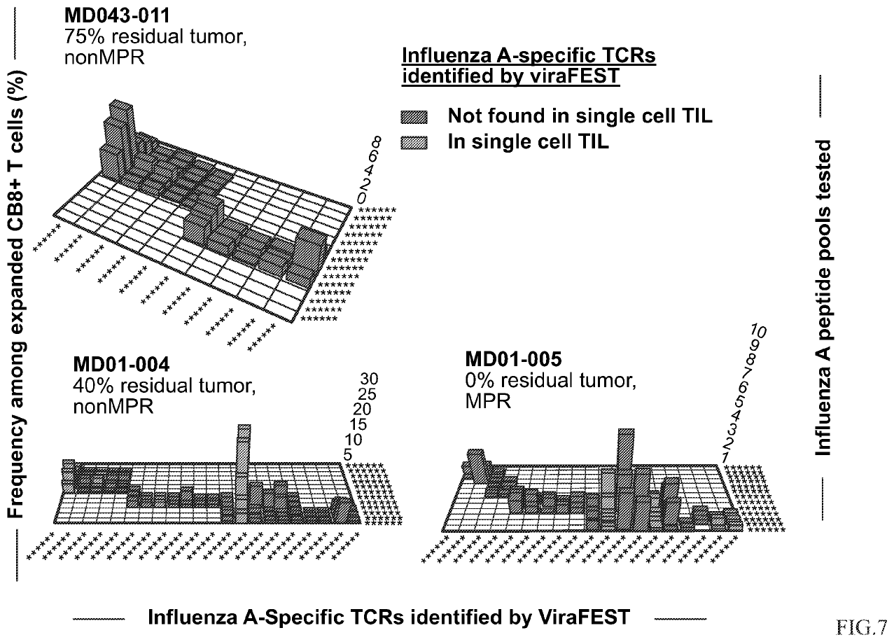

To define the prevalence of MANA-specific CD8 T cells in our cohort, MANAFEST was performed on nine patients treated in the clinical trial, consisting of four MPR and five non-MPR (results from one patient were as described in Forde et al., N. Engl. J. Med., 378:1976-1986 (2018)). Putative MANA, peptide pools representing flu matrix and nucleoproteins, and a pool of MHC class I-restricted CMV, EBV, and flu epitopes were queried for CD8⁺ T cell reactivity (Table 6 (FIG. 13) and Table 7). Among seven (three MPR and four non-MPR) of the nine patients, 72 total unique MANA-specific TCRs were identified (FIG. 2A, FIG. 4, Table 8 (FIG. 14), and Table 9). Ten clonotypes for which the TCRα could be confidently identified from the single cell analysis were selected for validation of MANA recognition using the TCR cloning and Jurkat/NFAT luciferase reporter system. 70% of tested clonotypes were validated as MANA-specific (FIG. 2B, FIG. 4, and FIGS. 5A, 5B, and 5C). Furthermore, binding assays on MANA validated in MD01-005 and MD01-004 displayed high MHC class I affinity and stability (FIG. 5A and 5B). Pathologic response was not associated with the prevalence or frequency of T cells recognizing MANA (Table 9) or intratumoral representation (FIG. 6), suggesting the mere frequency of MANA-specific CD8⁺ T cells did not determine pathologic responsiveness. In fact, more MANA-specific TILs were observed in non-MPR TIL than MPR TIL. An example of a MANAFEST assay output is shown in FIG. 2A (MD01-004, non-MPR) in which 41 neoantigen-specific and 2 CMV/EBV/flu (CEF)-specific TCRβ CDR3 clonotypes were identified. Four of these clones were specific for the hotspot p53 R248L-derived MANA (MD01-004-MANA12), whose specificities were validated by TCR cloning into the Jurkat/NFAT-luciferase system (FIG. 2B). Peptide dose-response curves were comparable to the positive control EBV-specific TCR, suggesting these TCRs were capable of strong ligand-dependent signaling (sometimes referred to as functional avidity). Endogenous processing and HLA A*68:01-restricted presentation of MD01-004-MANA12 were confirmed by transfection of HLA*A6801 and R248L-mutated p53 into a COS-7 cell line and co-culture with a MD01-004-MANA12-reactive TCR (FIG. 5D). Additionally, clones specific for p53 R248L-derived MANA were found at appreciable frequency in the pre- and post-treatment tumor (FIG. 2B), despite the tumor not attaining MPR. They were also observed at lower frequency in adjacent NL, TDLN, and peripheral blood (FIG. 3). Notably, these MANA-specific clones were detected at very low frequency (median: 0.001%, range: 0-0.038%) in the peripheral blood across all available timepoints, thereby highlighting the sensitivity of the MANAFEST assay.

TABLE 7
MANAFEST TCR sequencing summary statistics

| | Assay | | | | # of |
|---|---|---|---|---|---|
| | time- | | | | unique |
| | point | MANA | | | clono- |
| | (relative | tested | SEQ | | types |
| Patient | to | in | ID | | from |
| study ID | surgery) | culture | NO | MANA ID | TCRseq |
| MD01-005 | D + 44 | HVIENIYF | 56 | MD01-005_2 | 8412 |
| MD01-005 | D + 44 | DVAAHLQPL | 57 | MD01-005_3 | 4845 |
| MD01-005 | D + 44 | ETPNLDLM | 58 | MD01-005_4 | 7879 |
| MD01-005 | D + 44 | SVFNTWNPM | 59 | MD01-005_5 | 5261 |
| MD01-005 | D + 44 | EVQQFLRY | 60 | MD01-005_6 | 9514 |
| MD01-005 | D + 44 | EVIVPLSGW | 49 | MD01-005_7 | 9367 |
| MD01-005 | D + 44 | ETMQCSELY | 61 | MD01-005_8 | 7471 |
| MD01-005 | D + 44 | ETMQCSELYHM | 62 | MD01-005_9 | 6763 |
| MD01-005 | D + 44 | ETMQCSEL | 63 | MD01-005_10 | 6034 |
| MD01-005 | D + 44 | ITRTVSANTV | 64 | MD01-005_18 | 6819 |
| MD01-005 | D + 44 | ATKNNKVIMA | 65 | MD01-005_19 | 6720 |
| MD01-005 | D + 44 | VAHFQLQMLK | 66 | MD01-005_20 | 8012 |
| MD01-005 | D + 44 | EEDTFSYLI | 67 | MD01-005_23 | 7548 |
| MD01-005 | D + 44 | AHFQLQML | 68 | MD01-005_24 | 6886 |
| MD01-005 | D + 44 | LHAMIQAAGKL | 69 | MD01-005_25 | 6281 |
| MD01-005 | D + 44 | LHEAQPWFEFL | 70 | MD01-005_26 | 6596 |
| MD01-005 | D + 44 | LHEAQPWFEF | 71 | MD01-005_27 | 5957 |
| MD01-005 | D + 44 | EHLSCPDNFL | 72 | MD01-005_28 | 6477 |
| MD01-005 | D + 44 | NHARIDAAKV | 73 | MD01-005_29 | 7700 |
| MD01-005 | D + 44 | QHQPNPFEV | 74 | MD01-005_30 | 5520 |
| MD01-005 | D + 44 | TQLEKEAL | 75 | MD01-005_33 | 5944 |
| MD01-005 | D + 44 | TRARNEYLLSL | 76 | MD01-005_34 | 5718 |
| MD01-005 | D + 44 | NPMWVVLL | 77 | MD01-005_35 | 6131 |
| MD01-005 | D + 44 | KHILVWAL | 78 | MD01-005_36 | 7272 |
| MD01-005 | D + 44 | SQSDYIPM | 79 | MD01-005_37 | 6470 |
| MD01-005 | D + 44 | VHDYFSVI | 80 | MD01-005_38 | 5333 |
| MD01-005 | D + 44 | IYFPAAQTM | 81 | MD01-005_43 | 5195 |
| MD01-005 | D + 44 | FSYLIWSNPRY | 82 | MD01-005_44 | 5740 |
| MD01-005 | D + 44 | YSWSAQRQAL | 83 | MD01-005_45 | 5599 |
| MD01-005 | D + 44 | FAVWTLAETI | 84 | MD01-005_46 | 5925 |
| MD01-005 | D + 44 | FASLALARRYL | 85 | MD01-005_47 | 8051 |
| MD01-005 | D + 44 | DVIQQDELDSY | 86 | MD01-005_48 | 6985 |
| MD01-005 | D + 44 | KNRSSGTVSA | 87 | MD01-005_49 | 6349 |
| MD01-005 | D + 44 | KLKRFNLSA | 88 | MD01-005_50 | 5819 |
| MD01-005 | D + 44 | KSFAVWTLA | 89 | MD01-005_51 | 5444 |
| MD01-005 | D + 44 | KWRLSLCTV | 90 | MD01-005_52 | 7218 |
| MD01-005 | D + 44 | RSRPVAATAK | 91 | MD01-005_53 | 5256 |
| MD01-005 | D + 44 | RSRPVAATA | 92 | MD01-005_54 | 5610 |
| MD01-005 | D + 44 | TAKQAHLTTLK | 93 | MD01-005_55 | 4997 |
| MD01-005 | D + 44 | SHCPSAMGI | 94 | MD01-005_56 | 5048 |
| MD01-005 | D + 44 | FHASEGWL | 95 | MD01-005_57 | 5819 |
| MD01-005 | D + 44 | THEVIVPL | 96 | MD01-005_58 | 5842 |
| MD01-005 | D + 44 | SRHCLQPL | 97 | MD01-005_59 | 3327 |
| MD01-005 | D + 44 | FASLALARRY | 98 | MD01-005_60 | 913 |
| MD01-005 | D + 44 | SVFNTWNPMWV | 99 | MD01-005_61 | 2546 |
| MD01-005 | D + 44 | LTHEVIVPL | 100 | MD01-005_62 | 3832 |
| MD01-005 | D + 44 | YTVMARKSPV | 101 | MD01-005_63 | 2207 |
| MD043-003 | D + 121 | LSEKGIEDY | 102 | MD043-003_1 | 2372 |
| MD043-003 | D + 121 | MSDVRTVF | 103 | MD043-003_2 | 11614 |
| MD043-003 | D + 121 | NSDEPVNLTF | 104 | MD043-003_3 | 9178 |
| MD043-003 | D + 121 | MSDVRTVFL | 105 | MD043-003_4 | 6417 |
| MD043-003 | D + 121 | ANDVNDALGY | 106 | MD043-003_6 | 2332 |
| MD043-003 | D + 121 | LLASVAPRY | 107 | MD043-003_7 | 8335 |
| MD043-003 | D + 121 | ALMAVIVLV | 108 | MD043-003_11 | 1429 |
| MD043-003 | D + 121 | ALMAVIVLVAL | 109 | MD043-003_12 | 7235 |
| MD043-003 | D + 121 | FLNGLEETAGV | 110 | MD043-003_13 | 10252 |
| MD043-003 | D + 121 | ALMAVIVL | 111 | MD043-003_14 | 3718 |
| MD043-003 | D + 121 | LMAVIVLV | 112 | MD043-003_15 | 8100 |
| MD043-003 | D + 121 | VLVALMAV | 113 | MD043-003_17 | 7404 |
| MD043-003 | D + 121 | MLAACAGEV | 114 | MD043-003_19 | 12755 |
| MD043-003 | D + 121 | YPMCSGEKAY | 115 | MD043-003_21 | 10220 |
| MD043-003 | D + 121 | MPSNIQNF | 116 | MD043-003_22 | 9067 |
| MD043-003 | D + 121 | LPVAVLVALM | 117 | MD043-003_25 | 10244 |
| MD043-003 | D + 121 | MTSGVYAF | 118 | MD043-003_26 | 9261 |
| MD043-003 | D + 121 | LPTPTYPL | 119 | MD043-003_28 | 12076 |
| MD043-003 | D + 121 | YSMSDVRTVF | 120 | MD043-003_29 | 2552 |
| MD043-003 | D + 121 | DANDVNDALGY | 121 | MD043-003_30 | 10469 |
| MD043-003 | D + 121 | AEAGAEAASL | 122 | MD043-003_31 | 11061 |
| MD043-003 | D + 121 | AEAASLNASL | 123 | MD043-003_32 | 1822 |
| MD043-003 | D + 121 | LENCAEVMRLL | 124 | MD043-003_34 | 3373 |
| MD043-003 | D + 121 | AETQSRFQLL | 125 | MD043-003_35 | 7423 |
| MD043-003 | D + 121 | LENCAEVM | 126 | MD043-003_36 | 8204 |
| MD043-003 | D + 121 | LENCAEVMRL | 127 | MD043-003_37 | 11116 |
| MD043-003 | D + 121 | SENSDEPVNL | 128 | MD043-003_38 | 12313 |
| MD043-003 | D + 121 | TEDEIYSRICL | 129 | MD043-003_39 | 11559 |
| MD043-003 | D + 121 | AESEAHRDSM | 130 | MD043-003_40 | 10305 |
| MD043-003 | D + 121 | YSMSDVRTVFL | 131 | MD043-003_41 | 9418 |
| MD043-003 | D + 121 | YSMSDVRTV | 132 | MD043-003_42 | 10733 |
| MD043-003 | D + 121 | FASGADVQV | 133 | MD043-003_43 | 7321 |
| MD043-003 | D + 121 | MPRQPSCPL | 134 | MD043-003_63 | 10902 |
| MD043-003 | D + 121 | TPLCQHLAAL | 135 | MD043-003_64 | 8635 |
| MD043-008 | D − 14 | SLHEFHLV | 136 | MD043-008_1 | 7247 |
| MD043-008 | D − 14 | HLVDLSRRFLV | 137 | MD043-008_2 | 7043 |
| MD043-008 | D − 14 | RLSDETLIDIV | 138 | MD043-008_3 | 6674 |
| MD043-008 | D − 14 | VLFDTQDPL | 139 | MD043-008_4 | 5953 |
| MD043-008 | D − 14 | VLFDTQDPLNA | 140 | MD043-008_5 | 3903 |
| MD043-008 | D − 14 | LMSAAAIYTV | 141 | MD043-008_6 | 5919 |
| MD043-008 | D − 14 | CLMSAAAIYTV | 142 | MD043-008_7 | 6544 |
| MD043-008 | D − 14 | ILAGLCLMSA | 143 | MD043-008_8 | 5887 |
| MD043-008 | D − 14 | LLLPELCSA | 144 | MD043-008_9 | 6801 |
| MD043-008 | D − 14 | KMALLQYL | 145 | MD043-008_10 | 5656 |
| MD043-008 | D − 14 | LMSAAAIY | 146 | MD043-008_11 | 4685 |
| MD043-008 | D − 14 | CLMSAAAIY | 147 | MD043-008_12 | 3594 |
| MD043-008 | D − 14 | LLLPELCSAFY | 148 | MD043-008_13 | 5631 |
| MD043-008 | D − 14 | HLGKPGHLSY | 149 | MD043-008_14 | 6244 |
| MD043-008 | D − 14 | MSLCVLLY | 150 | MD043-008_15 | 5317 |
| MD043-008 | D − 14 | DMSLCVLLY | 151 | MD043-008_16 | 4583 |
| MD043-008 | D − 14 | LDMSLCVLLY | 152 | MD043-008_17 | 4754 |
| MD043-008 | D − 14 | SLDMSLCVLLY | 153 | MD043-008_18 | 5434 |
| MD043-008 | D − 14 | TTYRDWLGLDY | 154 | MD043-008_19 | 6571 |
| MD043-008 | D − 14 | LMCQKFLARY | 155 | MD043-008_20 | 4641 |
| MD043-008 | D − 14 | APTGNFCPQPL | 156 | MD043-008_21 | 6725 |
| MD043-008 | D − 14 | CPQPLLNSSM | 157 | MD043-008_22 | 3735 |
| MD043-008 | D − 14 | TPNYSVSML | 158 | MD043-008_23 | 4387 |
| MD043-008 | D − 14 | TPNYSVSM | 159 | MD043-008_24 | 5374 |
| MD043-008 | D − 14 | YPNGTSSL | 160 | MD043-008_25 | 5005 |
| MD043-008 | D − 14 | KPGHLSYAL | 161 | MD043-008_26 | 3580 |
| MD043-008 | D − 14 | FTRKLLGSAL | 162 | MD043-008_27 | 5003 |
| MD043-008 | D − 14 | GPASYPIPV | 163 | MD043-008_28 | 6553 |
| MD043-008 | D − 14 | LPTESPHSSL | 164 | MD043-008_29 | 4529 |
| MD043-008 | D − 14 | IPHFTATSDAF | 165 | MD043-008_30 | 6195 |
| MD043-008 | D − 14 | VEIKAVPEGF | 166 | MD043-008_31 | 4343 |
| MD043-008 | D − 14 | DEVSATETCY | 167 | MD043-008_32 | 4327 |
| MD043-008 | D − 14 | GELDNQLTTY | 168 | MD043-008_33 | 4151 |
| MD043-008 | D − 14 | AELGQVLIY | 169 | MD043-008_34 | 3953 |
| MD043-008 | D − 14 | AELGQVLI | 170 | MD043-008_35 | 5558 |
| MD043-008 | D − 14 | AELGQVLIYL | 171 | MD043-008_36 | 4401 |
| MD043-008 | D − 14 | PELCSAFY | 172 | MD043-008_37 | 4800 |
| MD043-008 | D − 14 | EEFLNHSKAW | 173 | MD043-008_38 | 7314 |
| MD043-008 | D − 14 | TEEFLNHSKAW | 174 | MD043-008_39 | 6529 |
| MD043-008 | D − 14 | IENLHDDSCY | 175 | MD043-008_40 | 4882 |
| MD043-008 | D − 14 | SRMHRGGLRL | 176 | MD043-008_41 | 4702 |
| MD043-008 | D − 14 | MYHSRMHRGGL | 177 | MD043-008_42 | 5150 |
| MD043-008 | D − 14 | FFFTRKLL | 178 | MD043-008_43 | 5196 |
| MD043-008 | D − 14 | IRLQILRQVSL | 179 | MD043-008_44 | 4077 |
| MD043-008 | D − 14 | FYYTGVGM | 180 | MD043-008_45 | 4947 |
| MD043-008 | D − 14 | FYYTGVGML | 181 | MD043-008_46 | 3735 |
| MD043-008 | D − 14 | FYYTGVGMLI | 182 | MD043-008_47 | 5955 |
| MD043-008 | D − 14 | YYTGVGML | 183 | MD043-008_48 | 4042 |
| MD043-008 | D − 14 | YYTGVGMLI | 184 | MD043-008_49 | 5595 |
| MD043-008 | D − 14 | FYPATFGIL | 185 | MD043-008_50 | 5992 |
| MD043-008 | D − 14 | YAPPQDGPASY | 186 | MD043-008_51 | 5474 |
| MD043-008 | D − 14 | FTATSDAF | 187 | MD043-008_52 | 4821 |
| MD043-008 | D − 14 | FQMDDYSLCVL | 188 | MD043-008_53 | 6371 |
| MD043-008 | D − 14 | MTFSNPPDWL | 189 | MD043-008_54 | 5041 |
| MD043-008 | D − 14 | YAAHLLDIAM | 190 | MD043-008_55 | 4947 |
| MD043-008 | D − 14 | FFYAAHLL | 191 | MD043-008_56 | 4234 |
| MD043-008 | D − 14 | FYAAHLLDIAM | 192 | MD043-008_57 | 7088 |
| MD043-008 | D − 14 | FMDSCTMRF | 193 | MD043-008_58 | 4652 |
| MD043-008 | D − 14 | KSMERDCATF | 194 | MD043-008_59 | 5787 |
| MD01-004 | D + 21 | RTWRRTRR | 195 | MD01-004_01 | 10375 |
| MD01-004 | D + 21 | RTWRRTRRGR | 196 | MD01-004_02 | 9922 |
| MD01-004 | D + 21 | RTWRRTRRGRR | 197 | MD01-004_03 | 11966 |
| MD01-004 | D + 21 | RTRRGRRSSR | 198 | MD01-004_04 | 9713 |
| MD01-004 | D + 21 | RSSRTLSR | 199 | MD01-004_05 | 9031 |
| MD01-004 | D + 21 | VMYDGFSVQR | 200 | MD01-004_06 | 8929 |
| MD01-004 | D + 21 | CVKVCAYIR | 201 | MD01-004_07 | 8981 |
| MD01-004 | D + 21 | KSTSISTAMR | 202 | MD01-004_08 | 7928 |
| MD01-004 | D + 21 | GASSIWYR | 203 | MD01-004_09 | 9723 |
| MD01-004 | D + 21 | AGASSIWYR | 204 | MD01-004_10 | 7655 |
| MD01-004 | D + 21 | YVMYDGFSVQR | 205 | MD01-004_11 | 13002 |
| MD01-004 | D + 21 | NSSCMGGMNLR | 1 | MD01-004_12 | 9183 |
| MD01-004 | D + 21 | ELFLVKAKIHK | 206 | MD01-004_13 | 11302 |
| MD01-004 | D + 21 | STSISTAMR | 207 | MD01-004_14 | 10062 |
| MD01-004 | D + 21 | TSISTAMR | 208 | MD01-004_15 | 10850 |
| MD01-004 | D + 21 | FIFTSIAGIR | 209 | MD01-004_16 | 8630 |
| MD01-004 | D + 21 | FTSIAGIR | 210 | MD01-004_17 | 6833 |
| MD01-004 | D + 21 | FTNRKVPYCFK | 211 | MD01-004_18 | 6093 |
| MD01-004 | D + 21 | EAFHQSCFR | 212 | MD01-004_19 | 8912 |
| MD01-004 | D + 21 | HPNVILNSLY | 213 | MD01-004_20 | 8245 |
| MD01-004 | D + 21 | FPNVVSGL | 214 | MD01-004_21 | 9899 |
| MD01-004 | D + 21 | MAENTEGDLNF | 215 | MD01-004_22 | 9485 |
| MD01-004 | D + 21 | MLVELTPPY | 216 | MD01-004_23 | 7595 |
| MD01-004 | D + 21 | EPSDVTETLM | 217 | MD01-004_24 | 6746 |
| MD01-004 | D + 21 | EPSDVTETL | 218 | MD01-004_25 | 8694 |
| MD01-004 | D + 21 | SPAMTSTSFFF | 219 | MD01-004_26 | 7945 |
| MD01-004 | D + 21 | MTSTSFFF | 220 | MD01-004_27 | 6631 |
| MD01-004 | D + 21 | MAIEDILF | 221 | MD01-004_28 | 6022 |
| MD01-004 | D + 21 | IPEELEYF | 222 | MD01-004_29 | 4958 |
| MD01-004 | D + 21 | MPICPTYNEV | 223 | MD01-004_30 | 4557 |
| MD01-004 | D + 21 | CAYIRKQVEKI | 223 | MD01-004_31 | 5540 |
| MD01-004 | D + 21 | LAQEGTTVI | 224 | MD01-004_32 | 7055 |
| MD01-004 | D + 21 | HPNVILNSLYV | 225 | MD01-004_33 | 11278 |
| MD01-004 | D + 21 | LPDHFGLGPV | 226 | MD01-004_34 | 8888 |
| MD01-004 | D + 21 | MAIEDILFV | 227 | MD01-004_35 | 10310 |
| MD01-004 | D + 21 | IPEELEYFI | 228 | MD01-004_36 | 5052 |
| MD01-004 | D + 21 | EPQNFIDSLI | 229 | MD01-004_37 | 9296 |
| MD01-004 | D + 21 | CPTYNEVHL | 230 | MD01-004_38 | 9098 |
| MD01-004 | D + 21 | MYDGFSVQRL | 231 | MD01-004_39 | 11006 |
| MD01-004 | D + 21 | MYDGFSVQRLV | 232 | MD01-004_40 | 8422 |
| MD01-004 | D + 21 | AYDASTFRGL | 233 | MD01-004_41 | 7408 |
| MD01-004 | D + 21 | FTDCGRPPL | 234 | MD01-004_42 | 6671 |
| MD01-004 | D + 21 | KFDLFARL | 235 | MD01-004_43 | 4954 |
| MD01-004 | D + 21 | STYLIAQSI | 236 | MD01-004_44 | 6837 |
| MD01-004 | D + 21 | KSTSISTAMRL | 237 | MD01-004_45 | 8566 |
| MD01-004 | D + 21 | QTFGKMFFV | 238 | MD01-004_46 | 6488 |
| MD01-004 | D + 21 | WAYDASTFRGL | 239 | MD01-004_47 | 6516 |
| MD01-004 | D + 21 | STHPPGASL | 240 | MD01-004_48 | 7184 |
| MD01-004 | D + 21 | RADPRAGPSV | 241 | MD01-004_49 | 8889 |
| MD01-004 | D + 21 | MTSTSFFFTL | 242 | MD01-004_50 | 7303 |
| MD01-004 | D + 21 | RSAEPQNFI | 243 | MD01-004_51 | 7542 |
| MD01-004 | D + 21 | LTSSDDLLI | 244 | MD01-004_52 | 7889 |
| MD043-011 | D − 14 | KYMLNSVLENF | 245 | MD043-011_01 | 7977 |
| MD043-011 | D − 14 | YMLNSVLENF | 246 | MD043-011_02 | 8241 |
| MD043-011 | D − 14 | GYACAEPSF | 247 | MD043-011_03 | 4740 |
| MD043-011 | D − 14 | FFAAQAGAWKI | 248 | MD043-011_04 | 10389 |
| MD043-011 | D − 14 | SFFAAQAGAW | 249 | MD043-011_05 | 6864 |
| MD043-011 | D − 14 | YMLKAKSQF | 250 | MD043-011_06 | 9612 |
| MD043-011 | D − 14 | RYFVPKML | 251 | MD043-011_07 | 10540 |
| MD043-011 | D − 14 | ATLNGRMYF | 252 | MD043-011_08 | 11654 |
| MD043-011 | D − 14 | YTISFLFW | 253 | MD043-011_09 | 4760 |
| MD043-011 | D − 14 | DYTISFLFW | 254 | MD043-011_10 | 7551 |
| MD043-011 | D − 14 | KYMLNSVL | 255 | MD043-011_11 | 7219 |
| MD043-011 | D − 14 | RYPAKVTL | 256 | MD043-011_12 | 10782 |
| MD043-011 | D − 14 | FFAAQAGAW | 257 | MD043-011_13 | 3924 |
| MD043-011 | ID − 14 | EYMLKAKSQF | 258 | MD043-011_14 | 7099 |
| MD043-011 | D − 14 | KESFGPQAL | 259 | MD043-011_15 | 9032 |
| MD043-011 | D − 14 | CEVAPNNVV | 260 | MD043-011_16 | 5433 |
| MD043-011 | D − 14 | KEMHPNKLNAV | 261 | MD043-011_17 | 1852 |
| MD043-011 | D − 14 | CEVAPNNV | 262 | MD043-011_18 | 3343 |
| MD043-011 | D − 14 | KQFFYNII | 263 | MD043-011_19 | 3030 |
| MD043-011 | D − 14 | SQLQGLQL | 264 | MD043-011_20 | 1106 |
| MD043-011 | D − 14 | TEYKLVVVGAC | 265 | MD043-011_21 | 2022 |
| MD043-011 | D − 14 | FEDGPYAV | 266 | MD043-011_22 | 623 |
| MD043-011 | D − 14 | AQAGAWKI | 267 | MD043-011_23 | 1990 |
| MD043-011 | D − 14 | AQAGAWKIYAV | 51 | MD043-011_24 | 2723 |
| MD043-011 | D − 14 | AERLVGPGY | 268 | MD043-011_25 | 2709 |
| MD043-011 | D − 14 | IEYMLKAKSQF | 269 | MD043-011_26 | 2317 |
| MD043-011 | D − 14 | SDYTISFLFW | 270 | MD043-011_27 | 10837 |
| MD043-011 | D − 14 | GELGWENPNQW | 271 | MD043-011_28 | 2829 |
| MD043-011 | D − 14 | SEMTAVTQKI | 272 | MD043-011_29 | 3030 |
| MD043-011 | D − 14 | SEMTAVTQKIV | 273 | MD043-011_30 | 3215 |
| MD043-011 | D − 14 | AQAGAWKIY | 52 | MD043-011_31 | 7000 |
| MD043-011 | D − 14 | NKMDMNQW | 274 | MD043-011_32 | 12195 |
| MD043-011 | D − 14 | YTSSEVSTV | 275 | MD043-011_33 | 5938 |
| MD043-011 | D − 14 | YTSSEVSTVEL | 276 | MD043-011_34 | 5611 |
| MD043-011 | D − 14 | YSPDILPTV | 277 | MD043-011_35 | 3833 |
| MD043-011 | D − 14 | FAAQAGAWKIY | 53 | MD043-011_36 | 5945 |
| MD043-011 | D − 14 | FAAQAGAWKI | 278 | MD043-011_37 | 8014 |
| MD043-011 | D − 14 | FAAQAGAW | 279 | MD043-011_38 | 4933 |
| MD043-011 | D − 14 | KTATLNGRMYF | 280 | MD043-011_39 | 8230 |
| MD043-011 | D − 14 | KTATLNGRM | 281 | MD043-011_40 | 5929 |
| MD043-011 | D − 14 | YTISFLFWIL | 282 | MD043-011_41 | 3883 |
| MD043-011 | D − 14 | YTISFLFWI | 283 | MD043-011_42 | 13198 |
| MD043-011 | D − 14 | IALRPSGTM | 284 | MD043-011_43 | 7339 |
| MD043-011 | D − 14 | IALRPSGTML | 285 | MD043-011_44 | 5416 |
| MD043-011 | D − 14 | FAVEAHQCI | 286 | MD043-011_45 | 2936 |
| MD043-011 | D − 14 | MSSLPCPL | 287 | MD043-011_46 | 3147 |
| MD043-011 | D − 14 | YACAEPSF | 288 | MD043-011_47 | 10633 |
| MD043-011 | D − 14 | IVDPDPVL | 289 | MD043-011_48 | 2971 |
| MD043-011 | D − 14 | RALKEKAQPL | 290 | MD043-011_49 | 6372 |
| MD01-019 | D + 38 | YLNSRQFPM | 291 | MD01-019_01 | 4547 |
| MD01-019 | D + 38 | SIMALSTSI | 292 | MD01-019_02 | 3219 |
| MD01-019 | D + 38 | SLTDISTL | 293 | MD01-019_03 | 4704 |
| MD01-019 | D + 38 | FLISYWSEQI | 294 | MD01-019_04 | 4375 |
| MD01-019 | D + 38 | SMLSLPRV | 295 | MD01-019_05 | 5301 |
| MD01-019 | D + 38 | AVASVLPLWPA | 296 | MD01-019_06 | 5247 |
| MD01-019 | D + 38 | MLLVIIVSVGI | 297 | MD01-019_07 | 2920 |
| MD01-019 | D + 38 | SQHQVLFFL | 298 | MD01-019_10 | 5055 |
| MD01-019 | D + 38 | RLRTDLFSK | 299 | MD01-019_11 | 4252 |
| MD01-019 | D + 38 | KQRTSSEK | 300 | MD01-019_12 | 10161 |
| MD01-019 | D + 38 | RLKYNLQGYK | 301 | MD01-019_13 | 6999 |
| MD01-019 | D + 38 | RSRRSTTA | 302 | MD01-019_14 | 4772 |
| MD01-019 | D + 38 | RMRAMATA | 303 | MD01-019_15 | 4574 |
| NY016-007 | D + 30 | LTSPIVCF | 304 | NY016-007_1 | 4236 |
| NY016-007 | D + 30 | LARASPALASL | 305 | NY016-007_2 | 4820 |
| NY016-007 | D + 30 | LRNGALTSPI | 306 | NY016-007_3 | 4793 |
| NY016-007 | D + 30 | VLRNGALTSPI | 307 | NY016-007_4 | 3590 |
| NY016-007 | D + 30 | LRNGALTSPIV | 308 | NY016-007_5 | 4288 |
| NY016-007 | D + 30 | LARASPAL | 309 | NY016-007_6 | 4906 |
| NY016-007 | D + 30 | SLARASPAL | 310 | NY016-007_7 | 4390 |
| NY016-007 | D + 30 | LRSLTFSLV | 311 | NY016-007_8 | 3722 |
| NY016-007 | D + 30 | SAITSKVSTV | 312 | NY016-007_9 | 6575 |
| NY016-007 | D + 30 | AITSKVSTV | 313 | NY016-007_10 | 4669 |
| NY016-007 | D + 30 | SAITSKVSTV | 314 | NY016-007_11 | 5002 |
| NY016-007 | D + 30 | ASLARASPA | 315 | NY016-007_12 | 4526 |
| NY016-007 | D + 30 | SLARASPA | 316 | NY016-007_13 | 4351 |
| NY016-007 | D + 30 | ASLARASPAL | 317 | NY016-007_14 | 3413 |
| NY016-007 | D + 30 | QASLARASPA | 318 | NY016-007_15 | 3735 |
| NY016-007 | D + 30 | LARASPALA | 319 | NY016-007_16 | 5266 |
| NY016-007 | D + 30 | KLRSLTFSLV | 320 | NY016-007_17 | 2321 |
| NY016-014 | D + 30 | LLADATVEL | 321 | NY016-014_1 | 3258 |
| NY016-014 | D + 30 | LLADATVELSL | 322 | NY016-014_2 | 3250 |
| NY016-014 | D + 30 | HMAFSPAV | 323 | NY016-014_3 | 3886 |
| NY016-014 | D + 30 | YLDSIVFL | 324 | NY016-014_4 | 3682 |
| NY016-014 | D + 30 | FLEDLSPL | 325 | NY016-014_5 | 3939 |
| NY016-014 | D + 30 | YLDSIVFLEDL | 326 | NY016-014_6 | 3374 |
| NY016-014 | D + 30 | FLEDLSPLEA | 327 | NY016-014_7 | 3012 |
| NY016-014 | D + 30 | LLLHGAEPKL | 328 | NY016-014_8 | 2973 |
| NY016-014 | D + 30 | TLIDVPKV | 329 | NY016-014_9 | 3612 |
| NY016-014 | D + 30 | TMACINLA | 330 | NY016-014_10 | 2921 |
| NY016-014 | D + 30 | LSKDIMFHFK | 331 | NY016-014_11 | 4462 |
| NY016-014 | D + 30 | VTMACINLASK | 332 | NY016-014_12 | 4308 |
| NY016-014 | D + 30 | TMACINLASK | 333 | NY016-014_13 | 4043 |
| NY016-014 | D + 30 | MSYDNNLFIK | 334 | NY016-014_14 | 2860 |
| NY016-014 | D + 30 | KTWKEKTLK | 335 | NY016-014_15 | 5046 |
| NY016-014 | D + 30 | VTLIDVPK | 336 | NY016-014_16 | 5284 |
| NY016-014 | D + 30 | MPLVHMAF | 337 | NY016-014_21 | 5254 |
| NY016-014 | D + 30 | MPLVHMAFSPA | 338 | NY016-014_22 | 5131 |
| NY016-014 | D + 30 | YPDYLDSIVF | 339 | NY016-014_23 | 4721 |
| NY016-014 | D + 30 | YPDYLDSIVFL | 340 | NY016-014_24 | 4822 |
| NY016-014 | D + 30 | MSYDNNLF | 341 | NY016-014_25 | 4269 |
| NY016-014 | D + 30 | MPLVHMAF | 342 | NY016-014_31 | 4186 |
| NY016-014 | D + 30 | MAFSPAVDV | 343 | NY016-014_32 | 5182 |
| NY016-025 | D − 3 | AVQWLRPK | 344 | NY016-025_01 | 6087 |
| NY016-025 | D − 3 | HVMPDTPDILK | 345 | NY016-025_02 | 6160 |
| NY016-025 | D − 3 | KVMYILFY | 346 | NY016-025_03 | 6036 |
| NY016-025 | D − 3 | VQNAVQWLRPK | 347 | NY016-025_04 | 5922 |
| NY016-025 | D − 3 | VQNAVQWLR | 348 | NY016-025_05 | 6275 |
| NY016-025 | D − 3 | TLFQIIYDNLR | 349 | NY016-025_06 | 6535 |
| NY016-025 | D − 3 | CLASLHPR | 350 | NY016-025_07 | 5801 |
| NY016-025 | D − 3 | RSLGCLASLH | 351 | NY016-025_08 | 5860 |
| NY016-025 | D − 3 | KLLHEYWMSLR | 352 | NY016-025_09 | 5149 |
| NY016-025 | D − 3 | LLHEYWMSLR | 353 | NY016-025_10 | 6186 |
| NY016-025 | D − 3 | EVKEEDEPF | 354 | NY016-025_11 | 5870 |
| NY016-025 | D − 3 | QVNKVMYILFY | 355 | NY016-025_12 | 6117 |
| NY016-025 | D − 3 | EVQNAVQWL | 356 | NY016-025_13 | 5838 |
| NY016-025 | D − 3 | LHEYWMSL | 357 | NY016-025_14 | 5991 |
| NY016-025 | D − 3 | YKLLHEYWMSL | 358 | NY016-025_15 | 5071 |
| NY016-025 | D − 3 | MEESNNSTL | 359 | NY016-025_16 | 5662 |
| NY016-025 | D − 3 | MEESNNSTLFI | 360 | NY016-025_17 | 5565 |

TABLE 9
MANAFEST assay results summary

| | | | No. of | | | | | | No. of | Jurkat/ |
|---|---|---|---|---|---|---|---|---|---|---|
| | | | non- | No. of | | | | | MANA- | NFAT |
| | | | synonymous | predicted | MANA- | No. of | | | specific | validation |
| | | % | mutations | non- | FEST | putative | No. of | % of | TCRβ | of MANA |
| | Histologic | residual | per | redundant | Time- | MANAs | positive | MANAs | clones | specific |
| Study ID | subtype | tumor | exome | MANAs | point^{c} | tested | MANAs | positive | identified | clonotypes |
| NY016-025 | Adenosquamous | 0 | 27 | 75 | D − 3 | 17 | 4 | 23.5% | 4 | NA |
| MD01-005*^{,b} | Squamous | 0 | 256 | 158 | D + 44 | 47 | 3 | 6.3% | 4 | 2 |
| MD043-003^{a} | Adeno | 5 | 66 | 297 | D + 121 | 34 | 2 | 5.9% | 2 | NA |
| MD043-008* | Adeno | 10 | 310 | 213 | D − 14 | 59 | 0 | 0.0% | 0 | NA |
| MD01-004* | Adenosquamous | 40 | 99 | 63 | D + 21 | 52 | 27 | 51.9% | 41 | 4 |
| NY016-007* | Squamous | 60 | 5 | 1 | D + 30 | 17 | 2 | 11.8% | 2 | NA |
| MD043-011* | Adeno | 75 | 75 | 46 | D − 14 | 49 | 3 | 6.1% | 3 | 1 |
| MD01-019* | Adeno | 95 | 105 | 59 | D − 14 | 13 | 0 | 0.0% | 0 | NA |
| NY016-014* | Adeno | 95 | 26 | 19 | D + 30 | 23 | 12 | 52.1% | 16 | NA |

*WES and predicted neoantigens previously reported in Forde et al., N. Engl. J. Med., 378: 1976-1986 (2018))
^{a}No pre-treatment biopsy available for WES. WES performed on resected tumor
^{b}MANAFEST results reported in Forde et al., N. Engl. J. Med., 378: 1976-1986 (2018)) and Danilova et al., Can. Immunol. Res., 6: 888-899 (2018)
^{c}Relative to surgical resection

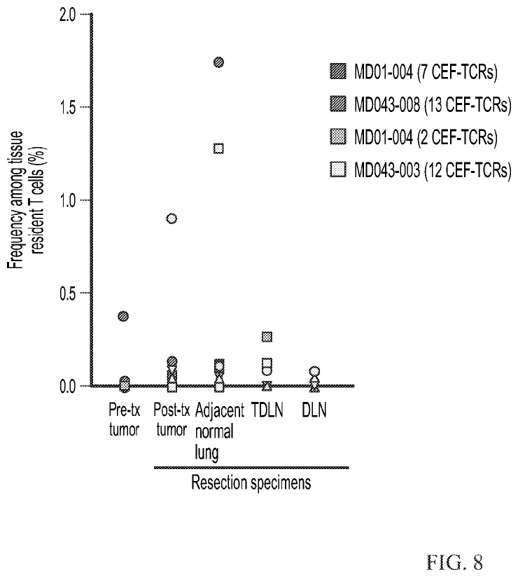

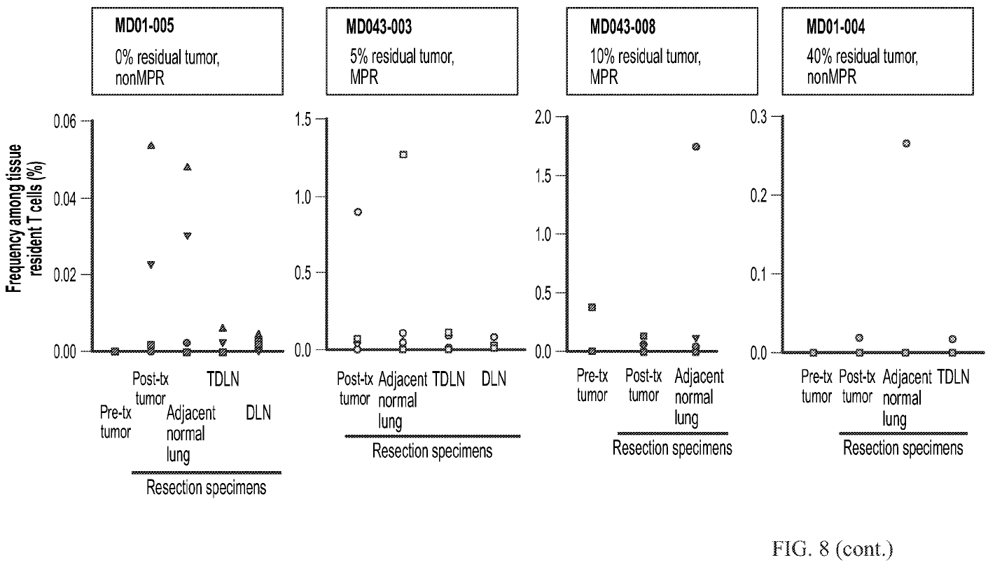

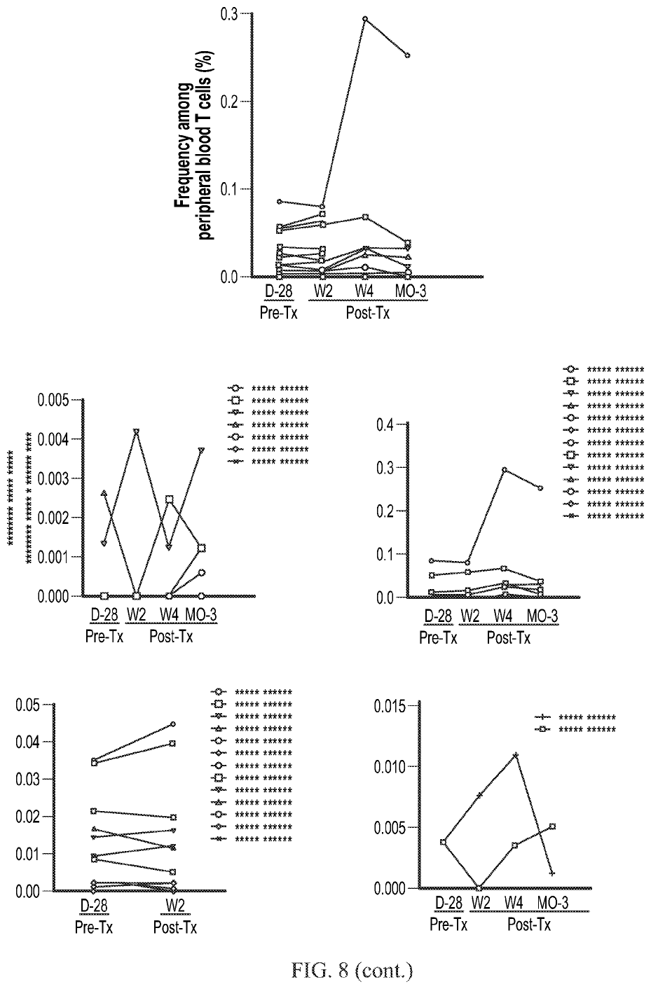

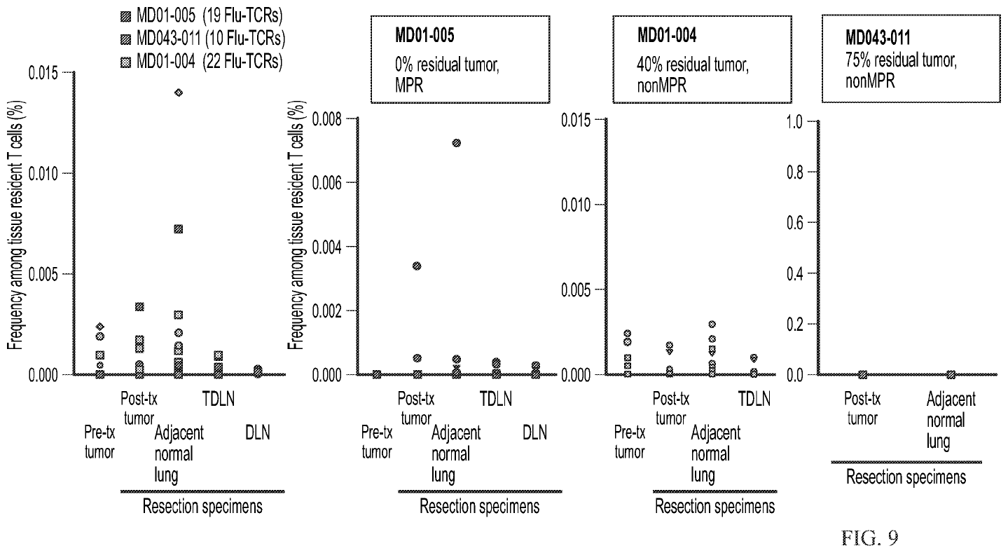

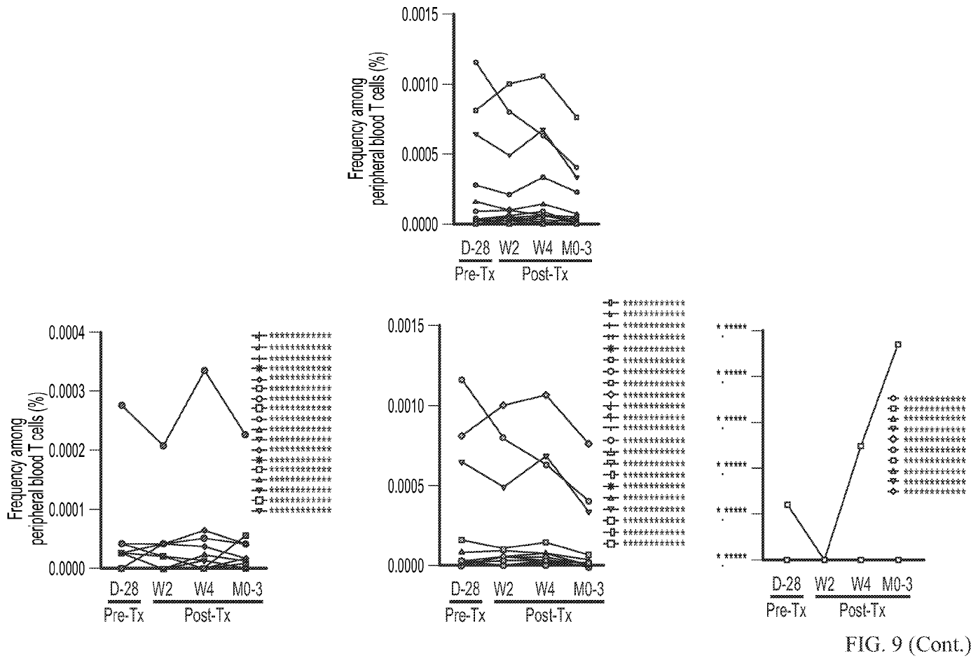

Additionally, viral-specific TCRs, identified by culture with CEF (positive control in the MANAFEST assay) or influenza peptide pools, were detected in 5 of the 9 patients tested (FIG. 4, FIG. 7, and Table 8 (FIG. 14)). A total of 88 unique viral-specific TCRs were identified; 54 of these were specific for flu and 34 of these were CEF-specific T cell clones (of which 6 could be mapped to public EBV-reactive TCRβ clonotypes, 7 to public flu-reactive TCRβ clonotypes). No CMV-reactive TCRs were mapped from our viral-specific TCRs. No consistent pattern was observed for the frequency of viral T cells in the tissue or peripheral blood (FIG. 8 and FIG. 9).

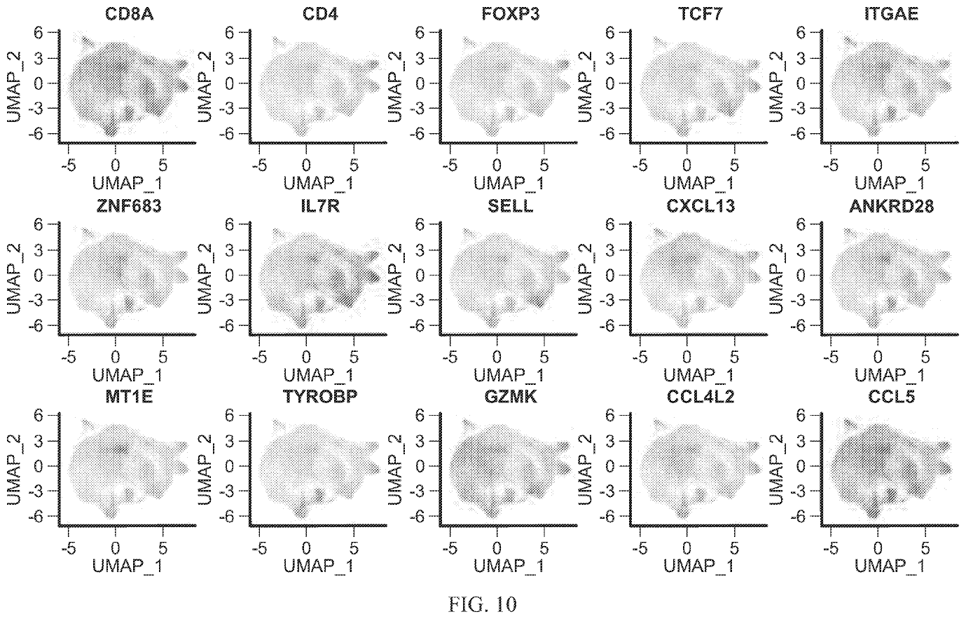

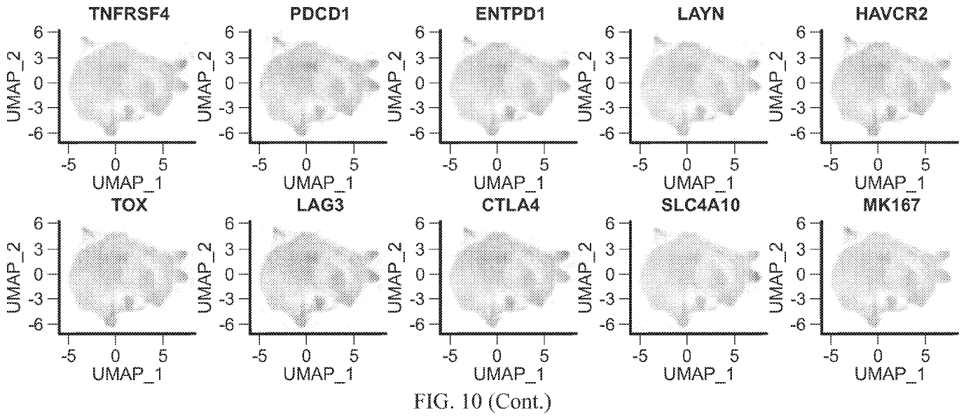

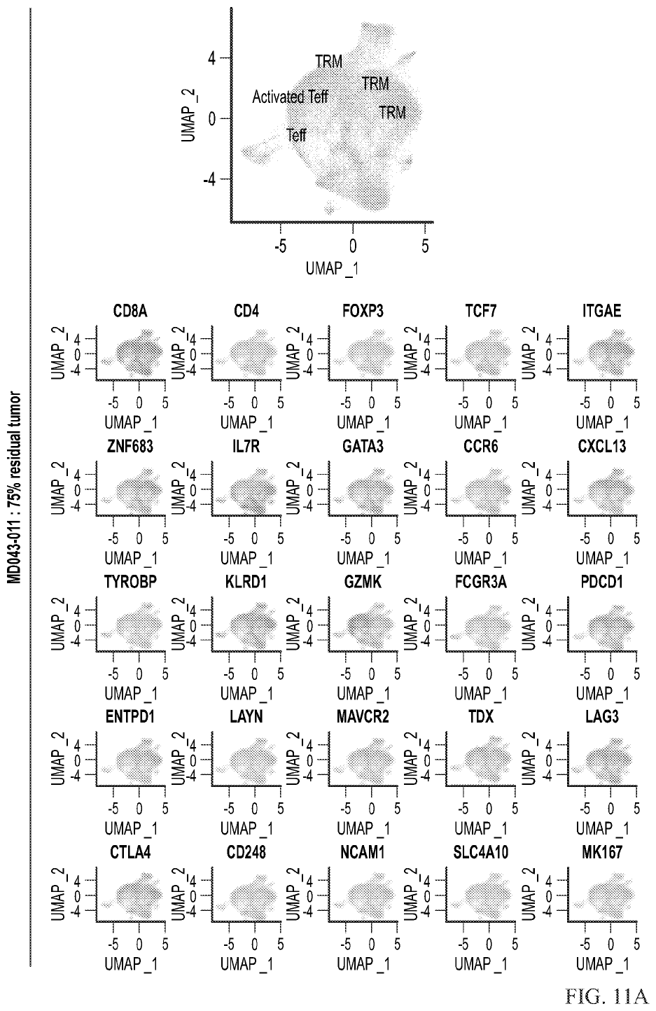

The transcriptional programming of neoantigen- and viral-specific CD8⁺ T cells was next evaluated. To do this, a more refined clustering of all CD8⁺ T cells (n=235,851) was performed and 15 unique clusters were identified, 3 of which with gene expression programs consistent with T_{eff} cells and 2 additional clusters co-expressing CD4 and CD8 and 6 with gene expression programs associated with TRM T cells, characterized by HOBIT expression, LINCO2246 expression, and high CD103 expression (FIG. 2C and FIG. 10). Selective genes were visualized by UMAP (FIG. 10). Among all patients tested, a total of 28 MANA-specific clonotypes (1,350 total cells from 3 MPR and 3 non-MPR) were found in the CD8 single cell analysis; 21 of these (890 cells) were in the tumor (Table 8 (FIG. 14)). Of the viral-specific T cell clonotypes, 28 flu-specific (1,009 cells) and 2 EBV-specific (281 cells) clones were found in the CD8 single cell analysis.

Overlay of these clonotypes onto the CD8⁺ T cell UMAP demonstrated a striking distinction between the clonotypes with different antigen specificities. EBV-reactive T cells primarily resided in T_{eff} clusters, whereas flu- and MANA-specific T cells largely occupied distinct TRM clusters. This is notable considering that influenza is a respiratory virus and thus, flu-specific T cells are the quintessential lung-resident memory T cells. None of the patients in this study were symptomatic for influenza in the 6 weeks preceding surgery. It is thus not surprising that flu-specific CD8 cells were TRM rather than T_{eff}. While flu-specific cells were most numerous in normal lung, MANA-specific CD8 cells were more common in the tumor (FIG. 2E), likely owing to their exposure to more tumor antigen in the tumor microenvironment than in normal lung. Indeed, there were significantly more MANA-specific CD8 cells among the proliferating subset of TIL than in adjacent NL.

Surprisingly there were significant shared gene expression programs between MANA- and EBV-specific T cells, in particular genes encoding T cell activation and CTL activity, such as HLA-DR, GZMH, and NKG7 (FIGS. 2F and 2G). However, genes encoding other cytolytic granule molecules, such as GZMK, were almost absent in MANA-specific TIL. Also, transcription factors critical to CTL activity, Eomes and TBX21 (Tbet), were present in EBV-specific CD8 cells but virtually absent in most of the MANA-specific cells. These findings demonstrate that MANA-specific T cells in the tumor have a partial but incomplete effector program, possibly down-modulated among MANA-specific CD8 cells by higher levels of checkpoint molecules, such as PD-1, CTLA-4, HAVCR2 (Tim3), LAG3, TIGIT, and ENTPD1 (CD39). In fact, each of these checkpoints was more highly expressed among MANA-specific CD8 cells than either flu- or EBV-specific CD8 cells, with CD39 being the most highly differentially expressed (FIG. 2G). MANA-specific cells express higher levels of PDRM1, which encodes Blimp-1 and has been reported to participate in coordinated transcriptional activation of multiple of these checkpoint genes, including PD-1, LAG3, TIGIT and HAVCR2. Tox, a chromatin modifier important for exhaustion/anergy programs of chronic virus-specific and tumor-specific T cells, was only marginally increased in MANA-specific cells but its homolog, Tox2, which has also been reported to drive T cell anergy/exhaustion, showed much greater differential expression between MANA-specific and EBV-specific CD8 cells. ZNF683 (HOBIT), whose expression must be turned off in order for TRM to differentiate to T_{eff} upon antigen encounter, was also upregulated in MANA-specific TIL, even relative to flu-specific TRM. Additionally, flu-specific TRM were distinguished from MANA-specific TRM by extremely low levels of both activation (including MHC II) and effector CTL programs and multiple checkpoint molecules such as ENTPD1, TNFRSF9, and CTLA-4, but had the highest levels of genes encoding stem/memory molecules, such as TCF7 and IL7R (FIG. 2H). Neither of these molecules are expressed at significant levels in MANA-specific T cells (FIGS. 2F and 2G) and represent a significant element of the differential gene expression that separates flu- and MANA-specific T cells into distinct TRM clusters. Culture with titrating concentrations of IL7 in vitro induce much higher levels of IL7R-regulated genes in flu-specific TIL relative to MANA-specific TIL (FIG. 21).

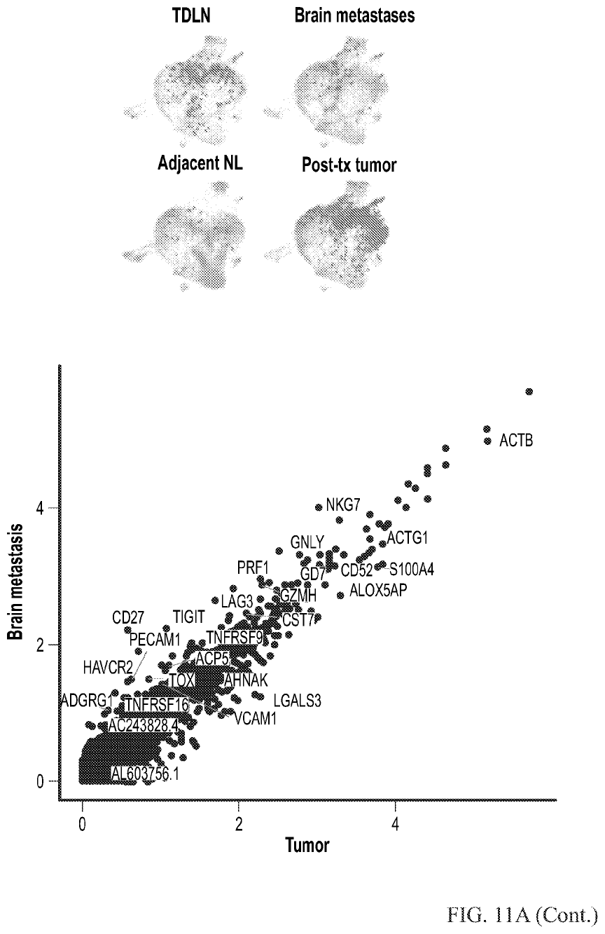

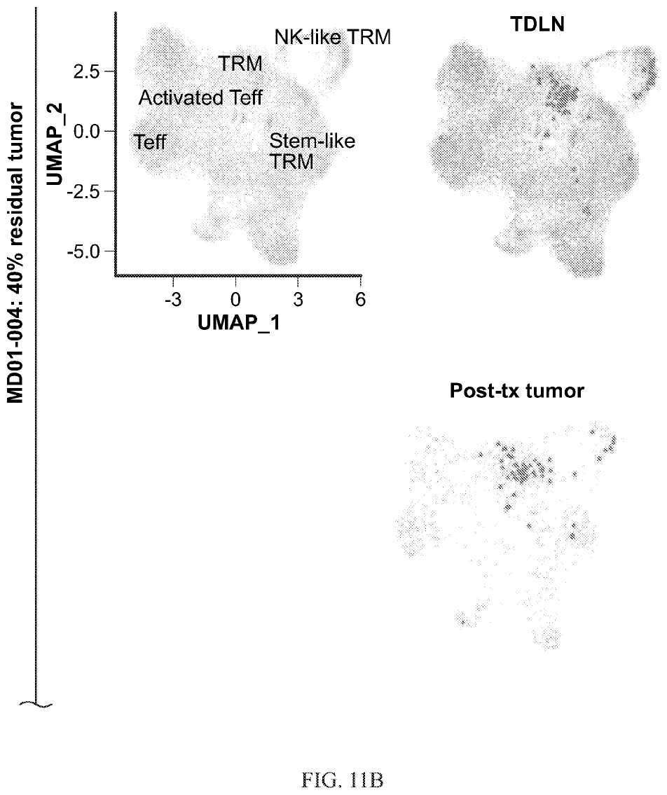

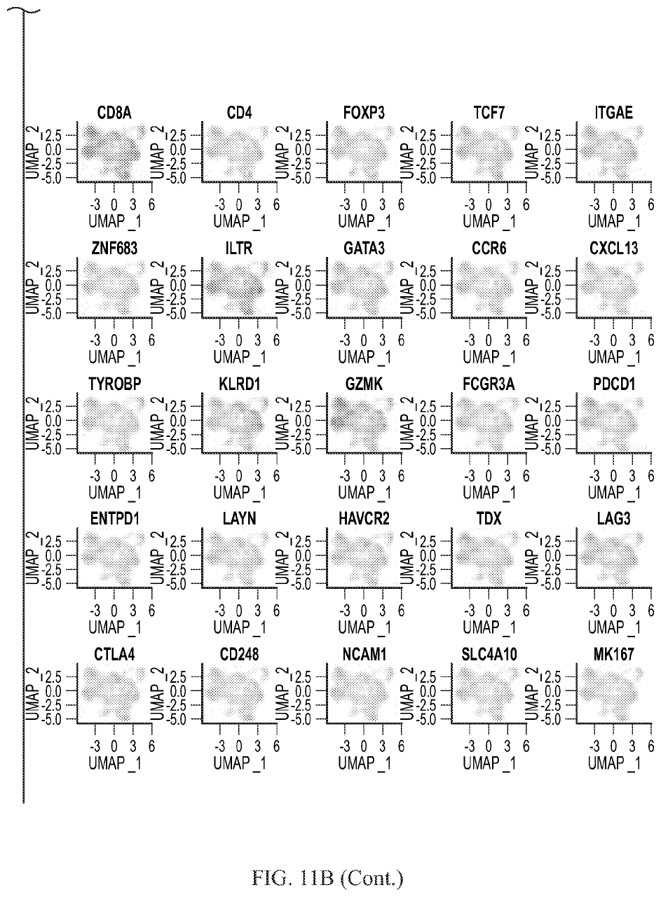

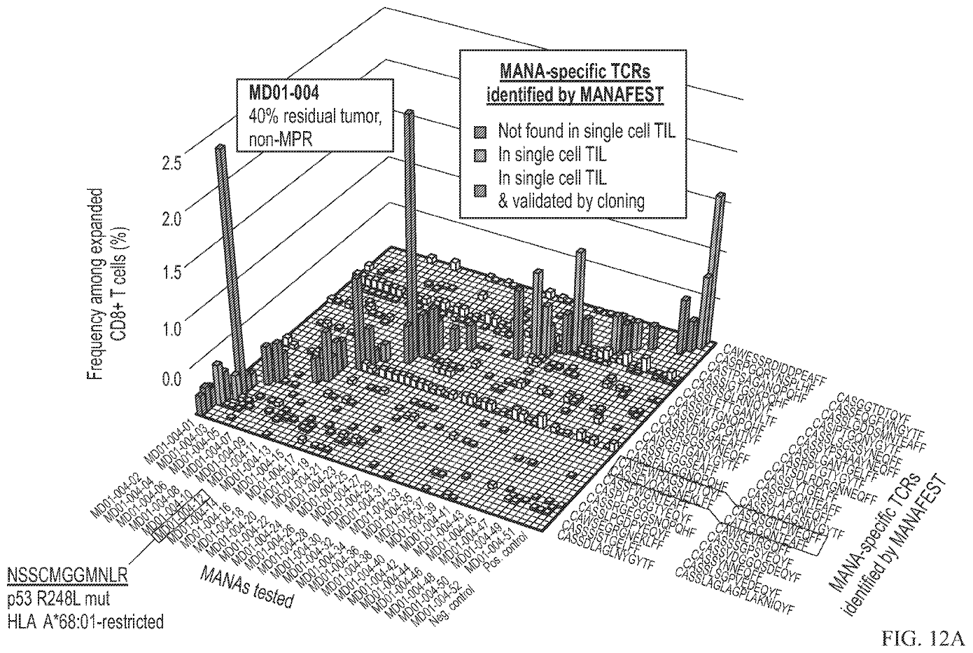

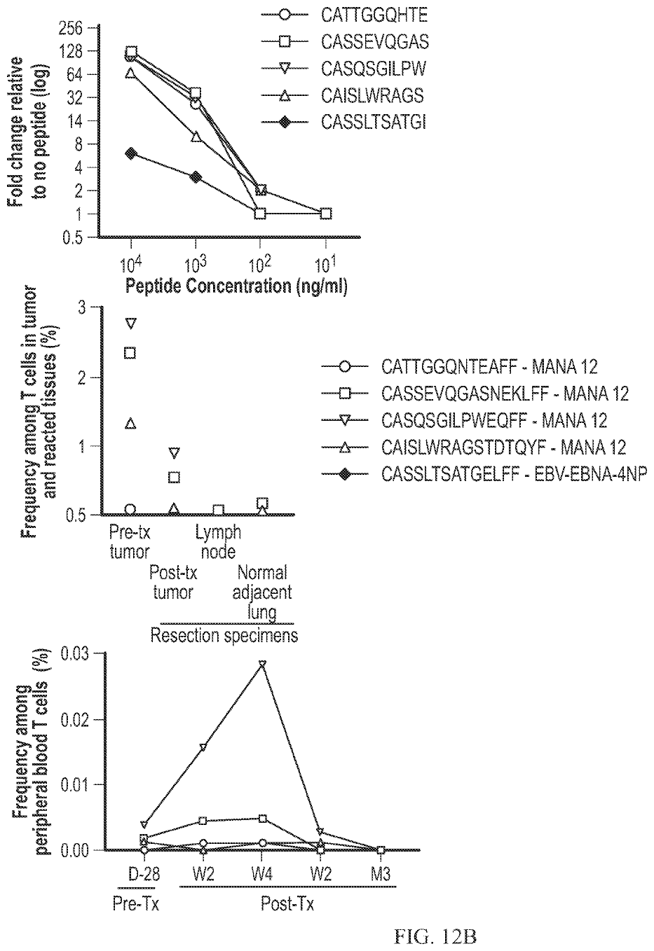

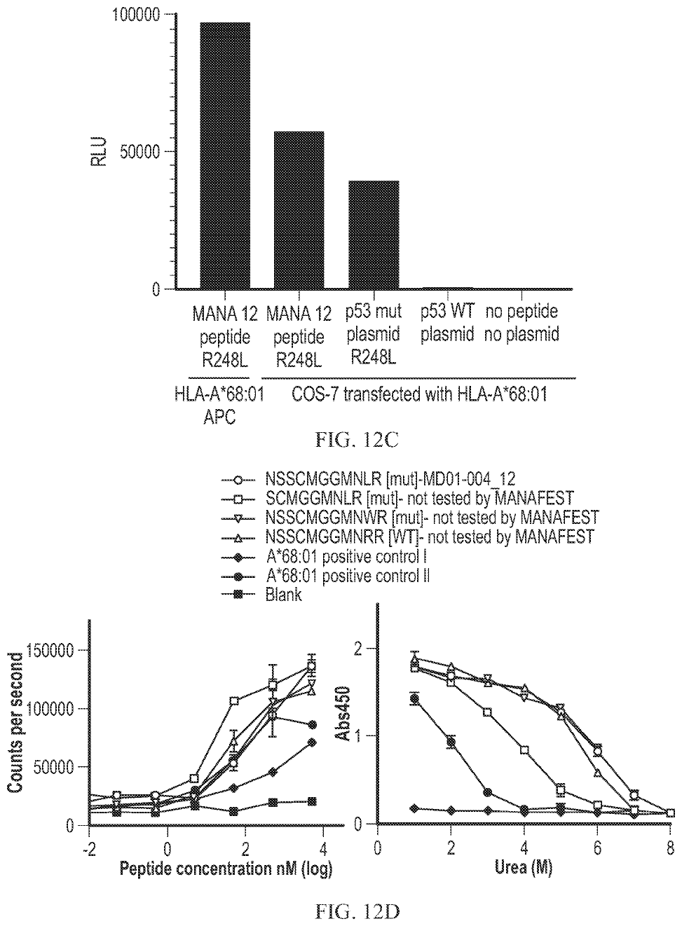

Critical to the understanding of ICB sensitivity vs resistance is the expression profiling of MPR vs non-MPR CD8 TIL. The neoadjuvant clinical trial format allowed us to make this distinction pathologically, which has been reported to be more sensitive than classical radiologic assessment, which has been reported to underestimate therapeutically relevant responses. Profiling of MANA-specific CD8+ T cells demonstrated significant differences between pathologic MPR vs. non-MPR tumors (FIG. 3). Unsupervised clustering of 6 clones (26 transcriptomes total) of MANA-specific TIL from 3 MPR patients vs 15 clones (864 transcriptomes total) of those from 3 non-MPR demonstrated 100% segregation of MPR transcriptomes from non-MPR transcriptomes (FIG. 3A). IL7R is higher in MPR than non-MPR CD8 MANA-specific clones (FIG. 3B). A composite checkpoint score was created consisting of 6 of the best characterized (and clinically targeted) checkpoints: CTLA4, PDCD1, LAG3, HAVCR2, TIGIT and ENTPD1. The checkpoint score was significantly higher in non-MPR MANA-specific TIL than MPR MANA-specific TIL (FIG. 3C). In non-MPR MANA-specific TIL, all six checkpoints comprising the checkpoint score are within the top 12 genes correlated with the checkpoint score (FIGS. 3D and 3E), as compared to only one—ENTPD1—in the top 30 associated genes in MPR TIL. This finding emphasizes the strong coordinate up-regulation of checkpoints in non-MPR. In one non-MPR, MANA-specific cells were identified upon single cell profiling of CD8 TIL from a resected brain metastasis arising 24 months after the resection of the primary tumor (FIG. 11A). Relative to the primary tumor, even higher levels of three checkpoints—LAG3, TIGIT and HAVCR2—were expressed on MANA-specific CD8 TIL from the metastasis and shared with TIL from the primary tumor (FIG. 11A). CXCL13 is the most highly expressed checkpoint-associated gene in non-MPR MANA-specific TIL, as was also found to be highly expressed in MANA-specific cells relative to virus-specific cells among CD8 TIL (FIGS. 2F and 2H). While CXCL13 is known as a Tfh chemokine that attracts B cells to follicles, it is also part of the genetic program in chronic virus-induced CD8 exhaustion, though its role in this process is unknown.

A number of genes encoding T cell inhibitory molecules are also more highly expressed among MANA-specific TIL from non-MPR vs MPR (FIG. 3E). These include the killer inhibitory receptor, KIR2DL4, and a subunit of the HLA-E binding receptor—KLRD1 (CD94)—which has been shown to inhibit CD8 activity. While these inhibitory receptors are well studied in NK cells, they also inhibit CD8 T cell activity. Additional inhibitors of T cell activation highly up-regulated in non-MPR were DTX1 (Deltex1), AKAP5, LAYN (Laylin), and ADGRG1. DTX1 has been shown to be an NFAT target that, together with EGR2, drives T cell anergy. AKAPs direct protein kinase A (PKA) to subcellular locations. In T cells, PKA inhibits T cell activation via C-terminal Src kinase (Csk) activation, which in turn inhibits lymphocyte-specific protein tyrosine kinase (Lck) activity through phosphorylation at Y505. Laylin is a membrane protein on T_{reg} and CD8 T cells that appears to inhibit CTL activation, though by as yet unknown mechanisms. ADGRG1 is a HOBIT-induced gene that has been shown to inhibit NK cell activity but has not been previously reported in CD8 T cells.

In pathologic complete responder MD01-005 (no viable tumor in the resection specimen), MANA-specific T cell transcriptional programming in tumor, adjacent NL, TDLN, and peripheral blood was able to be characterized. All MANA-specific clones in the tumor fell into TRM clusters, whereas a significant proportion of these were in T_{eff} clusters in the TDLN and adjacent NL (FIG. 3F, top). This clone was enriched via FACS sorting of peripheral blood at different time points after initiation of anti-PD-1 (by sorting with Mab specific for its Vβ) and found an intriguing pattern. Two weeks after initiation of anti-PD-1, almost all MANA-specific T cells in the peripheral blood fell into a tight TRM cluster, while at 4 weeks (time of surgery) a third of these cells were in T_{eff} clusters. By 3 months after resection they were below limits of detection in the blood (FIG. 3F, bottom). While all these tissue compartments were only available for one MPR, these findings are consistent with our hypothesis that successful neoadjuvant PD-1 blockade results in enhanced activation of MANA-specific T cells in TDLN. Without being bound by theory, it's hypothesized that, upon activation, functional effector MANA-specific T cells enter the blood and traffic into tissues, including normal lung, in search of micro-metastatic tumor. Indeed, analysis of TDLN from two non-MPR patients failed to demonstrate any MANA-specific CD8 cells in a T_{eff} population (FIGS. 11A and 11B).

Overall, it was found that global T cell gene expression programs are poorly associated with pathologic response to PD-1 blockade. However, transcriptomic analysis of validated MANA-specific TIL demonstrated clear differences associated with response, with TIL from non-responding tumors displaying higher levels of checkpoints and additional inhibitory molecules such as Deltex1, APK5, and ADGRG1, and multiple killer inhibitory receptors.

Thus, together these results demonstrate that T cell targeting of MANA can be used to improve the outcome of ICB and can overcome resistance to ICB.

### Example 2: Identification of MANAbody Clones Specific for a P53 R248L Neoantgien

TP53 is the most commonly mutated cancer driver gene, but despite extensive efforts, no drug targeting mutant TP53 has been approved for treatment of the large number of patients whose tumor contain p53 mutations.

This Example describes the identification of antibodies highly specific to a R248L TP53 mutation.

The MANAFEST (Mutation Associated NeoAntigen Functional Expansion of Specific T-cells) assay was used to evaluate T cell responsiveness to MANA and viral antigens. Briefly, pools of MHC class I-restricted CMV, EBV, and flu peptide epitopes (CEFX, jpt Peptide Technologies), pools representing the matrix protein and nucleoprotein from H1N1 and H3N2 (jpt Peptide Technologies), and putative neoantigenic peptides defined by the ImmunoSelect-R pipeline were used to stimulate 250,000 T cells in vitro for 10 days as previously described. T cells were also cultured without peptide to use as a reference for non-specific clonotypic expansion. On day 10, T cell receptor sequencing was performed on each individual peptide-stimulated T cell culture by the Sidney Kimmel Comprehensive Cancer Center FEST and TCR Immunogenomics Core (FTIC) facility or Adaptive Biotechnologies. Bioinformatic analysis of productive clones was performed to identify antigen-specific T-cell clonotypes meeting the following criteria: 1) significant expansion (Fisher's exact test with Benjamini-Hochberg correction for FDR, p<0.05) compared to T cells cultured without peptide, 2) significant expansion compared to every other peptide-stimulated culture (FDR<0.0001) except for conditions stimulated with similar neoantigens derived from the same mutation, 3) an odds ratio >5 compared to the “no peptide” control, and 4) present in at least 10% of the cultured wells to ensure adequate distribution among culture wells.

### Example 3: Treating ICB Resistant Cancers

T cells expressing one or more TCRs that can bind to a p53 R248L peptide are administered to a human having an ICB resistant cancer. The administered T cells can infiltrate the tumor microenvironment to target (e.g., target and destroy) cancer cells expressing the p53 R248L peptide.

### Example 4: ICB Resistant Cancers

Nuclei acid that encode a TCR that can bind to a p53 R248L peptide is introduced into T cells such that the T cells encode the TCR and the TCR is presented on the surface of the T cells.

The T cells expressing the TCR that can bind to a p53 R248L peptide are administered to a human having an ICB resistant cancer. The administered T cells can infiltrate the tumor microenvironment to target (e.g., target and destroy) cancer cells expressing the p53 R248L peptide.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A T cell receptor (TCR) that can bind to a modified p53 polypeptide comprising a R to L substitution at amino acid residue 248 (R248L).

2. The TCR of claim 1, wherein said modified p53 polypeptide comprises a p53 R248L peptide comprising or consisting essentially of the amino acid sequence set forth in any one of SEQ ID NOs:1-40.

3. The TCR of claim 1, wherein said TCR comprises an alpha (α) chain comprising a TCRα-CDR3 set forth in any one of SEQ ID NOs:41-44.

4. The TCR of claim 1, wherein said TCR comprises a beta (β) chain comprising a TCRβ-CDR3 set forth in any one of SEQ ID NOs:45-48.

5. The TCR of claim 1, wherein said TCR comprises:
an α chain that includes a TCRα-CDR3 set forth in any one of SEQ ID NOs:41-44, and α βchain that includes a TCRβ-CDR3 set forth in any one of SEQ ID NOs:45-48.

6. A T cell comprising the TRC of claim 1.

7. The T cell of claim 6, wherein said T cell is a human T cell.

8. The T cell of claim 6, wherein said T cell is a non-human T cell.

9. A nucleic acid encoding the TRC of claim 1.

10. The nucleic acid of claim 9, wherein said nucleic acid is in the form of a vector.

11. The nucleic acid of claim 10, wherein said vector is an expression vector.

12. The nucleic acid of claim 10, wherein said vector is a viral vector.

13. A T cell comprising the nucleic acid of claim 9, wherein said nucleic acid encodes said TCR.

14. The T cell of claim 13, wherein said T cell is a human T cell.

15. The T cell of claim 13, wherein said T cell is a non-human T cell.

16. A method for treating a mammal having cancer, said method comprising administering to said mammal the T cell of claim 6, wherein said cancer comprises a cancer cell expressing said modified p53 polypeptide.

17. The method of claim 16, wherein said cancer cell expressing said modified p53 polypeptide presents a p53 R248L peptide in a peptide-HLA complex.

18. The method of claim 17, wherein said p53 R248L peptide comprising or consisting essentially of the amino acid sequence set forth in any one of SEQ ID NOs:1-40.

19. The method of claim 16, wherein said mammal is a human.

20. The method of claim 16, wherein said cancer is selected from the group consisting of a non-small cell lung cancer (NSCLC), a colon adenocarcinoma, a rectal adenocarcinoma, a head and neck squamous cell carcinoma, a pancreatic adenocarcinoma, melanomas, a urothelial carcinoma, a uterine corpus endometrial carcinoma, and a uterine carcinoma.

21. The method of claim 16, wherein said method further comprises administering to said mammal a checkpoint inhibitor.

22. The method of claim 21, wherein said checkpoint inhibitor is selected from the group consisting of an anti-CTLA-4 (cytotoxic T-lymphocyte-associated protein 4) antibody, an anti-PD-1 (programmed death 1) antibody, an anti-PD-L1 (programmed death 1 ligand) antibody, an anti-LAG3 (lymphocyte activation gene 3) antibody, an anti-Tim3 (T cell immunoglobulin and mucin domain-containing protein 3) antibody, an anti-TIGIT (T cell immunoreceptor with Ig and ITIM domains) antibody, an anti-VISTA (V-domain Ig suppressor of T cell activation) antibody, an anti-CD47 (cluster of differentiation 47) antibody, an anti-SIRPalpha (signal regulatory protein alpha) antibody, an anti-B7-H3 (B7 homolog 3) antibody, an anti-B7-H4 (B7 homolog 4) antibody, an anti-neuritin antibody, an anti-neuropilin antibody, an anti-IL-35 (interleukin 35), an IDO (indoleamine-pyrrole 2,3-dioxygenase) inhibitor, an A2AR (adenosine A2A receptor) inhibitor, an arginase inhibitor, and a glutaminase inhibitor.

23. The method of claim 16, wherein said method further comprises administering to said mammal a co-stimulatory molecule.

24. The method of claim 23, wherein said co-stimulator molecule is an agonist of a co-stimulatory receptor.

25. The method of claim 24, wherein said agonist of a co-stimulatory receptor is selected from the group consisting of an anti-GITR (glucocorticoid-induced TNFR-related) antibody, an anti-CD27 (cluster of differentiation 27) antibodies antibody, an anti-4-1BB (CD137; cluster of differentiation 137) antibody, an anti-OX40 (CD134; cluster of differentiation 134) antibody, an anti-ICOS (inducible T-cell costimulator) antibody, and an anti-CD40 (cluster of differentiation 40) antibody.
